(19) United States
(12) Patent Application Publication
(10) Pub. No.: US 2025/0051801 A1
(43) Pub. Date: Feb. 13, 2025

(54) VIRAL PARTICLE WITH SURFACE STIMULATING MOLECULES

(71) Applicant: Umoja Biopharma, Inc., Seattle, WA (US)

(72) Inventors: Christopher NICOLAI, Seattle, WA (US); Jim QIN, Seattle, WA (US); Wai-hang LEUNG, Seattle, WA (US); Ryan LARSON, Seattle, WA (US); Alyssa SHEIH, Seattle, WA (US); Byoung RYU, Seattle, WA (US)

(21) Appl. No.: 18/931,597

(22) Filed: Oct. 30, 2024

Related U.S. Application Data
(63) Continuation of application No. PCT/US2023/066636, filed on May 5, 2023.
(60) Provisional application No. 63/339,332, filed on May 6, 2022.
(60) Provisional application No. 63/348,180, filed on Jun. 2, 2022.
(60) Provisional application No. 63/351,064, filed on Jun. 10, 2022.
(60) Provisional application No. 63/487,734, filed on Mar. 1, 2023.

(51) Int. Cl. C12N 15/86; C07K 14/54; C07K 14/705; C07K 16/28

(52) U.S. Cl. CPC C12N 15/86; C07K 14/54; C07K 14/70528; C07K 14/70532; C07K 16/2809; C07K 2317/622; C12N 2740/15033; C12N 2760/20241

(57) ABSTRACT

Provided are viral particles for activating and transducing immune cells in vitro or in vivo, and compositions and methods for using said viral particles.

### CROSS REFERENCE TO RELATED APPLICATIONS

This is a continuation of PCT/US2023/066636, filed May 5, 2023, which claims priority to and the benefit of U.S. Provisional Application No. 63/339,332, filed May 6, 2022, U.S. Provisional Application No. 63/348,180, filed Jun. 2, 2022, U.S. Provisional Application No. 63/351,064, filed Jun. 10, 2022, and U.S. Provisional Application No. 63/487,734, filed Mar. 1, 2023, the contents of each of which are incorporated herein by reference in their entireties.

### INCORPORATION BY REFERENCE OF SEQUENCE LISTING

This application contains a Sequence Listing which has been submitted digitally in .XML ST26 format and is hereby incorporated by reference in its entirety. Said .XML copy, created on Oct. 25, 2024, is named 061479-505C01US_SeqList.xml, and is 294,817 bytes in size.

### BACKGROUND

Cellular therapy generally employs the transduction of immune cells ex vivo to generate a population of therapeutic cells to be introduced into the patient. For example, T cells from an autologous or allogenic source can be transduced ex vivo with a vector encoding a chimeric antigen receptor. The resulting CAR T-cells are then infused into the patient.

While it would be desirable to generate therapeutic cells in vivo by delivering a vector to the patient, current methodologies for in vivo transduction of immune cells suffer from technical, logistical, consistency, cost, and efficacy challenges. The in vivo approach has not been widely pursued because of the technical challenges associated with it, the main hurdle being the need to activate T cells in the body in order to effectively engineer them as well as the need to “control” expansion of these engineered cells once transduced.

Ex-vivo manufacture of cellular therapy requires a complex series of steps, starting with collection of the patient's peripheral blood mononuclear cells via a leukapheresis procedure, followed by genetic modification of the patient's T cells in a cGMP facility that introduces delays, risks, and complex logistics into patient care. This is followed by the administration of lymphodepleting chemotherapy prior to infusion of the final drug product. Accordingly, there remains a need for improved cellular therapy.

### SUMMARY OF THE INVENTION

The present disclosure is based, at least in part, on the discovery that a viral particle can be manufactured to express cell surface proteins on the viral envelope to simultaneously prime and transduce immune cells. Specifically, as demonstrated herein, viral particles expressing a TCR targeting molecule and a co-stimulatory molecule, enhances cell activation and transduction of a nucleotide encoding a polypeptide of interest in vivo compared to viral particles only expressing a TCR targeting molecule. Further, it has been shown including an adhesion molecule further enhances cell activation and transduction in vivo. Without wishing to be bound by theory, viral particles engineered to express with a TCR targeting molecule (e.g., CD3 binding protein) and a co-stimulatory molecule (e.g., CD80 or CD86) activate signal one and signal two necessary for T cell activation. Further, it is believed an adhesion molecule stabilizes the interaction between the immune cell and the viral particle thereby recreating an immunological synapse to allow for sufficient cell activation and transduction of a nucleotide. The disclosure also shows in vivo activation and expansion of non-transduced T cells. Without wishing to be bound by theory, the particles described herein are capable of driving activation and expansion of tumor infiltrating lymphocytes and tumor reactive T cells present in tumor draining or metastatic lymph nodes, indicating such particles may be effective at low doses.

Accordingly, in some aspects, the present disclosure provides a viral particle comprising a viral envelope comprising on the surface of the viral envelope at least one T-cell adhesion molecule, at least one co-stimulatory protein, or combination thereof, and an immune cell-activating protein. In some embodiments, the T-cell adhesion molecule, co-stimulatory protein, and immune-cell activating protein are each recombinant proteins.

In some or any of the foregoing or related aspects, the at least one T-cell adhesion molecule is selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof. In some aspects, the at least one T cell-adhesion molecule is CD58.

In some or any of the foregoing or related aspects, the at least one co-stimulatory molecule is selected from CD45, CD2, CD4, CD5, CD8, CD9, CD16, CD22, CD33, CD28, CD37, CD64, CD80, CD86, CD134, CD137, CD154, OX40, 4-1BB, CD40L, and any combination thereof. In some aspects, the at least one co-stimulatory molecule is CD80, CD86, or CD80 and CD86.

In some or any of the foregoing or related aspects, the immune cell-activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, TCR α chain, TCR β chain, TCR ζ chain, TCR γ chain, TCR δ chain, CD3 ε TCR subunit, CD3 γ TCR subunit, CD3 δ TCR subunit, or NKp80. In some aspects, the immune cell-activating protein is a protein that specifically binds CD3.

In some or any of the foregoing or related aspects, the immune cell-activating protein is an antibody or antigen binding fragment thereof that binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80. In some aspects, the immune cell-activating protein is an antibody or antigen binding fragment thereof that binds CD3. In some aspects, the antibody or antigen binding fragment thereof that binds CD3 is an anti-CD3 scFv.

In some or any of the foregoing or related aspects, the T-cell adhesion molecule is CD58 and the co-stimulatory molecule is CD80. In other aspects, the T-cell adhesion molecule is CD58 and the co-stimulatory molecule is CD86.

In some or any of the foregoing or related aspects, the T-cell adhesion molecule is CD58, the immune cell-activating protein is an anti-CD3 antibody or antigen binding fragment thereof, and the co-stimulatory molecule is CD80. In other aspects, the T-cell adhesion molecule is CD58, the immune cell-activating protein is an anti-CD3 antibody or antigen binding fragment thereof, and the co-stimulatory molecule is CD86.

In some or any of the foregoing or related aspects, the viral particle comprises a payload. In some aspects, the payload is a nucleic acid. In some aspects, the nucleic acid is a non-coding nucleic acid, optionally wherein the non-coding nucleic acid is an siRNA, an miRNA, or an shRNA. In some aspects, the nucleic acid comprises a nucleotide sequence encoding a polypeptide of interest.

In some or any of the foregoing or related aspects, the viral particle comprises a vector genome comprising at least one nucleotide sequence encoding a polypeptide of interest.

In some aspects, the disclosure provides a viral particle comprising (i) a viral envelope comprising on the surface of the viral envelope (a) an immune cell-activating protein, wherein the immune cell-activating protein binds a T cell receptor, (b) a co-stimulatory molecule, and (c) a T cell adhesion molecule, and (ii) a vector genome comprising at least one nucleotide sequence encoding a polypeptide of interest. In some aspects, (a) the immune cell-activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80, (b) the co-stimulatory molecule is selected from CD45, CD2, CD4, CD5, CD8, CD9, CD16, CD22, CD33, CD28, CD37, CD64, CD80, CD86, CD134, CD137, CD154, OX40, 4-1BB, CD40L, and any combination thereof, and (c) the T cell adhesion molecule is selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof. In some aspects, (a) the immune cell-activating protein is an antibody that specifically binds CD3, or an antigen binding fragment thereof, (b) the co-stimulatory molecule is CD80 or CD86, and (c) the T cell adhesion molecule is CD58.

In some or any of the foregoing or related aspects, the viral envelope comprises a membrane-bound cytokine. In some aspects, the membrane-bound cytokine is selected from IL-2, IL-7, IL-12, IL-15, IL-18, or IL-21.

In some or any of the foregoing or related aspects, the viral envelope comprises a viral envelope protein. In some aspects, the viral envelope protein is a VSV-G envelope protein, a measles virus envelope protein, a nipha virus envelope protein, or a cocal virus G protein. In some aspects, the viral envelope comprises a Cocal glycoprotein or functional variant thereof. In some aspects, the Cocal glycoprotein comprises an R354Q mutation compared to SEQ ID NO: 5. In some aspects, the Cocal glycoprotein comprises a K47Q mutation compared to SEQ ID NO: 5. In some aspects, the Cocal glycoprotein comprises an amino acid sequence at least 80%, at least 85%, at least 90%, at least 95%, at least 96%, at least 97%, at least 98%, or at least 99% identical to any one of SEQ ID NOs: 5, 13, and 19. In some aspects, the Cocal glycoprotein comprises an amino acid sequence selected from SEQ ID NOs: 5, 13, and 19.

In some or any of the foregoing or related aspects, the antibody that binds anti-CD3 or antigen binding fragment thereof comprises an amino acid sequence at least 80%, at least 85%, at least 90%, at least 95%, at least 96%, at least 97%, at least 98%, or at least 99% identical to SEQ ID NO: 2 or 12. In some aspects, the antibody that binds anti-CD3 or antigen binding fragment thereof comprises SEQ ID NO: 2 or SEQ ID NO: 12.

In some or any of the foregoing or related aspects, the viral particle comprises a nucleotide sequence encoding a multipartite cell-surface receptor. In some aspects, the multipartite cell-surface receptor comprises a FKBP-rapamycin complex binding domain (FRB domain) and a FK506 binding protein domain (FKBP). In some aspects, the multipartite cell-surface receptor is a rapamycin-activated cell-surface receptor.

In some or any of the foregoing or related aspects, the viral particle comprises a nucleotide sequence encoding a chimeric antigen receptor (CAR). In some aspects, the viral particle comprises a nucleotide sequence encoding a rapamycin activated cell-surface receptor and a nucleotide sequence encoding a CAR. In some aspects, the viral particle comprises a vector genome comprising from 5′ to 3′: a nucleotide sequence encoding a CAR and a nucleotide sequence encoding a multipartite cell-surface receptor. In some aspects, the nucleotide sequences are operably linked. In some aspects, the CAR comprises an antigen binding domain specific for a cancer-associated antigen, and wherein the multipartite cell-surface receptor is a rapamycin-activated cell-surface receptor.

In some or any of the foregoing or related aspects, the CAR comprises an antigen binding domain specific for a cancer-associated antigen. In some aspects, the cancer associated antigen is CD19, BCMA, GPRC5D, ROR1, FcRL5/FcRH5, alpha-fetoprotein, or Her2. In other aspects, the CAR is a universal CAR. In some aspects, the CAR comprises a hapten binding domain.

In some aspects, the disclosure provides a viral particle comprising (i) a viral envelope comprising on the surface of the viral envelope (a) an immune cell-activating protein that specifically binds CD3, (b) a co-stimulatory molecule, wherein the co-stimulatory molecule binds CD28, and (c) a T cell adhesion molecule, and (ii) a vector genome comprising (a) a nucleotide sequence encoding a rapamycin-activated cell-surface receptor, and (b) a nucleotide sequence encoding a CAR, wherein the CAR comprises an antigen binding domain specific for a cancer-associated antigen, optionally wherein the nucleotide sequences are operably linked.

In some or any of the foregoing or related aspects, CD58 comprises an amino acid sequence at least 80%, at least 85%, at least 90%, at least 95%, at least 96%, at least 97%, at least 98%, or at least 99% identical to SEQ ID NO: 17. In some aspects, CD58 comprises the amino acid sequence of SEQ ID NO: 17.

In some or any of the foregoing or related aspects, CD80 comprises an amino acid sequence at least 80%, at least 85%, at least 90%, at least 95%, at least 96%, at least 97%, at least 98%, or at least 99% identical to SEQ ID NO: 20. In some aspects, CD80 comprises the amino acid sequence of SEQ ID NO: 20.

In some or any of the foregoing or related aspects, CD86 comprises an amino acid sequence at least 80%, at least 85%, at least 90%, at least 95%, at least 96%, at least 97%, at least 98%, or at least 99% identical to SEQ ID NO: 23. In some aspects, CD86 comprises the amino acid sequence of SEQ ID NO: 23.

In some or any of the foregoing or related aspects, the multipartite cell-surface receptor comprises an amino acid sequence at least 80%, at least 85%, at least 90%, at least 95%, at least 96%, at least 97%, at least 98%, or at least 99% identical to SEQ ID NOs: 77, 78, or 77 and 78. In some aspects, the multipartite cell-surface receptor comprises the amino acid sequence of SEQ ID NOs: 77, 78, or 77 and 78. In some aspects, the multipartite cell-surface receptor is encoded by a nucleotide sequence at least 80%, at least 85%, at least 90%, at least 95%, at least 96%, at least 97%, at least 98%, or at least 99% identical to SEQ ID NOs: 83, 84, or 83 and 84. In some aspects, the multipartite cell-surface receptor is encoded by the nucleotide sequence of SEQ ID NOs: 83, 84, or 83 and 84.

In some or any of the foregoing or related aspects, the vector genome comprises a promoter. In some aspects, the promoter is an MND promoter, a CAG promoter, an SV40 promoter, an SV40/CD43 promoter, or an EF-1α promoter. In some aspects, the promoter is an inducible promoter.

In some or any of the foregoing or related aspects, the viral particle is a lentiviral particle.

In some or any of the foregoing or related aspects, the viral particle transduces T cells in vivo. In other aspects, the viral particle transduces T cells ex vivo. In some aspects, the viral particle activates a T cell population comprising at least a 50% CD25(+) cells, at least a 70% CD25(+) cells, or at least 90% CD25(+) cells.

In some aspects, the disclosure provides a pharmaceutical composition comprising a viral particle described herein, and a pharmaceutically acceptable carrier.

In some aspects, the disclosure provides a method of transducing a population of T cells in vivo in a subject, comprising administering to the subject a viral particle or pharmaceutical composition thereof, wherein the viral particle comprises a nucleotide sequence encoding a polypeptide of interest, wherein the polypeptide of interest is expressed in the population of T cells after administration. In some aspects, the population of T cells secretes (i) at least 2×10⁴ pg/ml of TNFα, (ii) at least 2×10⁴ pg/ml of IL-2, (iii) at least 2×10⁵ pg/ml of IFNγ, or (iv) any combination of (i)-(iii), at least three days after administration of the lentiviral particle.

In other aspects, the disclosure provides a method of generating an immune cell expressing a chimeric antigen receptor in a subject in need thereof, comprising administering a viral particle or pharmaceutical composition described herein to the subject, wherein the viral particle comprises a nucleotide sequence encoding the chimeric antigen receptor.

In further aspects, the disclosure provides a method of treating a disease or disorder in a subject in need thereof, comprising administering a viral particle or pharmaceutical composition described herein to the subject, wherein the viral particle comprises a nucleotide sequence encoding a therapeutic polypeptide.

In some or any of the foregoing or related aspects, the viral particle is administered by intraperitoneal, subcutaneous, or intranodal injection. In some aspects, the viral particle is administered by intra-nodal injection, via inguinal lymph node.

In some or any of the foregoing or related aspects, the subject in need thereof has a disease or disorder, wherein the disease or disorder comprises B-cell malignancy, relapsed/refractory CD19-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

In some aspects, the disclosure provides a kit comprising a container comprising a viral particle described herein, and optionally a pharmaceutically acceptable carrier, and instructions for transducing T cells in vivo in a subject, comprising administering the viral particle to the subject. In some aspects, the subject has a disease or disorder. In some aspects, the instructions comprise administering the viral particle by intraperitoneal, subcutaneous, or intranodal injection.

In other aspects, the disclosure provides a kit comprising a container comprising a viral particle described herein, and optionally a pharmaceutically acceptable carrier, and instructions for treating a subject in need thereof, comprising administering the viral particle to subject. In some aspects, the subject has a disease or disorder. In some aspects, the instructions comprise administering the viral particle by intraperitoneal, subcutaneous, or intranodal injection.

In some aspects, the disclosure provides a viral particle described herein for use in a method of transducing T cells in vivo in a subject, comprising administering the viral particle to the subject. In other aspects, the disclosure provides a viral particle described herein for use in a method of treating a subject with a disease or a disorder, comprising administering the viral particle to the subject.

In some aspects, the disclosure provides use a viral particle described herein for the manufacture of a medicament for transducing T cells in vivo in a subject, comprising administering the viral particle to the subject. In other aspects, the disclosure provides use of a viral particle described herein for the manufacture of a medicament for treating a subject with a disease or a disorder, comprising administering the viral particle to the subject.

In some aspects, the disclosure provides a method of transducing a population of T cells ex vivo in a subject, comprising contacting a population of T cells with a viral particle or pharmaceutical composition thereof, wherein the viral particle comprises a nucleotide sequence encoding a polypeptide of interest, and wherein the polypeptide of interest is expressed in the population of T cells after administration, wherein the contacting is performed ex vivo. In some aspects, the contacting is performed during a closed-loop manufacturing process. In some aspects, the T cells have not been previously contacted with an exogenous activation agent during the manufacturing process.

### BRIEF DESCRIPTION OF THE DRAWINGS

FIG. 1 is a schematic that shows T cell activation using an exemplary viral particle with an immune cell-activating protein (e.g., CD3scFv), a viral envelope protein (e.g., Cocal), and one or two costimulatory molecules.

FIG. 2A shows activation of CD8+ T cells as measured by % CD25+ cells with a viral particle displaying CD3scfv or CD3scfv+CD80.

FIG. 2B shows activation of CD8+ T cells as measured by % CD25+ cells with a viral particle displaying CD3scfv only, CD3scfv+CD80 or CD3scfv+CD58.

FIGS. 2C-2D show the level of CAR expression in CD8+ T cells as determined by % CAR expression (FIG. 2C) or total CAR+ CD8+ T cells (FIG. 2D) generated using viral particles with CD3scfv only or CD3scfv+CD80.

FIGS. 2E-2F show the level of CAR expression in CD3+ T cells as determined by % CAR expression (FIG. 2E) or total CAR+CD3+ T cells (FIG. 2F) generated using viral particles with CD3scfv only, CD3scfv+CD80 or CD3scfv+CD58.

FIGS. 2G-2H show fold expansion of CAR+ CD8+ T cells generated with viral particles with CD3scfv only or CD3scfv+CD80 stimulated with IL-2 (FIG. 2G) or rapamycin (FIG. 2H).

FIG. 3A shows the percent CD25(+) CD8 T cells after incubation with a viral particle displaying CD3scfv only, CD3scfv+CD80, CD3scfv+CD58, or CD3scfv+CD80+CD58.

FIG. 3B shows the geometric mean fluorescent intensity (gMFI) of CD25(+) CD8 T cells after incubation with a viral particle displaying CD3scfv only, CD3scfv+CD80, CD3scfv+CD58, or CD3scfv+CD80+CD58.

FIGS. 3C-3E show production of cytokines 3 days after incubation with particles displaying CD3scfv only, CD3scfv+CD80, CD3scfv+CD58, or CD3scfv+CD80+CD58. IFN-γ (FIG. 3C), IL-2 (FIG. 3D), and TNF-α (FIG. 3E) levels were measured.

FIGS. 3F-3G show CAR expression in CD3+ T cells generated with viral particles displaying CD3scfv only, CD3scfv+CD80, CD3scfv+CD58, or CD3scfv+CD80+CD58 (mixed particles). % CAR expression (FIG. 3F) and total CAR+ T cells (FIG. 3G) was measured.

FIGS. 3H-3I show CAR expression in CD8+ T cells generated with viral particles displaying CD3scfv only, CD3scfv+CD80, CD3scfv+CD58, or CD3scfv+CD80+CD58 (same particle). % CAR expression (FIG. 3H) and total CAR+ T cells (FIG. 3I) was measured.

FIGS. 3J-3L show staining of Cocal (FIG. 3J), CD80 (FIG. 3K) or CD58 (FIG. 3L) on CD8+ T cells incubated with viral particles displaying CD3scfv only, CD3scfv+CD80, CD3scfv+CD58, or CD3scfv+CD80+CD58.

FIG. 3M shows a principal components analysis with 3 main clusters of differentiation based on particle costimulatory molecule makeup using CCR7, CD45RO, CD45RA, CD27, CD25, CAR+, total cell, CD4, and CD8 markers.

FIG. 3N shows CD3scfv+CD80 particles generate CAR+ T cells with a predominantly central memory (T_{cm}) phenotype compared to CD3scfv only, which produced effector T cells (T_{eff}).

FIG. 3O shows CD3scfv+CD80, CD3scfv+CD58, or CD3scfv+CD80+CD58 particles generate CAR+ T cells with a predominantly central memory (T_{cm}) phenotype compared to CD3scfv only, which produced effector T cells (T_{eff}) central memory T cells (T_{cm}).

FIG. 4A shows the number of K562.CD19 cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. The particles were added to PBMCs at an MOI of 10 along with Tumor cells at PBMC:Tumor ratio of 5:1 and put directly on the incucyte. CD3scfv+CD80+CD58 CAR T cells were generated using a mixture of individual particles.

FIG. 4B shows the number of Raji cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. The particles were added to PBMCs at an MOI of 10 along with Tumor cells at PBMC:Tumor ratio of 5:1 and put directly on the incucyte. CD3scfv+CD80+CD58 CAR T cells were generated using a mixture of individual particles.

FIG. 4C shows the number of K562.CD19 cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with either K562.CD19 at E:T ratios of 0.5 and 1, respectively. CD3scfv+CD80+CD58 CAR T cells were generated using a mixture of individual particles.

FIG. 4D shows the number of Raji cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with either Raji cells at E:T ratios of 0.5 and 1, respectively. CD3scfv+CD80+CD58 CAR T cells were generated using a mixture of individual particles.

FIG. 4E shows the number of K562.CD19 cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with K562.CD19 cells at E:T ratios of 1:1, respectively. CD3scfv+CD80+CD58 CAR T cells were generated using a single particle with both costimulatory and adhesion molecules.

FIG. 4F shows the number of Nalm6 cells over several days after incubation with anti-CD19 CAR+ T cells generated with viral particles encoding an anti-CD19 CAR and displaying CD3scfv only, CD3scfv+CD80, CD3scfv+CD58, or CD3scfv+CD80+CD58 particles. 7 days after transduction at an MOI of 10, the total CAR+ cells were calculated and incubated with Nalm6 cells at E:T ratios of 1:1, respectively. CD3scfv+CD80+CD58 CAR T cells were generated using a single particle with both costimulatory and adhesion molecules.

FIG. 5A shows the number of CAR T cells in blood samples of NSG MHCI/II KO mice 11 days after injection of PMBCs and viral particles displaying CD3scfv only or CD3scfv+CD80 particles.

FIGS. 5B-5C show the tumor burden in NSG MHCI/II KO mice over 100 days after administration with viral particles displaying CD3scfv only (FIG. 5B) or CD3scfv+CD80 (FIG. 5C).

FIGS. 6A-6B show number of cells expressing a CAR 3 days (FIG. 6A) or 7 days (FIG. 7B) after transduction of PBMCs from 3 healthy donors with viral particles displaying CD3scfv only or CD3scfv+CD80+CD58 particles.

FIGS. 7A-7C show expression of CAR in cells transduced with viral particles pseudotyped with mutant VSVG envelope proteins. SupT1 cells (FIG. 7A) or PBMCs from two healthy donors (FIGS. 7B-7C) were cultured with viral particles having an anti-CD19 CAR payload and displaying mutant VSVG envelope proteins with or without CD3scfv+CD80+CD58. CAR expression was assessed in CD4+ T cells (FIG. 7B) and CD8+ T cells (FIG. 7C) after transduction of the PBMCs.

FIG. 8 shows the number of CAR negative T cells in the blood of mice after administration of particles at indicated doses encoding an anti-CD19 CAR and displaying CD3scfv only or CD3scfv+CD80+CD58.

FIGS. 9A-9C show particle-T cell binding (FIG. 9A), activation (FIG. 9B), and transduction of T cells (FIG. 9C) in vitro after incubation with particles displaying CD3scfv only or CD3scfv+CD80+CD58.

FIGS. 9D-9H show production of cytokines after incubation with particles displaying CD3scfv only, CD3scfv+CD80, CD3scfv+CD58, or CD3scfv+CD80+CD58. IFN-γ (FIGS. 9D and 9E), IL-2 (FIGS. 9F and 9G), and TNF-α (FIG. 9H) levels were measured.

FIG. 10 shows CD3scfv+CD80+CD58 particles generate CAR+ T cells with a less differentiated cell phenotype measured by expression of CCR7+CD28+CD27+ cell markers as compared to CD3scfv only particles, which produced a more differentiated cell phenotype measured by expression of CD57+.

FIG. 11 shows the number of Nalm6 cells after anti-CD19 CAR+ T cells were serial-stimulated with Nalm6 tumor cells every 2-3 days. anti-CD19 CAR+ T cells were generated with viral particles encoding an anti-CD19 CAR transgene and displaying CD3scfv only or CD3scfv+CD80+CD58 particles. Arrows denote stimulation with Nalm6 tumor cells.

FIG. 12A shows the study design and timeline. FIG. 12B shows the number of cells expressing activation markers CD25 or CD71 four days after transduction with viral particles displaying CD3scfv only or CD3scfv+CD80+CD58+. FIG. 12C shows the number of T cells expressing an anti-CD19 CAR in the blood 11 days after transduction with viral particles displaying CD3scfv only or CD3scfv+CD80+CD58+.

FIGS. 13A-13B show the tumor burden in NSG MHCI/II KO mice after administration with viral particles displaying CD3scfv only (FIG. 13A) or CD3scfv+CD80+CD58 (FIG. 13B).

FIG. 14A shows the study design and timeline.

FIG. 14B is a graph showing staining of Cocal on CD3+ T cells incubated with engineered particles displaying CD58, CD80, and anti-CD3 scFv tri-fusion polypeptide.

FIG. 14C is a graph showing staining of Cocal on engineered particle bound T cells The left peak shows CD3− T cells and the right peak shows CD3+ T cells. The engineered particles display a CD58, CD80, and anti-CD3 scFv tri-fusion polypeptide.

FIG. 14D shows CD25 expression in CD8+ T cells on day 3 after transduction with viral particles displaying CD58, CD80, and anti-CD3 scFv tri-fusion polypeptide.

FIG. 14E shows CAR expression in CD8+ T cells on day 7 after transducion with viral particles displaying CD58, CD80, and anti-CD3 scFv tri-fusion polypeptide.

FIG. 15 shows the number of Nalm6 cells after anti-CD19 CAR+ T cells were serial-stimulated with Nalm6 tumor cells every 2-3 days. anti-CD19 CAR+ T cells were generated with viral particles encoding an anti-CD19 CAR transgene and displaying CD3scfv+CD80+CD58 tri-fusion polypeptide particles. Arrows denote stimulation with Nalm6 tumor cells. Error bars denote mean±SEM.

FIG. 16A shows the study design and timeline. FIG. 16B shows the number of cells expressing activation marker CD25 four days after transduction with viral particles displaying CD3scfv+CD80+CD58+ tri-fusion polypeptide. FIG. 16C shows the number of cells expressing activation marker CD71 four days after transduction with viral particles displaying CD3scfv+CD80+CD58+ tri-fusion polypeptide. FIG. 16D shows production of IFN-γ 4 days after incubation with particles displaying CD3scfv+CD80+CD58 tri-fusion polypeptide. FIG. 16E shows the number of T cells expressing an anti-CD19 CAR in the blood 11 days after transduction with viral particles displaying CD3scfv+CD80+CD58+ tri-fusion polypeptide at a viral dose of 10 Million or 50 Million transducing units (TU). FIG. 16F shows the tumor burden in NSG MHCI/II KO mice after administration of viral particles displaying CD3scfv+CD80+CD58+ tri-fusion polypeptide at a viral dose of 10 Million or 50 Million transducing units (TU).

FIG. 17A shows the study design and timeline. FIG. 17B shows the number of T cells from Donor 1 and Donor 2 expressing an anti-CD19 CAR in the blood 14 days after extracorporeal incubation with viral particles. FIG. 17C shows the tumor burden in Donor 1 and Donor 2 NSG MHCI/II KO mice after administration of T cells produced by via extracorporeal incubation with viral particles. N=7 animals, error bars denote mean±SEM. FIG. 17D shows the study design and timeline for rechallenge study. FIG. 17E shows the tumor burden in Donor 1 and Donor 2 NSG MHCI/II KO mice after administration of T cells produced by via extracorporeal incubation with viral particles and tumor cell rechallenge at Day 49. Error bars denote mean±SEM. FIG. 17F is a schematic that shows an illustrative fusion protein comprising a CD58 extracellular region and α-CD3 scFv fused to the N-terminus of a CD80 via a linker.

### DETAILED DESCRIPTION

In some embodiments, the disclosure provides a viral particle comprising a viral envelope comprising an immune cell-activating protein, a co-stimulatory molecule, a T cell adhesion molecule, and any combination thereof. In some embodiments, the viral particle is a delivery agent for a molecule of interest. In some embodiments, the molecule is a nucleotide sequence encoding a polypeptide of interest. In some embodiments, the molecule is a non-coding nucleic acid. In some embodiments, the non-coding nucleic acid is cDNA, shRNA, microRNA, or siRNA.

In some embodiments, the viral particles described herein activate and transduce immune cells in vivo. In some embodiments, the viral particles described herein activate and transduce immune cells in vitro. In some embodiments, the viral particles activate and transduce immune cells simultaneously.

In some embodiments, the viral particles and methods provided herein eliminate the need for pre-activation of the immune cells prior to administration of the viral particle. In some embodiments, the method comprises no pre-activation of the immune cells in the subject prior to administration of the viral particle (e.g., no pre-activation within about 1, 2, 3, 4, 5, 6, or 7 days, or within about 1, 2, 3, 4, 5, 6, 7, 8, 9, 10, 11, or 12 weeks prior to administration of the viral particle). In some embodiments, pre-activation of the immune cells comprises activating the CD3 and/or CD28 signaling in the immune cells (e.g., T cells), optionally by administering anti-CD3 and/or anti-CD28 antibodies, respectively. Accordingly, in some embodiments, the method of the disclosure does not comprise administering separate CD3 and/or CD28 activating agents prior to administration of the viral particle.

### Viral Particles

In some embodiments, the disclosure provides a viral particle comprising a viral envelope and a payload. In some embodiments, the viral envelope comprises an immune cell-activating protein, a co-stimulatory molecule, a T cell adhesion molecule, or any combination thereof.

In some embodiments, the viral particle comprises a polynucleotide. In some embodiments, the polynucleotide encodes at least one therapeutic polypeptide. The term “therapeutic polypeptide” refers to a polypeptide which is being developed for therapeutic use, or which has been developed for therapeutic use. In some embodiments, the therapeutic polypeptide is expressed in target cells (e.g., host T cells) for therapeutic use. In some embodiments, the therapeutic polypeptide comprises a T cell receptor, a chimeric antigen receptor, or a cytokine receptor.

In some embodiments, the viral particle is a retroviral particle. In some embodiments, the viral particle is a lentiviral particle. In some embodiments, the viral particle is an adeno-associated virus particle.

As used herein, the term “viral particle” refers to a macromolecular complex capable of transferring a payload (e.g., nucleic acid) into a cell. Viral vectors contain structural and/or functional genetic elements that are primarily derived from a virus. The term “retroviral vector” refers to a viral vector containing structural and functional genetic elements, or portions thereof, that are primarily derived from a retrovirus. The term “lentiviral vector” refers to a viral vector containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In some embodiments, a hybrid vector refers to a vector or transfer plasmid comprising retroviral, e.g., lentiviral, sequences for reverse transcription, replication, integration and/or packaging.

### Viral Envelope

In some embodiments, the viral vector comprises a viral envelope comprising a polypeptide on the envelope surface.

In some embodiments, the viral envelope comprises one or more transduction enhancers. In some embodiments, the transduction enhancers include T cell activation receptors, NK cell activation receptors, and/or co-stimulatory molecules. In some embodiments, one or more transduction enhancers comprise one or more of anti-CD3scFv, CD86, CD80, and/or CD58. In some embodiments, the transduction enhancers comprise at least an anti-CD3 scFv, and CD58. In some embodiments, the transduction enhancers comprise at least an anti-CD3 scFv, and CD80. In some embodiments, the transduction enhancers comprise at least an anti-CD3 scFv, and CD86. In some embodiments, the transduction enhancers comprise at least an anti-CD3 scFv, a CD80, and CD58. In some embodiments, the transduction enhancers comprise at least an anti-CD3 scFv, a CD86, and CD58.

In some embodiments, the viral particle comprises a cell surface receptor that binds to a ligand on a target host cell, allowing host cell transduction. In some embodiments, the viral particle comprises a heterologous viral envelope glycoprotein yielding a pseudotyped viral particle. For example, the viral envelope glycoprotein may be derived from RD114 or one of its variants, VSV-G, Gibbon-ape leukemia virus (GALV), or is the Amphotropic envelope, Measles envelope or baboon retroviral envelope glycoprotein. In some embodiments, the viral envelope glycoprotein is a VSV G protein from the Cocal strain (Cocal glycoprotein) or a functional variant thereof.

In some embodiments, the viral envelope comprises more than one polypeptide on the surface. In some embodiments, the more than one polypeptide binds to a target immune cells and replicates an immunological synapse. In some embodiments, the viral envelope comprises an immune cell-activating protein, a co-stimulatory molecule, and an adhesion molecule, wherein the immune cell-activating protein, co-stimulatory molecule, and adhesion molecule each bind a target immune cell.

### Immune Cell-Activating Agents

In some embodiments, the transduction enhancer comprises a mitogenic stimulus, which is incorporated into a retroviral or lentiviral capsid, such that the virus both activates and transduces T cells. This removes the need to add vector and mitogen. In some embodiments, the transduction enhancer comprises a mitogenic transmembrane protein and/or one or more costimulatory and/or adhesion molecules, which get(s) incorporated into the retrovirus when it buds from the producer/packaging cell membrane. In some embodiments, the transduction enhancers are expressed as separate cell surface molecules on the producer cell rather than being part of the viral envelope glycoprotein.

The viral vector described herein may comprise a mitogenic transduction enhancer in the viral envelope. In some embodiments, the mitogenic transduction enhancer is derived from the host cell during retroviral vector production. In some embodiments, the mitogenic transduction enhancer is made by the packaging cell and expressed at the cell surface. When the nascent retroviral vector buds from the host cell membrane, the mitogenic transduction enhancer may be incorporated in the viral envelope as part of the packaging cell-derived lipid bilayer. In some embodiments, the mitogenic enhancer is an antibody or fragment thereof. In some embodiments, the mitogenic enhancer is a single domain antibody, for example, a camelid antibody. In some embodiments, the mitogenic enhancer is an scFv. In some embodiments, the mitogenic enhancer is a nanobody.

In some embodiments, the transduction enhancer is host-cell derived. The term “host-cell derived” indicates that the mitogenic transduction enhancer is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. In some embodiments, the packaging-cell derived mitogenic transduction enhancer of the present invention does not comprise the surface envelope subunit (SU).

In some embodiments, the mitogenic transduction enhancer has the structure: M-S-TM, in which M is a mitogenic domain; S is an optional spacer domain and TM is a transmembrane domain.

The mitogenic domain is the part of the mitogenic transduction enhancer which causes T-cell activation. It may bind or otherwise interact, directly or indirectly, with a T cell, leading to T cell activation. In some embodiments, the mitogenic domain binds a T cell surface antigen, such as CD3, CD28, CD134 and CD137.

CD3 is a T-cell co-receptor. It is a protein complex composed of four distinct chains. In mammals, the complex contains a CD3y chain, a CD35 chain, and two CD3e chains. These chains associate with the T-cell receptor (TCR) and the z-chain to generate an activation signal in T lymphocytes. The TCR, z-chain, and CD3 molecules together comprise the TCR complex. In some embodiments, the mitogenic domain binds to a CD3 e chain.

In some embodiments, the mitogenic domain comprises all or part of an antibody or other molecule which specifically binds a T-cell surface antigen. In some embodiments, the antibody activates the TCR or CD28. In some embodiments, the antibody binds the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

In some embodiments, the mitogenic domain comprises the binding domain from OKT3, 15E8, TGN1412, CD28.2, 10F3, UCHT1, YTH12.5 or TR66.

In some embodiments, the mitogenic domain comprises all or part of a co-stimulatory molecule such as OX40L and 41BBL. For example, the mitogenic domain may comprise the binding domain from OX40L or 41BBL.

OKT3, also known as Muromonab-CD3 is a monoclonal antibody targeted at the CD3e chain. It is clinically used to reduce acute rejection in patients with organ transplants. It was the first monoclonal antibody to be approved for clinical use in humans. The CDRs of OKT3 are as follows 1

CDRH1:
(SEQ ID NO. 136)
GYTFTRY
CDRH2:
(SEQ ID NO. 137)
NPSRGY
CDRH3:
(SEQ ID NO. 138)
YYDDHYCLDY
CDRL1:
(SEQ ID NO. 139)
SASSSVSYMN
CDRL2:
(SEQ ID NO. 140)
DTSKLAS
CDRL3:
(SEQ ID NO. 141)
QQWSSNPFT

In some embodiments, the viral envelope comprises an immune cell-activating protein. In some embodiments, the immune cell-activating protein specifically binds a receptor on an immune cell. In some embodiments, the immune cell-activating protein provides signal one for T cell activation.

In some embodiments, the immune cell-activating protein specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80. In some embodiments, the immune cell-activating protein specifically binds CD3γ, CD3δ, or CD3ε. In some embodiments, the immune cell-activating protein specifically binds CD3γ, CD3δ, CD3ε, CD9, CD5, CD22, CD33, CD37, CD64, CD45, CD28H, LFA-1, DNAM-1, CD27, ICOS, LIGHT, GITR, CD30, SLAM, Ly-9, CD84, Ly108, CD16, CD56, NKG2D, NKp46, NKp44, NKp30, CD244, NKp80, TCRα chain, TCRβ chain, TCRγ chain, or TCRδ chain. In some embodiments, the immune cell-activating protein specifically binds CD3γ, CD3δ, or CD3ε. In some embodiments, the immune cell-activating protein specifically binds CD3.

In some embodiments, the immune cell-activating protein is an antibody or antigen binding fragment thereof that specifically binds a receptor on an immune cell. In some embodiments, the immune cell-activating protein is an antibody or antigen binding fragment thereof that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80. In some embodiments, the immune cell-activating protein is an antibody or antigen binding fragment thereof that specifically binds CD28, CD2, CD3γ, CD3δ, CD3ε, CD4, CD8, CD9, CD5, CD22, CD33, CD37, CD64, CD45, CD28H, LFA-1, OX40, 4-1BB, CD40L, DNAM-1, CD27, ICOS, LIGHT, GITR, CD30, SLAM, Ly-9, CD84, Ly108, CD16, CD56, NKG2D, NKp46, NKp44, NKp30, CD244, NKp80, TCRα chain, TCRβ chain, TCRγ chain, or TCRδ chain. some embodiments, the immune cell-activating protein is an antibody or antigen binding fragment thereof that specifically binds CD3γ, CD3δ, or CD3ε. In some embodiments, immune cell-activating protein is an antibody or antigen binding fragment thereof that specifically binds CD3.

Antibodies targeting the polypeptides described herein are known to those of skill in the art. Methods for generating antibodies are known to those of skill in the art.

In some embodiments, the viral envelope comprises an anti-CD3ε antibody, or antigen-binding fragment thereof. In some embodiments, the anti-CD3ε antibody, or antigen-binding fragment thereof is coupled to a transmembrane domain. An illustrative anti-CD3ε antibody is OKT3. OKT3, also known as Muromonab-CD3, is a monoclonal antibody targeted at the CD3ε chain.

In some embodiments, the viral envelope comprises a single chain Fv fragment (scFv) of an anti-CD3 antibody.

In some embodiments, the viral envelope comprises an anti-CD3 scFv comprising an amino acid sequence at least 75%, at least 80%, at least 85%, at least 90%, at least 95%, at least 99%, or 100% identical to SEQ ID NO: 2. In some embodiments, the viral envelope comprises an anti-CD3 scFv comprising the amino acid sequence of SEQ ID NO: 2.

In some embodiments, the viral envelope comprises an anti-CD3 scFv comprising the following complementary determining regions (CDR): SASSSVSYMN (CDR-L1; SEQ ID NO: 133), DTSKLASG (CDR-L2; SEQ ID NO: 134), QQWSSNPFT (CDR-L3; SEQ ID NO: 135), RYTMH (CDR-H1; SEQ ID NO: 48), YINPSRGYTNYNQKVKD (CDR-H2; SEQ ID NO: 36), and YYDDHYCLDY (CDR-H3; SEQ ID NO: 38).

In some embodiments, the nucleotide sequence encoding an anti-CD3 scFv comprises at least 75%, at least 80%, at least 85%, at least 90%, at least 95%, at least 99%, or 100% identical to SEQ ID NO: 7. In some embodiments, the nucleotide sequence encoding an anti-CD3 scFv comprises SEQ ID NO: 7.

In some embodiments, the viral envelope comprises an anti-CD3 scFv comprising an amino acid sequence at least 75%, at least 80%, at least 85%, at least 90%, at least 95%, at least 99%, or 100% identical to SEQ ID NO: 12. In some embodiments, the viral envelope comprises an anti-CD3 scFv comprising the amino acid sequence of SEQ ID NO: 12.

In some embodiments, the viral envelope comprises an anti-CD3 scFv comprising the following CDRs: SASSSVSYMN (CDR-L1; SEQ ID NO: 133), DTSKLASG (CDR-L2; SEQ ID NO: 134), QQWSSNPFT (CDR-L3; SEQ ID NO: 135), RYTMH (CDR-H1; SEQ ID NO: 48), YINPSRGYTNYNQKVKD (CDR-H2; SEQ ID NO: 36), and YYDDHYCLDY (CDR-H3; SEQ ID NO: 38).

In some embodiments, the nucleotide sequence encoding an anti-CD3 scFv comprises at least 75%, at least 80%, at least 85%, at least 90%, at least 95%, at least 99%, or 100% identical to SEQ ID NO: 15. In some embodiments, the nucleotide sequence encoding an anti-CD3 scFv comprises SEQ ID NO: 15.

### Co-Stimulatory Molecules

In some embodiments, the viral envelope comprises at least one co-stimulatory molecule. In some embodiments, the co-stimulatory molecule specifically binds a receptor on an immune cell. In some embodiments, the co-stimulatory provides signal two for cell activation.

As used herein, the term “costimulatory molecule” refers to a molecule capable of generating a costimulatory signal to T cells. Lymphocytes, such as T cells and natural killer (NK) cells, typically require several signals and interactions with antigen presenting cells (APCs) for optimal priming to gain full effector functions. For T cells these include signaling through the T cell receptor (TCR), costimulatory molecules (such as CD28 and CD2), cytokines, as well as various adhesion molecules necessary to allow sufficient time for proper synapse formation and signal transduction. NK cells require similar types of stimulation but may rely on different activating receptors, such as NKG2D, NKp46, and DNAM-1. For T cells, proper costimulation, in addition to TCR stimulation, is especially important for effective priming and many studies have shown that TCR stimulation alone can lead to functional anergy and unresponsiveness. Costimulatory signals augment T and NK cell function by enhancing cell metabolism, cytokine production, differentiation, and long-term persistence. Costimulation is an important factor for cell proliferation, differentiation and survival. In some embodiments, costimulatory molecules include, but are not limited to, CD45, CD2, CD4, CD5, CD8, CD9, CD16, CD22, CD33, CD28, CD37, CD64, CD80, CD86, CD134, CD137, and CD154. In some embodiments, the costimulatory molecule may also be an adhesion molecule. In some embodiments, the costimulatory molecule includes, but is not limited to, binding agents, such as scFvs, antibodies, single-domain antibodies, antibody fragments, nanobodies that bind to any of the costimulatory or adhesion molecules described herein. In some embodiments, these binding agents may include anti-CD28, anti-CD2, anti-CD45, anti-CD4, anti-CD5, anti-CD8, anti-CD9, anti-CD16, anti-CD22, anti-CD33, anti-CD37, anti-CD64, anti-CD80, anti-CD86, anti-CD137, anti-CD154, anti-CD28H, anti-LFA-1, anti-OX40, anti-4-1BB, anti-CD40L, anti-DNAM-1, anti-CD27, anti-ICOS, anti-LIGHT, anti-GITR, anti-CD30, anti-SLAM, anti-Ly-9, anti-CD84, anti-Ly108, anti-NKG2D, anti-NKp46, anti-NKp44, anti-NKp30, anti-CD244, anti-NKp80, anti-TCRα chain, anti-TCRβ chain, anti-TCRγ chain, and anti-TCRδ chain agents.

In some embodiments, the co-stimulation molecule is a ligand for CD28. CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). In some embodiments, the co-stimulation molecule is an antibody, or fragment thereof, that binds to CD28. Examples of such antibodies include: 15E8 and TGN1412. Other suitable antibodies include: CD28.2 and 10F3.

15E8 is a mouse monoclonal antibody to human CD28. Its CDRs are as follows:

CDRH1:
(SEQ ID NO. 142)
GFSLTSY
CDRH2:
(SEQ ID NO. 143)
WAGGS
CDRH3:
(SEQ ID NO. 144)
DKRAPGKLYYGYPDY
CDRL1:
(SEQ ID NO. 145)
RASESVEYYVTSLMQ
CDRL2:
(SEQ ID NO. 146)
AASNYES
CDRL3:
(SEQ ID NO. 147)
QQTRKVPST

TGN1412 (also known as CD28-SuperMAB) is a humanised monoclonal antibody that not only binds to, but is a strong agonist for, the CD28 receptor. Its CDRs are as follows.

CDRH1:
(SEQ ID NO. 148)
GYTFSY
CDRH2:
(SEQ ID NO. 149)
YPGNVN
CDRH3:
(SEQ ID NO. 150)
SHYGLDWNFDV
CDRL1:
(SEQ ID NO. 151)
HASQNIYVLN
CDRL2:
(SEQ ID NO. 152)
KASNLHT
CDRL3:
(SEQ ID NO. 153)
QQGQTYPYT

In some embodiments, the co-stimulation molecule is CD86. CD86, also known as B7-2, is a ligand for CD28. In some embodiments, the ligand for CD28 is CD86. In some embodiments, the co-stimulation molecule is CD80. CD80 is an additional ligand for CD28. In some embodiments, the ligand for CD28 is CD80. In some embodiments, the ligand for CD28 is an anti-CD28 antibody or an anti-CD28 scFv. In some embodiments, the anti-CD28 antibody or an anti-CD28 scFv is coupled to a transmembrane domain for display on the surface of the viral envelope.

In some embodiments, the co-stimulation molecule is a CD86 polypeptide comprising the amino acid sequence of SEQ ID NO: 23. In some embodiments, the co-stimulation molecule is a CD86 polypeptide comprising an amino acid sequence at least 80%, at least 85%, at least 90%, at least 95%, at least 96%, at least 97%, at least 98% or at least 99% identical to SEQ ID NO: 23.

In some embodiments, the CD86 polypeptide is encoded by the nucleotide sequence of SEQ ID NO: 24. In some embodiments, the CD86 polypeptide is encoded by a nucleotide sequence at least 80%, at least 85%, at least 90%, at least 95%, at least 96%, at least 97%, at least 98% or at least 99% identical to SEQ ID NO: 24.

In some embodiments, the co-stimulation molecule is a CD80 polypeptide comprising the amino acid sequence of SEQ ID NO: 20. In some embodiments, the co-stimulation molecule is a CD80 polypeptide comprising an amino acid sequence at least 80%, at least 85%, at least 90%, at least 95%, at least 96%, at least 97%, at least 98% or at least 99% identical to SEQ ID NO: 20.

In some embodiments, the CD80 polypeptide is encoded by the nucleotide sequence of SEQ ID NO: 21. In some embodiments, the CD80 polypeptide is encoded by a nucleotide sequence at least 80%, at least 85%, at least 90%, at least 95%, at least 96%, at least 97%, at least 98% or at least 99% identical to SEQ ID NO: 21.

CD134, also known as OX40, is a member of the TNFR-superfamily of receptors which is expressed on activated T cells. OX40 may promote cell division and survival. 0X40 is a secondary costimulatory molecule, expressed after 24 to 72 hours following activation; its ligand, OX40L, is also not expressed on resting antigen presenting cells, but is following their activation. In some embodiments, the viral particle comprises a ligand for OX40, or functional fragment thereof, coupled to its native transmembrane domain or a heterologous transmembrane domain.

CD137, also known as 4-1BB, is a member of the tumor necrosis factor (TNF) receptor family. CD137 is expressed on activated T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity. In some embodiments, the viral particle comprises a ligand for 4-1BB, or functional fragment thereof, coupled to its native transmembrane domain or a heterologous transmembrane domain. 4-1BBL is a cytokine that belongs to the tumor necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

Viral particles comprising one or more activation or co-stimulation molecule(s) may be made by engineering the packaging cell line by methods provided by WO 2016/139463; or by expression of the T-cell activation or co-stimulation molecule(s) from a polycistronic helper vector as described in Int'l Pat. Pub. No. WO 2020/106992 A1, both of which are incorporated herein by reference in their entireties.

### Adhesion Molecules

In some embodiments, the viral particle comprises an adhesion molecule. As used herein, the term “adhesion molecule” refers to a subset of cell surface molecules involved in the binding of cells with other cells. Adhesion cells may help to form more stable interactions, such as an immunological synapse, between immune cells. The immunological synapse is a stable adhesive junction between a polarized immune effector cell and an antigen-bearing cell. In some embodiments, the adhesion molecule may provide a costimulatory signal to the target cell. In some embodiments, adhesion molecules include, but are not limited to, CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, and B7-H6.

Costimulatory and adhesion molecules of the present disclosure include, but are not limited to, CD80, CD86, CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, and B7 family members such as B7-H2, B7-H6, and B7-H5. Binding proteins, such as scfvs and nanobodies, for the ligands of these are also incorporated including anti-CD28, anti-CD2, anti-CD28H, anti-LFA-1, anti-OX40, anti-4-1BB, anti-CD40L, anti-DNAM-1, anti-CD27, anti-ICOS, anti-LIGHT, anti-GITR, anti-CD30, anti-SLAM, anti-Ly-9, anti-CD84, anti-Ly108, anti-NKG2D, anti-NKp46, anti-NKp44, anti-NKp30, anti-CD244, and anti-NKp80. Membrane-bound cytokines include, but are not limited to, IL-2, IL-7, IL-12, IL-15, IL-18, and IL-21. The fusion construct of the present disclosure may also comprise B7-H3, B7x, and/or TMIGD2. The fusion proteins as disclosed herein may include one or more domains that engage with costimulatory and/or adhesion molecules presented on the surface of a T cell, including CD28, CD28H, CD2, CD3, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80.

In some embodiments, the adhesion molecule includes, but is not limited to, binding agents, such as scFvs, antibodies, single-domain antibodies, antibody fragments, and nanobodies that bind to any of the adhesion or costimulatory molecules described herein. In some embodiments, these binding agents may include anti-CD28, anti-CD2, anti-CD28H, anti-LFA-1, anti-OX40, anti-4-1BB, anti-CD40L, anti-DNAM-1, anti-CD27, anti-ICOS, anti-LIGHT, anti-GITR, anti-CD30, anti-SLAM, anti-Ly-9, anti-CD84, anti-Ly108, anti-NKG2D, anti-NKp46, anti-NKp44, anti-NKp30, anti-CD244, anti-NKp8O, anti-TCRα chain, anti-TCRβ chain, anti-TCRγ chain, and anti-TCRδ chain agents.

In some embodiments, the costimulatory and/or adhesion molecule shares at least 80%, at least 85%, at least 90%, at least 95%, at least 96%, at least 97%, at least 98% or at least 99% identity to a sequence in Table 1.

TABLE 1

| | | SEQ ID | |
|---|---|---|---|
| Name | Sequence | NO: | Region |
| CD80 | VIHVTKEVKEVATLSCGHNVSVEELAQTRIYWQKEKK | 173 | Val35- |
| Extracellular | MVLTMMSGDMNIWPEYKNRTIFDITNNLSIVILALRPS | | Asn242 |
| Domain | DEGTYECVVLKYEKDAFKREHLAEVTLSVKADFPTPSI | | |
| | SDFEIPTSNIRRIICSTSGGFPEPHLSWLENGEELNAINTT | | |
| | VSQDPETELYAVSSKLDFNMTTNHSFMCLIKYGHLRV | | |
| | NQTFNWNTTKQEHFPDN | | |
| CD86 | APLKIQAYFNETADLPCQFANSQNQSLSELVVFWQDQ | 174 | Ala24- |
| Extracellular | ENLVLNEVYLGKEKFDSVHSKYMGRTSFDSDSWTLRL | | Pro247 |
| Domain | HNLQIKDKGLYQCIIHHKKPTGMIRIHQMNSELSVLAN | | |
| | FSQPEIVPISNITENVYINLTCSSIHGYPEPKKMSVLLRT | | |
| | KNSTIEYDGVMQKSQDNVTELYDVSISLSVSFPDVTSN | | |
| | MTIFCILETDKTRLLSSPFSIELEDPQPPPDHIP | | |
| CD58 | FSQQIYGVVYGNVTFHVPSNVPLKEVLWKKQKDKVA | 175 | Phe29- |
| Extracellular | ELENSEFRAFSSFKNRVYLDTVSGSLTIYNLTSSDEDEY | | Val120 |
| Domain | EMESPNITDTMKFFLYV | | |
| HHLA2 (a/k/a | IFPLAFFIYVPMNEQIVIGRLDEDIILPSSFERGSEVVIHW | 176 | Ile23- |
| B7-H7) | KYQDSYKVHSYYKGSDHLESQDPRYANRTSLFYNEIQ | | Lys345 |
| Extracellular | NGNASLFFRRVSLLDEGIYTCYVGTAIQVITNKVVLKV | | |
| Domain | GVFLTPVMKYEKRNTNSFLICSVLSVYPRPIITWKMDN | | |
| | TPISENNMEETGSLDSFSINSPLNITGSNSSYECTIENSLL | | |
| | KQTWTGRWTMKDGLHKMQSEHVSLSCQPVNDYFSPN | | |
| | QDFKVTWSRMKSGTFSVLAYYLSSSQNTIINESRFSWN | | |
| | KELINQSDFSMNLMDLNLSDSGEYLCNISSDEYTLLTIH | | |
| | TVHVEPSQETASHNK | | |
| ICAM-1 | QTSVSPSKVILPRGGSVLVTCSTSCDQPKLLGIETPLPK | 177 | Gln28- |
| Extracellular | KELLLPGNNRKVYELSNVQEDSQPMCYSNCPDGQSTA | | Glu480 |
| Domain | KTFLTVYWTPERVELAPLPSWQPVGKNLTLRCQVEGG | | |
| | APRANLTVVLLRGEKELKREPAVGEPAEVTTTVLVRR | | |
| | DHHGANFSCRTELDLRPQGLELFENTSAPYQLQTFVLP | | |
| | ATPPQLVSPRVLEVDTQGTVVCSLDGLFPVSEAQVHLA | | |
| | LGDQRLNPTVTYGNDSFSAKASVSVTAEDEGTQRLTC | | |
| | AVILGNQSQETLQTVTIYSFPAPNVILTKPEVSEGTEVT | | |
| | VKCEAHPRAKVTLNGVPAQPLGPRAQLLLKATPEDNG | | |
| | RSFSCSATLEVAGQLIHKNQTRELRVLYGPRLDERDCP | | |
| | GNWTWPENSQQTPMCQAWGNPLPELKCLKDGTFPLPI | | |
| | GESVTVTRDLEGTYLCRARSTQGEVTRKVTVNVLSPR | | |
| | YE | | |
| OX40-L | QVSHRYPRIQSIKVQFTEYKKEKGFILTSQKEDEIMKVQ | 178 | Gln51- |
| Extracellular | NNSVIINCDGFYLISLKGYFSQEVNISLHYQKDEEPLFQ | | Leu183 |
| Domain | LKKVRSVNSLMVASLTYKDKVYLNVTTDNTSLDDFH | | |
| | VNGGELILIHQNPGEFCVL | | |
| 4-1BBL | REGPELSPDDPAGLLDLRQGMFAQLVAQNVLLIDGPLS | 179 | Arg71- |
| Extracellular | WYSDPGLAGVSLTGGLSYKEDTKELVVAKAGVYYVF | | Glu254 |
| Domain | FQLELRRVVAGEGSGSVSLALHLQPLRSAAGAAALAL | | |
| | TVDLPPASSEARNSAFGFQGRLLHLSAGQRLGVHLHTE | | |
| | ARARHAWQLTQGATVLGLFRVTPEIPAGLPSPRSE | | |
| CD40 | EPPTACREKQYLINSQCCSLCQPGQKLVSDCTEFTETEC | 180 | Glu21- |
| Extracellular | LPCGESEFLDTWNRETHCHQHKYCDPNLGLRVQQKGT | | Arg193 |
| Domain | SETDTICTCEEGWHCTSEACESCVLHRSCSPGFGVKQIA | | |
| | TGVSDTICEPCPVGFFSNVSSAFEKCHPWTSCETKDLV | | |
| | VQQAGTNKTDVVCGPQDRLR | | |
| CD155 | WPPPGTGDVVVQAPTQVPGFLGDSVTLPCYLQVPNME | 181 | Trp21- |
| Extracellular | VTHVSQLTWARHGESGSMAVFHQTQGPSYSESKRLEF | | Asn343 |
| Domain | VAARLGAELRNASLRMFGLRVEDEGNYTCLFVTFPQG | | |
| | SRSVDIWLRVLAKPQNTAEVQKVQLTGEPVPMARCVS | | |
| | TGGRPPAQITWHSDLGGMPNTSQVPGFLSGTVTVTSL | | |
| | WILVPSSQVDGKNVTCKVEHESFEKPQLLTVNLTVYYP | | |
| | PEVSISGYDNNWYLGQNEATLTCDARSNPEPTGYNWS | | |
| | TTMGPLPPFAVAQGAQLLIRPVDKPINTTLICNVTNALG | | |
| | ARQAELTVQVKEGPPSEHSGISRN | | |
| CD70 | QRFAQAQQQLPLESLGWDVAELQLNHTGPQQDPRLY | 182 | Gln39- |
| Extracellular | WQGGPALGRSFLHGPELDKGQLRIHRDGIYMVHIQVT | | Pro193 |
| Domain | LAICSSTTASRHHPTTLAVGICSPASRSISLLRLSFHQGC | | |
| | TIASQRLTPLARGDTLCTNLTGTLLPSRNTDETFFGVQ | | |
| | WVRP | | |
| HVEM | LPSCKEDEYPVGSECCPKCSPGYRVKEACGELTGTVCE | 183 | Leu39- |
| Extracellular | PCPPGTYIAHLNGLSKCLQCQMCDPAMGLRASRNCSR | | Ser199 |
| Domain | TENAVCGCSPGHFCIVQDGDHCAACRAYATSSPGQRV | | |
| | QKGGTESQDTLCQNCPPGTFSPNGTLEECQHQTKCSW | | |
| | LVTKAGAGTSSS | | |
| GITRL | QLETAKEPCMAKFGPLPSKWQMASSEPPCVNKVSDW | 184 | Gln72- |
| Extracellular | KLEILQNGLYLIYGQVAPNANYNDVAPFEVRLYKNKD | | Ser199 |
| Domain | MIQTLTNKSKIQNVGGTYELHVGDTIDLIFNSEHQVLK | | |
| | NNTYWGILLANPQFIS | | |
| CD30L | QRTDSIPNSPDNVPLKGGNCSEDLLCILKRAPFKKSWA | 185 | Gln63- |
| Extracellular | YLQVAKHLNKTKLSWNKDGILHGVRYQDGNLVIQFPG | | Asp234 |
| Domain | LYFIICQLQFLVQCPNNSVDLKLELLINKHIKKQALVTV | | |
| | CESGMQTKHVYQNLSQFLLDYLQVNTTISVNVDTFQYI | | |
| | DTSTFPLENVLSIFLYSNSD | | |
| SLAMF2 | QGHLVHMTVVSGSNVTLNISESLPENYKQLTWFYTFD | 186 | Gln27- |
| Extracellular | QKIVEWDSRKSKYFESKFKGRVRLDPQSGALYISKVQK | | Arg219 |
| Domain | EDNSTYIMRVLKKTGNEQEWKIKLQVLDPVPKPVIKIE | | |
| | KIEDMDDNCYLKLSCVIPGESVNYTWYGDKRPFPKEL | | |
| | QNSVLETTLMPHNYSRCYTCQVSNSVSSKNGTVCLSPP | | |
| | CTLAR | | |
| Ly-9 | KDSAPTVVSGILGGSVTLPLNISVDTEIENVIWIGPKNA | 187 | Lys48- |
| Extracellular | LAFARPKENVTIMVKSYLGRLDITKWSYSLCISNLTLN | | Lys454 |
| Domain | DAGSYKAQINQRNFEVTTEEEFTLFVYEQLQEPQVTM | | |
| | KSVKVSENFSCNITLMCSVKGAEKSVLYSWTPREPHAS | | |
| | ESNGGSILTVSRTPCDPDLPYICTAQNPVSQRSSLPVHV | | |
| | GQFCTDPGASRGGTTGETVVGVLGEPVTLPLALPACR | | |
| | DTEKVVWLFNTSIISKEREEAATADPLIKSRDPYKNRV | | |
| | WVSSQDCSLKISQLKIEDAGPYHAYVCSEASSVTSMTH | | |
| | VTLLIYRRLRKPKITWSLRHSEDGICRISLTCSVEDGGN | | |
| | TVMYTWTPLQKEAVVSQGESHLNVSWRSSENHPNLTC | | |
| | TASNPVSRSSHQFLSENICSGPERNTK | | |
| CD84 | KDSEIFTVNGILGESVTFPVNIQEPRQVKIIAWTSKTSVA | 188 | Lys22- |
| Extracellular | YVTPGDSETAPVVTVTHRNYYERIHALGPNYNLVISDL | | Gly225 |
| Domain | RMEDAGDYKADINTQADPYTTTKRYNLQIYRRLGKPK | | |
| | ITQSLMASVNSTCNVTLTCSVEKEEKNVTYNWSPLGEE | | |
| | GNVLQIFQTPEDQELTYTCTAQNPVSNNSDSISARQLC | | |
| | ADIAMGFRTHHTG | | |
| Ly108 (a/k/a | QSSLTPLMVNGILGESVTLPLEFPAGEKVNFITWLFNET | 189 | Gln22- |
| SLAMF6) | SLAFIVPHETKSPEIHVTNPKQGKRLNFTQSYSLQLSNL | | Lys225 |
| Extracellular | KMEDTGSYRAQISTKTSAKLSSYTLRILRQLRNIQVTN | | |
| Domain | HSQLFQNMTCELHLTCSVEDADDNVSFRWEALGNTLS | | |
| | SQPNLTVSWDPRISSEQDYTCIAENAVSNLSFSVSAQKL | | |
| | CEDVKIQYTDTK | | |
| MICA | EPHSLRYNLTVLSWDGSVQSGFLTEVHLDGQPFLRCD | 190 | Glu24- |
| Extracellular | RQKCRAKPQGQWAEDVLGNKTWDRETRDLTGNGKD | | His306 |
| Domain | LRMTLAHIKDQKEGLHSLQEIRVCEIHEDNSTRSSQHF | | |
| | YYDGELFLSQNLETKEWTMPQSSRAQTLAMNVRNFLK | | |
| | EDAMKTKTHYHAMHADCLQELRRYLKSGVVLRRTVP | | |
| | PMVNVTRSEASEGNITVTCRASGFYPWNITLSWRQDG | | |
| | VSLSHDTQQWGDVLPDGNGTYQTWVATRICQGEEQR | | |
| | FTCYMEHSGNHSTHPVPSGKVLVLQSH | | |
| MICB | EPHSLRYNLMVLSQDGSVQSGFLAEGHLDGQPFLRYD | 191 | Glu24- |
| Extracellular | RQKRRAKPQGQWAEDVLGAETWDTETEDLTENGQDL | | Asp266 |
| Domain | RRTLTHIKDQKGVPQSSRAQTLAMNVTNFWKEDAMK | | |
| | TKTHYRAMQADCLQKLQRYLKSGVAIRRTVPPMVNV | | |
| | TCSEVSEGNITVTCRASSFYPRNITLTWRQDGVSLSHNT | | |
| | QQWGDVLPDGNGTYQTWVATRIRQGEBQRFTCYMEH | | |
| | SGNHGTHPVPSGKALVLQSQRTD | | |
| ULBP1 | MAAAASPAFLLCLPLLHLLSGWSRAGWVDTHCLCYDF | 192 | |
| | IITPKSRPEPQWCEVQGLVDERPFLHYDCVNHKAKAFA | | |
| | SLGKKVNVTKTWEEQTETLRDVVDFLKGQLLDIQVEN | | |
| | LIPIEPLTLQARMSCEHEAHGHGRGSWQFLENGQKFLL | | |
| | FDSNNRKWTALHPGAKKMTEKWEKNRDVTMFFQKIS | | |
| | LGDCKMWLEEFLMYWEQMLDPTKPPSLAPGTTQPKA | | |
| | MATTLSPWSLLIIFLCFILAGR | | |
| ULBP2 | MAAAAATKILLCLPLLLLLSGWSRAGRADPHSLCYDIT | 193 | |
| | VIPKFRPGPRWCAVQGQVDEKTFLHYDCGNKTVTPVS | | |
| | PLGKKLNVTTAWKAQNPVLREVVDILTEQLRDIQLEN | | |
| | YTPKEPLTLQARMSCEQKAEGHSSGSWQFSFDGQIFLL | | |
| | FDSEKRMWTTVHPGARKMKEKWENDKVVAMSFHYF | | |
| | SMGDCIGWLEDFLMGMDSTLEPSAGAPLAMSSGTTQL | | |
| | RATATTLILCCLLIILPCFILPGI | | |
| ULBP3 | MAAAASPAILPRLAILPYLLFDWSGTGRADAHSLWYN | 194 | |
| | FTIIHLPRHGQQWCEVQSQVDQKNFLSYDCGSDKVLS | | |
| | MGHLEEQLYATDAWGKQLEMLREVGQRLRLELADTE | | |
| | LEDFTPSGPLTLQVRMSCECEADGYIRGSWQFSFDGRK | | |
| | FLLFDSNNRKWTVVHAGARRMKEKWEKDSGLTTFFK | | |
| | MVSMRDCKSWLRDFLMHRKKRLEPTAPPTMAPGLAQ | | |
| | PKAIATTLSPWSFLIILCFILPGI | | |
| ULBP4 | HSLCFNFTIKSLSRPGQPWCEAQVFLNKNLFLQYNSDN | 195 | His31- |
| Extracellular | NMVKPLGLLGKKVYATSTWGELTQTLGEVGRDLRML | | Asp225 |
| Domain | LCDIKPQIKTSDPSTLQVEMFCQREAERCTGASWQFAT | | |
| | NGEKSLLFDAMNMTWTVINHEASKIKETWKKDRGLE | | |
| | KYFRKLSKGDCDHWLREFLGHWEAMPEPTVSPVNAS | | |
| | DIHWSSSSLPD | | |
| ULBP5 | GLADPHSLCYDITVIPKFRPGPRWCAVQGQVDEKTFLH | 196 | Gly26- |
| Extracellular | YDCGSKTVTPVSPLGKKLNVTTAWKAQNPVLREVVDI | | Arg223 |
| Domain | LTEQLLDIQLENYIPKEPLTLQARMSCEQKAEGHGSGS | | |
| | WQLSFDGQIFLLFDSENRMWTTVHPGARKMKEKWEN | | |
| | DKDMTMSFHYISMGDCTGWLEDFLMGMDSTLEPSAG | | |
| | APPTMSSGTAQPR | | |
| ULBP6 | MAAAAIPALLLCLPLLFLLFGWSRARRDDPHSLCYDIT | 197 | |
| | VIPKFRPGPRWCAVQGQVDEKTFLHYDCGNKTVTPVS | | |
| | PLGKKLNVTMAWKAQNPVLREVVDILTEQLLDIQLEN | | |
| | YTPKEPLTLQARMSCEQKAEGHSSGSWQFSIDGQTFLL | | |
| | FDSEKRMWTTVHPGARKMKEKWENDKDVAMSFHYIS | | |
| | MGDCIGWLEDFLMGMDSTLEPSAGAPLAMSSGTTQLR | | |
| | ATATTLILCCLLIILPCFILPGI | | |
| B7-H2 (a/k/a | DTQEKEVRAMVGSDVELSCACPEGSRFDLNDVYVYW | 198 | Asp19- |
| ICOSL) | QTSESKTVVTYHIPQNSSLENVDSRYRNRALMSPAGM | | Thr256 |
| Extracellular | LRGDFSLRLFNVTPQDEQKFHCLVLSQSLGFQEVLSVE | | |
| Domain | VTLHVAANFSVPVVSAPHSPSQDELTFTCTSINGYPRPN | | |
| | VYWINKTDNSLLDQALQNDTVFLNMRGLYDVVSVLRI | | |
| | ARTPSVNIGCCIENVLLQQNLTVGSQTGNDIGERDKITE | | |
| | NPVSTGEKNAAT | | |
| B7-H6 | DLKVEMMAGGTQITPLNDNVTIFCNIFYSQPLNITSMGI | 199 | Asp25- |
| Extracellular | TWFWKSLTFDKEVKVFEFFGDHQEAFRPGAIVSPWRL | | Ser262 |
| Domain | KSGDASLRLPGIQLEEAGEYRCEVVVTPLKAQGTVQLE | | |
| | VVASPASRLLLDQVGMKENEDKYMCESSGFYPEAINIT | | |
| | WEKQTQKFPHPIEISEDVITGPTIKNMDGTFNVTSCLKL | | |
| | NSSQEDPGTVYQCVVRHASLHTPLRSNFTLTAARHSLS | | |
| | ETEKTDNFS | | |
| B7-H5 | FKVATPYSLYVCPEGQNVTLTCRLLGPVDKGHDVTFY | 200 | Phe33- |
| Extracellular | KTWYRSSRGEVQTCSERRPIRNLTFQDLHLHHGGHQA | | Ala194 |
| Domain | ANTSHDLAQRHGLESASDHHGNFSITMRNLTLLDSGL | | |
| | YCCLVVEIRHHHSEHRVHGAMELQVQTGKDAPSNCV | | |
| | VYPSSSQDSENITAA | | |
| B7-H3 | LEVQVPEDPVVALVGTDATLCCSFSPEPGFSLAQLNLI | 201 | Leu29- |
| Extracellular | WQLTDTKQLVHSFAEGQDQGSAYANRTALFPDLLAQ | | Ala248 |
| Domain | GNASLRLQRVRVADEGSFTCFVSIRDFGSAAVSLQVAA | | |
| | PYSKPSMTLEPNKDLRPGDTVTITCSSYRGYPEAEVFW | | |
| | QDGQGVPLTGNVTTSQMANEQGLFDVHSVLRVVLGA | | |
| | NGTYSCLVRNPVLQQDAHGSVTITGQPMTFPPEA | | |
| B7x (a/k/a | LIIGFGISGRHSITVTTVASAGNIGEDGILSCTFEPDIKLS | 202 | Leu25- |
| B7-H4) | DIVIQWLKEGVLGLVHEFKEGKDELSEQDEMFRGRTA | | Ser259 |
| Extracellular | VFADQVIVGNASLRLKNVQLTDAGTYKCYIITSKGKG | | |
| Domain | NANLEYKTGAFSMPEVNVDYNASSETLRCEAPRWFPQ | | |
| | PTVVWASQVDQGANFSEVSNTSFELNSENVTMKVVSV | | |
| | LYNVTINNTYSCMIENDIAKATGDIKVTESEIKRRSHLQ | | |
| | LLNSKASTTENLYFQG | | |
| TMIGD2 | LSVQQGPNLLQVRQGSQATLVCQVDQATAWERLRVK | 203 | Leu23- |
| Extracellular | WTKDGAILCQPYITNGSLSLGVCGPQGRLSWQAPSHLT | | Gly150 |
| Domain | LQLDPVSLNHSGAYVCWAAVEIPELEEAEGNITRLFVD | | |
| | PDDPTQNRNRIASFPG | | |
| IL-2 | APTSSSTKKTQLQLEHLLLDLQMILNGINNYKNPKLTR | 204 | |
| | MLTFKFYMPKKATELKHLQCLEEELKPLEEVLNLAQS | | |
| | KNFHLRPRDLISNINVIVLELKGSETTFMCEYADETATI | | |
| | VEFLNRWITFCQSIISTLT | | |
| IL-7 | DCDIEGKDGKQYESVLMVSIDQLLDSMKEIGSNCLNNE | 205 | |
| | FNFFKRHICDANKEGMFLFRAARKLRQFLKMNSTGDF | | |
| | DLHLLKVSEGTTILLNCTGQVKGRKPAALGEAQPTKSL | | |
| | EENKSLKEQKKLNDLCFLKRLLQEIKTCWNKILMGTK | | |
| | EH | | |
| IL-12 subunit | RNLPVATPDPGMFPCLHHSQNLLRAVSNMLQKARQTL | 206 | |
| alpha | EFYPCTSEEIDHEDITKDKTSTVEACLPLELTKNESCLN | | |
| | SRETSFITNGSCLASRKTSFMMALCLSSIYEDLKMYQV | | |
| | EFKTMNAKLLMDPKRQIFLDQNMLAVIDELMQALNEN | | |
| | SETVPQKSSLEEPDFYKTKIKLCILLHAFRIRAVTIDRV | | |
| | MSYLNAS | | |
| IL-12 subunit | IWELKKDVYVVELDWYPDAPGEMVVLTCDTPEEDGIT | 207 | |
| beta | WTLDQSSEVLGSGKTLTIQVKEFGDAGQYTCHKGGEV | | |
| | LSHSLLLLHKKEDGIWSTDILKDQKEPKNKTFLRCEAK | | |
| | NYSGRFTCWWLTTISTDLTFSVKSSRGSSDPQGVTCGA | | |
| | ATLSAERVRGDNKEYEYSVECQEDSACPAAEESLPIEV | | |
| | MVDAVHKLKYENYTSSFFIRDIIKPDPPKNLQLKPLKNS | | |
| | RQVEVSWEYPDTWSTPHSYFSLTFCVQVQGKSKREKK | | |
| | DRVFTDKTSATVICRKNASISVRAQDRYYSSSWSEWAS | | |
| | VPCS | | |
| IL-15 | NWVNVISDLKKIEDLIQSMHIDATLYTESDVHPSCKVT | 208 | |
| | AMKCFLLELQVISLESGDASIHDTVENLIILANNSLSSN | | |
| | GNVTESGCKECEELEEKNIKEFLQSFVHIVQMFINTS | | |
| IL-18 | YFGKLESKLSVIRNLNDQVLFIDQGNRPLFEDMTDSDC | 209 | |
| | RDNAPRTIFIISMYKDSQPRGMAVTISVKCEKISTLSCE | | |
| | NKIISFKEMNPPDNIKDTKSDIIFFQRSVPGHDNKMQFE | | |
| | SSSYEGYFLACEKERDLFKLILKKEDELGDRSIMFTVQ | | |
| | NED | | |
| IL-21 | HKSSSQGQDRHMIRMRQLIDIVDQLKNYVNDLVPEFLP | 210 | |
| | APEDVETNCEWSAFSCFQKAQLKSANTGNNERIINVSI | | |
| | KKLKRKPPSTNAGRRQKHRLTCPSCDSYEKKPPKEFLE | | |
| | RFKSLLQKMIHQHLSSRTHGSEDS | | |

In some embodiments, the costimulatory and/or adhesion molecule is linked to a transmembrane domain. In some embodiments, the transmembrane domain may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain may be derived from the transmembrane domain of CD28. In some embodiments, the transmembrane domain of may be derived from the transmembrane domain of CD8, for example, CD8a.

Without wishing to be bound by theory, reducing foreign junctions (i.e., between an adhesion molecule and a transmembrane domain) in the foreign nucleic acid incorporated into a lentiviral particle may reduce the immunogenicity of a subject to a lentiviral particle.

In some embodiments, the adhesion molecule binds to CD2. CD2 is also known as T11, LFA-2, and the erythrocyte rosette receptor and is a surface protein expressed on T lymphocytes and NK cells. CD2 is a natural ligand for CD58. In addition to performing adhesion functions, engagement of CD2 provides a costimulatory signal that may enhance activation and effector functions. In some embodiments, the lentiviral particle comprises a molecule that binds to CD2. In some embodiments, the lentiviral particle comprises an antibody, single domain antibody, antibody fragment, and/or nanobody specific for CD2. In some embodiments, the lentiviral particle comprises CD58, or a functional portion thereof that binds to CD2.

In some embodiments, the adhesion molecule is CD58. In some embodiments, the co-stimulation molecule is a CD58 polypeptide comprising the amino acid sequence of SEQ ID NO: 17. In some embodiments, the co-stimulation molecule is a CD58 polypeptide comprising an amino acid sequence at least 80%, at least 85%, at least 90%, at least 95%, at least 96%, at least 97%, at least 98% or at least 99% identical to SEQ ID NO: 17.

In some embodiments, the CD58 polypeptide is encoded by the nucleotide sequence of SEQ ID NO: 18. In some embodiments, the CD58 polypeptide is encoded by a nucleotide sequence at least 80%, at least 85%, at least 90%, at least 95%, at least 96%, at least 97%, at least 98% or at least 99% identical to SEQ ID NO: 18.

### Additional Non-Viral Proteins

In some embodiments, the viral particle comprises at least one non-viral protein. In some embodiments, the viral particle comprises at least one non-viral protein in addition to those described supra.

In some embodiments, the viral particle comprises a targeting ligand. In some embodiments, the viral particle comprises CD19, or a functional fragment thereof, coupled to its native transmembrane domain or a heterologous transmembrane domain. In some embodiments, CD19 acts as a ligand for blinatumomab, thus providing an adapter for coupling the particle to T-cells via the anti-CD3 moiety of blinatumomab. In some embodiments, another type of particle surface ligand can serve to couple an appropriately surface engineered lentiviral particle to a T-cell using a multispecific antibody comprising a binding moiety for the particle surface ligand. In some embodiments, the multispecific antibody is a bispecific antibody, for example, a Bispecific T-cell engager (BiTE).

In some embodiments, the non-viral protein is a cytokine. In some embodiments, the cytokine may be selected from the group consisting of IL-2, IL-7, IL-12, IL-15, IL-18, IL-21, and any combination thereof. Where the non-viral protein used is a soluble protein (such as an scFv or a cytokine) it may be tethered to the surface of the viral particle by fusion to a transmembrane domain, such as the transmembrane domain of CD8. Alternatively, it may be indirectly tethered to the lentiviral particle by use of a transmembrane protein engineered to bind the soluble protein. Further inclusion of one or more cytoplasmic residues may increase the stability of the fusion protein.

The mitogenic transduction enhancer and/or cytokine-based transduction enhancer may comprise a “spacer sequence” to connect the antigen-binding domain with the transmembrane domain. A flexible spacer allows the antigen-binding domain to orient in different directions to facilitate binding. As used herein, the term “coupled to” refers to a chemical linkage, a direct C-terminal to N-terminal fusion of two protein; chemical linkage to a non-peptide space; chemical linkage to a polypeptide space; and C-terminal to N-terminal fusion of two protein via peptide bonds to a polypeptide spacer, e.g., a spacer sequence.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an lgG1 Fc region, an lgG1 hinge or a CD8 stalk. A human lgG1 spacer may be altered to remove Fe binding motifs. In some embodiments, the spacer sequence may be derived from a human protein.

In some embodiments, the spacer sequence comprises a CD8 derived hinge.

In some embodiments, the spacer sequence comprises a ‘short’ hinge. The short hinge is described as hinge region comprising fewer nucleotides relative to CAR hinge regions known in the art.

In some embodiments, the viral particle comprises a polypeptide comprising a CD8 hinge that shares at least 75%, at least 80%, at least 85%, at least 90%, at least 95%, at least 99%, or 100% identity to SEQ ID NO: 3.

In some embodiments, the viral particle comprises a nucleic acid sequence encoding a CD8 hinge that shares at least 75%, at least 80%, at least 85%, at least 90%, at least 95%, at least 99%, or 100% identity to SEQ ID NO: 8.

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

The viral particle of the present invention may comprise a cytokine-based transduction enhancer in the viral envelope. In some embodiments, the cytokine-based transduction enhancer is derived from the host cell during viral particle production. In some embodiments, the cytokine-based transduction enhancer is made by the host cell and expressed at the cell surface. When the nascent viral particle buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain (e.g., a spacer sequence) and TM is the transmembrane domain. The spacer domain and transmembrane domains are as defined above.

The cytokine domain may comprise a T-cell activating cytokine, such as from IL2, IL7 and IL15, or a functional fragment thereof. As used herein, a “functional fragment” of a cytokine is a fragment of a polypeptide that retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding.

### Viral Envelope Proteins

In some embodiments, the viral envelope comprises a viral envelope protein. In some embodiments, a viral envelope protein is a VSV-G envelope protein, a measles virus envelope protein, a nipha virus envelope protein, or a cocal virus G protein. In some embodiments, the viral particle comprises a modified VSV G protein that lacks LDLR binding affinity. In some embodiments, these mutations comprise mutations at positions 47 (for example, K47Q) and/or 354 (for example, R354A).

In some embodiments, the viral envelope protein is a VSV G protein from the Cocal strain (Cocal glycoprotein). In some embodiments, the VSV G protein is a Cocal envelope protein containing a mutation at position 354 (R354). In some embodiments, the VSV G protein is a Cocal envelope protein containing a mutation at position 47 (K47). In some embodiments, the VSV G protein is a Cocal envelope variant containing the R354Q mutation relative to SEQ ID NO: 5. In some embodiments, the VSV G protein is a Cocal envelope variant containing the K47Q mutation relative to SEQ ID NO: 5. In some embodiments, this variant may be referred to as “blinded” Cocal envelope. Illustrative Cocal envelope variants are provided in, e.g., US 2020/0216502 A1, which is incorporated herein by reference in its entirety.

In some embodiments, the viral particle comprises a Cocal glycoprotein comprising an amino acid sequence with at least 75%, at least 80%, at least 85%, at least 90%, at least 95%, at least 99%, or 100% identity to SEQ ID NO: 5. In some embodiments, the viral particle comprises SEQ ID NO: 5.

In some embodiments, a nucleotide sequence encoding the viral particle comprises a nucleotide sequence encoding a Cocal glycoprotein. In some embodiments, the nucleotide sequence encoding a Cocal glycoprotein has at least 75%, at least 80%, at least 85%, at least 90%, at least 95%, at least 99%, or 100% identity to SEQ ID NO: 10. In some embodiments, the nucleotide sequence encoding a Cocal glycoprotein comprises the sequence of SEQ ID NO: 10.

In some embodiments, a nucleotide sequence encoding the viral particle comprises a nucleotide sequence encoding a Cocal glycoprotein. In some embodiments, the nucleotide sequence encoding a Cocal glycoprotein has at least 75%, at least 80%, at least 85%, at least 90%, at least 95%, at least 99%, or 100% identity to SEQ ID NO: 104. In some embodiments, the nucleotide sequence encoding a Cocal glycoprotein comprises the sequence of SEQ ID NO: 104.

Various fusion glycoproteins can be used to pseudotype lentiviral particles. While the most commonly used example is the envelope glycoprotein from vesicular stomatitis virus (VSV-G), many other viral proteins have also been used for pseudotyping of lentiviral particles. See Joglekar et al. Human Gene Therapy Methods 28:291-301 (2017). The present disclosure contemplates substitution of various fusion glycoproteins. Notably, some fusion glycoproteins result in higher viral particle efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, anti-CD3 scFv, (CD3), Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the viral particle is a Nipah virus (NiV) envelope pseudotyped lentivirus particle (“Nipah envelope pseudotyped vector”). In some embodiments, the Nipah envelope pseudotyped vector is pseudotyped using Nipah virus envelope glycoproteins NiV-F and NiV-G. In some embodiments, the NiV-F and/or NiV-G glycoproteins on such Nipah envelope pseudotyped vector are modified variants. In some embodiments, the NiV-F and/or NiV-G glycoproteins on such Nipah envelope pseudotyped vector are modified to include an antigen binding domain. In some embodiments, the antigen is EpCAM, CD4, or CD8. In some embodiments, the Nipah envelope pseudotyped vector can efficiently transduce cells expressing EpCAM, CD4, or CD8. See U.S. Pat. No. 9,486,539 and Bender et al. PLoS Pathog. (2016) June; 12(6): e1005641.

### Exemplary Viral Envelopes

In some embodiments, the viral particle comprises a viral envelope comprising (i) an immune cell-activating protein, (ii) a co-stimulatory molecule, (iii) an adhesion molecule, or (iv) any combination of (i)-(iii). In some embodiments, the viral particle comprises a viral envelope comprising (i) an immune cell-activating protein, (ii) a co-stimulatory molecule, (iii) an adhesion molecule, or (iv) any combination of at least two of (i)-(iii). In some embodiments, the viral particle comprises a viral envelope comprising (i) an immune cell-activating protein and (ii) a co-stimulatory molecule. In some embodiments, the viral particle comprises a viral envelope comprising (i) an immune cell-activating protein and (ii) an adhesion molecule. In some embodiments, the viral particle comprises a viral envelope comprising (i) an immune cell-activating protein, (ii) a co-stimulatory molecule, and (iii) an adhesion molecule. In some embodiments, the viral particle comprises a viral envelope comprising (i) an immune cell-activating protein, (ii) a co-stimulatory molecule, and (iii) a plurality of adhesion molecules.

In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, or (iv) any combination of (i)-(iii). In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, or (iv) any combination of at least two of (i)-(iii). In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, and (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, and (ii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR 3 chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, and (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof.

In some embodiments, the viral particle comprises a viral envelope comprising (i) an immune cell-activating protein that specifically binds CD3, (ii) a co-stimulatory molecule selected from CD80, CD86, and a combination thereof, (iii) an adhesion molecule comprising a CD58 polypeptide, or (iv) any combination of (i)-(iii). In some embodiments, the viral particle comprises a viral envelope comprising (i) an immune cell-activating protein that specifically binds CD3, (ii) a co-stimulatory molecule selected from CD80, CD86, and a combination thereof, (iii) an adhesion molecule comprising a CD58 polypeptide, or (iv) any combination of at least two of (i)-(iii). In some embodiments, the viral particle comprises a viral envelope comprising (i) an immune cell-activating protein that specifically binds CD3, and (ii) a co-stimulatory molecule selected from CD80, CD86, and a combination thereof. In some embodiments, the viral particle comprises a viral envelope comprising (i) an immune cell-activating protein that specifically binds CD3, and (ii) an adhesion molecule comprising a CD58 polypeptide. In some embodiments, the viral particle comprises a viral envelope comprising (i) an immune cell-activating protein that specifically binds CD3, (ii) a co-stimulatory molecule selected from CD80, CD86, and a combination thereof, and (iii) an adhesion molecule comprising a CD58 polypeptide.

In some embodiments, the viral particle comprises a viral envelope comprising (i) an immune cell-activating protein that specifically binds CD3, (ii) a co-stimulatory molecule comprising a CD80 polypeptide, (iii) an adhesion molecule comprising a CD58 polypeptide, or (iv) any combination of (i)-(iii). In some embodiments, the viral particle comprises a viral envelope comprising (i) an immune cell-activating protein that specifically binds CD3, (ii) a co-stimulatory molecule comprising a CD80 polypeptide, (iii) an adhesion molecule comprising a CD58 polypeptide, or (iv) any combination of at least two of (i)-(iii). In some embodiments, the viral particle comprises a viral envelope comprising (i) an immune cell-activating protein that specifically binds CD3, and (ii) a co-stimulatory molecule comprising a CD80 polypeptide. In some embodiments, the viral particle comprises a viral envelope comprising (i) an immune cell-activating protein that specifically binds CD3, and (ii) an adhesion molecule comprising a CD58 polypeptide. In some embodiments, the viral particle comprises a viral envelope comprising (i) an immune cell-activating protein that specifically binds CD3, (ii) a co-stimulatory molecule comprising a CD80 polypeptide, and (iii) an adhesion molecule comprising a CD58 polypeptide.

In some embodiments, the viral particle comprises a viral envelope comprising (i) an immune cell-activating protein that specifically binds CD3, (ii) a co-stimulatory molecule comprising a CD86 polypeptide, (iii) an adhesion molecule comprising a CD58 polypeptide, or (iv) any combination of (i)-(iii). In some embodiments, the viral particle comprises a viral envelope comprising (i) an immune cell-activating protein that specifically binds CD3, (ii) a co-stimulatory molecule comprising a CD86 polypeptide, (iii) an adhesion molecule comprising a CD58 polypeptide, or (iv) any combination of at least two of (i)-(iii). In some embodiments, the viral particle comprises a viral envelope comprising (i) an immune cell-activating protein that specifically binds CD3, and (ii) a co-stimulatory molecule comprising a CD86 polypeptide. In some embodiments, the viral particle comprises a viral envelope comprising (i) an immune cell-activating protein that specifically binds CD3, and (ii) an adhesion molecule comprising a CD58 polypeptide. In some embodiments, the viral particle comprises a viral envelope comprising (i) an immune cell-activating protein that specifically binds CD3, (ii) a co-stimulatory molecule comprising a CD86 polypeptide, and (iii) an adhesion molecule comprising a CD58 polypeptide.

In some embodiments, the viral particle comprises a viral envelope comprising (i) an antibody that specifically binds CD3 or an antigen binding fragment thereof, (ii) a co-stimulatory molecule comprising a CD80 polypeptide, (iii) an adhesion molecule comprising a CD58 polypeptide, or (iv) any combination of (i)-(iii). In some embodiments, the viral particle comprises a viral envelope comprising (i) an antibody that specifically binds CD3 or an antigen binding fragment thereof, (ii) a co-stimulatory molecule comprising a CD80 polypeptide, (iii) an adhesion molecule comprising a CD58 polypeptide, or (iv) any combination of at least two of (i)-(iii). In some embodiments, the viral particle comprises a viral envelope comprising (i) an antibody that specifically binds CD3 or an antigen binding fragment thereof, and (ii) a co-stimulatory molecule comprising a CD80 polypeptide. In some embodiments, the viral particle comprises a viral envelope comprising (i) an antibody that specifically binds CD3 or an antigen binding fragment thereof, and (ii) an adhesion molecule comprising a CD58 polypeptide. In some embodiments, the viral particle comprises a viral envelope comprising (i) an antibody that specifically binds CD3 or an antigen binding fragment thereof, (ii) a co-stimulatory molecule comprising a CD80 polypeptide, and (iii) an adhesion molecule comprising a CD58 polypeptide.

In some embodiments, the viral particle comprises a viral envelope comprising (i) an antibody that specifically binds CD3 or an antigen binding fragment thereof, (ii) a co-stimulatory molecule comprising a CD86 polypeptide, (iii) an adhesion molecule comprising a CD58 polypeptide, or (iv) any combination of (i)-(iii). In some embodiments, the viral particle comprises a viral envelope comprising (i) an antibody that specifically binds CD3 or an antigen binding fragment thereof, (ii) a co-stimulatory molecule comprising a CD86 polypeptide, (iii) an adhesion molecule comprising a CD58 polypeptide, or (iv) any combination of at least two of (i)-(iii). In some embodiments, the viral particle comprises a viral envelope comprising (i) an antibody that specifically binds CD3 or an antigen binding fragment thereof, and (ii) a co-stimulatory molecule comprising a CD86 polypeptide. In some embodiments, the viral particle comprises a viral envelope comprising (i) an antibody that specifically binds CD3 or an antigen binding fragment thereof, and (ii) an adhesion molecule comprising a CD58 polypeptide. In some embodiments, the viral particle comprises a viral envelope comprising (i) an antibody that specifically binds CD3 or an antigen binding fragment thereof, (ii) a co-stimulatory molecule comprising a CD86 polypeptide, and (iii) an adhesion molecule comprising a CD58 polypeptide.

In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, (iii) an adhesion molecule comprising a CD58 polypeptide comprising the amino acid sequence of SEQ ID NO: 17, or (iv) any combination of (i)-(iii). In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, (iii) an adhesion molecule comprising a CD58 polypeptide comprising the amino acid sequence of SEQ ID NO: 17, or (iv) any combination of at least two of (i)-(iii). In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, and (ii) a co-stimulatory molecule comprising a CD80 polypeptide comprising the amino acid sequence of SEQ ID NO: 20. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, and (ii) an adhesion molecule comprising a CD58 polypeptide comprising the amino acid sequence of SEQ ID NO: 17. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, and (iii) an adhesion molecule comprising a CD58 polypeptide comprising the amino acid sequence of SEQ ID NO: 17.

In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, (iii) an adhesion molecule comprising a CD58 polypeptide comprising the amino acid sequence of SEQ ID NO: 17, or (iv) any combination of (i)-(iii). In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, (iii) an adhesion molecule comprising a CD58 polypeptide comprising the amino acid sequence of SEQ ID NO: 17, or (iv) any combination of at least two of (i)-(iii). In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, and (ii) a co-stimulatory molecule comprising a CD86 polypeptide comprising the amino acid sequence of SEQ ID NO: 23. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, and (ii) an adhesion molecule comprising a CD58 polypeptide comprising the amino acid sequence of SEQ ID NO: 17. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, and (iii) an adhesion molecule comprising a CD58 polypeptide comprising the amino acid sequence of SEQ ID NO: 17.

In some embodiments, the viral particle comprises a viral envelope comprising (i) an immune cell-activating protein, (ii) a co-stimulatory molecule, (iii) an adhesion molecule, or (iv) any combination of (i)-(iii), and a viral envelope protein. In some embodiments, the viral particle comprises a viral envelope comprising (i) an immune cell-activating protein, (ii) a co-stimulatory molecule, (iii) an adhesion molecule, or (iv) any combination of at least two of (i)-(iii), and a viral envelope protein. In some embodiments, the viral particle comprises a viral envelope comprising (i) an immune cell-activating protein, (ii) a co-stimulatory molecule, and (iii) a viral envelope protein. In some embodiments, the viral particle comprises a viral envelope comprising (i) an immune cell-activating protein, (ii) an adhesion molecule, and (iii) a viral envelope protein. In some embodiments, the viral particle comprises a viral envelope comprising (i) an immune cell-activating protein, (ii) a co-stimulatory molecule, (iii) an adhesion molecule, and (iv) a viral envelope protein.

In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, or (iv) any combination of (i)-(iii), and a viral envelope protein. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, or (iv) any combination of at least two of (i)-(iii), and a viral envelope protein. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, and (iii) a viral envelope protein. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, and (iii) a viral envelope protein. In some embodiments, the viral particle comprises a viral envelope comprising (i) an immune cell-activating protein that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80, (ii) a co-stimulatory molecule selected from TCR α chain, TCR β chain, TCR ζ chain, CD3 ε TCR subunit, CD3 γ TCR subunit, CD3 δ TCR subunit, CD45, CD2, CD4, CD5, CD8, CD9, CD16, CD22, CD33, CD28, CD37, CD64, CD80, CD86, CD134, CD137, CD154, and any combination thereof, (iii) an adhesion molecule selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof, and (iv) a viral envelope protein.

In some embodiments, the viral particle comprises a viral envelope comprising (i) an immune cell-activating protein that specifically binds CD3, (ii) a co-stimulatory molecule selected from CD80, CD86, and a combination thereof, (iii) an adhesion molecule comprising a CD58 polypeptide, or (iv) any combination of (i)-(iii), and a viral envelope protein. In some embodiments, the viral particle comprises a viral envelope comprising (i) an immune cell-activating protein that specifically binds CD3, (ii) a co-stimulatory molecule selected from CD80, CD86, and a combination thereof, (iii) an adhesion molecule comprising a CD58 polypeptide, or (iv) any combination of at least two of (i)-(iii), and a viral envelope protein. In some embodiments, the viral particle comprises a viral envelope comprising (i) an immune cell-activating protein that specifically binds CD3, (ii) a co-stimulatory molecule selected from CD80, CD86, and a combination thereof, and (iii) a viral envelope protein. In some embodiments, the viral particle comprises a viral envelope comprising (i) an immune cell-activating protein that specifically binds CD3, (ii) an adhesion molecule comprising a CD58 polypeptide, and (iii) a viral envelope protein. In some embodiments, the viral particle comprises a viral envelope comprising (i) an immune cell-activating protein that specifically binds CD3, (ii) a co-stimulatory molecule selected from CD80, CD86, and a combination thereof, (iii) an adhesion molecule comprising a CD58 polypeptide, and (iv) a viral envelope protein.

In some embodiments, the viral particle comprises a viral envelope comprising (i) an immune cell-activating protein that specifically binds CD3, (ii) a co-stimulatory molecule comprising a CD80 polypeptide, (iii) an adhesion molecule comprising a CD58 polypeptide, or (iv) any combination of (i)-(iii), and a viral envelope protein. In some embodiments, the viral particle comprises a viral envelope comprising (i) an immune cell-activating protein that specifically binds CD3, (ii) a co-stimulatory molecule comprising a CD80 polypeptide, (iii) an adhesion molecule comprising a CD58 polypeptide, or (iv) any combination of at least two of (i)-(iii), and a viral envelope protein. In some embodiments, the viral particle comprises a viral envelope comprising (i) an immune cell-activating protein that specifically binds CD3, (ii) a co-stimulatory molecule comprising a CD80 polypeptide, and (iii) a viral envelope protein. In some embodiments, the viral particle comprises a viral envelope comprising (i) an immune cell-activating protein that specifically binds CD3, (ii) an adhesion molecule comprising a CD58 polypeptide, and (iii) a viral envelope protein. In some embodiments, the viral particle comprises a viral envelope comprising (i) an immune cell-activating protein that specifically binds CD3, (ii) a co-stimulatory molecule comprising a CD80 polypeptide, (iii) an adhesion molecule comprising a CD58 polypeptide, and (iv) a viral envelope protein.

In some embodiments, the viral particle comprises a viral envelope comprising (i) an immune cell-activating protein that specifically binds CD3, (ii) a co-stimulatory molecule comprising a CD86 polypeptide, (iii) an adhesion molecule comprising a CD58 polypeptide, or (iv) any combination of (i)-(iii), and a viral envelope protein. In some embodiments, the viral particle comprises a viral envelope comprising (i) an immune cell-activating protein that specifically binds CD3, (ii) a co-stimulatory molecule comprising a CD86 polypeptide, (iii) an adhesion molecule comprising a CD58 polypeptide, or (iv) any combination of at least two of (i)-(iii), and a viral envelope protein. In some embodiments, the viral particle comprises a viral envelope comprising (i) an immune cell-activating protein that specifically binds CD3, (ii) a co-stimulatory molecule comprising a CD86 polypeptide, and (iii) a viral envelope protein. In some embodiments, the viral particle comprises a viral envelope comprising (i) an immune cell-activating protein that specifically binds CD3, (ii) an adhesion molecule comprising a CD58 polypeptide, and (iii) a viral envelope protein. In some embodiments, the viral particle comprises a viral envelope comprising (i) an immune cell-activating protein that specifically binds CD3, (ii) a co-stimulatory molecule comprising a CD86 polypeptide, (iii) an adhesion molecule comprising a CD58 polypeptide, and (iv) a viral envelope protein.

In some embodiments, the viral particle comprises a viral envelope comprising (i) an antibody that specifically binds CD3 or an antigen binding fragment thereof, (ii) a co-stimulatory molecule comprising a CD80 polypeptide, (iii) an adhesion molecule comprising a CD58 polypeptide, or (iv) any combination of (i)-(iii), and a viral envelope protein. In some embodiments, the viral particle comprises a viral envelope comprising (i) an antibody that specifically binds CD3 or an antigen binding fragment thereof, (ii) a co-stimulatory molecule comprising a CD80 polypeptide, (iii) an adhesion molecule comprising a CD58 polypeptide, or (iv) any combination of at least two of (i)-(iii), and a viral envelope protein. In some embodiments, the viral particle comprises a viral envelope comprising (i) an antibody that specifically binds CD3 or an antigen binding fragment thereof, (ii) a co-stimulatory molecule comprising a CD80 polypeptide, and (iii) a viral envelope protein. In some embodiments, the viral particle comprises a viral envelope comprising (i) an antibody that specifically binds CD3 or an antigen binding fragment thereof, (ii) an adhesion molecule comprising a CD58 polypeptide, and (iii) a viral envelope protein.

In some embodiments, the viral particle comprises a viral envelope comprising (i) an antibody that specifically binds CD3 or an antigen binding fragment thereof, (ii) a co-stimulatory molecule comprising a CD80 polypeptide, (iii) an adhesion molecule comprising a CD58 polypeptide, and (iv) a viral envelope protein. In some embodiments, the viral particle comprises a viral envelope comprising (i) an antibody that specifically binds CD3 or an antigen binding fragment thereof, (ii) a co-stimulatory molecule comprising a CD86 polypeptide, (iii) an adhesion molecule comprising a CD58 polypeptide, or (iv) any combination of (i)-(iii), and a viral envelope protein. In some embodiments, the viral particle comprises a viral envelope comprising (i) an antibody that specifically binds CD3 or an antigen binding fragment thereof, (ii) a co-stimulatory molecule comprising a CD86 polypeptide, (iii) an adhesion molecule comprising a CD58 polypeptide, or (iv) any combination of at least two of (i)-(iii), and a viral envelope protein. In some embodiments, the viral particle comprises a viral envelope comprising (i) an antibody that specifically binds CD3 or an antigen binding fragment thereof, (ii) a co-stimulatory molecule comprising a CD86 polypeptide, and (iii) a viral envelope protein. In some embodiments, the viral particle comprises a viral envelope comprising (i) an antibody that specifically binds CD3 or an antigen binding fragment thereof, (ii) an adhesion molecule comprising a CD58 polypeptide, and (iii) a viral envelope protein. In some embodiments, the viral particle comprises a viral envelope comprising (i) an antibody that specifically binds CD3 or an antigen binding fragment thereof, (ii) a co-stimulatory molecule comprising a CD86 polypeptide, (iii) an adhesion molecule comprising a CD58 polypeptide, and (iv) a viral envelope protein.

In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, (iii) an adhesion molecule comprising a CD58 polypeptide comprising the amino acid sequence of SEQ ID NO: 17, or (iv) any combination of (i)-(iii), and a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, (iii) an adhesion molecule comprising a CD58 polypeptide comprising the amino acid sequence of SEQ ID NO: 17, or (iv) any combination of at least two of (i)-(iii), and a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, and (iii) a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) an adhesion molecule comprising a CD58 polypeptide comprising the amino acid sequence of SEQ ID NO: 17, and (iii) a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD80 polypeptide comprising the amino acid sequence of SEQ ID NO: 20, (iii) an adhesion molecule comprising a CD58 polypeptide comprising the amino acid sequence of SEQ ID NO: 17, and (iv) a viral envelope protein.

In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, (iii) an adhesion molecule comprising a CD58 polypeptide comprising the amino acid sequence of SEQ ID NO: 17, or (iv) any combination of (i)-(iii), and a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, (iii) an adhesion molecule comprising a CD58 polypeptide comprising the amino acid sequence of SEQ ID NO: 17, or (iv) any combination of at least two of (i)-(iii), and a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, and (iii) a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) an adhesion molecule comprising a CD58 polypeptide comprising the amino acid sequence of SEQ ID NO: 17, and (iii) a viral envelope protein. In some embodiments, the viral particle comprises a viral envelope comprising (i) an antibody that specifically binds CD3 or an antigen binding fragment thereof, wherein the antibody or antigen binding fragment comprises CDR-L1 of SEQ ID NO: 133, CDR-L2 of SEQ ID NO: 134, CDR-L3 of SEQ ID NO: 135, CDR-H1 of SEQ ID NO: 48, CDR-H2 of SEQ ID NO: 36, and CDR-H3 of SEQ ID NO: 38, (ii) a co-stimulatory molecule comprising a CD86 polypeptide comprising the amino acid sequence of SEQ ID NO: 23, (iii) an adhesion molecule comprising a CD58 polypeptide comprising the amino acid sequence of SEQ ID NO: 17, and (iv) a viral envelope protein.

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 107.

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 109.

In some embodiments, the viral particle comprises a polypeptide comprising a Gaussia luciferase signal peptide, operably linked to an anti-CD3 scFv, operably linked to a linker, operably linked to a Glycophorin A derived transmembrane domain and HIV envelope derived cytoplasmic tail that shares at least 75%, at least 80%, at least 85%, at least 90%, at least 95%, at least 99%, or 100% identity to SEQ ID NO: 111.

In some embodiments, the viral particle comprises a polypeptide comprising a Gaussia luciferase signal peptide, operably linked to an anti-CD3 scFv, operably linked to a linker, operably linked to a HIV envelope derived transmembrane domain and cytoplasmic tail that shares at least 75%, at least 80%, at least 85%, at least 90%, at least 95%, at least 99%, or 100% identity to SEQ ID NO: 113.

In some embodiments, the viral particle comprises a polypeptide comprising a Gaussia luciferase signal peptide, operably linked to an anti-CD3 scFv, operably linked to a triple G4Slinker, operably linked to a HIV envelope derived transmembrane domain and cytoplasmic tail that shares at least 75%, at least 80%, at least 85%, at least 90%, at least 95%, at least 99%, or 100% identity to SEQ ID NO: 115.

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain, cytoplasmic tail, and T2A self-cleaving peptide, operably linked to a Cocal envelope that shares at least 75%, at least 80%, at least 85%, at least 90%, at least 95%, at least 99%, or 100% identity to SEQ ID NO: 117.

In some embodiments, the viral particle comprises a polypeptide comprising a Gaussia luciferase signal peptide, operably linked to an anti-CD3 scFv, operably linked to a linker, operably linked to a Glycophorin A derived hinge, transmembrane domain, and cytoplasmic tail that shares at least 75%, at least 80%, at least 85%, at least 90%, at least 95%, at least 99%, or 100% identity to SEQ ID NO: 119.

In some embodiments, the viral particle comprises a polypeptide comprising a short hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 13.

In some embodiments, the viral particle comprises a polypeptide comprising a long hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 19.

In some embodiments, the viral particle comprises a polypeptide comprising a 218 linker operably linked to a human Glycophorin A ectodomain transmembrane domain operably linked to a cytoplasmic tail derived from a HIV viral envelope that shares at least 75%, at least 80%, at least 85%, at least 90%, at least 95%, at least 99%, or 100% identity to SEQ ID NO: 25.

In some embodiments, the viral particle comprises a polypeptide comprising a 218 linker operably linked to a HIV viral envelope transmembrane domain and cytoplasmic tail that shares at least 75%, at least 80%, at least 85%, at least 90%, at least 95%, at least 99%, or 100% identity to SEQ ID NO: 31.

In some embodiments, the viral particle comprises a polypeptide comprising a triple G4S linker operably linked to a HIV viral envelope transmembrane domain and cytoplasmic tail that shares at least 75%, at least 80%, at least 85%, at least 90%, at least 95%, at least 99%, or 100% identity to SEQ ID NO: 37.

In some embodiments, the viral particle comprises a polypeptide comprising a Ser-Gly peptide operably linked to small ectodomain, transmembrane and cytoplasmic tail sequences derived from human Glycophorin A that shares at least 75%, at least 80%, at least 85%, at least 90%, at least 95%, at least 99%, or 100% identity to SEQ ID NO: 97.

In some embodiments, the viral particle comprises a polypeptide comprising transmembrane domain and cytoplasmic tail sequences derived from human Glycophorin A that shares at least 75%, at least 80%, at least 85%, at least 90%, at least 95%, at least 99%, or 100% identity to SEQ ID NO: 105.

In some embodiments, the viral particle comprises a polypeptide comprising a short hinge operably linked to a Cocal glycoprotein transmembrane domain and cytoplasmic tail that shares at least 75%, at least 80%, at least 85%, at least 90%, at least 95%, at least 99%, or 100% identity to SEQ ID NO: 43.

In some embodiments, the viral particle comprises a polypeptide comprising a CD4 derived transmembrane domain and cytoplasmic tail operably linked to a T2A linker that shares at least 75%, at least 80%, at least 85%, at least 90%, at least 95%, at least 99%, or 100% identity to SEQ ID NO: 4.

In some embodiments, the viral particle comprises a polypeptide comprising a Gaussia luciferase derived signal peptide sequence that shares at least 75%, at least 80%, at least 85%, at least 90%, at least 95%, at least 99%, or 100% identity to SEQ ID NO: 11.

### Viral Particle Envelope Expression Cassettes

In some embodiments, the viral particles described herein are generated using a viral envelope expression cassette encoding at least one of the polypeptides described herein.

In some embodiments, the viral particles of the present disclosure are generated using a viral envelope expression cassette encoding, in 5′ to 3′ order:
(a) a CD8 derived signal peptide
(b) an anti-CD3 scFV
(c) a CD8 derived hinge domain
(d) a CD4 transmembrane domain and cytoplasmic tail
(e) a T2A linker
(f) a Cocal glycoprotein

In some embodiments, the viral particles of the present disclosure are generated using a viral envelope expression cassette encoding, in 5′ to 3′ order:
(a) a Gaussia luciferase derived signal peptide
(b) an anti-CD3 scFV
(c) a short hinge domain
(d) a Cocal glycoprotein derived transmembrane domain and cytoplasmic tail

In some embodiments, the viral particles of the present disclosure are generated using a viral envelope expression cassette encoding, in 5′ to 3′ order:
(a) a Gaussia luciferase derived signal peptide
(b) an anti-CD3 scFV
(c) a long hinge domain
(d) a Cocal glycoprotein derived transmembrane domain and cytoplasmic tail

In some embodiments, the viral particles of the present disclosure are generated using a viral envelope expression cassette encoding, in 5′ to 3′ order:
(a) a Gaussia luciferase derived signal peptide
(b) an anti-CD3 scFV
(c) a 218 linker
(d) a human Glycophorin A ectodomain derived transmembrane domain
(e) a HIV viral envelope derived cytoplasmic tail

In some embodiments, the viral particles of the present disclosure are generated using a viral envelope expression cassette encoding, in 5′ to 3′ order:
(a) a Gaussia luciferase derived signal peptide
(b) an anti-CD3 scFV
(c) a 218 linker
(d) a HIV viral envelope derived transmembrane domain and cytoplasmic tail

In some embodiments, the viral particles of the present are generated using comprise a viral envelope expression cassette encoding, in 5′ to 3′ order:
(a) a Gaussia luciferase derived signal peptide
(b) an anti-CD3 scFV
(c) a triple G4S linker
(d) a HIV viral envelope derived transmembrane domain and cytoplasmic tail

In some embodiments, the viral particles of the present disclosure are generated using a viral envelope expression cassette encoding, in 5′ to 3′ order:
(a) a Gaussia luciferase derived signal peptide
(b) an anti-CD3 scFV
(c) a short hinge domain
(d) a Cocal glycoprotein derived transmembrane domain and cytoplasmic tail
(e) a T2A linker
(f) a Cocal glycoprotein

In some embodiments, a viral expression cassette described herein comprises a nucleotide sequence encoding a co-stimulatory molecule and/or an adhesion molecule. In some embodiments, a viral expression cassette described herein comprises a nucleotide sequence encoding a CD86 polypeptide. In some embodiments, a viral expression cassette described herein comprises a nucleotide sequence encoding a CD80 polypeptide. In some embodiments, a viral expression cassette described herein comprises a nucleotide sequence encoding a CD58 polypeptide.

In some embodiments, the viral expression cassette comprises a nucleic acid encoding a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 108.

In some embodiments, the viral expression cassette comprises a nucleic acid encoding a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 110.

In some embodiments, the viral expression cassette comprises a nucleic acid encoding a Gaussia luciferase signal peptide, operably linked to an anti-CD3 scFv, operably linked to a linker, operably linked to a Glycophorin A derived transmembrane domain and HIV envelope derived cytoplasmic tail that shares at least 75%, at least 80%, at least 85%, at least 90%, at least 95%, at least 99%, or 100% identity to SEQ ID NO: 112.

In some embodiments, the viral expression cassette comprises a nucleic acid encoding a Gaussia luciferase signal peptide, operably linked to an anti-CD3 scFv, operably linked to a linker, operably linked to a HIV envelope derived transmembrane domain and cytoplasmic tail that shares at least 75%, at least 80%, at least 85%, at least 90%, at least 95%, at least 99%, or 100% identity to SEQ ID NO: 114.

In some embodiments, the viral expression cassette comprises a nucleic acid encoding a Gaussia luciferase signal peptide, operably linked to an anti-CD3 scFv, operably linked to a triple G4Slinker, operably linked to a HIV envelope derived transmembrane domain and cytoplasmic tail that shares at least 75%, at least 80%, at least 85%, at least 90%, at least 95%, at least 99%, or 100% identity to SEQ ID NO: 116.

In some embodiments, the viral expression cassette comprises a nucleic acid encoding a Gaussia luciferase signal peptide, operably linked to an anti-CD3 scFv, operably linked to a hinge domain, operably linked to a Cocal envelope derived transmembrane domain, cytoplasmic tail, and T2A self-cleaving peptide, operably linked to a Cocal envelope that shares at least 75%, at least 80%, at least 85%, at least 90%, at least 95%, at least 99%, or 100% identity to SEQ ID NO: 118.

In some embodiments, the viral expression cassette comprises a nucleic acid encoding a Gaussia luciferase signal peptide, operably linked to an anti-CD3 scFv, operably linked to a linker, operably linked to a Glycophorin A derived hinge, transmembrane domain, and cytoplasmic tail that shares at least 75%, at least 80%, at least 85%, at least 90%, at least 95%, at least 99%, or 100% identity to SEQ ID NO: 120.

In some embodiments, the viral expression cassette comprises a nucleic acid sequence encoding a short hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 16.

In some embodiments, the viral expression cassette comprises a nucleic acid sequence encoding a long hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 22.

In some embodiments, the viral expression cassette comprises a nucleic acid sequence encoding a 218 linker operably linked to a human Glycophorin A ectodomain transmembrane domain operably linked to a cytoplasmic tail derived from a HIV viral envelope that shares at least 75%, at least 80%, at least 85%, at least 90%, at least 95%, at least 99%, or 100% identity to SEQ ID NO: 28.

In some embodiments, the viral parti viral expression cassette cle comprises a nucleic acid sequence encoding a 218 linker operably linked to a HIV viral envelope transmembrane domain and cytoplasmic tail that shares at least 75%, at least 80%, at least 85%, at least 90%, at least 95%, at least 99%, or 100% identity to SEQ ID NO: 34.

In some embodiments, the viral expression cassette comprises a nucleic acid sequence encoding a triple G4S linker operably linked to a HIV viral envelope transmembrane domain and cytoplasmic tail that shares at least 75%, at least 80%, at least 85%, at least 90%, at least 95%, at least 99%, or 100% identity to SEQ ID NO: 40.

In some embodiments, the viral expression cassette comprises a nucleic acid sequence encoding a Ser-Gly peptide operably linked to small ectodomain, transmembrane and cytoplasmic tail sequences derived from human Glycophorin A that shares that shares at least 75%, at least 80%, at least 85%, at least 90%, at least 95%, at least 99%, or 100% identity to SEQ ID NO: 98.

In some embodiments, the viral expression cassette comprises a nucleic acid sequence encoding a hinge operably linked to a Glycophorin A transmembrane domain and cytoplasmic tail that shares at least 75%, at least 80%, at least 85%, at least 90%, at least 95%, at least 99%, or 100% identity to SEQ ID NO: 106.

In some embodiments, the viral expression cassette comprises a nucleic acid sequence encoding a short hinge operably linked to a Cocal glycoprotein transmembrane domain and cytoplasmic tail operably linked to a T2A linker that shares at least 75%, at least 80%, at least 85%, at least 90%, at least 95%, at least 99%, or 100% identity to SEQ ID NO: 47.

In some embodiments, the viral expression cassette comprises a nucleic acid sequence encoding a CD4 derived transmembrane domain and cytoplasmic tail operably linked to a T2A linker that shares at least 75%, at least 80%, at least 85%, at least 90%, at least 95%, at least 99%, or 100% identity to SEQ ID NO: 9.

In some embodiments, the viral expression cassette comprises a nucleic acid sequence encoding a Gaussia luciferase derived signal peptide that shares at least 75%, at least 80%, at least 85%, at least 90%, at least 95%, at least 99%, or 100% identity to SEQ ID NO: 14.

### Types of Particles

In some embodiments, a viral particle described herein is a retrovirus. Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Illustrative lentiviruses include but are not limited to: HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, the backbones are HIV-based vector backbones (i.e., HIV cis-acting sequence elements). Retroviral particles have been generated by multiply attenuating the HIV virulence genes, for example, the genes env, vif, vpr, vpu and nef are deleted, making the vector biologically safe.

Illustrative lentiviral particles include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these particles are configured to carry the essential sequences for selection of cells containing the particle, for incorporating foreign nucleic acid into a lentiviral particle, and for transfer of the nucleic acid into a target cell.

A commonly used lentiviral particles system is the so-called third-generation system. Third-generation lentiviral particles systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting particles replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. In an exemplary third-generation system, the Env gene is Cocal G protein (Cocal glycoprotein) and the promoter is the MND (myeloproliferative sarcoma virus enhancer, negative control region deleted, dl587rev primer-binding site substituted) promoter. In an exemplary third-generation system, the Env gene is Cocal G protein (Cocal glycoprotein) and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral particle systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral particle system into retroviral particles.

As used herein, the terms “retroviral particle” or “lentiviral particle” refers to a viral particle that includes a polynucleotide encoding a heterologous protein (e.g. a chimeric antigen receptor), one or more capsid proteins, and other proteins necessary for transduction of the polynucleotide into a target cell. Retroviral particles and lentiviral particles generally include an RNA genome (derived from the transfer plasmid), a lipid-bilayer envelope in which the Env protein is embedded, and other accessory proteins including integrase, protease, and matrix protein.

The ex vivo efficiency of a retroviral or lentiviral particle system may be assessed in various ways known in the art, including measurement of vector copy number (VCN) or vector genomes (vg) such as by quantitative polymerase chain reaction (qPCR), digital droplet PCR (ddPCR) or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to a hapten. In some embodiments, a sequence encoding a receptor that specifically binds to the hapten is operatively linked to a promoter. Illustrative promoters include, without limitation, a cytomegalovirus (CMV) promoter, a CAG promoter, an SV40 promoter, an SV40/CD43 promoter, an EF-1α promoter, and a MND promoter.

In some embodiments, the polynucleotide encoding the chimeric antigen receptor is operatively linked to one or more promoters. In some embodiments, the promoter is an inducible promoter. In some embodiments, the promoter is CMV. In some embodiments, the promoter is MND.

In some embodiments, the polynucleotide encoding the RACR is operatively linked to one or more promoters. In some embodiments, the promoter is an inducible promoter. In some embodiments, the promoter is CMV. In some embodiments, the promoter is MND.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a hapten-binding receptor. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR.

Gene delivery viral particles useful in the practice of the present disclosure can be constructed utilizing methodologies known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Examples of virus packaging cells include but are not limited to HeLa cells, SF9 cells (optionally with a baculovirus helper vector), 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

Illustrative examples of viral vectors usable in the compositions and methods of the present disclosure are disclosed in WO2016/139463; WO2017/165245; WO2018111834; each of which is incorporated herein in its entirety.

### Payload

In some embodiments, the viral particle comprises a payload. In some embodiments, the payload is conjugated to the surface of the particle. In some embodiments, the payload is encapsulated by the particle. In some embodiments, the viral particle delivers a payload to a target cell.

In some embodiments, the payload is a nucleic acid. In some embodiments, the nucleic acid is a coding nucleic acid. In some embodiments, the nucleic acid encodes a polypeptide of interest. In some embodiments, the polypeptide of interest is a therapeutic polypeptide. In some embodiments, the polypeptide of interest is a chimeric antigen receptor. In some embodiments, the nucleic acid is transduced into a target cell and the polypeptide of interest is expressed in the target cell. In some embodiments, the nucleic acid is a non-coding nucleic acid. In some embodiments, then nucleic acid is a therapeutic non-coding nucleic acid. Non-coding nucleic acids are known to those of skill in the art and include but are not limited to siRNA, miRNA, and shRNA.

In some embodiments, expression of a payload is driven by a promoter. In some embodiments, the promoter is the MND promoter (myeloproliferative sarcoma virus enhancer, negative control region deleted, dl587rev primer-binding site substituted), which is a viral-derived synthetic promoter that contains the U3 region of a modified Moloney murine leukemia virus (MoMuLV) LTR with myeloproliferative sarcoma virus enhancer13 and has high expression in human CD34+ stem cells, lymphocytes, and other tissues. In some embodiments, separate proteins are expressed, separated by 2A peptide sequences that induce ribosomal skipping and cleavage during translation. In some embodiments, the promoter is a CMV promoter. In some embodiments, the promoter is the EF1a promoter. In other embodiments, the promoter is an HTLV promoter.

### Chimeric Antigen Receptors

In some embodiments, the viral particles described herein are used to transduce a nucleic acid sequence (polynucleotide) encoding one or more chimeric antigen receptor (CARs) into a cell (e.g., a T lymphocyte). In some embodiments, the transduction of the viral particle results in expression of one or more CARs in the transduced cells.

CARs are artificial membrane-bound proteins that direct a T lymphocyte to an antigen and stimulate the T lymphocyte to kill cells displaying the antigen. See, e.g., Eshhar, U.S. Pat. No. 7,741,465. Generally, CARs are genetically engineered receptors comprising an extracellular domain that binds to an antigen, e.g., an antigen on a cell, an optional linker, a transmembrane domain, and an intracellular (cytoplasmic) domain comprising a costimulatory domain and/or a signaling domain that transmits an activation signal to an immune cell. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell. All other conditions being satisfied, when a CAR is expressed on the surface of, e.g., a T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because T lymphocytes require two signals, a primary activation signal and a costimulatory signal, in order to maximally activate, CARs can comprise a stimulatory and a costimulatory domain such that binding of the antigen to the extracellular domain results in transmission of both a primary activation signal and a costimulatory signal. Illustrative CARs are known in the art and may de designed in a modular fashion, e.g. as described in (see, e.g., Guedan S, Calderon H, Posey A D, Maus M V, Molecular Therapy—Methods & Clinical Development. 2019; 12: 145-156), incorporated by reference.

In some embodiments, a viral particle disclosed herein encodes a CAR comprising an extracellular domain, optionally a hinge domain, a transmembrane domain, and an intracellular signaling domain. In some embodiments, the intracellular signaling domain comprises a costimulatory domain and an activation domain. In some embodiments, the costimulatory and activation domains are a single domain, for example a single intracellular domain that provides both costimulation and activation signals to a cell. In other embodiments, the intracellular signaling domain comprises either a costimulatory domain or an activation domain. In some embodiments, the CAR comprises an extracellular domain, a CD8a hinge, a CD8a transmembrane domain, a 4-1BB costimulatory domain, and a CD3zeta signaling domain. In some embodiments, a viral particle disclosed herein encodes an extracellular domain, an CD28 hinge domain, a CD28 transmembrane domain, a CD28 co-stimulatory domain, and a CD3zeta signaling domain. In some embodiments, a viral particle disclosed herein encodes an extracellular domain, an IgG4 hinge domain, a CD28 transmembrane domain, a 4-1BB co-stimulatory domain, and a CD3zeta signaling domain. In some embodiments, a viral particle disclosed herein encodes a CAR comprising an extracellular domain, a CD8a hinge, a CD28 transmembrane domain, a 4-1BB costimulatory domain, and a CD3zeta signaling domain.

Illustrative CAR constructs suitable for CAR cells, including CAR T and CAR NK cells, are provided below:
(1) scFv-CD8_{TM}-4-1BB_{IC}-CD3ζs (see, e.g., Liu E, Tong Y, Dotti G, et al., Leukemia. 2018; 32: 520-531);
(2) scFv-CD28_{TM+IC}-CD3ζs (see, e.g., Han J, Chu J, Keung C W et al., Sci Rep. 2015; 5: 11483; Kruschinski A, Moosmann A, Poschke I et al., Proc Natl Acad Sci USA. 2008; 105: 17481-17486; and Chu J, Deng Y, Benson D M et al., Leukemia. 2014; 28: 917-927);
(3) scFv-DAP12_{TM+IC} (see, e.g., Muller N, Michen S, Tietze S et al., J Immunother. 2015; 38: 197-210);
(4) scFv-CD8_{TM}-2B4_{IC}-CD3ζs (see, e.g., Xu Y, Liu Q, Zhong M et al., J Hematol Oncol. 2019; 12: 49);
(5) scFv-2B4_{TM}+IC-CD3ζs (see, e.g., Altvater B, Landmeier S, Pscherer S et al., Clin Cancer Res. 2009; 15: 4857-4866);
(6) scFv-CD28_{TM+IC}-4-1BB_{IC}-CD3ζs (see, e.g., Kloss S, Oberschmidt O, Morgan M et al., Hum Gene Ther. 2017; 28: 897-913);
(7) scFv-CD16_{TM}-2B4_{IC}-CD3ζs (see, e.g., Li Y, Hermanson D L, Moriarity B S Kaufman D S, Cell Stem Cell. 2018; 23: 181-192);
(8) scFv-NKp44_{TM}-DAP10_{IC}-CD3ζs (see, e.g., Li Y, Hermanson D L, Moriarity B S Kaufman D S, Cell Stem Cell. 2018; 23: 181-192);
(9) scFv-NKp46_{TM}-2B4_{IC}-CD3ζs (see, e.g., Li Y, Hermanson D L, Moriarity B S Kaufman D S, Cell Stem Cell. 2018; 23: 181-192);
(10) scFv-NKG2D_{TM}-2B4_{IC}-CD3ζs (see, e.g., Li Y, Hermanson D L, Moriarity B S Kaufman D S, Cell Stem Cell. 2018; 23: 181-192);
(11) scFv-NKG2D_{TM}-4-1BB_{IC}-CD3ζs (see, e.g., Li Y, Hermanson D L, Moriarity B S Kaufman D S, Cell Stem Cell. 2018; 23: 181-192);
(12) scFv-NKG2D_{TM}-2B4_{IC}-DAP12_{IC}-CD3ζs (see, e.g., Li Y, Hermanson D L, Moriarity B S Kaufman D S, Cell Stem Cell. 2018; 23: 181-192);
(13) scFv-NKG2D_{TM}-2B4_{IC}-DAP10_{IC}-CD3ζs (see, e.g., Li Y, Hermanson D L, Moriarity B S Kaufman D S, Cell Stem Cell. 2018; 23: 181-192);
(14) scFv-NKG2D_{TM}-4-1BB_{IC}-2B4_{IC}-CD3ζS (see, e.g., Li Y, Hermanson D L, Moriarity B S Kaufman D S, Cell Stem Cell. 2018; 23: 181-192); and
(15) scFv-NKG2D_{TM}-CD3ζS (see, e.g., Li Y, Hermanson D L, Moriarity B S Kaufman D S, Cell Stem Cell. 2018; 23: 181-192).

### CAR Intracellular Domain

In some embodiments, the intracellular domain of the CAR is or comprises an intracellular domain or motif of a protein that is expressed on the surface of T lymphocytes and triggers activation and/or proliferation of said T lymphocytes. Such a domain or motif is able to transmit a signal that is necessary for the activation of a T lymphocyte in response to antigen binding to the CAR's extracellular portion. In some embodiments, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In some embodiments, the intracellular domain is a CD3ζ intracellular signaling domain. In some embodiments, the intracellular domain is from a lymphocyte receptor chain, a TCR/CD3 complex protein, an Fc receptor subunit or an IL-2 receptor subunit. In some embodiments, the intracellular signaling domain of CAR may be derived from the signaling domains of for example CD3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, the intracellular domain of the CAR is the zeta CD3 chain (CD3zeta).

In some embodiments, the viral particle comprises a polypeptide comprising a CAR whose intracellular domain comprises a CD3zeta domain that shares at least 75%, at least 80%, at least 85%, at least 90%, at least 95%, at least 99%, or 100% identity to SEQ ID NO: 54.

In some embodiments, the viral particle comprises a nucleic acid encoding the intracellular domain of a CAR comprising a CD3zeta domain that shares at least 75%, at least 80%, at least 85%, at least 90%, at least 95%, at least 99%, or 100% identity to SEQ ID NO: 66.

In some embodiments, the CAR additionally comprises one or more co-stimulatory domains or motifs, e.g., as part of the intracellular domain of the polypeptide. Co-stimulatory molecules are well-known cell surface molecules other than antigen receptors or Fc receptors that provide a second signal required for efficient activation and function of T lymphocytes upon binding to antigen. The one or more co-stimulatory domains or motifs can, for example, be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof. In some embodiments, the one or more co-stimulatory domains are selected from the group consisting of intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

In some embodiments, the co-stimulatory domain is an intracellular domain of 4-1BB, CD28, or OX40. Exemplary CAR constructs comprising a CD28 signaling domain are disclosed in U.S. Pat. No. 7,446,190, incorporated by reference. Exemplary CAR constructs comprising a 4-1BB signaling domain are disclosed in U.S. Pat. Nos. 9,856,322 and 8,399,964, each incorporated by reference.

In some embodiments, the viral particle encodes a CAR comprising an IgG4 linker operatively linked to a CD28 transmembrane domain operatively linked to a 4-1BB co-stimulatory domain operatively linked to a CD3zeta signaling domain.

In some embodiments, the viral particle encodes a CAR comprising an IgG4 linker operatively linked to a CD8a transmembrane domain operatively linked to a 4-1BB co-stimulatory domain operatively linked to a CD3zeta signaling domain.

In some embodiments, the viral particle encodes a CAR comprising an IgG4 linker operatively linked to a CD8a transmembrane domain operatively linked to a CD28 co-stimulatory domain operatively linked to a CD3zeta signaling domain.

In some embodiments, the viral particle encodes a CAR comprising an CD8a linker operatively linked to a CD8a transmembrane domain operatively linked to a 4-1BB co-stimulatory domain operatively linked to a CD3zeta signaling domain.

In some embodiments, the viral particle encodes a CAR comprising an CD28 linker operatively linked to a CD28 transmembrane domain operatively linked to a CD28 co-stimulatory domain operatively linked to a CD3zeta signaling domain.

In some embodiments, the viral particle comprises a polypeptide comprising a CAR whose intracellular domain comprises a co-stimulatory 4-1BB polypeptide sequence that shares at least 75%, at least 80%, at least 85%, at least 90%, at least 95%, at least 99%, or 100% identity to SEQ ID NO: 53.

In some embodiments, the viral particle comprises a nucleic acid encoding the intracellular domain of a CAR comprising a co-stimulatory 4-1BB sequence that shares at least 75%, at least 80%, at least 85%, at least 90%, at least 95%, at least 99%, or 100% identity to SEQ ID NO: 65.

In some embodiments, the viral particle comprises a polypeptide comprising a CAR whose intracellular domain comprises an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 80.

In some embodiments, the viral particle comprises a nucleic acid encoding the intracellular domain of a CAR comprising an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 86.

In some embodiments, the viral particle comprises a polypeptide comprising a CAR whose intracellular domain comprises an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 90.

In some embodiments, the viral particle comprises a nucleic acid encoding the intracellular domain of a CAR comprising an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 95.

In some embodiments, the intracellular domain can be further modified to encode a detectable, for example, a fluorescent, protein (e.g., green fluorescent protein) or any variants known thereof.

### CAR Transmembrane Region

The transmembrane region can be any transmembrane region that can be incorporated into a functional CAR, e.g., a transmembrane region from a CD28, CD4, or a CD8 molecule.

In some embodiments, the transmembrane domain of CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of CAR may be derived from the transmembrane domain of CD28. In some embodiments, the transmembrane domain of a CAR may be derived from the transmembrane domain of CD8, for example, CD8a.

### CAR Linker Region

The optional linker or hinge of CAR positioned between the extracellular domain and the transmembrane domain may be a polypeptide of about 2 to over 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Longer linkers may also be advantageous when the target antigen is closer to the cell surface.

In some embodiments, the linker is derived from a hinge region or portion of the hinge region of any immunoglobulin. In some embodiments, the linker is derived from an immunoglobulin, for example, IgG4. In some embodiments, the linker is derived from the extracellular domain of CD28. In other embodiments, the linker is derived from the extracellular domain of CD8.

In some embodiments, the linker is an IgG4 linker operably linked to a CD28 derived transmembrane domain that shares at least 75%, at least 80%, at least 85%, at least 90%, at least 95%, at least 99%, or 100% identity to SEQ ID NO: 52.

In some embodiments, the linker is an IgG4 linker operably linked to a CD28 derived transmembrane domain that shares at least 75%, at least 80%, at least 85%, at least 90%, at least 95%, at least 99%, or 100% identity to SEQ ID NO: 64.

### CAR Extracellular Domain

In some embodiments, the nucleic acid transduced into cells using the methods described herein comprises a sequence that encodes a polypeptide, wherein the extracellular domain of the polypeptide binds to an antigen of interest. In some embodiments, the extracellular domain comprises a receptor, or a portion of a receptor, that binds to said antigen. In some embodiments, the extracellular domain comprises, or is, an antibody or an antigen-binding portion thereof. In some embodiments, the extracellular domain comprises, or is, a single-chain Fv domain. The single-chain Fv domain can comprise, for example, a VL linked to VH by a flexible linker, wherein said VL and VH are from an antibody that binds said antigen.

In some embodiments, the extracellular domain of CAR may contain any polypeptide that binds the desired antigen (e.g. prostate neoantigen or antigen expressed on a tumor of interest). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In some embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. In some embodiments, in which the cancer is chronic lymphocytic leukemia (CLL), the B cells of the CLL have a normal karyotype. In some embodiments, in which the cancer is chronic lymphocytic leukemia (CLL), the B cells of the CLL carry a 17p deletion, an 11q deletion, a 12q trisomy, a 13q deletion or a p53 deletion.

In some embodiments, the antigen is expressed on a B-cell malignancy cell, relapsed/refractory CD19-expressing malignancy cell, diffuse large B-cell lymphoma (DLBCL) cell, Burkitt's type large B-cell lymphoma (B-LBL) cell, follicular lymphoma (FL) cell, chronic lymphocytic leukemia (CLL) cell, acute lymphocytic leukemia (ALL) cell, mantle cell lymphoma (MCL) cell, hematological malignancy cell, colon cancer cell, lung cancer cell, liver cancer cell, breast cancer cell, renal cancer cell, prostate cancer cell, ovarian cancer cell, skin cancer cell, melanoma cell, bone cancer cell, brain cancer cell, squamous cell carcinoma cell, leukemia cell, myeloma cell, B cell lymphoma cell, kidney cancer cell, uterine cancer cell, adenocarcinoma cell, pancreatic cancer cell, chronic myelogenous leukemia cell, glioblastoma cell, neuroblastoma cell, medulloblastoma cell, or a sarcoma cell.

In some embodiments, the antigen is a tumor-associated antigen (TAA) or a tumor-specific antigen (TSA). In some embodiments, without limitation, the tumor-associated antigen or tumor-specific antigen is B cell maturation antigen (BCMA), B cell Activating Factor (BAFF), GPRC5D, FCRL5/FCRH5, ROR1, L1-CAM, CD22, folate receptor, carboxy anhydrase IX (CAIX), claudin 18.2, FAP, mesothelin, IL13Ra2, Lewis Y, CCNA1, WT-1, TACI, CD38, SLAMF7, CD138, DLL3, transmembrane 4 L six family member 1 (TM4SF1), epithelial cell adhesion molecule (EpCAM), PD-1, PD-L1, CTLA-4, AXL, ROR2, glypican-3 (GPC3), CD133, CD147, EGFR, MUC1, GD2, Her2, prostate stem cell antigen (PSCA), prostate-specific membrane antigen (PSMA) alpha-fetoprotein (AFP), carcinoembryonic antigen (CEA), EGFRvIII, cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, vascular endothelial growth factor receptor (VEGFR), the dimeric form of the pyruvate kinase isoenzyme type M2 (tumor M2-PK), an abnormal ras protein, or an abnormal p53 protein. In some embodiments, the CAR comprises binding domains that target two or more antigens as disclosed herein, in any combination. Exemplary antigen combinations include CD19 and CD3, BCMA and CD3, GPRC5D and CD3, FCRL5 and CD3, CD38 and CD3, CD19 and CD20, CD19 and CD22, BCMA and GPRC5D, or CD20 and CD22. In some embodiments, the CAR comprises binding domains that target two or more antigens on the same target protein, for example two epitopes in CD19, BCMA, or any other antigen disclosed herein.

In other embodiments, the CAR is a universal CAR and does not itself specifically target a tumor antigen. For example, the CAR could comprise a tag-specific scFv such that an exogenous agent comprising the tag and a tumor-targeting domain could direct the universal CAR T cell to the target tumor.

In some embodiments, the CAR is a second-generation CAR comprised of an anti-fluorescein scFv linked to the 4-1BB costimulatory domain and the CD3zeta intracellular signaling domain.

In some embodiments, the antigen is CD19. CAR T therapies targeting CD19 have been approved by the FDA and include Yescarta, Tecartus, Kymriah and Breyanzi. CARs targeting CD19 are described, for example, in US Publication No. 20160152723, U.S. Pat. Nos. 10,736,918, 10,357,514, and 7,446,190, each incorporated by reference.

In some embodiments, a CAR comprises an extracellular domain comprising a FMC63 scFv binding domain for CD19 binding. In some embodiments, the CAR is a second-generation CAR comprised of the FMC63 mouse anti-human CD19 scFv linked to the 4-1BB costimulatory domain and the CD3zeta intracellular signaling domain. In some embodiments, a CAR comprises a binding domain for CD19, a CD8a hinge, a CD8a transmembrane domain, a 4-1BB costimulatory domain, and a CD3zeta signaling domain. In some embodiments, a CAR comprises a binding domain for CD19, an IgG4 hinge, a CD28 transmembrane domain, a 4-1BB costimulatory domain, and a CD3zeta signaling domain. In some embodiments, a CAR comprises a binding domain for CD19, a CD28 hinge, a CD28 transmembrane domain, a CD28 costimulatory domain, and CD3zeta signaling domain. In some embodiments, a CAR comprises an extracellular domain comprising a FMC63 scFv binding domain for CD19 binding, a CD8a hinge, a CD8a transmembrane domain, a 4-1BB costimulatory domain, and a CD3zeta signaling domain. In some embodiments, a CAR comprises an extracellular domain comprising a FMC63 scFv binding domain for CD19 binding, an IgG4 hinge, a CD28 transmembrane domain, a 4-1BB costimulatory domain, and a CD3zeta signaling domain. In some embodiments, a CAR comprises an extracellular domain comprising a FMC63 scFv binding domain for CD19 binding, a CD28 hinge, a CD28 transmembrane domain, a CD28 costimulatory domain, and CD3zeta signaling domain.

In some embodiments, the viral particle comprises a polypeptide comprising a CAR whose extracellular domain comprises a signal peptide that shares at least 75%, at least 80%, at least 85%, at least 90%, at least 95%, at least 99%, or 100% identity to SEQ ID NO: 50.

In some embodiments, the viral particle comprises a polynucleotide encoding a CAR whose extracellular domain comprises a αCD19 scFv (CD19 VL linked to a CD19 VH) that shares at least 75%, at least 80%, at least 85%, at least 90%, at least 95%, at least 99%, or 100% identity to SEQ ID NO: 51.

The complementary determining regions (CDR) of this scFv are RASQDISKYLN, (CDR-Li; SEQ ID NO: 39), HTSRLHS (CDR-L2; SEQ ID NO: 41), QQGNTLPYT (CDR-L3; SEQ ID NO: 42), DYGV (CDR-H1; SEQ ID NO: 44), VIWGSETTYYNSALKS (CDR-H2; SEQ ID NO: 45), HYYYGGSYAMDY (CDR-H3; SEQ ID NO: 46). In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 51.

In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 51 or 89.

In some embodiments, the viral particle comprises a nucleic acid encoding a signal peptide for the extracellular domain of CAR that shares at least 75%, at least 80%, at least 85%, at least 90%, at least 95%, at least 99%, or 100% identity to SEQ ID NO: 62.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO: 63.

In some embodiments, the viral particle comprises a polypeptide comprising a CAR whose extracellular domain comprises a αCD19 scFv that shares at least 75%, at least 80%, at least 85%, at least 90%, at least 95%, at least 99%, or 100% identity to SEQ ID NO: 79.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO: 85.

In some embodiments, the viral particle comprises a polynucleotide encoding a CAR whose extracellular domain comprises a αCD19 scFv that shares at least 75%, at least 80%, at least 85%, at least 90%, at least 95%, at least 99%, or 100% identity to SEQ ID NO: 89.

The complementary determining regions (CDR) of this scFv are RASQDISKYLN, (CDR-Li; SEQ ID NO: 39), HTSRLHS (CDR-L2; SEQ ID NO: 41), QQGNTLPYT (CDR-L3; SEQ ID NO: 42), DYGV (CDR-H1; SEQ ID NO: 44), VIWGSETTYYNSALKS (CDR-H2; SEQ ID NO: 45), HYYYGGSYAMDY (CDR-H3; SEQ ID NO: 46). In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 89.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO: 94.

In some embodiments, the CAR is a second-generation CAR comprised of the FMC63 mouse anti-human CD19 scFv linked to the CD28 costimulatory domain and the CD3zeta intracellular signaling domain. In some embodiments, the CAR is a second-generation CAR comprised of the FMC63 mouse anti-human CD19 scFv linked to a CD8 transmembrane domain, 4-1BB costimulatory domain, and the CD3zeta intracellular signaling domain.

In some embodiments, the CAR is a anti-FITC CAR and the ligand is composed of a fluorescein or fluorescein isothiocyanate (FITC) moiety conjugated to an agent that binds to a desired target cell (such as a cancer cell). Exemplary ligands are described in the section above. In some embodiments, the ligand is FITC-folate.

In some embodiments, the CAR comprises an scFv domain. In some embodiments, the scFv domain comprises anti-fluorescein isothiocyanate (FITC) E2. In some embodiments, the scFv domain comprises a light chain variable domain (VL), a linker, and a heavy chain variable domain (VH).

In some embodiments, the scFv VL comprises a nucleotide sequence at least 80%, 85%, 90%, 95%, 96%, 97%, 98%, 99% or 100% identical to the nucleotide sequence of SEQ ID NOs: 157 or 164. In some embodiments, the scFv VL comprises a nucleotide sequence at least 80% identical to the nucleotide sequence of SEQ ID NOs: 157 or 164. In some embodiments, the scFv VL comprises a nucleotide sequence at least 85% identical to the nucleotide sequence of SEQ ID NOs: 157 or 164. In some embodiments, the scFv VL comprises a nucleotide sequence at least 90% identical to the nucleotide sequence of SEQ ID NOs: 157 or 164. In some embodiments, the scFv VL comprises a nucleotide sequence at least 95% identical to the nucleotide sequence of SEQ ID NOs: 157 or 164. In some embodiments, the scFv VL comprises a nucleotide sequence at least 96% identical to the nucleotide sequence of SEQ ID NOs: 157 or 164. In some embodiments, the scFv VL comprises a nucleotide sequence at least 97% identical to the nucleotide sequence of SEQ ID NOs: 157 or 164. In some embodiments, the scFv VL comprises a nucleotide sequence at least 98% identical to the nucleotide sequence of SEQ ID NOs: 157 or 164. In some embodiments, the scFv VL comprises a nucleotide sequence at least 99% identical to the nucleotide sequence of SEQ ID NOs: 157 or 164. In some embodiments, the scFv VL comprises a nucleotide sequence at least 100% identical to the nucleotide sequence of SEQ ID NOs: 157 or 164. In some embodiments, the scFv VL comprises the nucleotide sequence of SEQ ID NOs: 157 or 164. In some embodiments, the scFv VL consists of the nucleotide sequence of SEQ ID NOs: 157 or 164.

In some embodiments, the scFv VL comprises an amino acid sequence at least 80%, 85%, 90%, 95%, 96%, 97%, 98%, 99% or 100% identical to the amino acid sequence of SEQ ID NO: 158. In some embodiments, the scFv VL comprises an amino acid sequence at least 80% identical to the amino acid sequence of SEQ ID NO: 158. In some embodiments, the scFv VL comprises an amino acid sequence at least 85% identical to the amino acid sequence of SEQ ID NO: 158. In some embodiments, the scFv VL comprises an amino acid sequence at least 90% identical to the amino acid sequence of SEQ ID NO: 158. In some embodiments, the scFv VL comprises an amino acid sequence at least 95% identical to the amino acid sequence of SEQ ID NO: 158. In some embodiments, the scFv VL comprises an amino acid sequence at least 96% identical to the amino acid sequence of SEQ ID NO: 158. In some embodiments, the scFv VL comprises an amino acid sequence at least 97% identical to the amino acid sequence of SEQ ID NO: 158. In some embodiments, the scFv VL comprises an amino acid sequence at least 98% identical to the amino acid sequence of SEQ ID NO: 158. In some embodiments, the scFv VL comprises an amino acid sequence at least 99% identical to the amino acid sequence of SEQ ID NO: 158. In some embodiments, the scFv VL comprises an amino acid sequence at least 100% identical to the amino acid sequence of SEQ ID NO: 158. In some embodiments, the scFv VL comprises the amino acid sequence of SEQ ID NO: 158. In some embodiments, the scFv VL consists the amino acid sequence of SEQ ID NO: 158.

In some embodiments, the scFv VH comprises a nucleotide sequence at least 80%, 85%, 90%, 95%, 96%, 97%, 98%, 99% or 100% identical to the nucleotide sequence of SEQ ID NOs: 161 or 166. In some embodiments, the scFv VH comprises a nucleotide sequence at least 80% identical to the nucleotide sequence of SEQ ID NOs: 161 or 166. In some embodiments, the scFv VH comprises a nucleotide sequence at least 85% identical to the nucleotide sequence of SEQ ID NOs: 161 or 166. In some embodiments, the scFv VH comprises a nucleotide sequence at least 90% identical to the nucleotide sequence of SEQ ID NOs: 161 or 166. In some embodiments, the scFv VH comprises a nucleotide sequence at least 95% identical to the nucleotide sequence of SEQ ID NOs: 161 or 166. In some embodiments, the scFv VH comprises a nucleotide sequence at least 96% identical to the nucleotide sequence of SEQ ID NOs: 161 or 166. In some embodiments, the scFv VH comprises a nucleotide sequence at least 97% identical to the nucleotide sequence of SEQ ID NOs: 161 or 166. In some embodiments, the scFv VH comprises a nucleotide sequence at least 98% identical to the nucleotide sequence of SEQ ID NOs: 161 or 166. In some embodiments, the scFv VH comprises a nucleotide sequence at least 99% identical to the nucleotide sequence of SEQ ID NOs: 161 or 166. In some embodiments, the scFv VH comprises a nucleotide sequence at least 100% identical to the nucleotide sequence of SEQ ID NOs: 161 or 166. In some embodiments, the scFv VH comprises the nucleotide sequence of SEQ ID NOs: 161 or 166. In some embodiments, the scFv VH consists of the nucleotide sequence of SEQ ID NOs: 161 or 166.

In some embodiments, the scFv VH comprises an amino acid sequence at least 80%, 85%, 90%, 95%, 96%, 97%, 98%, 99% or 100% identical to the amino acid sequence of SEQ ID NO: 162. In some embodiments, the scFv VH comprises an amino acid sequence at least 80% identical to the amino acid sequence of SEQ ID NO: 162. In some embodiments, the scFv VH comprises an amino acid sequence at least 85% identical to the amino acid sequence of SEQ ID NO: 162. In some embodiments, the scFv VH comprises an amino acid sequence at least 90% identical to the amino acid sequence of SEQ ID NO: 162. In some embodiments, the scFv VH comprises an amino acid sequence at least 95% identical to the amino acid sequence of SEQ ID NO: 162. In some embodiments, the scFv VH comprises an amino acid sequence at least 96% identical to the amino acid sequence of SEQ ID NO: 162. In some embodiments, the scFv VH comprises an amino acid sequence at least 97% identical to the amino acid sequence of SEQ ID NO: 162. In some embodiments, the scFv VH comprises an amino acid sequence at least 98% identical to the amino acid sequence of SEQ ID NO: 162. In some embodiments, the scFv VH comprises an amino acid sequence at least 99% identical to the amino acid sequence of SEQ ID NO: 162. In some embodiments, the scFv VH comprises an amino acid sequence at least 100% identical to the amino acid sequence of SEQ ID NO: 162. In some embodiments, the scFv VH comprises the amino acid sequence of SEQ ID NO: 162. In some embodiments, the scFv VH consists the amino acid sequence of SEQ ID NO: 162.

In some embodiments, the scFv linker comprises a nucleotide sequence at least 80%, 85%, 90%, 95%, 96%, 97%, 98%, 99% or 100% identical to the nucleotide sequence of SEQ ID NOs: 159 or 165. In some embodiments, the scFv linker comprises a nucleotide sequence at least 80% identical to the nucleotide sequence of SEQ ID NOs: 159 or 165. In some embodiments, the scFv linker comprises a nucleotide sequence at least 85% identical to the nucleotide sequence of SEQ ID NOs: 159 or 165. In some embodiments, the scFv linker comprises a nucleotide sequence at least 90% identical to the nucleotide sequence of SEQ ID NOs: 159 or 165. In some embodiments, the scFv linker comprises a nucleotide sequence at least 95% identical to the nucleotide sequence of SEQ ID NOs: 159 or 165. In some embodiments, the scFv linker comprises a nucleotide sequence at least 96% identical to the nucleotide sequence of SEQ ID NOs: 159 or 165. In some embodiments, the scFv linker comprises a nucleotide sequence at least 97% identical to the nucleotide sequence of SEQ ID NOs: 159 or 165. In some embodiments, the scFv linker comprises a nucleotide sequence at least 98% identical to the nucleotide sequence of SEQ ID NOs: 159 or 165. In some embodiments, the scFv linker comprises a nucleotide sequence at least 99% identical to the nucleotide sequence of SEQ ID NOs: 159 or 165. In some embodiments, the scFv linker comprises a nucleotide sequence at least 100% identical to the nucleotide sequence of SEQ ID NOs: 159 or 165. In some embodiments, the scFv linker comprises the nucleotide sequence of SEQ ID NOs: 159 or 165. In some embodiments, the scFv linker consists the nucleotide sequence of SEQ ID NOs: 159 or 165.

In some embodiments, the scFv linker comprises an amino acid sequence at least 80%, 85%, 90%, 95%, 96%, 97%, 98%, 99% or 100% identical to the amino acid sequence of SEQ ID NO: 160. In some embodiments, the scFv linker comprises an amino acid sequence at least 80% identical to the amino acid sequence of SEQ ID NO: 160. In some embodiments, the scFv linker comprises an amino acid sequence at least 85% identical to the amino acid sequence of SEQ ID NO: 160. In some embodiments, the scFv linker comprises an amino acid sequence at least 90% identical to the amino acid sequence of SEQ ID NO: 160. In some embodiments, the scFv linker comprises an amino acid sequence at least 95% identical to the amino acid sequence of SEQ ID NO: 160. In some embodiments, the scFv linker comprises an amino acid sequence at least 96% identical to the amino acid sequence of SEQ ID NO: 160. In some embodiments, the scFv linker comprises an amino acid sequence at least 97% identical to the amino acid sequence of SEQ ID NO: 160. In some embodiments, the scFv linker comprises an amino acid sequence at least 98% identical to the amino acid sequence of SEQ ID NO: 160. In some embodiments, the scFv linker comprises an amino acid sequence at least 99% identical to the amino acid sequence of SEQ ID NO: 160. In some embodiments, the scFv linker comprises an amino acid sequence at least 100% identical to the amino acid sequence of SEQ ID NO: 160. In some embodiments, the scFv linker comprises the amino acid sequence of SEQ ID NO: 160. In some embodiments, the scFv linker consists the amino acid sequence of SEQ ID NO: 160.

In some embodiments, the scFv comprises a nucleotide sequence at least 80%, 85%, 90%, 95%, 96%, 97%, 98%, 99% or 100% identical to the nucleotide sequence of SEQ ID NOs: 155 or 163. In some embodiments, the scFv comprises a nucleotide sequence at least 80% identical to the nucleotide sequence of SEQ ID NOs: 155 or 163. In some embodiments, the scFv comprises a nucleotide sequence at least 85% identical to the nucleotide sequence of SEQ ID NOs: 155 or 163. In some embodiments, the scFv comprises a nucleotide sequence at least 90% identical to the nucleotide sequence of SEQ ID NOs: 155 or 163. In some embodiments, the scFv comprises a nucleotide sequence at least 95% identical to the nucleotide sequence of SEQ ID NOs: 155 or 163. In some embodiments, the scFv comprises a nucleotide sequence at least 96% identical to the nucleotide sequence of SEQ ID NOs: 155 or 163. In some embodiments, the scFv comprises a nucleotide sequence at least 97% identical to the nucleotide sequence of SEQ ID NOs: 155 or 163. In some embodiments, the scFv comprises a nucleotide sequence at least 98% identical to the nucleotide sequence of SEQ ID NOs: 155 or 163. In some embodiments, the scFv comprises a nucleotide sequence at least 99% identical to the nucleotide sequence of SEQ ID NOs: 155 or 163. In some embodiments, the scFv comprises a nucleotide sequence at least 100% identical to the nucleotide sequence of SEQ ID NOs: 155 or 163. In some embodiments, the scFv comprises the nucleotide sequence of SEQ ID NOs: 155 or 163. In some embodiments, the scFv consists of the nucleotide sequence of SEQ ID NOs: 155 or 163.

In some embodiments, the scFv comprises an amino acid sequence at least 80%, 85%, 90%, 95%, 96%, 97%, 98%, 99% or 100% identical to the amino acid sequence of SEQ ID NO: 156. In some embodiments, the scFv comprises an amino acid sequence at least 80% identical to the amino acid sequence of SEQ ID NO: 156. In some embodiments, the scFv comprises an amino acid sequence at least 85% identical to the amino acid sequence of SEQ ID NO: 156. In some embodiments, the scFv comprises an amino acid sequence at least 90% identical to the amino acid sequence of SEQ ID NO: 156. In some embodiments, the scFv comprises an amino acid sequence at least 95% identical to the amino acid sequence of SEQ ID NO: 156. In some embodiments, the scFv comprises an amino acid sequence at least 96% identical to the amino acid sequence of SEQ ID NO: 156. In some embodiments, the scFv comprises an amino acid sequence at least 97% identical to the amino acid sequence of SEQ ID NO: 156. In some embodiments, the scFv comprises an amino acid sequence at least 98% identical to the amino acid sequence of SEQ ID NO: 156. In some embodiments, the scFv comprises an amino acid sequence at least 99% identical to the amino acid sequence of SEQ ID NO: 156. In some embodiments, the scFv comprises an amino acid sequence at least 100% identical to the amino acid sequence of SEQ ID NO: 156. In some embodiments, the scFv comprises the amino acid sequence of SEQ ID NO: 156. In some embodiments, the scFv consists of the amino acid sequence of SEQ ID NO: 156.

In some embodiments, the anti-fluorescein E2 scFv comprises a CDRL1, CDRL2, and CDRL3 having at least 80% amino acid identity, at least 90% amino acid identity or at least 95% amino acid identity to: TSNIGNNYVS (SEQ ID NO: 167), LMIYDVSKRPS (SEQ ID NO: 168), and AAWDDSLSEF (SEQ ID NO: 169), respectively, and CDRH1, CDRH2, and CDRH3 having at least 80% amino acid identity, at least 90% amino acid identity or at least 95% amino acid identity to: FTFGSFSMS (SEQ ID NO: 170), WVAGLSARSSLTHY (SEQ ID NO: 171), and RRSYDSSGYWGHFYSYMDV (SEQ ID NO: 172), respectively.

In some embodiments, the antigen is BCMA. CAR T therapies targeting BCMA have been approved by the FDA and include Abecma and Carvykti. CARs targeting BCMA are described, for example, in US Publication No. 2020/0246381; U.S. Pat. No. 10,918,665; US Publication No. 2019/0161553, each of which is herein incorporated by reference. In some embodiments, a CAR comprises a binding domain for BCMA, a CD8a hinge, a CD8a transmembrane domain, a 4-1BB costimulatory domain, and a CD3zeta signaling domain. In some embodiments, a CAR comprises a binding domain for BCMA, an IgG4 hinge, a CD28 transmembrane domain, a 4-1BB costimulatory domain, and a CD3zeta signaling domain. In some embodiments, a CAR comprises a binding domain for BCMA, a CD28 hinge, a CD28 transmembrane domain, a CD28 costimulatory domain, and CD3zeta signaling domain.

In some embodiments, the antigen is G protein-coupled receptor class C group 5 member D (GPRC5D). CARs targeting GRC5D are described, for example, in US Publication Nos. 2018/0118803 and 2021/10393689, each of which is herein incorporated by reference. In some embodiments, a CAR comprises a binding domain for GRC5D, a CD8a hinge, a CD8a transmembrane domain, a 4-1BB costimulatory domain, and a CD3zeta signaling domain. In some embodiments, a CAR comprises a binding domain for GRC5D, an IgG4 hinge, a CD28 transmembrane domain, a 4-1BB costimulatory domain, and a CD3zeta signaling domain. In some embodiments, a CAR comprises a binding domain for GRC5D, a CD28 hinge, a CD28 transmembrane domain, a CD28 costimulatory domain, and CD3zeta signaling domain.

In some embodiments, the antigen is Fe Receptor-like 5 (FcRL5). CARs targeting FcRL5 are described, for example, in US Publication No. US 2017/0275362, which is herein incorporated by reference. In some embodiments, a CAR comprises a binding domain for FcRL5, a CD8a hinge, a CD8a transmembrane domain, a 4-1BB costimulatory domain, and a CD3zeta signaling domain. In some embodiments, a CAR comprises a binding domain for FcRL5, an IgG4 hinge, a CD28 transmembrane domain, a 4-1BB costimulatory domain, and a CD3zeta signaling domain. In some embodiments, a CAR comprises a binding domain for FcRL5, a CD28 hinge, a CD28 transmembrane domain, a CD28 costimulatory domain, and CD3zeta signaling domain.

In some embodiments, the antigen is receptor tyrosine kinase-like orphan receptor 1 (ROR1). CARs targeting ROR1 are described, for example, in US Publication No. 2022/0096651, which is herein incorporated by reference. In some embodiments, a CAR comprises a binding domain for ROR1, a CD8a hinge, a CD8a transmembrane domain, a 4-1BB costimulatory domain, and a CD3zeta signaling domain. In some embodiments, a CAR comprises a binding domain for ROR1, an IgG4 hinge, a CD28 transmembrane domain, a 4-1BB costimulatory domain, and a CD3zeta signaling domain. In some embodiments, a CAR comprises a binding domain for ROR1, a CD28 hinge, a CD28 transmembrane domain, a CD28 costimulatory domain, and CD3zeta signaling domain.

In some embodiments, the CAR is a second-generation CAR comprised an anti-BCMA scFv linked to the 4-1BB costimulatory domain and the CD3zeta intracellular signaling domain. In some embodiments, the CAR is a second-generation CAR comprised an anti-GPRC5D scFv linked to the 4-1BB costimulatory domain and the CD3zeta intracellular signaling domain. In some embodiments, the CAR is a second-generation CAR comprised an anti-ROR1 scFv linked to the 4-1BB costimulatory domain and the CD3zeta intracellular signaling domain.

In some embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In some embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In some embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAAO205, Mart2, Mum-1, 2, and 3, neo-PAP, myosin class I, OS-9, pm1-RARα fusion protein, PTPRK, K-ras, N-ras, triosephosphate isomerase, Gage 3, 4, 5, 6, 7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD20, CD22, CD27, CD30, CD70, CD123, CD133, B-cell maturation antigen, CS1, GPCR5, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In some embodiments, said tumor-associated antigen or tumor-specific antigen is integrin αvβ3 (CD61), galactin, K-Ras (V-Ki-ras2 Kirsten rat sarcoma viral oncogene), or Ral-B. Other tumor-associated and tumor-specific antigens are known to those in the art.

Antibodies, and scFvs, that bind to TSAs and TAAs include antibodies and scFVs that are known in the art, as are nucleotide sequences that encode them.

In some embodiments, the antigen is an antigen not considered to be a TSA or a TAA, but which is nevertheless associated with tumor cells, or damage caused by a tumor. In some embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in some embodiments, the antigen is a hypoxia-associated factor, e.g., HIF-1α, HIF-1β, HIF-2a, HIF-2β, HIF-3α, or HIF-3β. Tumors can also cause localized damage to normal tissue, causing the release of molecules known as damage associated molecular pattern molecules (DAMPs; also known as alarmins). In some embodiments, therefore, the antigen is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In some embodiments of the polypeptides described herein, the extracellular domain is joined to said transmembrane domain directly or by a linker, spacer or hinge polypeptide sequence, e.g., a sequence from CD28 or a sequence from CTLA4.

In some embodiments, the extracellular domain that binds the desired antigen may be derived from antibodies or their antigen binding fragments generated using the technologies described herein.

### Universal CARs

In some embodiments, the viral particle described herein comprises a nucleotide sequence encoding a universal CAR. Universal CARs allow for targeting to a cancer cell without the need to change the antigen specificity of the CAR. Universal CARs are described, for example, in US Publication Nos. US 2016/0348073, US 2018/0085399, US 2019/0256597, and US 2014/0349402, each of which is herein incorporated by reference.

In some embodiments, the viral particle described herein comprises a nucleotide sequence encoding a universal, modular, ant-tag chimeric antigen receptor (UniCAR). This system allows for retargeting of UniCAR engrafted immune cells against multiple antigens (see e.g., US Patent Publication US20170240612 A1 incorporated herein by reference in its entirety; Cartellieri et al., (2016) Blood Cancer Journal 6, e458 incorporated herein by reference in its entirety).

In some embodiments, the viral particle described herein comprises a nucleotide sequence encoding a switchable CAR and/or CAR effector cell (CAR-EC) switches. In this system, the CAR-EC switches have a first region that is bound by a CAR on the CAR-EC and a second region that binds a cell surface molecule on a target cell, thereby stimulating an immune response from the CAR-EC that is cytotoxic to the bound target cell. In some embodiments, the CAR-EC switch may act as an “on-switch” for CAR-EC activity. Activity may be “turned off” by reducing or ceasing administration of the switch. These CAR-EC switches may be used with CAR-ECs disclosed herein, as well as existing CAR T-cells, for the treatment of a disease or condition, such as cancer, wherein the target cell is a malignant cell. Such treatment may be referred to herein as switchable immunotherapy (U.S. Pat. No. 9,624,276 B2 incorporated herein by reference in its entirety).

In some embodiments, the viral particle comprises a nucleotide sequence encoding a universal immune receptor (e.g., switchable CAR, sCAR) that binds a peptide neo-epitope (PNE). In some embodiments, the peptide neo-epitope (PNE) has been incorporated at defined different locations within an antibody targeting an antigen (antibody switch). Therefore, sCAR-T-cell specificity is redirected only against PNE, not occurring in the human proteome, thus allowing an orthogonal interaction between the sCAR-T-cell and the antibody switch. In this way, sCAR-T cells are strictly dependent on the presence of the antibody switch to become fully activated, thus excluding CAR T-cell off-target recognition of endogenous tissues or antigens in the absence of the antibody switch (Arcangeli et al., (2016) Transl Cancer Res 5 (Suppl 2):S174-S177 incorporated herein by reference in its entirety). Other examples of switchable CARs is provided by US Patent Application US20160272718A1 incorporated herein by reference in its entirety.

As used herein, the term “tag” encompasses a universal immune receptor, a tag, a switch, or an Fc region of an inmmunoglobulin as described supra. In some embodiments, a viral particle comprises a nucleotide sequence encoding a CAR comprising a tag binding domain. In some embodiments, the CAR binds fluorescein isothiocyanate (FITC), streptavidin, biotin, dinitrophenol, peridinin chlorophyll protein complex, green fluorescent protein, phycoerythrin (PE), horse radish peroxidase, painitoylation, nitrosylation, alkalanine phosphatase, glucose oxidase, or maltose binding protein.

In some embodiments, the viral particle comprises a nucleotide sequence encoding a CAR to generate CAR cells to be used with a targeting small molecule. In some embodiments, the CAR targets a moiety that is not produced or expressed by cells of the subject being treated. This CAR system thus allows for focused targeting of the immune cells to target cells, such as cancer cells. The two-component CAR system has been previously described in e.g., US 2015/0320799; US 2019/0224237; and US 2020/0023009, each of which is herein incorporated by reference.

In some embodiments, the targeting small molecule comprises a ligand of a tumor cell receptor. By administration of a targeting small molecule along with the CAR-expressing immune cell, the immune cell response can be targeted to only those cells expressing the tumor receptor, thereby reducing off-target toxicity, and the activation of immune cells can be more easily controlled due to the rapid clearance of the targeting small molecule. As an added advantage, the CAR-expressing immune cell can be used as a universal cytotoxic cell to target a wide variety of tumors without the need to prepare separate CAR constructs. The targeting small molecule recognized by the CAR may also remain constant. It is only the ligand portion of the targeting small molecule that needs to be altered to allow the system to target cancer cells of different identity.

In some embodiments, a targeting small molecule comprises fluorescein linked to a ligand of a selected tumor cell receptor. In some embodiments, a targeting small molecule comprises FITC linked to a ligand of a selected tumor cell receptor. In some embodiments, the viral vector described herein encodes a CAR comprising an anti-fluorescein scFv. In some embodiments, the viral vector described herein encodes a CAR comprising an anti-FITC scFv. This CAR thus targets fluorescein or FITC instead of a tumor-associated antigen that might also be expressed by healthy, non-target cells. The two components are administered to a subject having cancer and the targeting small molecule is bound by the target tumor cells (through binding of the ligand portion of the molecule to cognate tumor cell receptor). The FITC portion of the targeting small molecule is then recognized and bound by the anti-FITC CAR expressed by the T cells (second component). Upon binding, the anti-FITC CAR-expressing immune cells are activated and the tumor cell is killed. As will be apparent to the skilled artisan, the immune cells cannot kill cells without first binding to a tumor cell. As it will be further apparent, immune cells will not bind to non-target cells because the recognition region of the CAR will only recognize and bind FITC, which is not produced or expressed by cells of the subject. The targeting small molecule thus acts as a bridge between the immune cells and the target tumor cells. As long as the targeted moiety of the targeting small molecule is a moiety not found in the host, the activity of the immune cells can be limited to the target cells. Further, the activation of the CAR-expressing immune cells can be regulated by limiting the amount of targeting small molecule administered to a subject, for example, by manipulating infusion of the targeting small molecule if a side effect is detected. Exemplary anti-fluorescein and anti-FITC CARs are described in US Patent Application US20200405760A1 incorporated herein by reference in its entirety.

In some embodiments, the targeting small molecule comprises 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody. In some embodiments, the viral vector comprises a nucleotide sequence encoding a CAR comprising an extracellular binding domain that binds 2,4-dinitrophenol (DNP), 2,4,6-trinitrophenol (TNP), biotin, digoxigenin, fluorescein, fluorescein isothiocyanate (FITC). NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody.

In some embodiments, the CAR system utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (K_{D}=14 nM; see Kularatne, S. A. et al., Mol Pharm. 2009. 6(3):780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013, ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27(4):655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

Illustrative conjugates of the disclosure include the following molecules: FITC-(PEG)₁₂-Folate, FITC-(PEG)₂₀-Folate, FITC-(PEG)₁₀₈-Folate, FITC-DUPA, FITC-(PEG)₁₂-DUPA, FITC-CCK2R ligand, FITC-(PEG)₁₂-CCK2R ligand, FITC-(PEG)₁₁-NK1R ligand and FITC-(PEG)₂-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 pM or 10 pM, even more preferably at least about 100 pM.

Examples of conjugates and methods of making them are provided in U.S. patent applications US 2017/0290900, US 2019/0091308, and US 2020/0023009, all of which are incorporated herein by reference.

### Rapamycin-Activated Cell-surface Receptor (RACR)

In some embodiments, the viral particle comprises a nucleotide sequence encoding a multipartite cell-surface receptor. In some embodiments, the multipartite cell-surface receptor is a proliferatory receptor.

In some embodiments, the multipartite cell-surface receptor is a rapamycin-activated cell-surface receptor (RACR).

In some embodiments, the multipartite cell-surface receptor is a chemically inducible cell-surface receptor.

In some embodiments, the multipartite cell-surface receptor comprises a polynucleotide sequence encoding FKBP-rapamycin complex binding domain (FRB domain) or a functional variant thereof. In some embodiments, the multipartite cell-surface receptor further comprises a polynucleotide sequence encoding a FK506 binding protein domain (FKBP) or a functional variant thereof. In some embodiments, the FKBP is FKBP12.

In some embodiments, the viral particle comprises a RACR polypeptide comprising a signal peptide operably linked to FKBP12 that shares at least 75%, at least 80%, at least 85%, at least 90%, at least 95%, at least 99%, or 100% identity to SEQ ID NO: 57.

In some embodiments, the viral particle comprises a RACR polypeptide comprising an IL-2R gamma transmembrane domain operably linked to a cytoplasmic domain that shares at least 75%, at least 80%, at least 85%, at least 90%, at least 95%, at least 99%, or 100% identity to SEQ ID NO: 58.

In some embodiments, the viral particle comprises a RACR polypeptide comprising a P2A self-cleaving peptide that shares at least 75%, at least 80%, at least 85%, at least 90%, at least 95%, at least 99%, or 100% identity to SEQ ID NO: 55.

In some embodiments, the viral particle comprises a RACR polypeptide comprising a signal peptide operably linked to FRB that shares at least 75%, at least 80%, at least 85%, at least 90%, at least 95%, at least 99%, or 100% identity to SEQ ID NO: 59.

In some embodiments, the viral particle comprises a RACR polypeptide comprising an IL-2R beta transmembrane domain operably linked to a cytoplasmic domain that shares at least 75%, at least 80%, at least 85%, at least 90%, at least 95%, at least 99%, or 100% identity to SEQ ID NO: 60.

In some embodiments, the viral particle comprises a nucleic acid encoding a signal peptide operably linked to FKBP12 that shares at least 75%, at least 80%, at least 85%, at least 90%, at least 95%, at least 99%, or 100% identity to SEQ ID NO: 70.

In some embodiments, the viral particle comprises a nucleic acid encoding an IL-2R gamma transmembrane domain operably linked to a cytoplasmic domain that shares at least 75%, at least 80%, at least 85%, at least 90%, at least 95%, at least 99%, or 100% identity to SEQ ID NO: 71.

In some embodiments, the viral particle comprises a nucleic acid encoding a P2A self-cleaving peptide that shares at least 75%, at least 80%, at least 85%, at least 90%, at least 95%, at least 99%, or 100% identity to SEQ ID NO: 72.

In some embodiments, the viral particle comprises a nucleic acid encoding a signal peptide operably linked to FRB that shares at least 75%, at least 80%, at least 85%, at least 90%, at least 95%, at least 99%, or 100% identity to SEQ ID NO: 73.

In some embodiments, the viral particle comprises a nucleic acid encoding an IL-2R beta transmembrane domain operably linked to a cytoplasmic domain that shares at least 75%, at least 80%, at least 85%, at least 90%, at least 95%, at least 99%, or 100% identity to SEQ ID NO: 74.

In some embodiments, the viral particle comprises a RACR polypeptide comprising a FKBP12 operably linked to an IL-2R gamma domain operably linked to a P2A peptide that shares at least 75%, at least 80%, at least 85%, at least 90%, at least 95%, at least 99%, or 100% identity to SEQ ID NO: 77.

In some embodiments, the viral particle comprises a RACR polypeptide comprising a FRB operably linked to an IL-2R beta domain operably linked to a P2A peptide that shares at least 75%, at least 80%, at least 85%, at least 90%, at least 95%, at least 99%, or 100% identity to SEQ ID NO: 78.

In some embodiments, the viral particle comprises a nucleic acid encoding a FKBP12 operably linked to an IL-2R gamma domain operably linked to a P2A that shares at least 75%, at least 80%, at least 85%, at least 90%, at least 95%, at least 99%, or 100% identity to SEQ ID NO: 83.

In some embodiments, the viral particle comprises a nucleic acid encoding a FRB operably linked to an IL-2R beta domain operably linked to a P2A that shares at least 75%, at least 80%, at least 85%, at least 90%, at least 95%, at least 99%, or 100% identity to SEQ ID NO: 84.

In some embodiments, the FKBP domain and FRB domain form a T cell activator protein complex. The complex formed by the FKBP and FRB domains promote growth and/or survival of a cell. In some embodiments, the complex formed by the FKJBP and FRB domains is controlled by a ligand.

In some embodiments, the ligand is rapamycin.

In some embodiments, the FRB domain and FKBP form a tripartite complex with rapamycin that sequesters rapamycin in the transduced cell.

In some embodiments, the ligand is a protein, an antibody, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, or AP1903, or metabolites, derivatives, and/or combinations thereof. In some embodiments, the ligand is an IMID-class drug (e.g., thalidomide, pomalidimide, lenalidomide or related analogues).

In some embodiments, the molecule is selected from FK1012, tacrolimus (FK506), FKCsA, rapamycin, coumermycin, gibberellin, HaXS, TMP-HTag, and ABT-737 or functional derivatives thereof.

In some embodiments, the FKBP domain is operably linked to an IL2R gamma domain. In some embodiments, the FRB domain is operably linked to an IL2R beta domain. In some embodiments, the IL2R gamma domain and IL2R beta domain heterodimerize. In some embodiments, the IL2R gamma domain and IL2R beta domain heterodimerize in the presence of a ligand to promote growth and/or survival of a cell. In some embodiments, the IL2R gamma domain and IL2R beta domain heterodimerize in the presence of rapamycin to promote growth and/or survival of a cell. In some embodiments, the IL2R gamma domain and IL2R beta domain heterodimerize in the presence of rapamycin to promote T cell activation.

### Cytosolic FRB

In some embodiments, vector genome comprises a nucleotide sequence that confers resistance to an immunosuppressive agent.

In some embodiments, the nucleotide that confers resistance to an immunosuppressive agent binds rapamycin. In some embodiments, the polynucleotide that confers resistance to an immunosuppressive agent encodes a cytosolic (“naked”) FRB domain. The naked FRB domain is an approximately 100 amino acid domain extracted from the mTOR protein kinase. It is expressed in the cytosol as a freely diffusible soluble protein. The purpose of the FRB domain is to reduce the inhibitory effects of rapamycin on mTOR in the transduced cells, which should allow for consistent activation of transduced T cells and give them a proliferative advantage over native T cells.

In some embodiments, the viral particle comprises a polypeptide comprising a cytosolic FRB domain that shares at least 75%, at least 80%, at least 85%, at least 90%, at least 95%, at least 99%, or 100% identity to SEQ ID NO: 56.

In some embodiments, the viral particle comprises a nucleic acid encoding a cytosolic FRB domain that shares at least 75%, at least 80%, at least 85%, at least 90%, at least 95%, at least 99%, or 100% identity to SEQ ID NO: 68.

In some embodiments, the viral particle comprises a polypeptide comprising a cytosolic FRB domain operably linked to a P2A peptide that shares at least 75%, at least 80%, at least 85%, at least 90%, at least 95%, at least 99%, or 100% identity to SEQ ID NO:

In some embodiments, the viral particle comprises a nucleic acid encoding a cytosolic FRB domain operable linked to a P2A that shares at least 75%, at least 80%, at least 85%, at least 90%, at least 95%, at least 99%, or 100% identity to SEQ ID NO: 82.

In some embodiments, the viral particle comprises a polypeptide comprising a cytosolic FRB domain operably linked to a P2A peptide that shares at least 75%, at least 80%, at least 85%, at least 90%, at least 95%, at least 99%, or 100% identity to SEQ ID NO: 88.

In some embodiments, the viral particle comprises a nucleic acid encoding a cytosolic FRB domain operable linked to a P2A that shares at least 75%, at least 80%, at least 85%, at least 90%, at least 95%, at least 99%, or 100% identity to SEQ ID NO: 93.

In some embodiments, expression of the chimeric antigen receptor is modulated by a degron fusion polypeptide and wherein suppression of the degron fusion polypeptide is chemically inducible by a ligand.

In some embodiments, expression of the chimeric antigen receptor is modulated by a FRB-degron fusion polypeptide and wherein suppression of the FRB-degron fusion polypeptide is chemically inducible by a ligand.

In some embodiments, the ligand is rapamycin or a rapalog as described herein.

### TGF-β Double Negative (TGF-β DN)

Tumor cells secrete transforming growth factor β (TGF-β) as a means to inhibit immunity while allowing for cancer progression. Blocking TGF-β signaling in T cells increases their ability to infiltrate, proliferate, and mediate antitumor responses (Kloss et al., Mol. Therapy 26(7):1855-1866 (2018)). The dominant-negative TGF-β (TGF-β DN) is truncated and lacks the intracellular domain necessary for downstream signaling

In some embodiments, the viral particle of the present disclosure comprises a polynucleotide sequence of a dominant-negative TGF-β. In some embodiments, the viral particle comprises a polypeptide comprising a dominant-negative TGF-β that shares at least 75%, at least 80%, at least 85%, at least 90%, at least 95%, at least 99%, or 100% identity to SEQ ID NO: 91.

In some embodiments, the viral particle comprises a nucleic acid encoding a dominant-negative TGF-β that shares at least 75%, at least 80%, at least 85%, at least 90%, at least 95%, at least 99%, or 100% identity to SEQ ID NO: 96.

### Exemplary Payloads

In some embodiments, the viral particles of the present disclosure comprise a polynucleotide sequence encoding, in any order, on a polycistronic transcript: a promoter, a therapeutic protein (e.g. CAR), optionally a cytosolic FRB domain or a portion thereof, and optionally a synthetic cytokine polypeptide (e.g. RACR). In some embodiments, the polycistronic transcript comprises a promoter and a CAR. Illustrative promoters include, without limitation, a cytomegalovirus (CMV) promoter, a CAG promoter, an SV40 promoter, an SV40/CD43 promoter, and a MND promoter.

In some embodiments, the polycistronic construct comprises in 5′ to 3′ order a nucleotide sequence encoding FRB, a nucleotide sequence encoding a synthetic cytokine polypeptide, and a nucleotide sequence encoding a CAR. In some embodiments, the nucleotide sequence encoding the synthetic cytokine polypeptide comprises in 5′ to 3′ order a first nucleotide sequence encoding FRB:IL2RG and a second nucleotide sequence encoding FKBP12:IL2RB. In some embodiments, the nucleotide sequence encoding the synthetic cytokine polypeptide comprises in 5′ to 3′ order a first nucleotide sequence encoding FKBP12:IL2RG and a second nucleotide sequence encoding sFRB:IL2RB.

In some embodiments, the viral particles of the present disclosure comprise a polynucleotide sequence encoding, in 5′ to 3′ order on a polycistronic transcript:
(a) a MND promoter;
(b) a CAR;
(c) a cytosolic FRB domain or a portion thereof;
(d) a RACR cell-surface receptor; and
(e) a WPRE sequence

In some embodiments, the viral particles of the present disclosure comprise a polynucleotide sequence encoding, in 5′ to 3′ order:
(a) a CAR;
(b) a cytosolic FRB domain or a portion thereof; and
(c) a RACR cell-surface receptor.

In some embodiments, the viral particle comprises a nucleic acid sequence that shares at least 75%, at least 80%, at least 85%, at least 90%, at least 95%, at least 99%, or 100% identity to SEQ ID NO: 35.

In some embodiments, the viral particle comprises a polypeptide sequence that shares at least 75%, at least 80%, at least 85%, at least 90%, at least 95%, at least 99%, or 100% identity to SEQ ID NO: 49.

In some embodiments, the viral particle comprises a nucleic acid sequence that shares at least 75%, at least 80%, at least 85%, at least 90%, at least 95%, at least 99%, or 100% identity to SEQ ID NO: 61.

In some embodiments, the viral particles of the present disclosure comprises a polynucleotide sequence encoding, in 5′ to 3′ order on a polycistronic transcript:
(a) a MND promoter;
(b) a cytosolic FRB domain or a portion thereof;
(c) a RACR cell-surface receptor;
(d) a CAR; and
(e) a WPRE sequence.

In some embodiments, the viral particles of the present disclosure comprises a polynucleotide sequence encoding, in 5′ to 3′ order:
(a) a cytosolic FRB domain or a portion thereof;
(b) a RACR cell-surface receptor; and
(c) a CAR.

In some embodiments, the viral particle comprises a polypeptide sequence that shares at least 75%, at least 80%, at least 85%, at least 90%, at least 95%, at least 99%, or 100% identity to SEQ ID NO: 75.

In some embodiments, the viral particle comprises a nucleic acid sequence that shares at least 75%, at least 80%, at least 85%, at least 90%, at least 95%, at least 99%, or 100% identity to SEQ ID NO: 81.

In some embodiments, the viral particles of the present disclosure comprise a polynucleotide sequence encoding, in 5′ to 3′ order on a polycistronic transcript:
(a) a MND promoter;
(b) a cytosolic FRB domain or a portion thereof;
(c) a CAR;
(d) TGF-β DN domain or portion thereof; and
(e) a WPRE sequence.

In some embodiments, the viral particles of the present disclosure comprise a polynucleotide sequence encoding, in 5′ to 3′ order:
(a) a cytosolic FRB domain or a portion thereof;
(b) a CAR; and
(c) a TGF-β DN domain or portion thereof.

In some embodiments, the viral particle comprises a polypeptide sequence that shares at least 75%, at least 80%, at least 85%, at least 90%, at least 95%, at least 99%, or 100% identity to SEQ ID NO: 87.

In some embodiments, the viral particle comprises a nucleic acid sequence that shares at least 75%, at least 80%, at least 85%, at least 90%, at least 95%, at least 99%, or 100% identity to SEQ ID NO: 92.

In some embodiments, the viral particles of the present disclosure comprise a polynucleotide sequence encoding, in 5′ to 3′ order:
(a) RSV promoter, (b) 5′ LTR, (c) HIV-1 packaging signal (Psi), (d) Rev response element (RRE) of HIV-1, (e) gp41 peptide, (f) cPPT/CTS, (g) MND promoter, (h) CMV2 extension, (i) Human CSF2R signal peptide, (j) anti-CD19 scFv, (k) IgG4 hinge domain, (1) human CD28 transmembrane domain, (m) 41BB, (n) CD3ζ, (o) P2A, (p) cytosolic FRB domain, (q) P2A, (r) neutrophil gelatinase-associated lipocalin, ER signaling domain, (s) FKBP12, (t) IL2RG, (u) transmembrane domain, (v) cytoplasmic domain, (w) P2A, (x) CD8a signal peptide, (y) Frb (DmrC) [T2098L mutation], (z) IL2RB, (aa) transmembrane domain, (bb) cytoplasmic domain, (cc) WPRE, and (dd) 3′ LTR, and the polynucleotide sequence shares at least 75%, at least 80%, at least 85%, at least 90%, at least 95%, at least 99%, or 100% identity to SEQ ID NO: 121.

In some embodiments, the viral particle comprises a nucleic acid sequence that shares at least 75%, at least 80%, at least 85%, at least 90%, at least 95%, at least 99%, or 100% identity to SEQ ID NO: 121.

In some embodiments, the viral particles of the present disclosure comprise a polynucleotide sequence encoding, in 5′ to 3′ order:
(a) human cytomegalovirus (CMV) immediate early enhancer and CMV promoter, (b) 5′ LTR from HIV-1, (c) HIV-1 packaging signal (Psi), (d) Rev response element (RRE) of HIV-1, (e) central polypurine tract and central termination (cPPT/CTS) sequence of HIV-1, (f) MND promoter (g) Human CSF2R signal peptide, (h) anti-CD19 scFv, (i) IgG4 hinge domain, (j) human CD28 transmembrane domain, (k) human CD28 transmembrane domain, (1) 41BB domain, (m) CD3ζ, (n) P2A, (o) cytosolic FRB domain, (p) P2A, (q) neutrophil gelatinase-associated lipocalin, ER signaling domain, (r) FKBP12, (s) IL2RG, (t) transmembrane domain, (u) cytoplasmic domain, (v) P2A, (w) CD8a signal peptide, (x) Frb (DmrC) [T2098L mutation], (y) IL2RB, (z) transmembrane domain, (aa) cytoplasmic domain, (bb) 3′ LTR, and (cc) synthetic polyA signal, and the polynucleotide sequence shares at least 75%, at least 80%, at least 85%, at least 90%, at least 95%, at least 99%, or 100% identity to SEQ ID NO: 122.

In some embodiments, the viral particle comprises a nucleic acid sequence that shares at least 75%, at least 80%, at least 85%, at least 90%, at least 95%, at least 99%, or 100% identity to SEQ ID NO: 122.

### Helper Plasmid

In some embodiments, the viral particles of the present disclosure comprise a polynucleotide sequence encoding, in 5′ to 3′ order on a polycistronic transcript:
(a) a gag protein; and
(b) a Pol protein.

In some embodiments, the viral particle comprises a Gag protein amino acid sequence that shares at least 75%, at least 80%, at least 85%, at least 90%, at least 95%, at least 99%, or 100% identity to SEQ ID NO: 99.

In some embodiments, the viral particle comprises a Pol protein amino acid sequence that shares at least 75%, at least 80%, at least 85%, at least 90%, at least 95%, at least 99%, or 100% identity to SEQ ID NO: 100.

In some embodiments, the viral particle comprises a gag-pol nucleic acid sequence that shares at least 75%, at least 80%, at least 85%, at least 90%, at least 95%, at least 99%, or 100% identity to SEQ ID NO: 101.

In some embodiments, the viral particle comprises a gag-pol nucleic acid sequence that shares at least 75%, at least 80%, at least 85%, at least 90%, at least 95%, at least 99%, or 100% identity to SEQ ID NO: 124.

In some embodiments, the viral particles of the present disclosure comprise a polynucleotide sequence encoding, in 5′ to 3′ order:
(a) human CMV enhancer and CMV promoter, (b) human beta-globin intron, (c) HIV-1 gag, (d) HIV-1 pol, (d) cPPT/CTS, (e) RRE, (f) beta-globin polyA signal, and the polynucleotide sequence shares at least 75%, at least 80%, at least 85%, at least 90%, at least 95%, at least 99%, or 100% identity to SEQ ID NO: 131.

In some embodiments, the viral particle comprises a gag-pol nucleic acid sequence that shares at least 75%, at least 80%, at least 85%, at least 90%, at least 95%, at least 99%, or 100% identity to SEQ ID NO: 131.

In some embodiments, the viral particle comprises a Rev protein amino acid sequence that shares at least 75%, at least 80%, at least 85%, at least 90%, at least 95%, at least 99%, or 100% identity to SEQ ID NO: 102.

In some embodiments, the viral particle comprises a Rev nucleic acid sequence that shares at least 75%, at least 80%, at least 85%, at least 90%, at least 95%, at least 99%, or 100% identity to SEQ ID NO: 103.

In some embodiments, the viral particle comprises a Rev nucleic acid sequence that shares at least 75%, at least 80%, at least 85%, at least 90%, at least 95%, at least 99%, or 100% identity to SEQ ID NO: 125.

In some embodiments, the viral particles of the present disclosure comprise a polynucleotide sequence encoding, in 5′ to 3′ order:
(a) RSV promoter, (b) HXB3 Rev, (c) HIV-1 polyA LTR, and the polynucleotide sequence shares at least 75%, at least 80%, at least 85%, at least 90%, at least 95%, at least 99%, or 100% identity to SEQ ID NO: 132.

In some embodiments, the viral particle comprises a gag-pol nucleic acid sequence that shares at least 75%, at least 80%, at least 85%, at least 90%, at least 95%, at least 99%, or 100% identity to SEQ ID NO: 132.

### Cocal Envelope Plasmids

In some embodiments, the viral particle is generated with a nucleic acid encoding a Cocal envelope and an anti-CD3 scFv.

In some embodiments, the viral particle is generated with a Cocal envelope and anti-CD3 scFv nucleic acid sequence that shares at least 75%, at least 80%, at least 85%, at least 90%, at least 95%, at least 99%, or 100% identity to SEQ ID NO: 128.

In some embodiments, the viral particles of the present disclosure are generated with a polynucleotide sequence encoding, in 5′ to 3′ order:
(a) MND promoter;
(b) CD8 derived signal peptide;
(c) anti-CD3 scFv;
(d) CD8 derived hinge;
(e) CD4 derived transmembrane domain and cytoplasmic tail;
(f) T2A;
(g) Cocal envelope;
(h) WPRE; and
(i) polyA signal, and the polynucleotide sequence shares at least 75%, at least 80%, at least 85%, at least 90%, at least 95%, at least 99%, or 100% identity to SEQ ID NO: 128.

In some embodiments, the viral particles of the present disclosure are generated with a polynucleotide sequence encoding, in 5′ to 3′ order:
(a) human CMV enhancer and CMV promoter, (b) human beta-globin intron, (c) anti-CD3 scFv, (d) Cocal envelope, (d) transmembrane domain, (e) cytoplasmic tail domain, (f) T2A peptide, (g) BGH polyA signal, and the polynucleotide sequence shares at least 75%, at least 80%, at least 85%, at least 90%, at least 95%, at least 99%, or 100% identity to SEQ ID NO: 129.

In some embodiments, the viral particles are generated with a nucleic acid sequence that shares at least 75%, at least 80%, at least 85%, at least 90%, at least 95%, at least 99%, or 100% identity to SEQ ID NO: 129.

In some embodiments, the viral particles of the present disclosure are generated with a polynucleotide sequence encoding, in 5′ to 3′ order:
(a) human CMV enhancer and CMV promoter, (b) human beta-globin intron, (c) Cocal envelope, (d) transmembrane domain, (e) cytoplasmic tail domain, (f) bovine growth hormone polyadenylation (BGH polyA) signal, and the polynucleotide sequence shares at least 75%, at least 80%, at least 85%, at least 90%, at least 95%, at least 99%, or 100% identity to SEQ ID NO: 123.

In some embodiments, the viral particles are generated with a nucleic acid sequence that shares at least 75%, at least 80%, at least 85%, at least 90%, at least 95%, at least 99%, or 100% identity to SEQ ID NO: 123.

In some embodiments, the viral particles of the present disclosure are generated with a polynucleotide sequence encoding, in 5′ to 3′ order:
(a) MND promoter, (b) Cocal envelope, (c) transmembrane domain, (d) cytoplasmic tail domain, (e) WPRE, (f) BGH polyA signal, and the polynucleotide sequence shares at least 75%, at least 80%, at least 85%, at least 90%, at least 95%, at least 99%, or 100% identity to SEQ ID NO: 130.

In some embodiments, the viral particles are generated with a nucleic acid sequence that shares at least 75%, at least 80%, at least 85%, at least 90%, at least 95%, at least 99%, or 100% identity to SEQ ID NO: 130.

### Anti-CD3 Plasmids

In some embodiments, the viral particles comprise an anti-CD3 amino acid sequence that shares at least 75%, at least 80%, at least 85%, at least 90%, at least 95%, at least 99%, or 100% identity to SEQ ID NO: 15.

In some embodiments, the viral particles are generated with an anti-CD3 nucleic acid sequence that shares at least 75%, at least 80%, at least 85%, at least 90%, at least 95%, at least 99%, or 100% identity to SEQ ID NO: 126.

In some embodiments, the viral particles of the present disclosure are generated with a polynucleotide sequence encoding, in 5′ to 3′ order:
(a) human CMV enhancer and CMV promoter, (b) human beta-globin intron, (c) Gaussia luc signal peptide, (d) anti-CD3 VL chain, (e) G4S linker, (f) anti-CD3 VH chain, (g) hinge domain, (h) transmembrane domain, (i) cytoplasmic tail domain, (j) BGH polyA signal, and the polynucleotide sequence shares at least 75%, at least 80%, at least 85%, at least 90%, at least 95%, at least 99%, or 100% identity to SEQ ID NO: 126.

In some embodiments, the viral particles are generated with an anti-CD3 nucleic acid sequence that shares at least 75%, at least 80%, at least 85%, at least 90%, at least 95%, at least 99%, or 100% identity to SEQ ID NO: 127.

In some embodiments, the viral particles of the present disclosure are generated with a polynucleotide sequence encoding, in 5′ to 3′ order:
(a) human CMV enhancer and CMV promoter, (b) human beta-globin intron, (c) Gaussia luc signal peptide, (d) anti-CD3 VL chain, (e) G4S linker, (f) anti-CD3 VH chain, (g) Glycophorin A transmembrane domain, (h) Glycophorin A cytoplasmic tail domain, (i) BGH polyA signal, and the polynucleotide sequence shares at least 75%, at least 80%, at least 85%, at least 90%, at least 95%, at least 99%, or 100% identity to SEQ ID NO: 127.

### Costimulatory and Adhesion Molecule Plasmids

In some embodiments, the viral particles comprise a CD58 amino acid sequence that shares at least 75%, at least 80%, at least 85%, at least 90%, at least 95%, at least 99%, or 100% identity to SEQ ID NO: 17.

In some embodiments, the viral particles are generated with a CD58 nucleic acid sequence that shares at least 75%, at least 80%, at least 85%, at least 90%, at least 95%, at least 99%, or 100% identity to SEQ ID NO: 18.

In some embodiments, the viral particles of the present disclosure are generated with a polynucleotide sequence comprising: (a) human CMV enhancer and CMV promoter, (b) human beta-globin intron, (c) endogenous signal peptide, (d) a CD58 polynucleotide, (e) BGH polyA signal, and the CD58 polynucleotide sequence shares at least 75%, at least 80%, at least 85%, at least 90%, at least 95%, at least 99%, or 100% identity to SEQ ID NO: 18.

In some embodiments, the viral particles comprise a CD80 amino acid sequence that shares at least 75%, at least 80%, at least 85%, at least 90%, at least 95%, at least 99%, or 100% identity to SEQ ID NO: 20.

In some embodiments, the viral particles are generated with a CD80 nucleic acid sequence that shares at least 75%, at least 80%, at least 85%, at least 90%, at least 95%, at least 99%, or 100% identity to SEQ ID NO: 21.

In some embodiments, the viral particles of the present disclosure are generated with a polynucleotide sequence comprising: (a) human CMV enhancer and CMV promoter, (b) human beta-globin intron, (c) endogenous signal peptide, (d) a CD80 polynucleotide, (e) BGH polyA signal, and the CD80 polynucleotide sequence shares at least 75%, at least 80%, at least 85%, at least 90%, at least 95%, at least 99%, or 100% identity to SEQ ID NO: 21.

In some embodiments, the viral particles comprise a CD86 amino acid sequence that shares at least 75%, at least 80%, at least 85%, at least 90%, at least 95%, at least 99%, or 100% identity to SEQ ID NO: 23.

In some embodiments, the viral particles are generated with a CD86 nucleic acid sequence that shares at least 75%, at least 80%, at least 85%, at least 90%, at least 95%, at least 99%, or 100% identity to SEQ ID NO: 24.

In some embodiments, the viral particles of the present disclosure are generated with a polynucleotide sequence comprising: (a) human CMV enhancer and CMV promoter, (b) human beta-globin intron, (c) endogenous signal peptide, (d) a CD86 polynucleotide, (e) BGH polyA signal, and the CD86 polynucleotide sequence shares at least 75%, at least 80%, at least 85%, at least 90%, at least 95%, at least 99%, or 100% identity to SEQ ID NO: 24.

### Gene Editing

Numerous gene-editing methods are known in the art and additional methods are continuously being created. The methods and compositions of the present disclosure are capable of delivering a variety of genetic payloads, including polynucleotides intended for insertion into the genome of the target cell and/or gene editing systems (CRISPR-Cas, meganucleases, homing endonucleases, zinc finger enzymes and the like). In embodiments, a polynucleotide (e.g. transgene), enzyme, and/or guide RNA are delivered in one, two, three or more vectors of the same type (e.g. lentivirus, AAV, etc.) or different types (including e.g. combinations of non-viral and virus vectors or different types of viral vectors). The methods and systems of the disclosure can be used for generating point mutation(s), insertions, deletions, etc. Random mutagenesis and multi-locus gene editing are also within the scope of the disclosure.

### Production/Packaging Cell Lines

The present disclosure provides a host cell for the production of viral particles according to the disclosure. In some embodiments, the host cell expresses one or more exogenous and/or recombinant transduction enhancers at the cell surface. In some embodiments, the host cell expresses an activating protein, a costimulatory molecule, and an adhesion molecule at the cell surface. In some embodiments, the host cell expresses one or more of anti-CD3scFv, CD86, CD80, and/or CD58. In some embodiments, the host cell expresses at least an anti-CD3 scFv, and CD58. In some embodiments, the host cell expresses at least an anti-CD3 scFv, and CD80. In some embodiments, the host cell expresses at least an anti-CD3 scFv, and CD86. In some embodiments, the host cell expresses at least an anti-CD3 scFv, a CD80, and CD58. In some embodiments, the host cell expresses at least an anti-CD3 scFv, a CD86, and CD58.

In some embodiments, the host cell is for the production of viral vectors according to the foregoing embodiments. In some embodiments, the host cell comprises tagging proteins useful for the purification of the viral particles.

In some embodiments, the host cell is a packaging cell and comprises one or more of the following genes: gag, pol, env and rev. In some embodiments, a packaging cell for a retroviral vector comprises gag, pol and env genes. In some embodiments, a packaging cell for a lentiviral vector comprises gag, pol, env and rev genes.

In some embodiments, the host cell is a producer cell and comprises gag, pol, env and optionally rev genes and a retroviral or lentiviral vector genome. In a typical recombinant retroviral or lentiviral vector for use in gene therapy, at least part of one or more of the gag-pol and env protein coding regions may be removed from the virus and provided by the packaging cell. This makes the viral vector replication-defective as the virus is capable of integrating its genome into a host genome but the modified viral genome is unable to propagate itself due to a lack of structural proteins.

Packaging cells are used to propagate and isolate quantities of viral vectors i.e to prepare suitable titres of the retroviral vector for transduction of a target cell.

In some embodiments, propagation and isolation may entail isolation of the retroviral gagpol and env (and in the case of lentivirus, rev) genes and their separate introduction into a host cell to produce a packaging cell line. The packaging cell line produces the proteins required for packaging retroviral DNA but it cannot bring about encapsidation due to the lack of a psi region. However, when a recombinant vector carrying a psi region is introduced into the packaging cell line, the helper proteins can package the psi-positive recombinant vector to produce the recombinant virus stock.

A summary of the available packaging lines is presented in “Retroviruses” (1997 Cold Spring Harbour Laboratory Press Eds: J M Coffin, SM Hughes, HE Varmus pp 449).

Packaging cells have also been developed in which the gag, pol and env (and, in the case of lentiviral vectors, rev) viral coding regions are carried on separate expression plasmids that are independently transfected into a packaging cell line, so that three recombinant events are required for wild type viral production.

Transient transfection avoids the longer time required to generate stable vector-producing cell lines and is used if the vector or retroviral packaging components are toxic to cells. Components typically used to generate retroviral/lentivial vectors include a plasmid encoding the Gag/Pol proteins, a plasmid encoding the Env protein (and, in the case of lentiviral vectors, the rev protein), and the retroviral/lentiviral vector genome. Vector production involves transient transfection of one or more of these components into cells containing the other required components. The packaging cells of the present invention may be any mammalian cell type capable of producing retroviral/lentiviral vector particles. The packaging cells may be 293T-cells, or variants of 293T-cells which have been adapted to grow in suspension and grow without serum.

In some embodiments, the packaging cells are made by transient transfection with
a) a transfer vector
b) a gagpol expression vector
c) an env expression vector. In some embodiments, the env gene is a heterologous, resulting in a pseudotyped retroviral vector. For example, in some embodiments, the env gene is from RD1 14 or one of its variants, VSV-G, including the cocal envelope, the Gibbon-ape leukaemia virus (GALV), the Amphotropic envelope or Measles envelope or baboon retroviral envelope glycoprotein, or any exemplary envelope protein discussed herein.

In the case of lentiviral vector, in some embodiments, transient transfection with a rev vector is also performed.

The present disclosure provides host cells expressing viral particles according to the foregoing embodiments. In some embodiments, the host cells express, at the cell surface, one or more transduction enhancers. In some embodiments, the present invention provides a host cell which is engineered to express, at the cell surface,
(a) an activating protein; and/or
(b) a costimulatory protein; and/or
(c) an adhesion molecule
such that a retroviral or lentiviral vector produced by the packaging cell is as described in the foregoing embodiments.

In some embodiments, the host cell also expresses, at the cell surface, a tagging protein which comprises: a binding domain which binds to a capture moiety; and a transmembrane domain, which tagging protein facilitates purification of the viral vector from cellular supernatant via binding of the tagging protein to the capture moiety, such that a retroviral or lentiviral vector produced by the packaging cell has the characteristics describing in the foregoing sections.

The tagging protein may also comprise a spacer between the binding domain and the transmembrane domain.

The term host cell may be used to describe a packaging cell or a producer cell. A packaging cell may comprise one or more of the following genes: gag, pol, env and/or rev. A producer cell may comprise gag, pol, env and optionally rev genes and also comprises a retroviral or lentiviral genome. In some embodiments, the host cell may be any suitable cell line stably expressing mitogenic and/or cytokine transduction enhancers. It may be transiently transfected with transfer vector, gagpol, env (and rev in the case of a lentivirus) to produce replication incompetent retroviral/lentiviral vector.

The present disclosure also provides a method for making a host cell according to the above, which comprises the step of transducing or transfecting a cell with a nucleic acid encoding one or more transduction enhancers. Also provided is a method for producing a viral vector according to the foregoing embodiments which comprises the step of expressing a retroviral or lentiviral genome in a cell according to the second aspect of the invention.

### Systems and Kits

In some embodiments, the present disclosure provides a system, therapeutic system, or composition, comprising:
(a) an adaptor molecule comprising a targeting moiety and a masked hapten, wherein the masked hapten comprises a masking moiety linked to a hapten; and
(b) a plurality of recombinant retroviral particles,
wherein each of the retroviral particles comprises a polynucleotide comprising, in 5′ to 3′ order:
(i) a 5′ long terminal repeat (LTR) or untranslated region (UTR),
(ii) a promoter,
(iii) a sequence encoding a receptor that specifically binds to the hapten, and
(iv) a 3′ LTR or UTR; and
wherein each of the retroviral particles comprises a viral envelope comprising
(i) a viral fusion glycoprotein, and
(ii) one or more transduction enhancers
wherein optionally each of the transduction enhancers is selected from the group consisting of a T-cell activation receptor, a NK-cell activation receptor, a co stimulatory molecule, and an adhesion molecule.

The present disclosure also provides a kit comprising the system and instructions for use of the system.

### Transgenic Immune Cells

The present disclosure provides a method for making an activated transgenic immune cell, which comprises the step of contacting an immune cell with a viral vector according to any of the foregoing embodiments. The immune cells may be transduced in vivo or ex vivo. In some embodiments, the viral vectors are administered to a living subject such that the immune cells are transduced in vivo without any need to isolate and manipulate host cells ex vivo. In some embodiments, immune cells are manipulated ex vivo and then returned to the subject in need thereof.

The immune cells generally are mammalian cells, and typically are human cells, more typically primary human cells, e.g., allogeneic or autologous donor cells. The cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immune systems, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, herein, the cells provided are cytotoxic T lymphocytes. A “Cytotoxic T lymphocyte” (CTL) may include but is not limited to, for example, a T lymphocyte that expresses CD8 on the surface thereof (e.g., a CD8+ T cell). In some embodiments, such cells are preferably “memory” T cells (TM cells) that are antigen-experienced. In some embodiments, the cell is a precursor T cell. In some embodiments, the precursor T cell is a hematopoietic stem cell. In some embodiments, the cell is a CD8+ T cytotoxic lymphocyte cell selected from the group consisting of naive CD8+ T cells, central memory CD8+ T cells, effector memory CD8+ T cells and bulk CD8+ T cells. In some embodiments, the cell is a CD4+ T helper lymphocyte cell that is selected from the group consisting of naive CD4+ T cells, central memory CD4+ T cells, effector memory CD4+ T cells, and bulk CD4+ T cells.

As used herein, any reference to a transgenic T cell or transduced T cell, or the use thereof, may also be applied to any of the other immune cell types disclosed herein.

The present disclosure also provides transgenic immune cells comprising one or more exogenous nucleic acid molecules. In some embodiments, the transgenic immune cells comprise polynucleotides encoding hapten-binding receptors. In some embodiments, the transgenic immune cells comprise polynucleotides encoding transduction enhancers. In some embodiments, the transgenic immune cells comprise polynucleotides encoding T cell activator proteins. In some embodiments, the transgenic immune cells comprise polynucleotides encoding hapten-binding receptors and polynucleotides encoding T cell activator proteins.

### Target Immune Cells

Non-limiting examples of cells that can be the target of the viral particle described herein include T lymphocytes, dendritic cells (DC), Treg cells, B cells, Natural Killer cells, and macrophages. In some embodiments, the viral particle described herein is capable of transducing an alpha beta T cell. In some embodiments, the viral particle described herein is capable of transducing an alpha beta T cell. In some embodiments, the viral particle described herein is capable of transducing a gamma delta T cell. In some embodiments, the viral particle described herein is capable of transducing an NK cell.

### T Cells

T cells (“T lymphocytes”) are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. These T cells are called alpha beta T cells. In other T cells, called gamma delta T cells, the TCR contains a gamma (γ) and a delta (δ) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

In some embodiments, the cells used in the methods provided herein are primary T lymphocytes (e.g., primary human T lymphocytes). The primary T lymphocytes used in the methods provided herein may be naive T lymphocytes or MHC-restricted T lymphocytes. In some embodiments, the T lymphocytes are CD4+. In other embodiments, the T lymphocytes are CD8+. In some embodiments, the primary T lymphocytes are tumor infiltrating lymphocytes (TILs). In some embodiments, the primary T lymphocytes have been isolated from a tumor biopsy or have been expanded from T lymphocytes isolated from a tumor biopsy. In some embodiments, the primary T lymphocytes have been isolated from, or are expanded from T lymphocytes isolated from, peripheral blood, cord blood, or lymph. In some embodiments, the T lymphocytes are allogeneic with respect to a particular individual, e.g., a recipient of said T lymphocytes. In certain other embodiments, the T lymphocytes are not allogeneic with respect to a certain individual, e.g., a recipient of said T lymphocytes. In some embodiments, the T lymphocytes are autologous with respect to a particular individual, e.g., a recipient of said T lymphocytes.

In some embodiments, primary T lymphocytes used in the methods described herein are isolated from a tumor, e.g., are tumor-infiltrating lymphocytes. In some embodiments, such T lymphocytes are specific for a tumor specific antigen (TSA) or tumor associated antigen (TAA). In some embodiments, primary T lymphocytes are obtained from an individual, optionally expanded, and then transduced, using the methods described herein, with a nucleic acid encoding one or more chimeric antigen receptors (CARs), and optionally then expanded. T lymphocytes can be expanded, for example, by contacting the T lymphocytes in culture with antibodies to CD3 and/or CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681. In some embodiments, the antibodies are anti-CD3 and/or anti-CD28, and the antibodies are not bound to a solid surface (e.g., the antibodies contact the T lymphocytes in solution). In some embodiments, either of the anti-CD3 antibody or anti-CD28 antibody is bound to a solid surface (e.g. bead, tissue culture dish plastic), and the other antibody is not bound to a solid surface (e.g., is present in solution).

### NK Cells

Natural killer (NK) cells are cytotoxic lymphocytes that constitute a major component of the innate immune system. NK cells typically comprise approximately 10 to 15% of the mononuclear cell fraction in normal peripheral blood. NK cells do not express T-cell antigen receptors (TCR), CD3 or surface immunoglobulins (Ig) B cell receptor, but usually express the surface markers CD16 (FcγRIII) and CD56 in humans. NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines Activated NK cells are referred to as lymphokine activated killer (LAK) cells. NK cells possess two types of surface receptors, labeled “activating receptors” and “inhibitory receptors,” that control the cells' cytotoxic activity.

Among other activities, NK cells play a role in the host rejection of tumors. Because many cancer cells have reduced or no class I MHC expression, they can become targets of NK cells. Natural killer cells can become activated by cells lacking, or displaying reduced levels of, major histocompatibility complex (MHC) proteins. In addition to being involved in direct cytotoxic killing, NK cells also serve a role in cytokine production, which can be important to control cancer and infection. Activated and expanded NK cells and LAK cells have been used in both ex vivo therapy and in vivo treatment of patients having advanced cancer, with some success against bone marrow related diseases, such as leukemia; breast cancer; and certain types of lymphoma.

In vivo Delivery of Polynucleotides

In some embodiments, the disclosure provides a method of delivering a nucleic acid to a cell in vivo. In some embodiments, the disclosure provides a method of delivering a nucleic acid to an immune cell in vivo. In some embodiments, the viral particles of the disclosure activate and transduce an immune cell in vivo.

In some embodiments, a nucleotide sequence encoding a CAR is administered to the subject which allows the production of the CAR in vivo. In some embodiments, the administration of such viral particles generates a similar effect in vivo as direct administration of the CAR. In some embodiments, the administration of such viral particles improves the in vivo transduction efficiency of a particle.

In some embodiments, in vivo delivery of such viral particles generates CAR expression over time (e.g., starting within hours and lasting several days). In some embodiments, in vivo delivery of such viral particles results in desirable pharmacokinetics, pharmacodynamics and/or safety profile of the encoded CAR.

In some embodiments, the nucleotide sequence may be optimized by one or more means to prevent immune activation, increase stability, reduce any tendency to aggregate, such as over time, and/or to avoid impurities. Such optimization may include the use of modified nucleosides, modified, and/or particular 5′ UTRs, 3′UTRs, and/or poly(A) tail modifications for improved intracellular stability and translational efficiency (see, e.g., Stadler et al., 2017, Nat. Med.). Such modifications are known in the art.

Strategies for in vivo delivery of polynucleotides (e.g., mRNA) are known in the art. For a summary of strategies, see Mol. Ther. 2019 Apr. 10; 27(4): 710-728, which is incorporated herein by reference in its entirety.

In some embodiments, the viral particle has a multi-step mechanism of action:
(a) the viral particle binds to T cells in vivo via an immune cell-activating protein (e.g., anti-CD3 scFv), a co-stimulatory molecule (e.g., a CD28 ligand), a cell adhesion molecule (e.g., CD58), or any combination thereof, activates the T cells and facilitates viral particle internalization through interaction with the Cocal glycoprotein
(b) the vector RNA genome is reverse-transcribed into DNA, shuttled to the nucleus, and integrated into the genome; and
(c) the transduced T cells express the polypeptide of interest.

In some embodiments, the viral particle has a multi-step mechanism of action:
(a) the viral particle binds to T cells in vivo via an immune cell-activating protein (e.g., anti-CD3 scFv), a co-stimulatory molecule (e.g., a CD28 ligand), a cell adhesion molecule (e.g., CD58), or any combination thereof, activates the T cells and facilitates viral particle internalization through interaction with the Cocal glycoprotein
(b) the vector RNA genome encoding a CAR is reverse-transcribed into DNA, shuttled to the nucleus, and integrated into the genome; and
(c) the transduced T cells express the CAR and target cells, while also expressing the FRB and RACR system for rapamycin-controlled cytokine signaling.

### Immune Cell Activation

In some embodiments, administration of the particle to a subject results in the activation of immune cells.

In some embodiments, activation of immune cells is measured by the level of one or more cell markers. In some embodiments, activation of immune cells is measured by the percentage of the immune cells that are positive for one or more cell markers. In some embodiments, the immune cells are T cells (T lymphocytes) or NK cells. In some embodiments, the immune cells are CD4+ T cells or CD8+ T cells. In some embodiments, the one or more cell markers are selected from the groups consisting of CD71, CD25, and any combination thereof.

In some embodiments, activation of immune cells is measured by the percentage of the immune cells that are CD71 positive. In some embodiments, administration of the viral particle increases the percentage of the CD71+ immune cells by at least 10%, at least 20%, at least 30%, at least 40%, at least 50%, at least 60%, at least 70%, at least 80%, or at least 90%. In some embodiments, activation of immune cells is measured by the level of CD71 expressed on the surface of the immune cells. In some embodiments, administration of the viral particle increases the level of CD71 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, activation of immune cells is measured by the percentage of the immune cells that are CD25 positive. In some embodiments, administration of the viral particle increases the percentage of the CD25+ immune cells by at least 10%, at least 20%, at least 30%, at least 40%, at least 50%, at least 60%, at least 70%, at least 80%, or at least 90%. In some embodiments, activation of immune cells is measured by the level of CD25 expressed on the surface of the immune cells. In some embodiments, administration of the viral particle increases the level of CD25 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, administration of the viral particle in a subject results in active proliferation of immune cells. In some embodiments, the proliferation of immune cells increase the number and/or susceptibility to transduction by vector.

In some embodiments, administration of the viral particle in a subject results in a decrease of numbers of immune cells (e.g., T cells) in the GO phase and/or an increase of numbers of immune cells (e.g., T cells) in the non-GO phase.

In some embodiments, administration of the viral particle in a subject increases the number and/or percentage of immune cells that are in a state of metabolic fitness for transduction of vector.

In some embodiments, administration of the viral particle in a subject results in the accumulation of immune cells in lymph nodes. In some embodiments, administration of the viral particle in a subject results in the accumulation of immune cells in tumor sites.

In some embodiments, the viral particle is a lentiviral particle. In some embodiments, the immune cells are T cells. In some embodiments, the immune cells here are a subset of immune cells in vivo that can be recognized by at least one antigen-specific binding domain of the CAR. In some embodiments, the immune cells reside in the lymph nodes.

In some embodiments, the viral particle activates non-transduced immune cells. In some embodiments, the viral particle expands non-transduced immune cells. In some embodiments, the viral particle activates and/or expands tumor infiltrating lymphocytes. In some embodiments, the viral particle activates and/or expands tumor reactive T cells present in tumor draining or metastatic lymph nodes.

### Administration Route

In some embodiments, the viral particle is administered via a route selected from the group consisting of parenteral, intravenous, intramuscular, subcutaneous, intratumoral, intraperitoneal, and intralymphatic. In some embodiments, the viral particle is administered multiple times. In some embodiments, the viral particle is administered by intralymphatic injection of the viral particle. In some embodiments, the viral particle is administered by intraperitoneal injection of the viral particle. In some embodiments, the viral particle is administered by intra-nodal injection—that is, the viral particle may be administered via injection into a lymph node, such as an inguinal lymph node. In some embodiments, the viral particle is administered by injection of the viral particle into tumor sites (i.e. intratumoral). In some embodiments, the viral particle is administered subcutaneously. In some embodiments, the viral particle is administered systemically. In some embodiments, the viral particle is administered intravenously. In some embodiments, the viral particle is administered intra-arterially. In some embodiments, the viral particle is a lentiviral particle.

In some embodiments, the viral particle is administered by intraperitoneal, subcutaneous, or intranodal injection. In some embodiments, the viral particle is administered by intraperitoneal injection. In some embodiments, the viral particle is administered by subcutaneous injection. In some embodiments, the viral particle is administered by intranodal injection.

In some embodiments, the transduced immune cells comprising the polynucleotide of the present disclosure is administered to the subject.

In some embodiments, the viral particle is administered as a single injection. In some embodiments, the viral particle is administered as at least 1, at least 2, at least 3, at least 4, at least 5, at least 6, at least 7, at least 8, at least 9, or at least 10 injections.

### Dosage Form and Dosing Regimen

### Viral Particle

A viral particle may be used to infect cells in vivo at an any effective dosage. In some embodiments, the viral particle is administered to a subject in vivo, by direct injection to the cell, tissue, organ or subject in need of therapy.

In some embodiments, a viral particle may be administered in connection with a cell. A viral particle may be connected with a cell by incubation of a viral particle with a cell such that the viral particle is associated with the cell. In some embodiments, the dose of the product to be delivered is determined based on the number of viral particle-bound cells.

Viral particles may also be delivered according to viral titer (TU/mL). The amount of lentivirus directly injected is determined by total TU and can vary based on both the volume that could be feasibly injected to the site and the type of tissue to be injected. In some embodiments, the viral titer delivered is about 1×10⁵ to 1×10⁶, about 1×10⁵ to 1×10⁷, 1×10⁵ to 1×10⁷, about 1×10⁶ to 1×10⁹, about 1×10⁷ to 1×10¹⁰, about 1×10⁷ to 1×10¹¹, or about 1×10⁹ to 1×10¹¹ TU or more per injection could be used. In some embodiments, the viral titer delivered is about 1×10⁶ to 1×10⁷, about 1×10⁶ to 1×10⁸, 1×10⁶ to 1×10⁹, about 1×10⁷ to 1×10¹⁰, about 1×10⁸ to 1×10¹¹, about 1×10⁸ to 1×10¹², or about 1×10¹⁰ to 1×10¹² or more per injection could be used. For example, a brain injection site may only allow for a very small volume of virus to be injected, so a high titer prep would be preferred, a TU of about 1×10⁶ to 1×10⁷, about 1×10⁶ to 1×10⁸, 1×10⁶ to 1×10⁹, about 1×10⁷ to 1×10¹⁰, about 1×10⁸ to 1×10¹¹, about 1×10⁸ to 1×10¹², or about 1×10¹⁰ to 1×10¹² or more per injection could be used. However, a systemic delivery could accommodate a much larger TU, a load of about 1×10⁸, about 1×10⁹, about 1×10¹⁰, about 1×11, about 1×10¹², about 1×10¹³, about 1×10¹⁴, or about 1×10¹⁵, could be delivered.

In some embodiments, the vector is administered at a dose of between about 1×10¹² and 5×10¹⁴ vector genomes (vg) of the vector per kilogram (vg) of total body mass of the subject (vg/kg). In some embodiments, the vector is administered at a dose of between about 1×10¹³ and 5×10¹⁴ vg/kg. In some embodiments, the vector is administered at a dose of between about 5×10¹³ and 3×10¹⁴ vg/kg. In some embodiments, the vector is administered at a dose of between about 5×10¹³ and 1×10¹⁴ vg/kg. In some embodiments, the vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the vector is administered at a dose of between about 1×10¹² and 5×10¹⁴ vector particles (vp) of the vector per kilogram (vp) of total body mass of the subject (vp/kg). In some embodiments, the vector is administered at a dose of between about 1×10¹³ and 5×10¹⁴ vp/kg. In some embodiments, the vector is administered at a dose of between about 5×10¹³ and 3×10¹⁴ vp/kg. In some embodiments, the vector is administered at a dose of between about 5×10³ and 1×10¹⁴ vp/kg. In some embodiments, the vector is administered at a dose of less than about 1×10¹² vp/kg, less than about 3×10¹² vp/kg, less than about 5×10¹² vp/kg, less than about 7×10¹² vp/kg, less than about 1×10¹³ vp/kg, less than about 3×10¹³ vp/kg, less than about 5×10¹³ vp/kg, less than about 7×10¹³ vp/kg, less than about 1×10¹⁴ vp/kg, less than about 3×10¹⁴ vp/kg, less than about 5×10¹⁴ vp/kg, less than about 7×10¹⁴ vp/kg, less than about 1×10¹⁵ vp/kg, less than about 3×10¹⁵ vp/kg, less than about 5×10¹⁵ vp/kg, or less than about 7×10¹⁵ vp/kg.

In some embodiments, administration of the viral particles of the present disclosure decreases the number of B cells in the subject by at least 1%, at least 2%, at least 3%, at least 5%, at least 7%, at least 10%, at least 20%, at least 30%, at least 40%, at least 50%, at least 60%, at least 70%, at least 80%, or at least 90%. In some embodiments, the decrease is evaluated by the number of B cells 1, 2, 3, 4, 5, 6, 7, 8, 9, 10, 11, or 12 weeks after the viral particle is administered, wherein the reference number is the number of B cells in a subject that was administered a vehicle control.

In some embodiments, administration of the viral particles of the present disclosure decreases the number of B cells in the subject by at least 95%.

In some embodiments, the B cells are in the peripheral blood of the subject. In some embodiments, the B cells are in the bone marrow of the subject. In some embodiments, the B cells are in the spleen of the subject

In some embodiments, the B cells are depleted in the subject for at least 7 days, at least 10 days, at least 20 days, at least 30 days, at least 40 days, at least 50 days, at least 60 days, at least 70 days, or at least 80 days after administering the viral particle.

In some embodiments, the B cells are depleted in the subject for at least 80 days after administering the viral particle.

### Rapamycin

Rapamune® (sirolimus, rapamycin) is available as an oral solution or tablet and is FDA approved for the following indications:
Prophylaxis of organ rejection on renal transplantation
Limitations of use in renal transplantation
Treatment of patients with lymphangioleiomyomatosis

Per the US Prescribing Information (USPI), rapamycin is available in 1 mg/mL oral solution or 0.5, 1, or 2 mg tablets and is to be administered once daily. Rapamycin may also be delivered in other dosage forms and/or by other administration routes.

In some embodiments, rapamycin is administered at a dose of between about 0.1 mg/m² and 100 mg/m² of surface area of the subject. In some embodiments, the subject is a human. In some embodiments, rapamycin is administered at a dose of between about 0.5 mg/m² and 50 mg/m². In some embodiments, rapamycin is administered at a dose of between about 0.5 mg/m² and 10 mg/m². In some embodiments, rapamycin is administered at a dose of between about 0.5 mg/m² and 3 mg/m². In some embodiments, rapamycin is administered at a dose of between about 0.5 mg/m² and 5 mg/m². In some embodiments, rapamycin is administered at a dose of between about 1 mg/m² and 5 mg/m². In some embodiments, rapamycin is administered at a dose of between about 2 mg/m² and 6 mg/m². In some embodiments, rapamycin is administered at a dose of about 1 mg/m². In some embodiments, rapamycin is administered at a dose of about 2 mg/m². In some embodiments, rapamycin is administered at a dose of about 3 mg/m². In some embodiments, rapamycin is administered at a dose of between about 2 mg/m² and 6 mg/m². In some embodiments, rapamycin is administered at a dose of between about 3 mg/m² and 9 mg/m². In some embodiments, rapamycin is administered at a dose of between about 4 mg/m² and 12 mg/m². In some embodiments, rapamycin is administered at a dose of between about 5 mg/m² and 15 mg/m². In some embodiments, rapamycin is administered at a dose of between about 6 mg/m² and 20 mg/m². In some embodiments, rapamycin is administered at a dose of between about 10 mg/m² and 50 mg/m². In some embodiments, the dose of rapamycin is the total dose within a 24-hour time period.

In some embodiments, rapamycin is administered at a dose of between about 0.001 mg/m² and 100 mg/m² of surface area of the subject. In some embodiments, the subject is a human. In some embodiments, rapamycin is administered at a dose of between about 0.001 mg/m² and 0.1 mg/m², between about 0.01 mg/m² and 1 mg/m², between about 0.1 mg/m² and 10 mg/m², between about 1 mg/m² and 100 mg/m², between about 0.001 mg/m² and 0.05 mg/m², between about 0.005 mg/m² and 0.25 mg/m², between about 0.01 mg/m² and 0.5 mg/m², between about 0.05 mg/m² and 2.5 mg/m², between about 0.1 mg/m² and 5 mg/m², between about 0.5 mg/m² and 25 mg/m², between about 1 mg/m² and 50 mg/m², between about 2 mg/m² and 100 mg/m², between about 0.001 mg/m² and 0.01 mg/m², between about 0.005 mg/m² and 0.05 mg/m², between about 0.01 mg/m² and 0.1 mg/m², between about 0.05 mg/m² and 0.5 mg/m², between about 0.1 mg/m² and 1 mg/m², between about 0.5 mg/m² and 5 mg/m², between about 1 mg/m² and 10 mg/m², between about 5 mg/m² and 50 mg/m², or between about 10 mg/m² and 100 mg/m², including all ranges and subranges in between. In some embodiments, rapamycin is administered at a dose of between about 0.001 mg/m² and 0.005 mg/m². In some embodiments, rapamycin is administered at a dose of between about 0.002 mg/m² and 0.01 mg/m². In some embodiments, rapamycin is administered at a dose of between about 0.003 mg/m² and 0.015 mg/m². In some embodiments, rapamycin is administered at a dose of between about 0.004 mg/m² and 0.02 mg/m². In some embodiments, rapamycin is administered at a dose of between about 0.005 mg/m² and 0.025 mg/m². In some embodiments, rapamycin is administered at a dose of between about 0.006 mg/m² and 0.03 mg/m². In some embodiments, rapamycin is administered at a dose of between about 0.007 mg/m² and 0.035 mg/m². In some embodiments, rapamycin is administered at a dose of between about 0.008 mg/m² and 0.04 mg/m². In some embodiments, rapamycin is administered at a dose of between about 0.009 mg/m² and 0.045 mg/m². In some embodiments, rapamycin is administered at a dose of between about 0.01 mg/m² and 0.05 mg/m². In some embodiments, rapamycin is administered at a dose of between about 0.02 mg/m² and 0.1 mg/m². In some embodiments, rapamycin is administered at a dose of between about 0.03 mg/m² and 0.15 mg/m². In some embodiments, rapamycin is administered at a dose of between about 0.04 mg/m² and 0.2 mg/m². In some embodiments, rapamycin is administered at a dose of between about 0.05 mg/m² and 0.25 mg/m². In some embodiments, rapamycin is administered at a dose of between about 0.06 mg/m² and 0.3 mg/m². In some embodiments, rapamycin is administered at a dose of between about 0.07 mg/m² and 0.35 mg/m². In some embodiments, rapamycin is administered at a dose of between about 0.08 mg/m² and 0.4 mg/m². In some embodiments, rapamycin is administered at a dose of between about 0.09 mg/m² and 0.45 mg/m². In some embodiments, rapamycin is administered at a dose of between about 0.1 mg/m² and 0.5 mg/m². In some embodiments, rapamycin is administered at a dose of between about 0.2 mg/m² and 1 mg/m². In some embodiments, rapamycin is administered at a dose of between about 0.3 mg/m² and 1.5 mg/m². In some embodiments, rapamycin is administered at a dose of between about 0.4 mg/m² and 2 mg/m². In some embodiments, rapamycin is administered at a dose of between about 0.5 mg/m² and 2.5 mg/m². In some embodiments, rapamycin is administered at a dose of between about 0.6 mg/m² and 3 mg/m². In some embodiments, rapamycin is administered at a dose of between about 0.7 mg/m² and 3.5 mg/m². In some embodiments, rapamycin is administered at a dose of between about 0.8 mg/m² and 4 mg/m². In some embodiments, rapamycin is administered at a dose of between about 0.9 mg/m² and 4.5 mg/m². In some embodiments, rapamycin is administered at a dose of between about 1 mg/m² and 5 mg/m². In some embodiments, rapamycin is administered at a dose of between about 2 mg/m² and 10 mg/m². In some embodiments, rapamycin is administered at a dose of between about 3 mg/m² and 15 mg/m². In some embodiments, rapamycin is administered at a dose of between about 4 mg/m² and 20 mg/m². In some embodiments, rapamycin is administered at a dose of between about 5 mg/m² and 25 mg/m². In some embodiments, rapamycin is administered at a dose of between about 6 mg/m² and 30 mg/m². In some embodiments, rapamycin is administered at a dose of between about 7 mg/m² and 35 mg/m². In some embodiments, rapamycin is administered at a dose of between about 8 mg/m² and 40 mg/m². In some embodiments, rapamycin is administered at a dose of between about 9 mg/m² and 45 mg/m². In some embodiments, rapamycin is administered at a dose of between about 10 mg/m² and 50 mg/m². In some embodiments, rapamycin is administered at a dose of between about 20 mg/m² and 100 mg/m². In some embodiments, rapamycin is administered at a dose of between about 0.001 mg/m² and 0.02 mg/m². In some embodiments, rapamycin is administered at a dose of between about 0.002 mg/m² and 0.04 mg/m². In some embodiments, rapamycin is administered at a dose of between about 0.003 mg/m² and 0.06 mg/m². In some embodiments, rapamycin is administered at a dose of between about 0.004 mg/m² and 0.08 mg/m². In some embodiments, rapamycin is administered at a dose of between about 0.005 mg/m² and 0.1 mg/m². In some embodiments, rapamycin is administered at a dose of between about 0.006 mg/m² and 0.12 mg/m². In some embodiments, rapamycin is administered at a dose of between about 0.007 mg/m² and 0.14 mg/m². In some embodiments, rapamycin is administered at a dose of between about 0.008 mg/m² and 0.16 mg/m². In some embodiments, rapamycin is administered at a dose of between about 0.009 mg/m² and 0.18 mg/m². In some embodiments, rapamycin is administered at a dose of between about 0.01 mg/m² and 0.2 mg/m². In some embodiments, rapamycin is administered at a dose of between about 0.02 mg/m² and 0.4 mg/m². In some embodiments, rapamycin is administered at a dose of between about 0.03 mg/m² and 0.6 mg/m². In some embodiments, rapamycin is administered at a dose of between about 0.04 mg/m² and 0.8 mg/m². In some embodiments, rapamycin is administered at a dose of between about 0.05 mg/m² and 1 mg/m². In some embodiments, rapamycin is administered at a dose of between about 0.06 mg/m² and 1.2 mg/m². In some embodiments, rapamycin is administered at a dose of between about 0.07 mg/m² and 1.4 mg/m². In some embodiments, rapamycin is administered at a dose of between about 0.08 mg/m² and 1.6 mg/m². In some embodiments, rapamycin is administered at a dose of between about 0.09 mg/m² and 1.8 mg/m². In some embodiments, rapamycin is administered at a dose of between about 0.1 mg/m² and 2 mg/m². In some embodiments, rapamycin is administered at a dose of between about 0.2 mg/m² and 4 mg/m². In some embodiments, rapamycin is administered at a dose of between about 0.3 mg/m² and 6 mg/m². In some embodiments, rapamycin is administered at a dose of between about 0.4 mg/m² and 8 mg/m². In some embodiments, rapamycin is administered at a dose of between about 0.5 mg/m² and 10 mg/m². In some embodiments, rapamycin is administered at a dose of between about 0.6 mg/m² and 12 mg/m². In some embodiments, rapamycin is administered at a dose of between about 0.7 mg/m² and 14 mg/m². In some embodiments, rapamycin is administered at a dose of between about 0.8 mg/m² and 16 mg/m². In some embodiments, rapamycin is administered at a dose of between about 0.9 mg/m² and 18 mg/m². In some embodiments, rapamycin is administered at a dose of between about 1 mg/m² and 20 mg/m². In some embodiments, rapamycin is administered at a dose of between about 2 mg/m² and 40 mg/m². In some embodiments, rapamycin is administered at a dose of between about 3 mg/m² and 60 mg/m². In some embodiments, rapamycin is administered at a dose of between about 4 mg/m² and 80 mg/m². In some embodiments, rapamycin is administered at a dose of between about 5 mg/m² and 100 mg/m². In some embodiments, the dose of rapamycin is the total dose within a 24-hour time period.

In some embodiments, a dose of rapamycin is administered every day. In some embodiments, a dose of rapamycin is administered about every 1, 2, 3, 4, 5, 6, 7, 8, 9, 10, 11, 12, 13, or 14 days. In some embodiments, a dose of rapamycin is administered about every 1, 2, 3, 4, 5, 6, 7, 8, 9, or 10 weeks. In some embodiments, a dose of rapamycin is administered about every 1, 2, 3, 4, 5, 6, 7, 8, 9, or 10 months.

In some embodiments, after the first administration of the viral particle, the first dose of rapamycin is administered about 1, 2, 3, 4, 5, 6, 7, 8, 9, 10, 11, 12, 13, or 14 days post first administration of the viral particle. In some embodiments, after the first administration of the viral particle, the first dose of rapamycin is administered about 1, 2, 3, 4, 5, 6, 7, 8, 9, 10, 11, 12, 13, or 14 weeks post first administration of the viral particle. In some embodiments, after the first administration of the viral particle, the first dose of rapamycin is administered about 1, 2, 3, 4, 5, 6, 7, 8, 9, 10, 11, 12, 13, or 14 months post first administration of the viral particle. In some embodiments, after the first administration of the viral particle, the first dose of rapamycin is administered between about 1-3 days, between about 2-6 days, between about 3-9 days, between about 4-12 days, between about 5-15 days, between about 1-3 weeks, between about 2-4 weeks, between about 3-6 weeks, or between about 4-8 weeks post first administration of the viral particle.

In some embodiments, administration of rapamycin increases the number of viral particle transduced immune cells (e.g., CAR T cells) in the subject, or in a particular organ/region of the subject. In some embodiments, the organ/region of the subject is blood. In some embodiments, the organ/region of the subject is spleen. In some embodiments, the organ/region of the subject is bone marrow. In some embodiments, administration of rapamycin increases the number of viral particle transduced immune cells (e.g., CAR T cells) by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold, in the subject. In some embodiments, the increase is evaluated by the number of viral particle transduced immune cells 1, 2, 3, 4, 5, 6, 7, 8, 9, 10, 11, or 12 weeks after the first dose of the rapamycin (once the viral particle is administered), wherein the reference number is the number of viral particle transduced immune cells on the day of the first dose of rapamycin. In some embodiments, the increase is evaluated by the number of viral particle transduced immune cells 1, 2, 3, 4, 5, 6, 7, 8, 9, 10, 11, or 12 months after the first dose of the rapamycin (once the viral particle is administered), wherein the reference number is the number of viral particle transduced immune cells on the day of the first dose of rapamycin.

In some embodiments, administration of rapamycin increases the percentage of viral particle transduced immune cells (e.g., CAR T cells) in the subject, or in a particular organ/region of the subject. In some embodiments, the organ/region of the subject is blood. In some embodiments, the organ/region of the subject is spleen. In some embodiments, the organ/region of the subject is bone marrow. In some embodiments, administration of rapamycin increases the percentage of viral particle transduced immune cells (e.g., CAR T cells) by at least 1%, at least 2%, at least 3%, at least 5%, at least 7%, at least 10%, at least 20%, at least 30%, at least 40%, at least 50%, at least 60%, at least 70%, at least 80%, or at least 90% in the subject. In some embodiments, the increase is evaluated by the percentage of viral particle transduced immune cells 1, 2, 3, 4, 5, 6, 7, 8, 9, 10, 11, or 12 weeks after the first dose of the rapamycin (once the viral particle is administered), wherein the reference percentage is the percentage of viral particle transduced immune cells on the day of the first dose of rapamycin. In some embodiments, the increase is evaluated by the percentage of viral particle transduced immune cells 1, 2, 3, 4, 5, 6, 7, 8, 9, 10, 11, or 12 months after the first dose of the rapamycin (once the viral particle is administered), wherein the reference percentage is the percentage of viral particle transduced immune cells on the day of the first dose of rapamycin. In some embodiments, the percentage is the percentage of viral particle transduced immune cells in total immune cells in the subject or in the particular organ/region of the subject. In some embodiments, the percentage is the percentage of viral particle transduced immune cells in immune cells of the same type (e.g., T cells) in the subject or in the particular organ/region of the subject.

### Pharmaceutical Compositions and Formulations

The formulations and compositions of the present disclosure may comprise a combination of any number of viral particles, and optionally one or more additional pharmaceutical agents (polypeptides, polynucleotides, compounds etc.) formulated in pharmaceutically acceptable or physiologically-acceptable compositions for administration to a cell, tissue, organ, or an animal, either alone, or in combination with one or more other modalities of therapy. In some embodiments, the one or more additional pharmaceutical agent further increases transduction efficiency of vectors.

In some embodiments, the present disclosure provides compositions comprising a therapeutically-effective amount of a viral particle, as described herein, formulated together with one or more pharmaceutically acceptable carriers (additives) and/or diluents. In some embodiments, the composition further comprises other agents, such as, e.g., cytokines, growth factors, hormones, small molecules or various pharmaceutically active agents.

In some embodiments, compositions and formulations of the viral particles used in accordance with the present disclosure may be prepared for storage by mixing a viral particle having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. An isotonic formulation is liquid, or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

Non-limiting examples of salts include salts of any combinations of the cations sodium potassium, calcium or magnesium with anions chloride, phosphate, citrate, succinate, sulphate or mixtures thereof. Non-limiting examples of amino acids comprise arginine, glycine, ornithine, lysine, histidine, glutamic acid, asparagic acid, isoleucine, leucine, alanine, phenylalanine, tyrosine, tryptophane, methionine, serine, proline. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Non-limiting examples of stabilizer includes amino acids and sugars as described above as well as commercially available cyclodextrins and dextrans of any kind and molecular weight as known in the art. Non-limiting examples of antioxidants include excipients such as methionine, benzylalcohol or any other excipient used to minimize oxidation.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the present disclosure is contemplated.

The compositions may further comprise one or more polypeptides, polynucleotides, vectors comprising same, compounds that increase the transduction efficiency of vectors, formulated in pharmaceutically acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules or various pharmaceutically active agents. There is virtually no limit to other components that may also be included in the compositions, provided that the additional agents do not adversely affect the ability of the composition to deliver the intended therapy.

The present disclosure also provides pharmaceutical compositions comprising an expression cassette or vector (e.g., therapeutic vector) disclosed herein and one or more pharmaceutically acceptable carriers, diluents or excipients. In some embodiments, the pharmaceutical composition comprises a lentiviral vector comprising an expression cassette disclosed herein, e.g., wherein the expression cassette comprises one or more polynucleotide sequences encoding one or more chimeric antigen receptor (CARs) and variants thereof.

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The vector can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the vectors according to the present disclosure.

In some embodiments, the viral particle (e.g., lentiviral particle), or a pharmaceutical composition comprising that viral particle, is effective when administered systemically. For example, the viral vectors of the disclosure, in some cases, demonstrate efficacy when administered intravenously to subject (e.g., a primate, such as a non-human primate or a human). In some embodiments, the viral vectors of the disclosure are capable of inducing expression of CAR in various immune cells when administered systemically (e.g., in T-cells, dendritic cells, NK cells).

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral vectors general contains salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In some embodiments, the formulation is stable for storage and use when frozen (e.g., at less than 0° C., about −60° C., or about −72° C.). In some embodiments, the formulation is a cryopreserved solution.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation.

In certain circumstances, it will be desirable to deliver the compositions disclosed herein parenterally, intravenously, intramuscularly, or intraperitoneally, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, are added. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In some embodiments, the solution intended for subcutaneous administration includes hyaluronidase. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

In some embodiments, the present disclosure provides formulations or compositions suitable for the delivery of viral vector systems (i.e., viral-mediated transduction) including, but not limited to, retroviral (e.g., lentiviral) vectors.

### Combination Therapy

The present disclosure further contemplates that one or more additional agents that improve the transduction efficiency of viral particle may be used.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone, vincristine, cyclophosphamide, hydroxy daunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

In some embodiments, the one or more agents to be administered with or after the viral particle comprises one or more adaptor molecules. In some embodiments, these adaptor molecules may comprise a targeting moiety and a hapten. In such embodiments, the viral particle may comprise a sequence encoding a hapten-specific CAR. Exemplary combinations are disclosed in WO 2021/076788 and US 20170290900, each of which is incorporated herein in its entirety.

### Ex-Vivo Manufacturing of Engineered Cell Therapies

In some aspects, the disclosure provides a method of delivering a nucleic acid to a cell ex vivo. In some embodiments, the disclosure provides a method of delivering a nucleic acid to an immune cell ex vivo. In some embodiments, the viral particles of the disclosure activate and transduce an immune cell ex vivo.

In some embodiments, the disclosure provides a method of delivering a nucleic acid to a cell in an ex vivo CAR T manufacturing process. Such methods typically involve the isolation of PBMCs from a patient via leukapheresis. These cells are washed and optionally further purified via one or more selection steps to isolate particular T cell populations of interest. In some aspects, these might include CD4+ and/or CD8+ T cells. The washed cells may be optionally activated and then transduced using a lentiviral vector. The washed and purified cells may be optionally activated and then transduced using a lentiviral vector. The purified cells may be optionally activated and then transduced using a lentiviral vector. The activation step may comprise contacting the cells with an exogenous activation agent such as anti-CD3 and anti-CD28 antibodies bound to a substrate or using unbound antibodies. Exemplary activation agents include anti-CD3 and anti-CD28-presenting beads and/or soluble polymers. After transduction, the cells may be optionally further washed and cultured until harvest. Methods of manufacturing engineered cell therapies, including CAR T cells, are known in the art (see e.g., Abou-el-Enein, M. et al. Blood Cancer Discov (2021), Vol 2(5): 408-422; Arcangeli, S. et al. Front. Immunol (19 Jun. 2020), Vol. 11 (1217) 1-13; Ghassemi, S. et al. Nat Biomed Eng (February 2022), Vol 6(2): 118-128; Vormittag, P. et al. Curr Opin Biotechnol (October 2018), Vol. 54: 164-181; each of which is herein incorporated by reference). Exemplary methods of autologous CAR T manufacturing are disclosed in US Patent Publication Nos. 2019/0269727, 2016/0122782, 2021/0163893, and US 2017/0037369, each of which is incorporated herein in its entirety.

In some embodiments, the disclosure provides a method of delivering a nucleic acid to a cell in an ex-vivo closed-loop manufacturing process. In some embodiments, an ex-vivo manufacturing process is an extracorporeal process. In exemplary embodiments, the lentiviral vectors disclosed herein permit delivery of a nucleic acid to a target cell during a closed-loop process. Exemplary methods of closed-loop and/or extracorporeal processes are disclosed in US Patent Publication No. 2021/0244871 and WO2022072885, both of which are incorporated herein in their entirety. In some embodiments, the lentiviral vectors as disclosed herein may be used to transduce cells ex vivo. For example, in exemplary closed-loop manufacturing processes, cells are obtained from a subject, washed, incubated and/or contacted with lentiviral particles, optionally washed again, and infused into the subject in a closed-loop system. In such embodiments, the lentiviral particles as disclosed herein are useful even without prior activation of the cells and are capable of binding to the cells in a short incubation and/or contacting step. In some embodiments, the incubation and/or contacting step is approximately or less than one hour. In some embodiments, the incubation and/or contacting step is approximately or less than two hours, approximately or less than three hours, approximately or less than four hours, or approximatey or less than five hours. In some embodiments, the incubation and/or contacting step is less than 12 hours or less than 24 hours. In some embodiments, a nucleic acid is delivered to a cell by transduction with a lentiviral vector such that the nucleic acid enters the cell ex-vivo. In some embodiments, a nucleic acid is delivered to a cell by contacting the lentiviral vector to the surface of the cell. In such embodiments, the nucleic acid may enter the cell ex-vivo or in vivo after the cells (complexed with the lentiviral vector) are infused back into the subject.

In some embodiments, the lentiviral vectors as disclosed herein eliminate the need for an ex-vivo activation step. In such embodiments, the isolated cells could be transduced directly after leukapheresis, washing, or selection. It is contemplated that the surface engineering described herein enables the lentiviral particles disclosed herein to activate and transduce cells in a single step. In such embodiments, the lentiviral particles disclosed herein may enable a short or truncated manufacturing process, reducing the time spent in ex-vivo manufacturing by eliminating one or more unit operations (e.g. activation prior to transduction) and/or reducing the amount of time necessary in post-transduction cell culture.

### Diseases

The disclosure also provides a viral particle that can be used for treatment of diseases, disorders or conditions. In some embodiments, the disease or disorder is cancer. In some embodiments, the cancer is a hematological malignancy or a solid tumor. In some embodiments, the subject is relapsed or refractory to treatment with a prior anti-cancer therapeutic.

In some embodiments, a therapeutic application of the viral particles disclosed herein is to treat malignancies that have failed other non-CAR T-cell treatment options.

### Hematological Malignancy

In some embodiments, the cancer is a hematological malignancy.

In some embodiments, the hematological malignancy is lymphoma, a B cell malignancy, Hodgkin's lymphoma, non-Hodgkin's lymphoma, a DLBLC, a FL, a MCL, a marginal zone B-cell lymphoma (MZL), a mucosa-associated lymphatic tissue lymphoma (MALT), a CLL, an ALL, an AML, Waldenstrom's Macroglobulinemia or a T-cell lymphoma.

In some embodiments, the solid tumor is a lung cancer, a liver cancer, a cervical cancer, a colon cancer, a breast cancer, an ovarian cancer, a pancreatic cancer, a melanoma, a glioblastoma, a prostate cancer, an esophageal cancer or a gastric cancer. WO2019057124A1 discloses cancers that are amenable to treatment with T cell redirecting therapeutics that bind CD19.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fins-related tyrosine kinase 3 (FLT3), nucleophosmin (NPM1), isocitrate dehydrogenase 1 (IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1 (U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the hematological malignancy is the ALL.

In some embodiments, the ALL is B-cell lineage ALL, T-cell lineage ALL, adult ALL or pediatric ALL.

In some embodiments, the subject with ALL has a Philadelphia chromosome or is resistant or has acquired resistance to treatment with a BCR-ABL kinase inhibitor.

The Ph chromosome is present in about 20% of adults with ALL and a small percentage of children with ALL and is associated with poor prognosis. At a time of relapse, patients with Ph+ positive ALL may be on tyrosine kinase inhibitor (TKI) regimen and may have therefore become resistant to the TKI. The method as described herein may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

In some embodiments, the subject has ALL with t(v;11q23) (MLL rearranged), t(1;19)(q23;pl3.3); TCF3-PBX1 (E2A-PBX1), t(12;21)(p13;q22); ETV6-RUNX1 (TEL-AML1) or t(5;14)(q31;q32); IL3-IGH chromosomal rearrangement.

Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma, MGUS, ALL, DLBLC, BL, FL, MCL, Waldenstrom's macroglobulinema, plasma cell leukemia, AL, precursor B-cell lymphoblastic leukemia, precursor B-cell lymphoblastic leukemia, myelodysplastic syndrome (MDS), CLL, B cell malignancy, CML, HCL, blastic plasmacytoid dendritic cell neoplasm, Hodgkin's lymphoma, non-Hodgkin's lymphoma, MZL, MALT, plasma cell leukemia, ALCL, leukemia, or lymphoma.

In some embodiments, the cancer is diffuse large B-cell lymphoma (DLBCL). In some embodiments, the cancer is Burkitt's type large B-cell lymphoma (B-LBL). In some embodiments, the cancer is follicular lymphoma (FL). In some embodiments, the cancer is chronic lymphocytic leukemia (CLL). In some embodiments, the cancer is acute lymphocytic leukemia (ALL). In some embodiments, the cancer is mantle cell lymphoma (MCL).

### Solid Tumor

In some embodiments, the cancer is a solid tumor.

In some embodiments, the solid tumor is a prostate cancer, a lung cancer, a non-small cell lung cancer (NSCLC), a liver cancer, a cervical cancer, a colon cancer, a breast cancer, an ovarian cancer, an endometrial cancer, a pancreatic cancer, a melanoma, an esophageal cancer, a gastric cancer, a stomach cancer, a renal carcinoma, a bladder cancer, a hepatocellular carcinoma, a renal cell carcinoma, an urothelial carcinoma, a head and neck cancer, a glioma, a glioblastoma, a colorectal cancer, a thyroid cancer, epithelial cancers, or adenocarcinomas.

In some embodiments, the prostate cancer is a relapsed prostate cancer. In some embodiments, the prostate cancer is a refractory prostate cancer. In some embodiments, the prostate cancer is a malignant prostate cancer. In some embodiments, the prostate cancer is a castration resistant prostate cancer.

### Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

A “comparison window,” as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used in the description of the invention and the appended claims, the singular forms “a”, “an”, and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (or).

As used herein, “administering” refers to local and systemic administration, e.g., including enteral, parenteral, pulmonary, and topical/transdermal administration. Routes of administration for pharmaceutical ingredients (e.g., vectors) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a pharmaceutical ingredient or composition to a mammal so that the pharmaceutical ingredient or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the pharmaceutical ingredient (e.g., vector) and/or analogs thereof and another active agent (e.g., multispecific antibody), refers to administration of the pharmaceutical ingredient and/or analogs and the active agent such that both can simultaneously achieve a physiological effect. The two agents, however, need not be administered together. In some embodiments, administration of one agent can precede administration of the other. Simultaneous physiological effect need not necessarily require presence of both agents in the circulation at the same time. However, in some embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “effective amount” or “pharmaceutically effective amount” refer to the amount and/or dosage, and/or dosage regime of one or more pharmaceutical ingredients (e.g., vectors) necessary to bring about the desired result.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In some embodiments, the reduction or elimination of one or more symptoms of pathology or disease can include, e.g., measurable and sustained decrease of tumor volume.

As used herein, the phrase “consisting essentially of” refers to the genera or species of active pharmaceutical agents recited in a method or composition, and further can include other agents that, on their own do not have substantial activity for the recited indication or purpose.

The terms “subject,” “individual,” and “patient” interchangeably refer to a mammal, preferably a human or a non-human primate, but also domesticated mammals (e.g., canine or feline), laboratory mammals, and agricultural mammals. In various embodiments, the subject can be a human (e.g., adult male, adult female, adolescent male, adolescent female, male child, female child).

The term “viral particle” as used herein refers a macromolecular complex capable of delivering a foreign nucleic acid molecule into a cell independent of another agent. A particle can be a viral particle or non-viral particle. Viral particle includes retroviral particle and lentiviral particle. Non-viral particles are limited to liposomes, nanoparticles, and other encapsulation systems for delivery of polynucleotides into cells.

The abbreviations “a” or “anti-” before the name of a gene refers to an antibody or antigen binding fragment of an antibody (such as an scFv) that specifically binds to a target. For example, αCD19 refers to an anti-CD19 antibody or antigen binding fragment thereof and αCD3 refers to an anti-CD3 antibody or antigen binding fragment thereof.

As used herein, the terms “expression cassette” or “vector genome” refer to a DNA segment that is capable in an appropriate setting of driving the expression of a polynucleotide (a “transgene” or “payload”) encoding a polypeptide (e.g., chimeric antigen receptor) that is incorporated in said expression cassette. When introduced into a host cell, an expression cassette inter alia is capable of directing the cell's machinery to transcribe the transgene into RNA, which is then usually further processed and finally translated into the polypeptide. The expression cassette can be comprised in a particle (e.g., viral particle). Generally, the term expression cassette excludes polynucleotide sequences 5′ to the 5′ ITR and 3′ to the 3′ ITR.

The terms “transgene” or “payload” refer to the transferred nucleic acid itself. The transgene may be a naked nucleic acid molecule (such as a plasmid) or RNA. The transgene may include a polynucleotide encoding one or more polypeptides (e.g., chimeric antigen receptor). The transgene may include a polynucleotide encoding one or more heterologous protein (e.g., a chimeric antigen receptor), one or more capsid proteins, and other proteins necessary for transduction of the polynucleotide into a target cell.

The term “derived” is used to indicate that the cells have been obtained from their biological source and grown or otherwise manipulated in vitro (e.g., cultured in a growth medium to expand the population and/or to produce a cell line).

The term “transduce” refers to introduction of a nucleic acid into a cell or host organism by way of a particle (e.g., a lentiviral particle). Introduction of a transgene into a cell by a viral particle can therefore be referred to as “transduction” of the cell. The transgene may or may not be integrated into the genomic nucleic acid of a transduced cell. If an introduced transgene becomes integrated into the nucleic acid (genomic DNA) of the recipient cell or organism it can be stably maintained in that cell. Alternatively, the introduced transgene may exist in the recipient cell or host organism extra-chromosomally, or only transiently. A “transduced cell” is therefore a cell into which the transgene has been introduced by way of transduction. Thus, a “transduced” cell is a cell into which, a polynucleotide has been introduced.

The term “transduction efficiency” is an expression of the proportion of cells that express or transduce a transgene when a cell culture is contacted with particles. In some embodiments, the efficiency can be expressed as the number of cells expressing a transgene when a given number of cells are contacted with a given number of particles. In some embodiments, “Relative transduction efficiency” is the proportion of cells transduced by a given number of viral particles in one condition relative to the proportion of cells transduced by that same number of particles in another condition comprising a similar number of cells of the same cell type. Relative transduction efficiency is most often used to compare the effects of a modulator of transduction efficiency on cells and/or animals treated or not treated with that modulator.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary.

### Example 1: Particle Generation

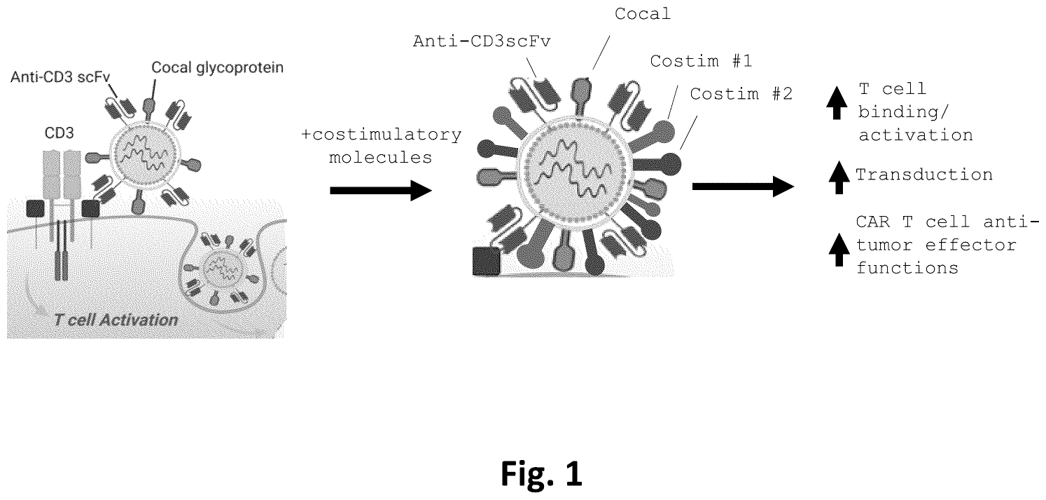

The purpose of this study was to investigate the impact of incorporating a costimulatory molecule, CD80, and/or an adhesion protein, CD58, onto the surface of a viral particle. A schematic of such viral particles is provided in FIG. 1.

### Virus Production

1.2×10⁶ 293T cells were seeded into TC-treated 6 well plates in a total volume of 2.5 ml Complete DMEM media. 24 hours later, cells were transfected. (protocol written for 1 well of 6 well plate; all reagents should be room temperature)

The following DNA was added to 500 ul serum free OptiMEM media: 2 μg transfer plasmid, 1 μg Gag/pol plasmid, 1 μg REV plasmid, 1 μg envelope plasmid. 15 ul (15 ug) PEI was then added to the media/DNA mix. Mixture was mixed well and incubated at room temperature for 20 minutes. The media/DNA/PEI mix was then added to 2.5 ml fresh Complete DMEM media. The seeding media in 293T-containing well was removed and replaced with fresh media containing the transfection reagents and placed in 37° C. humidified incubator. 48 hours later, the supernatant was collected and filtered through a 0.45 um PVDF filter. The supernatant was concentrated using Amicon-Ultra 15 100K column and centrifuged at 3000×g for 30 minutes at 4° C. The virus was then stored at 4° C. until use.

### 293T Transduction Titers

1×10⁵ 293T cells were seeded into TC-treated 12 well plates in 1 ml Complete DMEM media. 24 hours later, empty wells were counted 3× to calculate titer. Then add virus to wells in the amount: 2 ul, 1 ul, 0.5 ul, 0.2 ul, 0.1 ul, 0.05 ul virus per well. Virus was diluted 1:100 before adding to 293T cells. 3 days later, 293T cells were harvested for analysis by flow cytometry. Media was removed, cells were washed in PBS, cells were then washed in Trypsin and incubate for ˜3-5 minutes in 37° C. incubator. Cells were resuspended in 1 ml FACS buffer and ˜100-200 ul were added to a 96 well V bottom plate. Flow cytometry analysis was performed for mCherry expression.

### 293T Titer Calculation:

TU/ml=(#of cells at time of transduction×% mCherry+×100)/(vector volume in ul×1000)

Engineered particles packaging an anti-CD19 CAR containing either a CD3scFV alone or a CD3scFV+CD80, CD3scFV+CD58, or CD3scFV+CD80+CD58 were added to PBMCs from 2-3 donors.

### Example 2: Viral Particles Expressing Co-Stimulatory or Adhesion Molecules Enhance T Cell Activation and Transduction

To determine whether incorporation of a co-stimulatory molecule and/or adhesion molecule on a viral particle could enhance transduction of the viral particle, the viral particles generated in Example 1 were incubated with PBMCs.

### Virus Production

All solutions used were the same as those described in Example 1. 28×10⁶ 293T cells were seeded into 16× T175 flasks (8× per vector) with 28e6 293T cells each in a total volume of 25 ml Complete DMEM media. 24 hours later, cells were transfected. Virus was produced as described in Example 1. All viruses included a Cocal envelope protein.

### List of Virus Preps Made for Study:

1. CD3scfv only
2. CD3scfv+CD58
3. CD3scfv+CD80

### PBMC Transduction and Staining for Flow Cytometry

50×10⁶ PBMCs were thawed, diluted to 2×10⁶ cells/ml in complete media (e.g. RPMI or Optimem). IL-2 was added to a final concentration of 50IU/ml.

500 μl(1e6 cells) were added to the wells of a Non TC-treated 48 well plate. Vector was added to the wells at MOI=10, 5, and 2 based on the SupT1 ddPCR titer and the plates were placed in 37° C. incubator.

After 3 days, vector was washed out and replaced with 500 μl fresh RPMI media+IL-2 (50IU/ml). Cells were mixed and 100-300 μl were added to wells in a 96 well V-bottom plate for activation flow cytometry analysis. Cells were then washed with 200 μl FACS buffer. The cell pellets were resuspended in 50-100 μl PBS containing LiveDead Stain (1:1000) and incubate at 4° C. for 20 min followed by another wash in 200 μl FACS buffer. Cells were resuspended in 50 μl of FACS buffer +surface stain cocktail, incubated for 30 min at 4° C., washed in 200 μl FACS buffer.

### Results and Conclusions

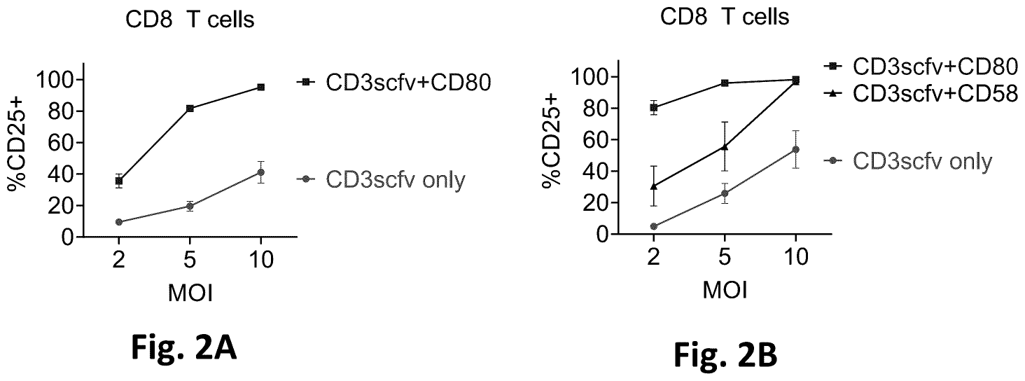

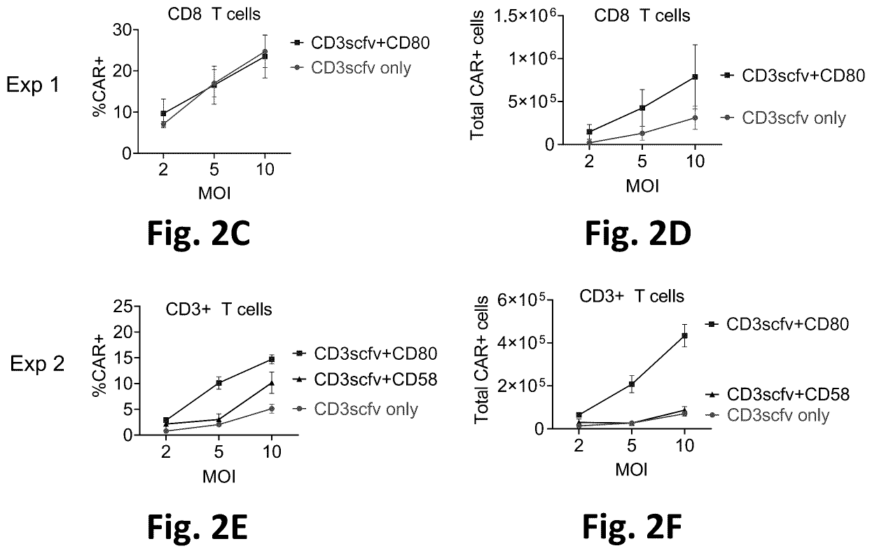

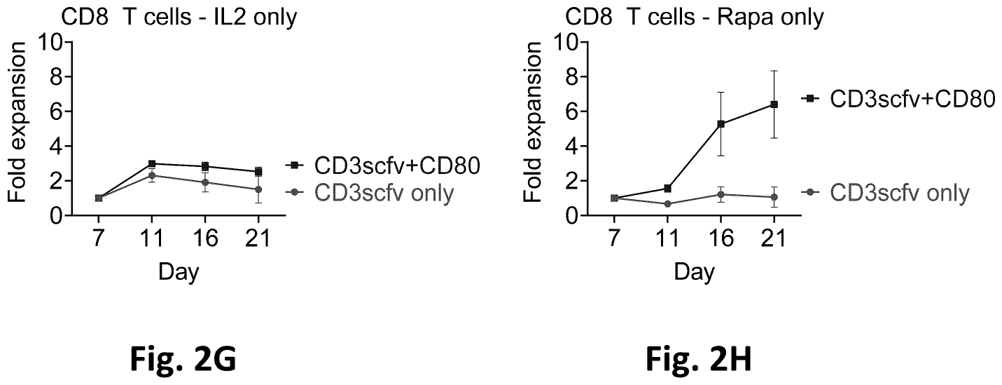

To assess if viral particles with co-stimulatory molecules can better activate human T cells, the vector particles were added to human PBMCs at several MOI's. 3 days later, the virus was removed and the cells were given fresh media and analyzed for the activation marker CD25. CD3scfv+CD58 and CD3scfv+CD80 particles potently activated CD8 T cells compared to CD3scfv only (FIG. 2A and FIG. 2B). Furthermore, CD25 upregulation was dose-dependent (FIG. 2A and FIG. 2B). CD3scfv only viral particles induced minimal levels of CD25 compared to the particles with CD80 or CD58 (FIG. 2A and FIG. 2B).

To examine transduction, 6 total days after vector addition, samples were analyzed for anti-CD19 CAR expression. Mirroring the CD25 expression on day 3, CD3scfv+CD58 and CD3scfv+CD80 particles were capable of transducing unstimulated PBMCs while CD3scfv only particles transduced unstimulated PBMCs to a lesser extent (FIGS. 2C-2F). Furthermore, transduction occurred in a dose-dependent manner for both CD3 and CD8 T cells (FIGS. 2C-2F). The data show that CD3scfv+CD58 and CD3scfv+CD80 particles efficiently activate and transduce unstimulated PBMCs in vitro compared to CD3scfv only. Importantly, the enhanced particles results in increased numbers of CAR+ T cells (FIGS. 2D-2F).

To determine if adding costimulatory molecules to particles enhances Rapa-mediated expansion of CAR+ cells in vitro, the fold expansion of CD8 T cells was determined using CD3scfv+CD80 particles compared to CD3scfv only particles. PMBCs were cultures in either IL-2 only media or Rapamycin-only media. The addition of the co-stimulatory molecule did not affect the fold expansion when cultured with IL-2 only (FIG. 2G) but the co-stimulatory molecule induced a dramatic expansion when cultured with Rapamycin-media (FIG. 2H). These results demonstrate that adding co-stimulatory molecules to particles enhances Rapamycin-mediated expansion of CAR+ cells in vitro.

This study demonstrated the ability of the CD3scfv+co-stimulatory molecules envelope construct to deliver payloads consisting of an anti-CD19 CAR to unstimulated PBMCs in vitro. The CD3scfv+CD58 and CD3scfv+CD80 particles induced activation of T cells as measured by CD25 expression and this activation correlated with transduction as measured by % of T cells expressing the anti-CD19 CAR and total CAR+ T cells. Furthermore, activation and transduction occurred in a dose-dependent manner. Co-stimulatory molecules also enhance Rapamycin-mediated expansion of CAR+ cells in vitro. This data further supports the use CD3scfv+CD58 and CD3scfv+CD80 particles to deliver CAR payloads to unstimulated PBMCs in vitro and in vivo.

### Example 3: Viral Particles Expressing Co-Stimulatory and/or Adhesion Molecules Enhance T Cell Activation, Transduction and Subsequent Tumor Cell Killing

To determine whether a combination of a co-stimulatory molecule (CD80), and an adhesion protein (CD58) could further enhance T cell activation and transduction, particles having both molecules were generated. These particles were examined for their ability to activate and transduce unstimulated human PBMCs compared to particles only having anti-CD3scFv.

### Virus Production

All solutions used were the same as those described in Example 1. 28×10⁶ 293T cells were seeded into 16× T175 flasks (8× per vector) with 28×10⁶ 293T cells each in a total volume of 25 ml Complete DMEM media. 24 hours later, cells were transfected. Virus was produced as described in Example 1.

### List of Virus Preps Made for Study:

1. CD3scfv only
2. CD3scfv+CD58
3. CD3scfv+CD80
4. CD3scfv+CD80+CD58

### PBMC Transduction and Analysis

PBMCs were transduced and analyzed for expression as described in Example 2.

Supernatant cytokine analysis was measured by Meso Scale Discovery (MSD) 3 days after transduction.

Total K562.CD19, Raji, and Nalm6 tumor cells were tracked over time on an IncuCyte® for up to 15 days.

### Results and Conclusions

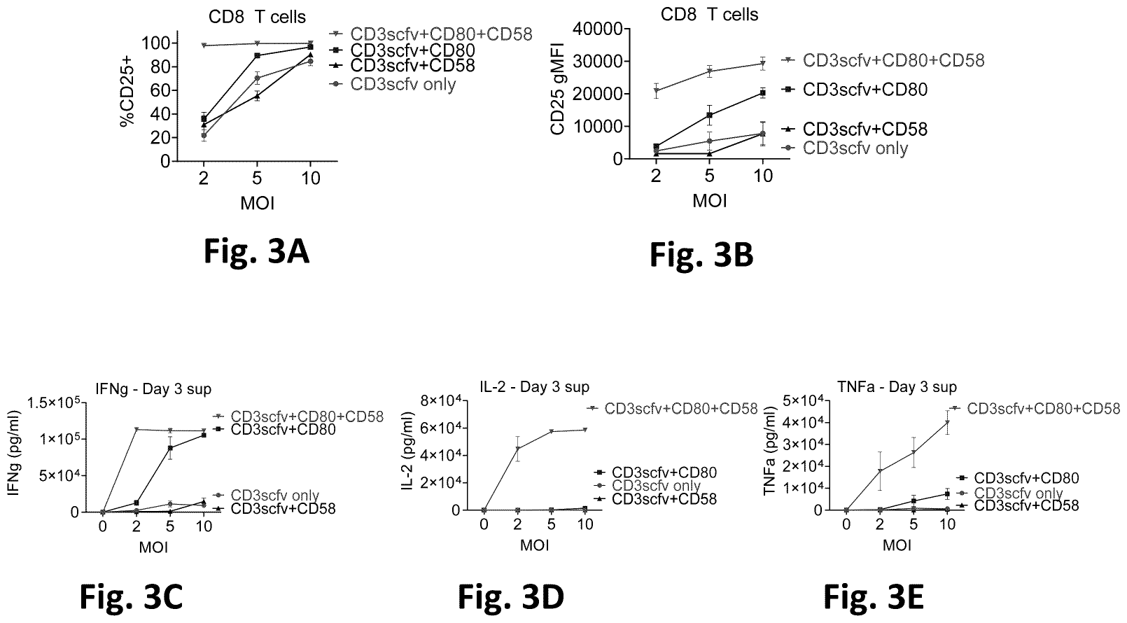

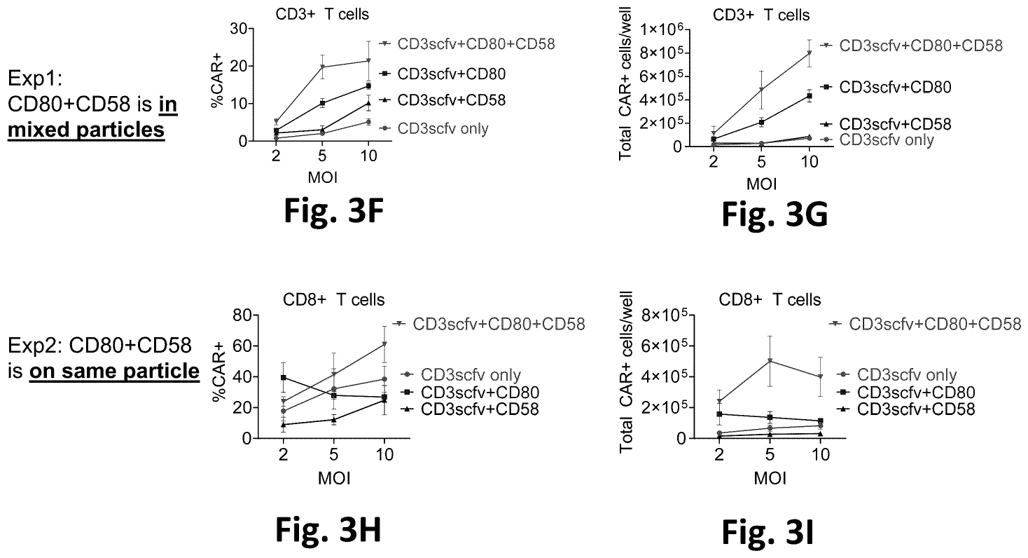

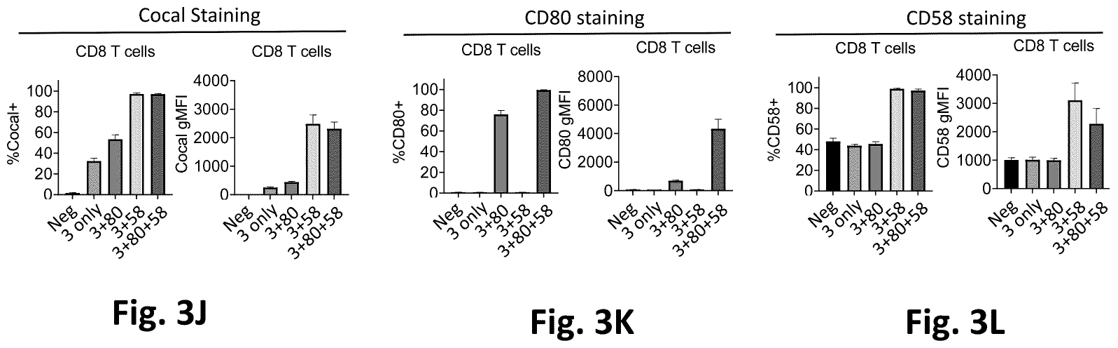

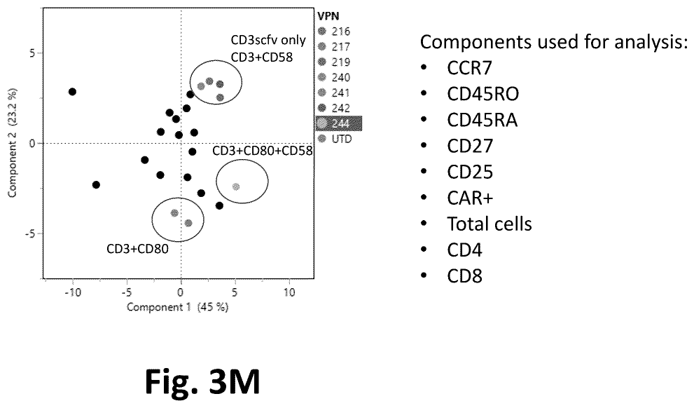

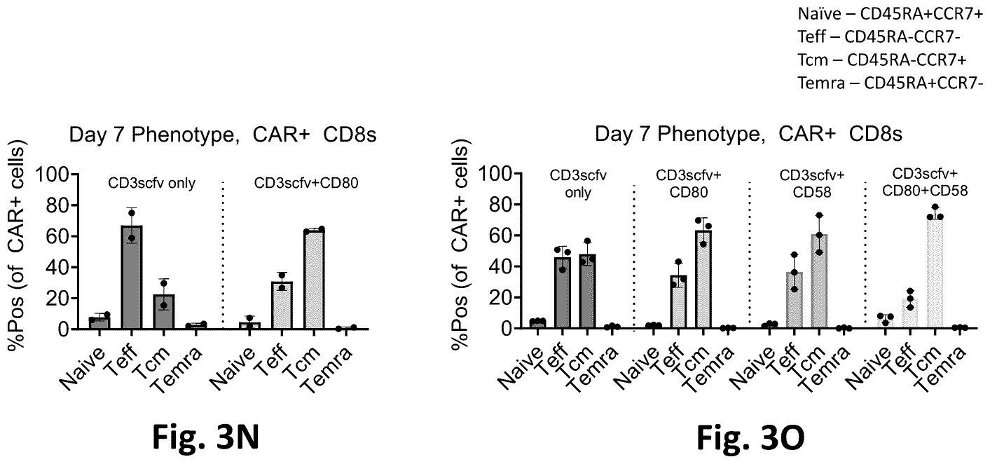

To assess whether viral particles with co-stimulatory molecules and adhesion molecules enhance T cell activation and transduction, the viral particles were added to human PBMCs at several MOI's. 3 days later, the virus was removed and the cells were given fresh media and analyzed for the activation marker CD25. CD3scfv+CD80+CD58 particles potently activated CD8 T cells compared to CD3scfv+CD58, CD3scfv+CD80, and CD3scfv only (FIG. 3A and FIG. 3B). Furthermore, CD25 upregulation was dose-dependent and CD3scfv+CD80+CD58 particles activated CD8 T cells a a much lower dose (FIG. 3A and FIG. 3B). CD3scfv+CD58, CD3scfv+CD80, and CD3scfv only viral particles induced minimal levels of CD25 compared to the CD3scfv+CD80+CD58 particles (FIG. 3A and FIG. 3B).

To further characterize T cell activation, 3 total days after vector addition, samples were analyzed for cytokine expression. Similar to CD25 expression, CD3scfv+CD80 and CD3scfv+CD80+CD58 particles were capable of inducing IFN-γ production unstimulated PBMCs at lower doses whereas CD3scfv+CD58 and CD3scfv only particles transduced unstimulated PBMCs to a lesser extent (FIG. 3C). Furthermore, CD3scfv+CD80+CD58 particles induced robust IL-2 and TNF-α whereas CD3scfv+CD58, CD3scfv+CD80, and CD3scfv only did not (FIG. 3D and FIG. 3E). The data show that CD3scfv+CD80+CD58 particles efficiently induce cytokine production in unstimulated PBMCs in vitro compared to CD3scfv+CD58, CD3scfv+CD80, and CD3scfv only.

To examine the role of CD80 and CD58 on transduction, 3 total days after vector addition, samples were analyzed for anti-CD19 CAR expression with CD3scfv+CD80 and CD3scfv+CD58 mixed particles (FIG. 3F and FIG. 3G) or CD3scfv+CD80+CD58 on the same particle (FIG. 3H and FIG. 3I) compared to CD3scfv+CD58, CD3scfv+CD80, and CD3scfv only. CD3scfv+CD80 and CD3scfv+CD58 mixed particles or CD3scfv+CD80+CD58 on the same particle were both capable of transducing unstimulated PBMCs to a greater extent than CD3scfv+CD58, CD3scfv+CD80, and CD3scfv only (FIG. 3F, FIG. 3G, FIG. 3H and FIG. 3I). Furthermore, transduction occurred in a dose-dependent manner for both CD3 and CD8 T cells (FIG. 3F, FIG. 3G, FIG. 3H and FIG. 3I). The data show that both CD58 and CD80 either in mixed particles or on the same particle better activate and transduce unstimulated PBMCs in vitro compared to CD3scfv+CD58, CD3scfv+CD80, and CD3scfv only.

To determine whether viral particles with co-stimulatory and/or adhesion molecules have enhanced particle binding to T cells, the particles were cultured with PBMCs for 6 hours and then were analyzed for particle-associated molecules on T cells (Cocal, CD80, and CD58). Both CD3scfv+CD58 and CD3scfv+CD80+CD58 increased Cocal staining (FIG. 3J) and only CD3scfv+CD80+CD58 demonstrated high stating for CD80 (FIG. 3K) and CD58 (FIG. 3L). The data show that the combination of CD3scfv+CD80+CD58 enhances particle binding to T cells.

To determine whether different T cell subtypes are generated by the viral particles, PBMCs cultured with the viral particles were profiled and gated on viable, CD3+ and CD8+. The cells were further analyzed by flow and principal component analysis was done based on parameters listed CCR7, CD45R, CD45RA, CD27, CD25, CAR+, total cells, CD4, and CD8. The analysis revealed that 3 main clusters of differentiation are produced by the different particles (FIG. 3M).

Next, T cell subtypes generated by the particles was profiled. The cells were assessed using CD45RA and CCR7 markers 7 days post transduction at an MOI of 10. Naïve T cells are CD45RA+CCR7+, effector T cells (T_{eff}) are CD45RA-CCR7−, central memory T cells (T_{cm}) are CD45RA-CCR7+, and terminally differentiated effector memory T cells (Tₑₘᵣₐ) are CD45RA+CCR7−. In a first experiment, CD3scfv only particles produced a majority of T_{eff} cells whereas CD3scfv+CD80 particles produced a majority of T_{cm} cells (FIG. 3N). In a second experiment, CD3scfv only particles produced both T_{eff} and T_{cm} cells, CD3scfv+CD80 particles produced a majority of T_{cm} cells, CD3scfv+CD58 particles produced a majority of T_{cm} cells, and CD3scfv+CD80+CD58 produced a majority of T_{cm} cells (FIG. 3O). The data show that both the addition of CD80 and/or CD58 to the particle consistently produces a T_{cm} cell phenotype. CD45RA-CCR7+T_{cm} cells are thought to have increased longevity and proliferative capacity and correlate with better antitumor responses in vivo.

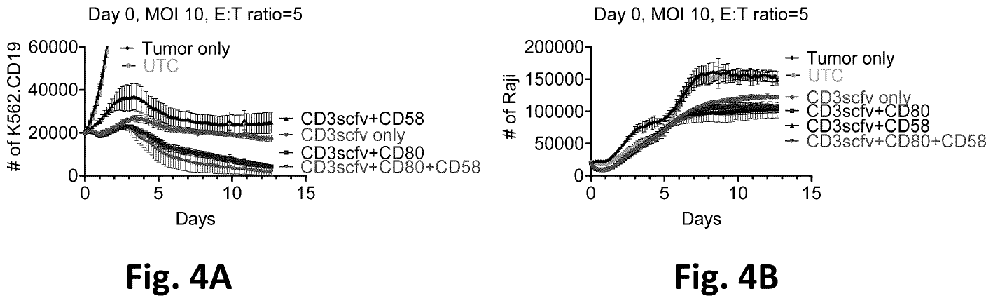

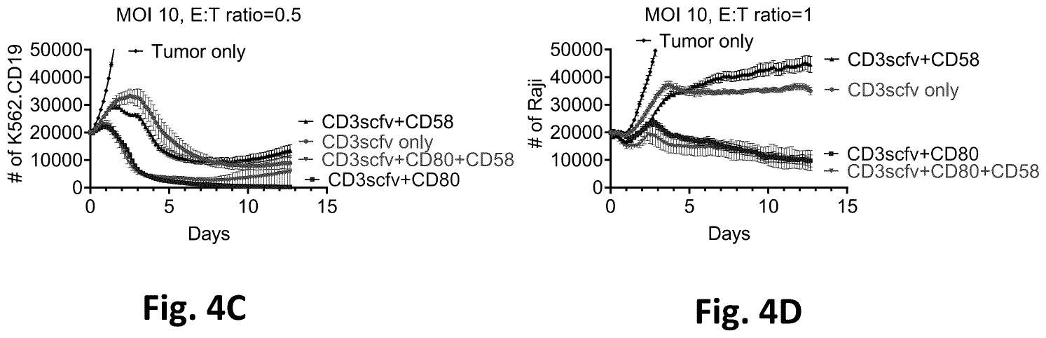

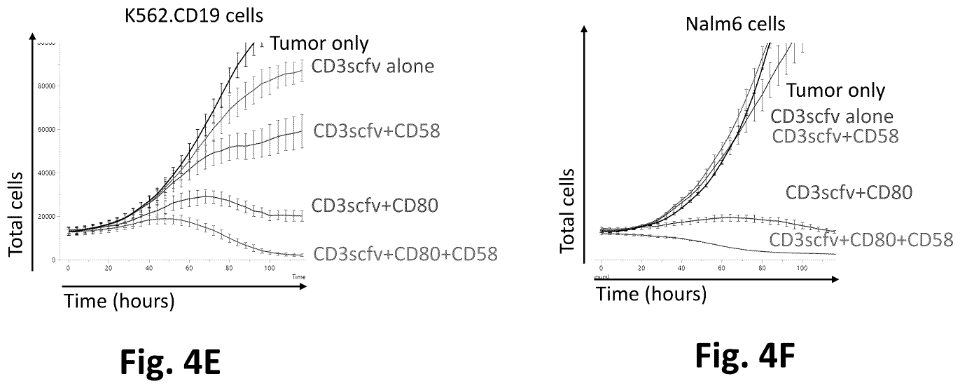

To assess the anti-tumor efficacy of CAR T cells generated using viral particles expressing co-stimulatory and/or adhesion molecules, PBMCs were transduced and cultured with tumor cells. Specifically, particles comprising a nucleotide sequence encoding an anti-CD19 CAR were added to PBMCs at an MOI of 10, along with tumor cells (K562.CD19 or Raji cells) at PBMC:Tumor ratio of 5:1 and put directly on an Incucyte. Tumor cell killing was measured over time. The highest killing was observed with particles composed of at least CD80 in addition to CD3scfv (FIG. 4A and FIG. 4B). In a subsequent experiment, tumor cell killing was measured 7 days after transduction with an MOI of 10. The total number of CAR+ cells were calculated and incubated with either K562.CD19 or Raji cells at E:T ratios of 0.5 and 1, respectively. CAR T cells generated using a mixture of individual particles with CD80 or CD58. Similarly, the highest killing was observed with particles composed of at least CD80 in addition to CD3scfv, including CD80+CD58 (FIG. 4C and FIG. 4D). An additional experiment determined the effect for CAR T cells generated with a single viral particle having both CD80 and CD58. Tumor cell killing was measured 7 days after transduction at an MOI 10. The total number of CAR+ cells were calculated and incubated with either K562.CD19 or Nalm6 cells at E:T ratios of 1:1, respectively. The CD80+CD58 dual particle provided the highest cytotoxic function (FIG. 4E and FIG. 4F).

This study demonstrated the CD3scfv+CD80+CD58 particles induced the highest differentiation of T cells and the highest cytokine production at the lowest MOI. CD3scfv+CD80+CD58 particles further had the highest T cell binding. Furthermore, this study demonstrated that CD3scfv+CD80+CD58 particles provided the highest cytolytic function in vitro.

### Example 4: Tumor Control from In vivo Transduction of T Cells by Viral Particles with Co-Stimulatory and/or Adhesion Molecules

This study assessed tumor control from in vivo transduction of T cells by a lentiviral particle with CD3scfv or CD3scfv+CD80. The lentiviral particle contains a polynucleotide encoding an anti-CD19 CAR. The lentiviral particle was delivered via intravenous injection into NSG MHCI/II KO mice. The mice used in the study were immune-compromised and contain engrafted human T cells and circulating human B cells.

### Study Design

### Virus Preparation, Animal Strain, Cell Lines

11 female NSG MHCI/II KO mice (Jackson laboratory) were and housed following institutional guidelines (Fred Hutchinson Cancer Research Center).

### Study Protocol

11 female NSG MHCI/II KO mice were acclimated for one week after receipt. At day −7, blood from all mice was collected for flow cytometry analysis to quantify degree of humanization. Mice were randomized according to their total human CD3 levels into the treatment groups described in Table 2.

TABLE 2
Study Treatment Groups

| | | | Administration | Virus Dose |
|---|---|---|---|---|
| Group | N | Virus type | route* | (Titre Unit) |
| 1 | 6 | CD3scfv | IV | 50 Million TU |
| | | | | Day 0 |
| 2 | 5 | CD3scfv + CD80 | IV | 50 Million TU |
| | | | | Day 0 |

### Study Timeline

At study Day 0 (SD0), 20×10⁶ PBMCs were injected into the intraperitoneal cavity. Mice were then dosed with virus particles according to the table above, followed with a challenge of 5×10⁵ luciferase+Nalm6 tumor cells intravenously. Tumor burden was measured for the duration of the study. At SD11, blood was collected and CAR T cells were measured.

At SD75, survivor mice were rechallenged with 5×10⁶ Nalm6 cells.

### Results and Conclusions

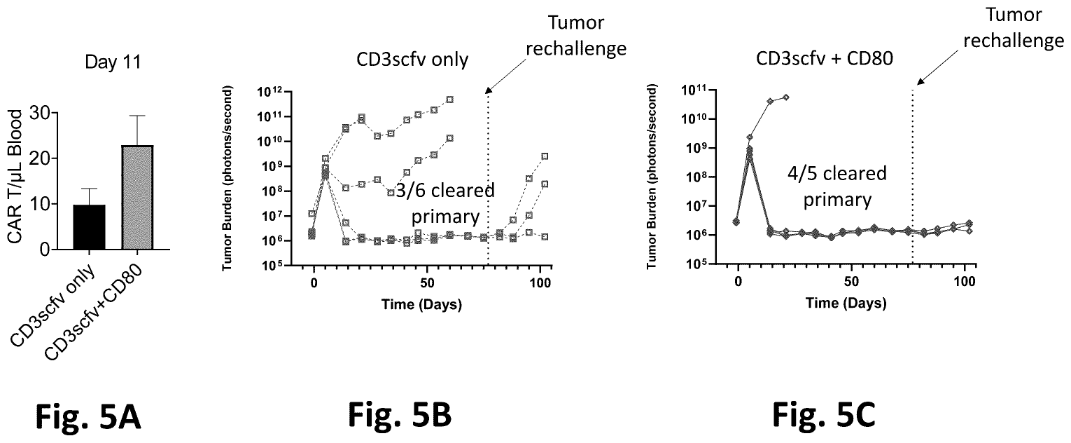

On Day 11 of the study, blood was collected from both groups. The level of CAR T cells in the blood was higher in the CD3scfv+CD80 particle-treated group compared to the CD3scfv particle-treated group (FIG. 5A). The CD3scfv+CD80 particle-treated group was also able to decrease tumor burden during the initial challenge and subsequent rechallenge compared to the CD3scfv particle-treated group (FIG. 5B and FIG. 5C).

In summary, when delivered intravenously, CD3scfv and CD3scfv+CD80 engineered lentivirus particles successfully transduced T cells in vivo. While both groups decreased tumor burden after initial challenge and subsequent rechallenge, particles with co-stimulatory molecule CD80 provided greater anti-tumor efficacy and anti-tumor immune response.

### Example 5: Efficient T Cell Transduction after Brief Incubation Period

This study assessed the ability for engineered viral particles as described herein to transduce T cells in a short incubation period. Without wishing to be bound by theory, this study provides proof of concept support that the engineered particles described may be useful in an extracorporeal intravenous system.

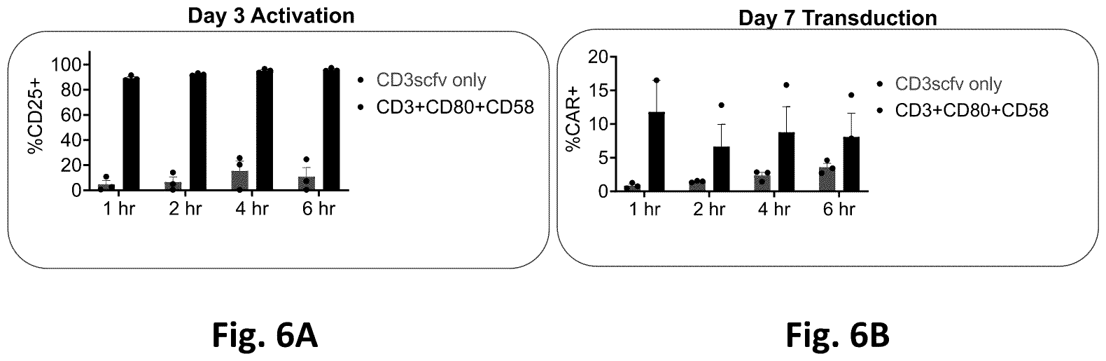

PBMCs from 3 healthy donors were thawed and cultured with vector particles containing an CD19 CAR-mCherry payload pseudotyped with either CD3scfv+cocal or CD3scfv+CD80+CD58+Cocal, generally as described in Example 2. After the indicated timepoint, cells were washed in serum-free media containing IL2, human ab serum, HEPES, and glutamine. Cells were then plated in 1 ml serum-free media with IL-2 in a 24 well non-TC-treated plate. 3 days later cells were harvested and CD25 expression was measured by flow cytometry on viable T cells (FIG. 6A). The remaining cells were washed and re-plated in 1 ml fresh media containing IL-2. 4 days later (Day 7 after transduction) viable T cells were analyzed by flow cytometry for CAR surface expression (FIG. 6B). % CAR was measured by staining for anti-CD19 mAb and mCherry expression.

As shown in FIGS. 6A-6B, vector particles comprising activation, costimulation, and adhesion molecules (e.g. CD3scFv+CD80+CD58 particles) efficiently transduced T cells after short incubation periods to a greater extent than particles comprising a CD3scFv without costimulation and adhesion components. These results indicate that vector particles as described herein may enable T cell transduction during short incubation periods ex vivo, for example in a closed-loop and/or extracorporeal system.

### Example 6: Transduction Rescue of Blinded Envelope Proteins

This study assessed the transduction potential of viral particles comprising a mutated envelope protein. Envelope proteins, such as VSV-G or cocal, can be mutated such that they cannot bind the LDL receptor. These modifications may enhance the specificity of viral particles and reduce or eliminate off-target transduction.

SupT1 cells were cultured with vector particles containing CD19 CAR-mCherry payload, generated generally as described in Example 2. Specifically, 0.02 uL of concentrated particles were added to 3.75×10⁴ SupT1 cells and assessed for CAR expression 3 days later. The cells were cultured in the following conditions:

| Condition | Particle Description |
|---|---|
| αCD3/CD58/CD80; | CD3scFv + CD80 + CD58; |
| no VSVG | no envelope |
| VSVG | VSV-G only |
| VSVG (R354Q) | VSV-G (R354Q) only |
| VSVG (K47Q) | VSV-G (K47Q) only |
| αCD3/CD58/CD80; VSVG | CD3scFv + CD80 + CD58; VSV-G |
| αCD3/CD58/CD80; VSVG | CD3scFv + CD80 + CD58; |
| (R354Q) | VSV-G (R354Q) |
| αCD3/CD58/CD80; VSVG | CD3scFv + CD80 + CD58; |
| (K47Q) | VSV-G (K47Q) |

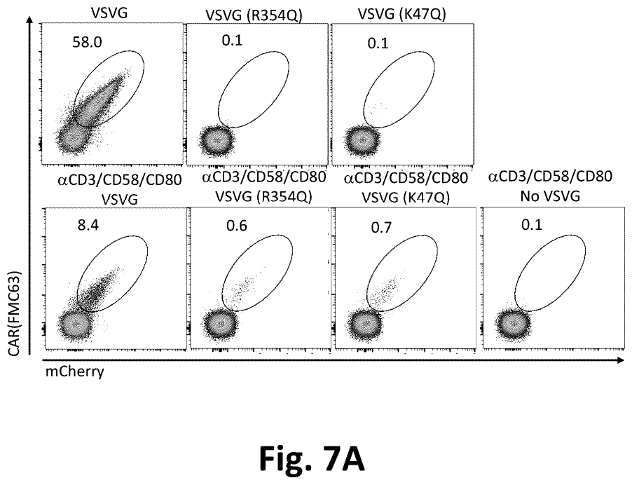

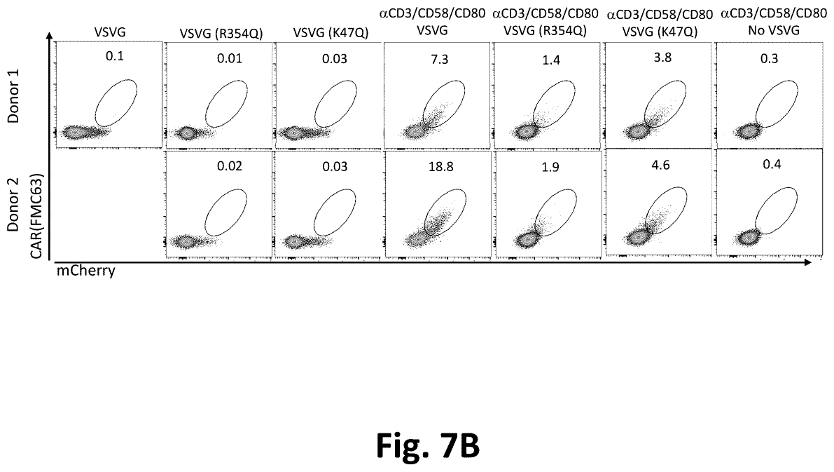

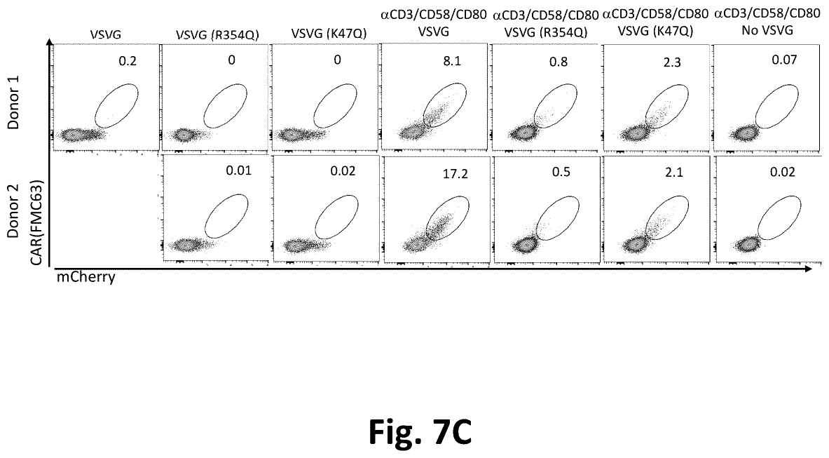

As shown in the top row of FIG. 7A viral particles comprising the blinded VSV-G mutant envelopes alone (without CD3scFv+CD80+CD58) exhibited greatly reduced transduction of SupT1 cells compared with a non-blinded VSV-G control. The bottom row depicted in FIG. 7A shows that the addition of activation, costimulation, and adhesion molecules in particles comprising blinded VSV-G mutant envelope proteins resulted in increased transduction.

Wishing to confirm the results seen using a T cell line, the experiment was replicated using PBMCs. On day 0, PBMCs from 2 donors were thawed and 2×10\hat{ }6 cells were placed in wells of a 24-well plate. Vector particles containing a CD19 CAR-mCherry payload, generated generally as described in Example 2, were added to each cell-containing well according to the following chart.

| Condition | Particle Description |
|---|---|
| αCD3/CD58/CD80; | Anti-CD3scFv + CD80 + CD58; |
| no VSVG | no envelope |
| VSVG | VSV-G only |
| VSVG (R354Q) | VSV-G (R354Q) only |
| VSVG (K47Q) | VSV-G (K47Q) only |
| αCD3/CD58/CD80; VSVG | Anti-CD3scFv + CD80 + CD58; VSV-G |
| αCD3/CD58/CD80; VSVG | Anti-CD3scFv + CD80 + CD58; |
| (R354Q) | VSV-G (R354Q) |
| αCD3/CD58/CD80; VSVG | Anti-CD3scFv + CD80 + CD58; |
| (K47Q) | VSV-G (K47Q) |

On day 3, the media was changed, and cells were re-plated with fresh media and samples were taken for assessment of transduction via flow cytometry. As shown in FIGS. 7B-7C, viral particles comprising blinded VSV-G envelopes resulted in reduced transduction compared with the non-blinded VSV-G control in both CD4 (FIG. 7B) and CD8 (FIG. 7C) T cells. In addition, the addition of CD3scFv+CD80+CD58 to viral particles resulted in increased transduction compared to viral particles without CD3scFv+CD80+CD58. In addition, viral particles comprising CD3scFv+CD80+CD58 without VSV-G also exhibited poor transduction.

On day 5, additional samples were taken for assessment of transduction via flow cytometry. Expression of CAR on day 5 was similar to expression on day 3 (data not shown).

The results of this study support the hypothesis that viral particles comprising a blinded envelope protein and activation, costimulation, and adhesion molecules are capable of transducing primary T cells.

### Example 7: In Vivo Expansion of Non-Transduced Cells

This study assessed expansion of non-transduced T cells after administration of a lentiviral particle with CD3scfv or CD3scfv+CD80+CD58. The lentiviral particle contains a polynucleotide encoding an anti-CD19 CAR. The lentiviral particle was delivered via intravenous injection into mice.

### Study Design

Mice were acclimated for one week after receipt. At day −7, blood from all mice was collected for flow cytometry analysis to quantify degree of humanization. Mice were randomized according to their total human CD3 levels into the treatment groups described in the table below.

| | Administration | Virus Dose | |
|---|---|---|---|
| Virus type | route | (Titre Unit) | |
| CD3scfv | IV | 100 | Million TU |
| CD3scfv | IV | 50 | Million TU |
| CD3scfv + CD58 + CD80 | IV | 100 | Million TU |
| CD3scfv + CD58 + CD80 | IV | 50 | Million TU |
| CD3scfv + CD58 + CD80 | IV | 25 | Million TU |

### Study Timeline

At study Day 0 (SD0) mice were then dosed with virus particles according to the table above. At SD11, blood was collected, and CAR negative T cells were measured.

### Results and Conclusions

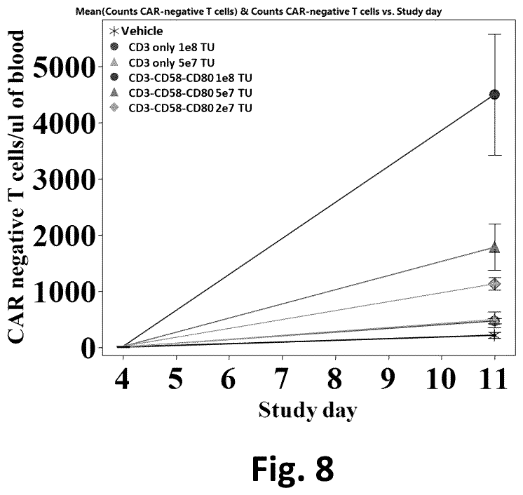

On Day 11 of the study, blood was collected from both groups. The level of CAR negative T cells in the blood was higher in the CD3scfv+CD58+CD80 particle-treated group and was dose-dependent compared to the CD3scfv particle-treated group (FIG. 8). These results indicate, when delivered intravenously, CD3scfv+CD58+CD80 engineered lentivirus particles appears to activate and expand even non-transduced T cells in vivo. Without wishing to be bound by theory, this activation of non-transduced T cells may enable a lower dose of engineered viral particles as non-transduced cells may exhibit anti-tumor activity.

### Example 8: Viral Particles Expressing Co-Stimulatory and Adhesion Molecules Enhance T Cell Activation and Tumor Control

To determine whether a combination of a co-stimulatory molecule (CD80), and an adhesion protein (CD58) enhance T cell activation, particles having both molecules were generated. These particles were examined for their ability to activate and transduce Nalm6 tumor cells compared to particles only having anti-CD3scFv.

### Virus Production

Virus was produced as described in Example 1.

### List of Virus Preps Made for Study:

1. CD3scfv only
2. CD3scfv+CD58 (separately expressed proteins)
3. CD3scfv+CD80 (separately expressed proteins)
4. CD3scfv+CD80+CD58 (separately expressed proteins)

anti-CD19 CAR+ T cells were generated using the viral particles indicated above. Anti-CD19 CAR T+ cells were cultured with Nalm6 tumor cells at various E:T ratios for 22 hours. Supernatant cytokine analysis was measured by Meso Scale Discovery (MSD) after transduction.

### Results and Conclusions

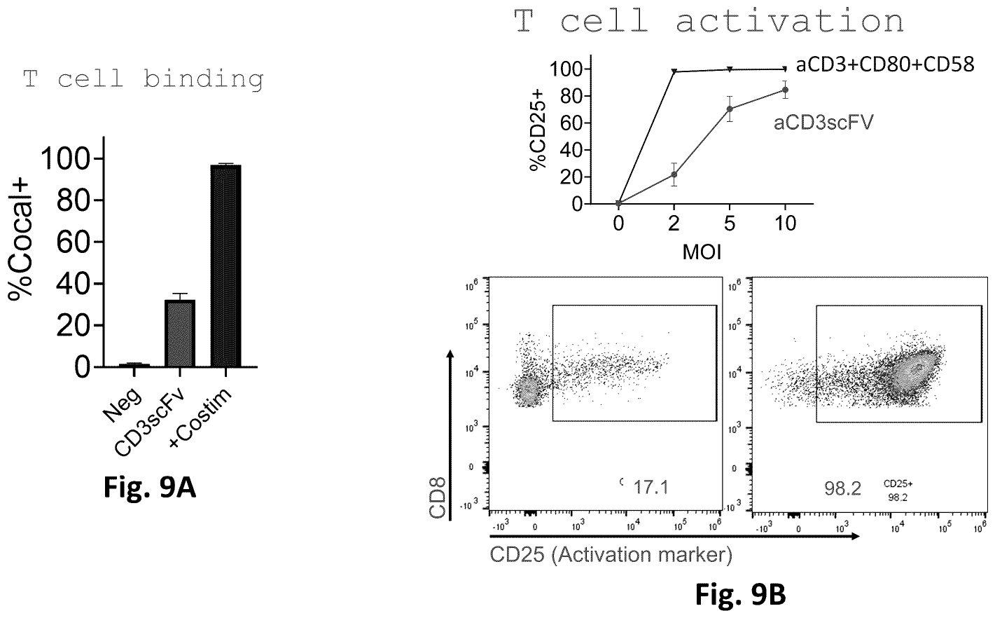

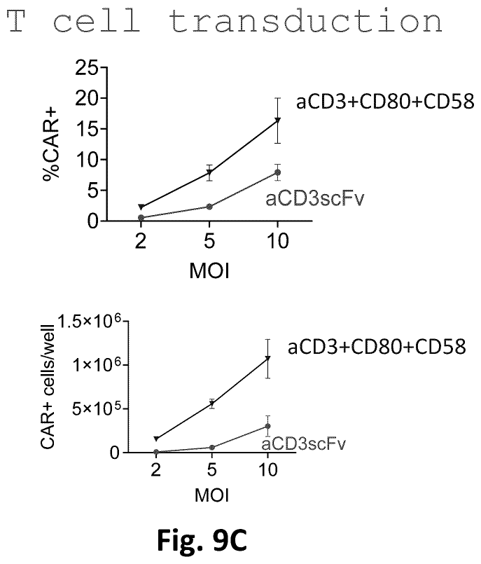

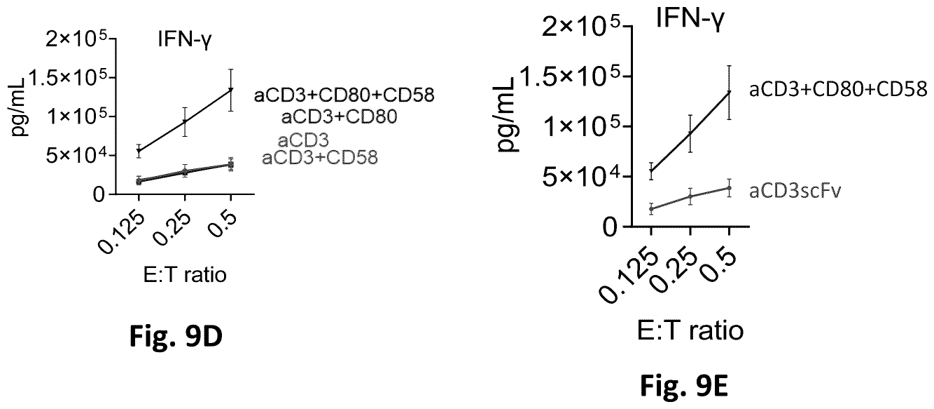

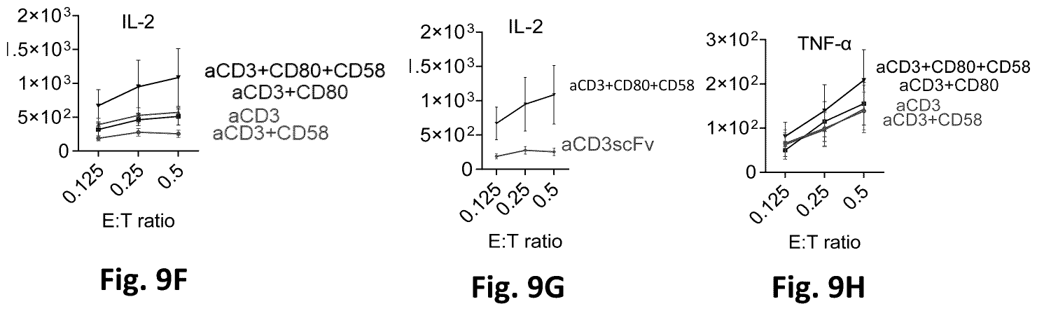

To assess whether viral particles with co-stimulatory molecules and adhesion molecules enhance T cell activation, samples were analyzed for cytokine expression. Vector Binding Assay with CD8 T cells from 3 PBMC donors was performed. Viral particles were cultured with PBMCs for 6 hours at MOI=10 followed by surface staining for Cocal glycoprotein on CD8 T cells (FIG. 9A). CD25 expression on CD8 T cells from 3 PBMC donors 3 days after transduction with engineered viral particles was analyzed (FIG. 9B). Flow cytometry plots taken from representative samples at MOI=2 are shown in FIG. 9B. Engineered viral particles packaging an anti-CD19 CAR were added to PBMCs from 3 donors. 7 days later CAR expression was assessed on CD8 T cells (FIG. 9C). CD3scfv+CD80+CD58 (separately expressed proteins) particles were capable of inducing IFN-γ production in Nalm6 tumor cells at lower doses whereas CD3scfv+CD58 (separately expressed proteins), CD3scfv+CD80 (separately expressed proteins), and CD3scfv only particles transduced Nalm6 tumor cells to a lesser extent (FIGS. 9D and 9E). CD3scfv+CD80+CD58 particles induced robust IL-2 and TNF-α production whereas CD3scfv+CD58, CD3scfv+CD80, and CD3scfv only did not (FIGS. 9F, 9G, and 9H). The data show that CD3scfv+CD80+CD58 particles efficiently induce cytokine production in Nalm6 tumor cells in vitro compared to CD3scfv+CD58, CD3scfv+CD80, and CD3scfv only. Engineered particles displaying CD3scfv+CD80+CD58 (separately expressed proteins) increased particle-T cell binding, activation, and transduction of T cells in vitro.

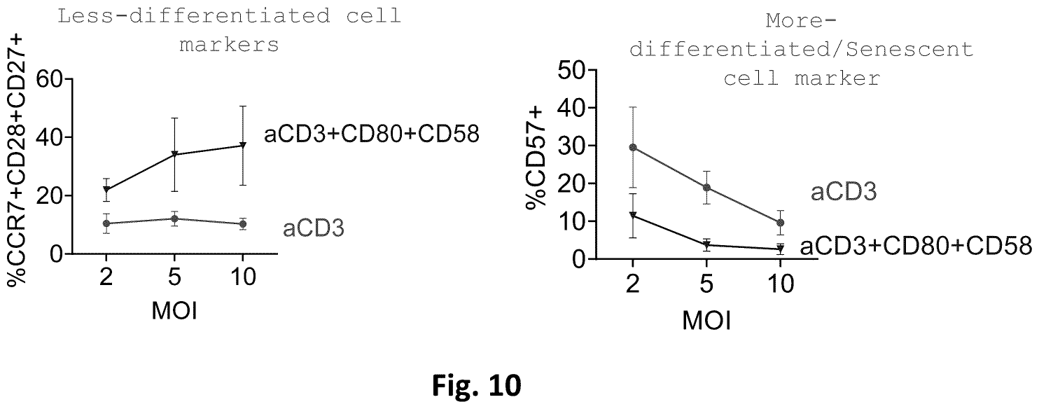

To determine whether different T cell subtypes are generated by the viral particles, PBMCs from 3 donors were cultured with viral particles packaging an anti-CD19 CAR were profiled and gated on viable and CD8+ T cells. After 7 days, the cells were analyzed by flow cytometry including cell surface markers CCR7, CD28, CD27, and CD57. CD3scfv only particles produced a more differentiated cell phenotype and CD3scfv+CD80+CD58 particles produced a less-differentiated cell phenotype (FIG. 10). CAR T cells generated with engineered particles displaying CD3scfv+CD80+CD58 (separately expressed proteins) showed a less differentiated phenotype and are more functional in vitro as compared to particled displaying only CD3scfv.

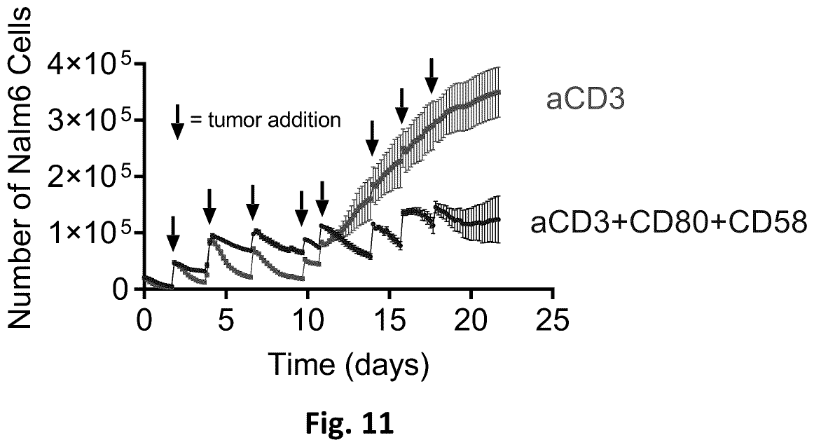

To assess the anti-tumor efficacy of CAR T cells generated using viral particles expressing co-stimulatory and adhesion molecules, PBMCs were transduced and cultured with Nalm6 tumor cells. Specifically, anti-CD19 CAR+ T cells were serial-stimulated with Nalm6 tumor cells every 2-3 days. Total Nalm6 tumor cells were measured over time using an IncuCyte® providing a measurement of tumor cell killing over time. The highest cytotoxic function was observed with particles composed of CD3scfv+CD80+CD58 (FIG. 11). This study demonstrated the CD3scfv+CD80+CD58 particles induced the highest cytokine production of T cells and provided the highest cytolytic function in vitro.

Tumor control from in vivo transduction of T cells by a lentiviral particle with CD3scfv or CD3scfv+CD80+CD58 was assessed. The lentiviral particle contains a polynucleotide encoding an anti-CD19 CAR transgene. The lentiviral particle was delivered via intravenous injection into NSG MHCI/II KO mice. The mice used in the study were immune-compromised and contain engrafted human T cells and circulating human B cells.

TABLE 3
Study Treatment Groups

| | | | Virus Dose | |
|---|---|---|---|---|
| | Viral Particle | Administration | (Transducing | |
| Group | Surface Molecules | route | Unit) | |
| 1 | Vehicle | IV | | |
| 2 | CD3scfv | IV | 50 | Million TU |
| 3 | CD3scfv | IV | 100 | Million TU |
| 4 | CD3scfv + CD80 + | IV | 25 | Million TU |
| | CD58 (separately | | | |
| | expressed proteins) | | | |
| 5 | CD3scfv + CD80 + | IV | 50 | Million TU |
| | CD58 (separately | | | |
| | expressed proteins) | | | |
| 6 | CD3scfv + CD80 + | IV | 100 | Million TU |
| | CD58 (separately | | | |
| | expressed proteins) | | | |

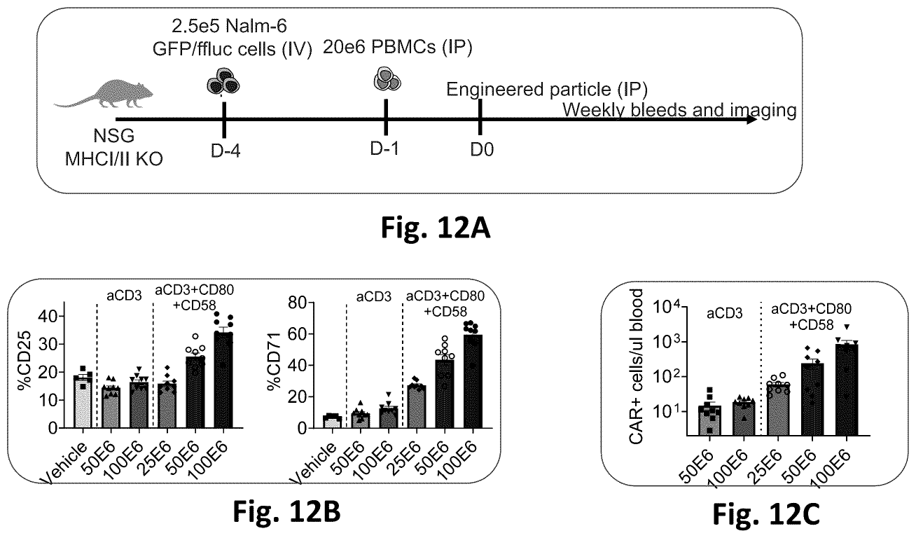

NSG MHCI/II KO mice were acclimated for one week after receipt. At Day −4, 2.5×10⁵ luciferase+Nalm6/ffluc cells were intravenously injected. At study Day −1, 20×10⁶ PBMCs were injected into the intraperitoneal cavity. At study Day 0, mice were dosed with virus particles according to Table 3 (FIG. 12A).

Four days after viral particle administration, cells were harvested and activation marker CD25 and CD71 expression was measured by flow cytometry on viable CD3+ T cells in the blood (FIG. 12B). On Day 11, total anti-CD19 CAR+CD3+ T cells found in the blood were analyzed by flow cytometry for CAR surface expression (FIG. 12C). The level of CAR T cells in the blood was higher in the CD3scfv+CD80+CD58 particle-treated group compared to the CD3scfv particle-treated group (FIG. 12C). Viral particles comprising activation, costimulation, and adhesion molecules (e.g. CD3scFv+CD80+CD58 particles) efficiently transduced T cells to a greater extent than particles comprising a CD3scFv without costimulation and adhesion components. These results indicate that viral particles as described herein may enable dose-dependent T cell transduction and activation in vivo.

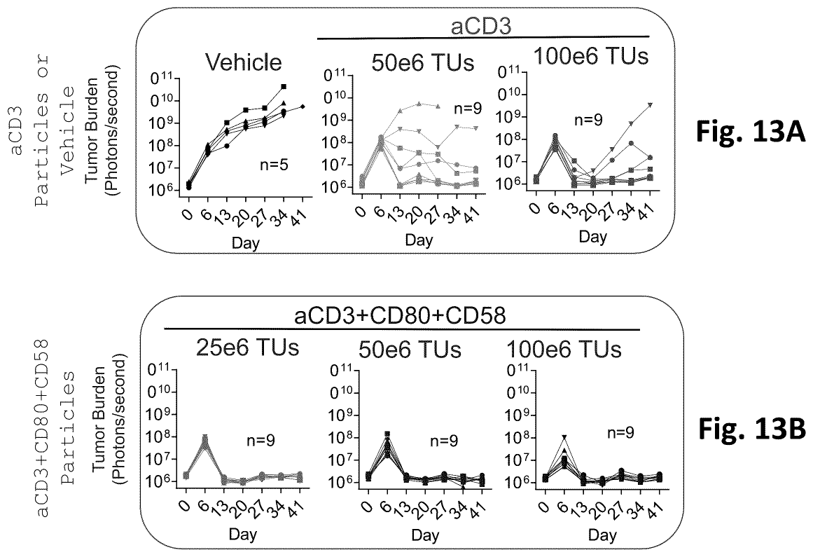

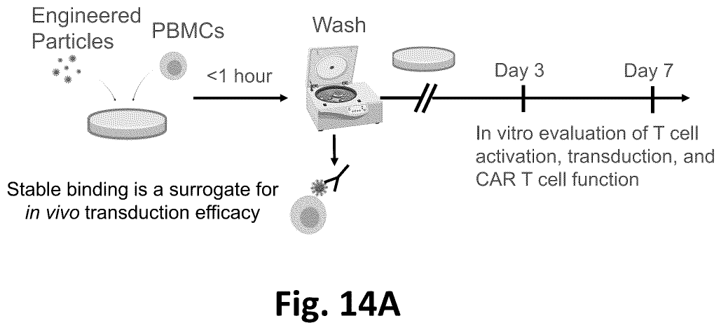

Tumor burden was assessed as total flux and measured for the duration of the study using an In vivo imaging system (IVIS®). The CD3scfv+CD80+CD58 particle-treated group demonstrated enhanced tumor control as compared to the CD3scfv particle-treated group (FIG. 13A and FIG. 13B).

In summary, when delivered intravenously, CD3scfv and CD3scfv+CD80+CD58 engineered lentivirus particles successfully transduced T cells in vivo. While both groups decreased tumor burden, particles with co-stimulatory molecule CD80 and adhesion molecule CD58 provided greater anti-tumor efficacy and anti-tumor immune response. CD3scfv+CD80+CD58 engineered lentivirus particles show dose-dependent T cell transduction and antitumor immunity in an in vivo xenograft model.

### Example 9: T Cell Activation and Transduction with Viral Particles Displaying a CD58, CD80, and Anti-CD3 scFv Tri-Fusion Polypeptide

This Example shows T cell activation and transduction with viral particles displaying a CD58, CD80, and anti-CD3 scFv tri-fusion polypeptide.

Human PBMCs from 3 normal donors were cultured in T cell growth (TCGM) media (RPMI1640+5% HuAB serum+1×GlutaMax+HEPES). For lentiviral transduction, viral particles were added to the PBMC cells.

To analyze T cell activation, cells were pelleted after 3 days and then analyzed by flow cytometry. T cell activation was measured by detection of hCD25 marker using an anti-CD25-PE/Cy7 antibody diluted 1:100 in Cell Staining Buffer. To measure CAR expression levels and transduction efficiencies, cells were pelleted after a 7-day production period following lentiviral transduction. Cells were then analyzed by flow cytometry. Anti-CD19 CAR surface expression was detected and all flow cytometric analysis was done on an Attune™ NxT Flow Cytometer and analyzed with FlowJo™. Day 7 transduced primary T cells expressing anti-CD19 CAR were counted, resuspended, and added to Nalm6 tumor cells. Killing of target Nalm6 cells was analyzed in an IncuCyte™ Live Cell Analysis System. Each well was imaged every 6 hours and the number of Nalm6 cells was quantified to assess the kinetics of T cell cytotoxicity. After 24 hours, supernatant from each well was collected for cytokine measurements according to manufacturer's protocol.

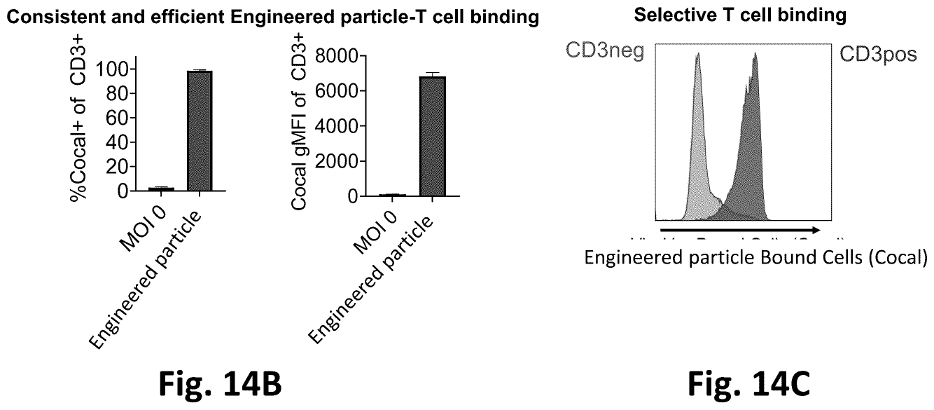

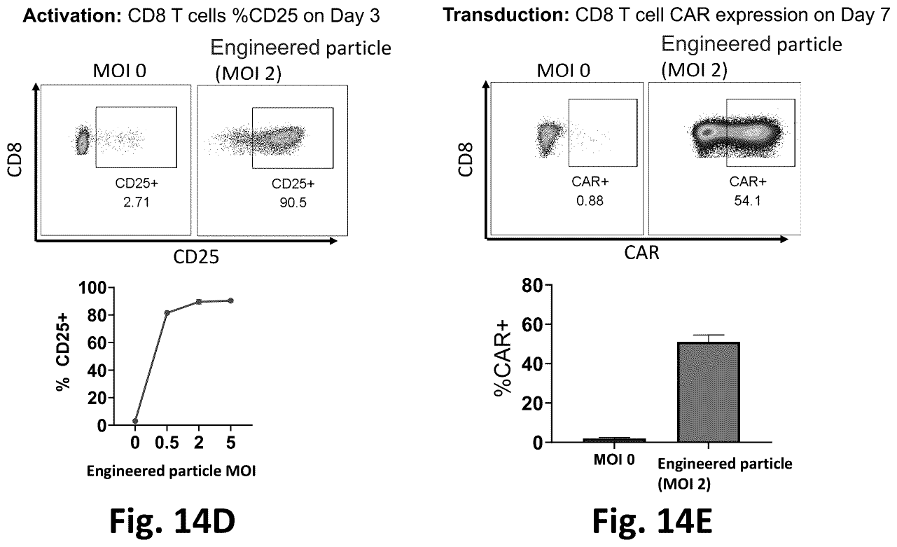

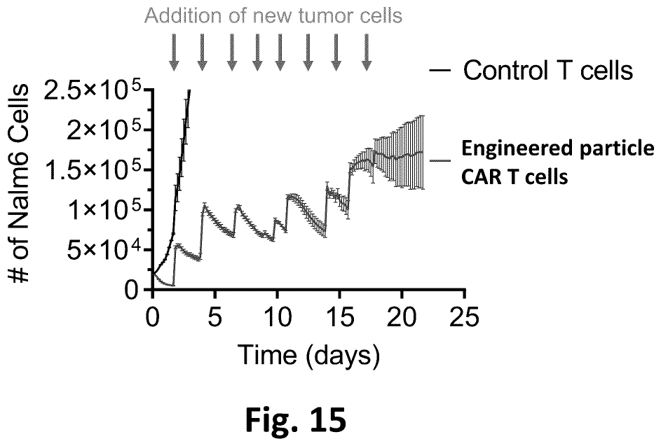

Healthy donor PBMCs (from three donors) were contacted for less than one hour with lentiviruses carrying an anti-CD19 CAR transgene and displaying surface engineered tri-fusion proteins at MOI 2 (FIG. 14A). Consistent and efficient binding of T cells to engineered lentiviral particles was observed and measured by percentage of CD3+ T cells positively staining for Cocal (FIG. 14B). Selective T cell binding was observed in a Cocal staing peak shift for CD3+ T cells relative to CD3− T cells (FIG. 14C). Activation was determined based on hCD25 staining on Day 3 (FIG. 14D), and CAR expression level was measured (FIG. 14E). The engineered lentiviral particles demonstrated robust avidity and selectivity for T cell binding following short duration (<1 hour) culture. Transduced PBMCs were cultured with Nalm6 tumor cells. Specifically, anti-CD19 CAR+ T cells were serial-stimulated with Nalm6 tumor cells every 2-3 days. Total Nalm6 tumor cells were measured over time using an IncuCyte® providing a measurement of tumor cell killing over time (FIG. 15). This assay measures the ability of the CAR T cells to expand and kill multiple tumor cells over time and showed that anti-CD19 CAR T cells generated with lentivirus particles displaying a CD58, CD80, and anti-CD3 scFv tri-fusion protein demonstrated serial killing in vitro.

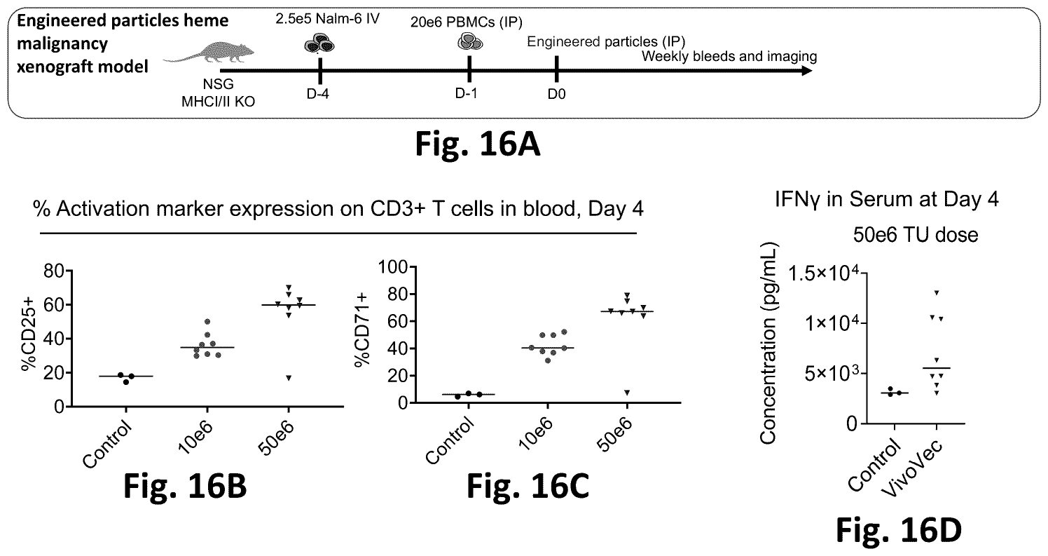

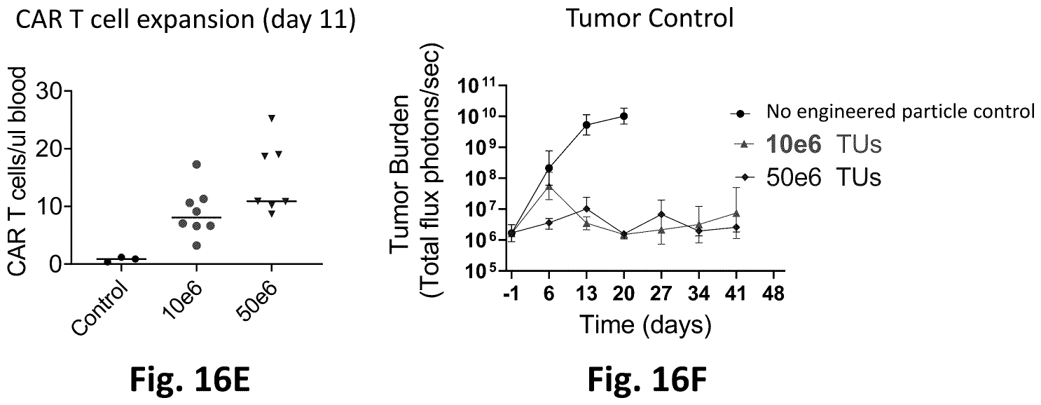

In a study of hematologic malignancy in a tumor xenograft model, on Day −4, 2.5×10⁵ Nalm6 cells were intravenously injected into NSG MHCI/II KO mice. At study Day −1, 20×10⁶ PBMCs were injected into the intraperitoneal cavity. At study Day 0, mice were dosed with virus particles displaying a CD58, CD80, and anti-CD3 scFv tri-fusion protein (FIG. 16A).

Four days after viral particle administration, cells were harvested and expression of activation markers CD25 (FIG. 16B) and CD71 (FIG. 16C) and cytokine IFN-γ production (FIG. 16D) were measured by flow cytometry on viable CD3+ T cells in the blood. CAR T cell expansion was analyzed at doses of 10 Million and 50 Million transducing units (TU). On Day 11, total anti-CD19 CAR+ T cells found in the blood were analyzed by flow cytometry for CAR surface expression (FIG. 16E). Tumor burden was assessed as total flux and measured for the duration of the study using an In vivo Imaging system (IVIS®) (FIG. 16F).

### Example 10: In Vivo Transduction of T Cells by a Lentiviral Particle Displaying a CD58, CD80, and Anti-CD3 scFv Tri-Fusion Polypeptide Via Extracorporeal Contact

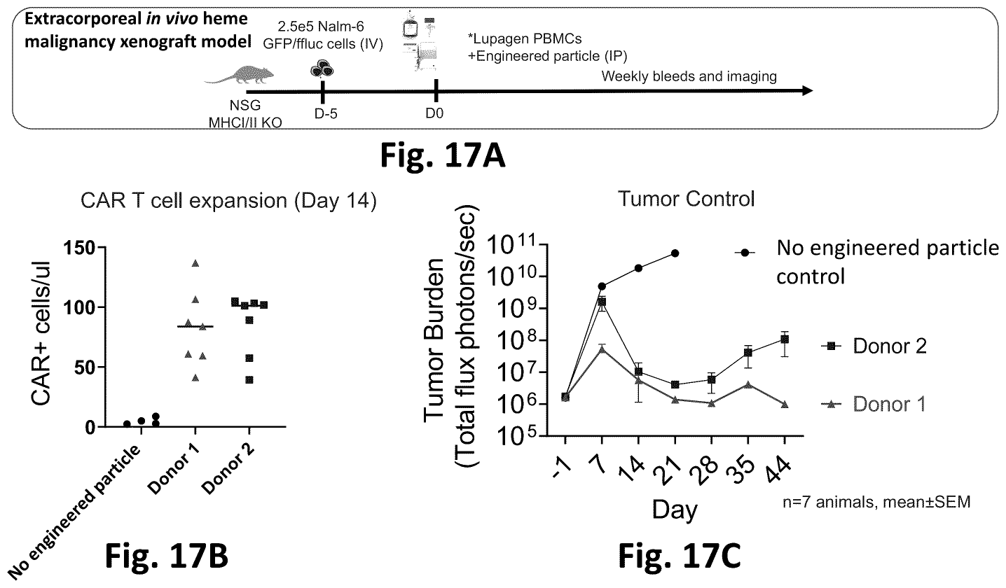

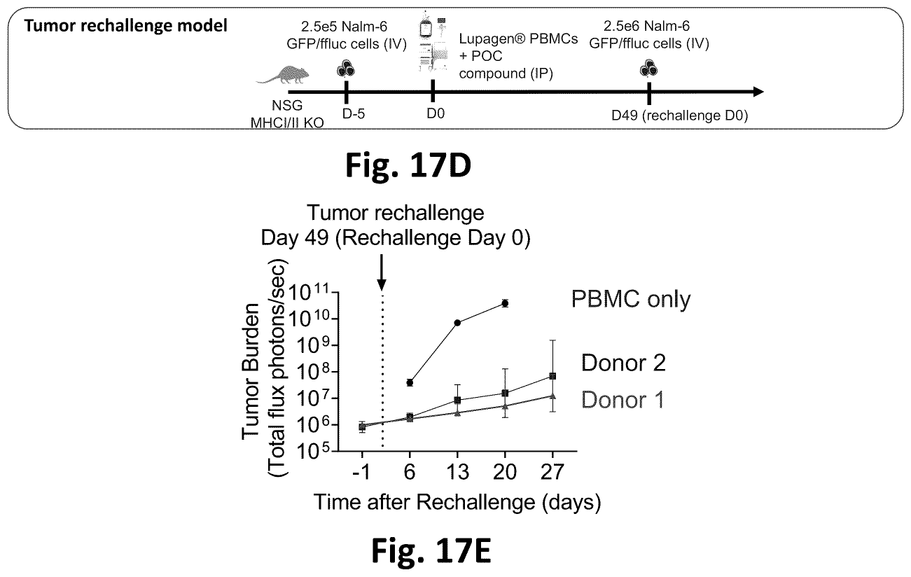

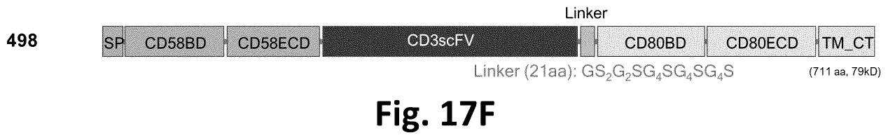

This Example analyzed transduction of T cells by a lentiviral particle displaying a CD58, CD80, and anti-CD3 scFv tri-fusion polypeptide (FIG. 17F) via extracorporeal contact in a hematologic malignancy xenograft model.

On study Day −5 NSG MHC I/II dKO mice were injected via tail vein injection with 2.5E5 Nalm6 cells expressing firefly luciferase (ffluc) (FIG. 17A). On study Day 0, Apheresis blood was washed on the Lupagen™ machine and viral particles at an MOI of 2 and T cells (50e6-100e6 cells) were placed under extracorporeal contact and incubated for 1 hour. Following incubation, the particle-bound cells were then washed of unbound particles to generate the “Final” material yielding a cell concentration ranging from 15-35e6 cells/ml. For Donor 1, 25e6 cells were injected and for Donor 2, 15e6 cells were injected. In vitro characterization of particle-T cell binding, T cell activation, and transduction were evaluated in parallel.

Particle-bound cells were assessed for CAR T cell expansion in Donor 1 and Donor 2 (FIG. 17B). Tumor burden was assessed as total flux and measured for the duration of the study for Donor 1 and Donor 2 using an In vivo Imaging system (IVIS®) (FIG. 17C). On study Day 49 (rechallange Day 0) the mice were injected via tail vein injection with an additional 2.5E5 Nalm6 cells expressing firefly luciferase (ffluc) to assess clearance of tumor re-challenge (FIG. 17D). Tumor burden was assessed as total flux and measured for the duration of the rechallange study for Donor 1 and Donor 2 using an In vivo Imaging system (IVIS®) (FIG. 17E). Viral particles displaying a CD58, CD80, and anti-CD3 scFv tri-fusion polypeptide generated anti-CD19 CAR T cells showed persistance following primary tumor clearance and protection against tumor rechallenge in vivo.

SEQUENCES

| | | |
|---|---|---|
| 1 | CD8 signal peptide | MALPVTALLLPLALLLHAARP |
| 2 | Anti-CD3 scFv (VL- | DIQMTQSPSSLSASVGDRVTITCSASSSVSYMNWYQQTPGKAPKRWIY |
| | G4S x 3 linker-VH) | DTSKLASGVPSRFSGSGSGTDYTFTISSLQPEDIATYYCQQWSSNPFTFG |
| | | QGTKLQITRTSGGGGSGGGGSGGGGSQVQLVQSGGGVVQPGRSLRLS |
| | | CKASGYTFTRYTMHWVRQAPGKGLEWIGYINPSRGYTNYNQKVKDR |
| | | FTISRDNSKNTAFLQMDSLRPEDTGVYFCARYYDDHYCLDYWGQGTP |
| | | VTVSSAAAKP |
| 3 | CD8 hinge | TTTPAPRPPTPAPTIASQPLSLRPEASRPAAGGAVHTRGLDFASD |
| 4 | CD4 TM and | MALIVLGGVAGLLLFIGLGIFFCVRCRHRRRQGSGEGRGSLLTCGDVEE |
| | cytoplasmic tail_ | NPGP |
| | T2A | |
| 5 | Cocal envelope | NFLLLTFIVLPLCSHAKFSIVFPQSQKGNWKNVPSSYHYCPSSSDQNWH |
| | amino acid | NDLLGITMKVKMPKTHKAIQADGWMCHAAKWITTCDFRWYGPKYIT |
| | | HSIHSIQPTSEQCKESIKQTKQGTWMSPGFPPQNCGYATVTDSVAVVV |
| | | QATPHHVLVDEYTGEWIDSQFPNGKCETEECETVHNSTVWYSDYKVT |
| | | GLCDATLVDTEITFFSEDGKKESIGKPNTGYRSNYFAYEKGDKVCKMN |
| | | YCKHAGVRLPSGVWFEFVDQDVYAAAKLPECPVGATISAPTQTSVDV |
| | | SLILDVERILDYSLCQETWSKIRSKQPVSPVDLSYLAPKNPGTGPAFTIIN |
| | | GTLKYFETRYIRIDIDNPIISKMVGKISGSQTERELWTEWFPYEGVEIGP |
| | | NGILKTPTGYKFPLFMIGHGMLDSDLHKTSQAEVFEHPHLAEAPKQLP |
| | | EEETLFFGDTGISKNPVELIEGWFSSWKSTVVTFFFAIGVFILLYVVARI |
| | | VIAVRYRYQGSNNKRIYNDIEMSRFRK |
| 6 | CD8 signal peptide | ATGGCACTGCCTGTGACAGCCCTGCTGCTGCCACTGGCCCTGCTGCT |
| | | GCACGCAGCACGCCCA |
| 7 | Anti-CD3 scFv (VL- | GATATCCAGATGACCCAGTCCCCAAGCTCCCTGAGCGCCTCCGTGG |
| | G4S x 3 linker-VH) | GCGACCGGGTGACAATCACCTGCAGCGCCTCTAGCTCCGTGTCCTA |
| | | CATGAACTGGTATCAGCAGACACCTGGCAAGGCCCCAAAGAGATG |
| | | GATCTACGATACCAGCAAGCTGGCCTCCGGCGTGCCTTCTAGGTTTT |
| | | CTGGCAGCGGCTCCGGCACAGATTATACATTCACCATCTCTAGCCT |
| | | GCAGCCAGAGGACATCGCCACCTACTATTGCCAGCAGTGGTCCTCT |
| | | AATCCCTTTACATTCGGCCAGGGCACCAAGCTGCAGATCACAAGAA |
| | | CCTCTGGAGGAGGAGGAAGCGGAGGAGGAGGATCCGGCGGCGGCG |
| | | GCTCTCAGGTGCAGCTGGTGCAGAGCGGAGGAGGAGTGGTGCAGC |
| | | CAGGCAGAAGCCTGAGGCTGTCCTGTAAGGCCTCTGGCTACACATT |
| | | CACCAGATATACAATGCACTGGGTGAGGCAGGCACCAGGCAAGGG |
| | | ACTGGAGTGGATCGGCTACATCAACCCCTCCAGGGGCTACACCAAC |
| | | TATAATCAGAAGGTGAAGGATCGGTTCACCATCAGCAGGGACAACT |
| | | CCAAGAATACCGCCTTCCTGCAGATGGACAGCCTGAGGCCAGAGG |
| | | ATACCGGCGTGTACTTTTGCGCCCGGTACTATGACGATCACTACTGT |
| | | CTGGATTATTGGGGCCAGGGAACACCAGTGACCGTGAGCTCCGCCG |
| | | CAGCAAAGCCT |
| 8 | CD8 hinge | ACCACAACCCCTGCCCCAAGGCCACCTACACCCGCCCCTACCATCG |
| | | CCTCTCAGCCACTGAGCCTGAGGCCAGAGGCATCCAGGCCTGCCGC |
| | | AGGGGGGGCCGTGCACACCCGGGGCCTGGACTTTGCCTCTGAT |
| 9 | CD4 TM and | ATGGCACTGATCGTGCTGGGAGGAGTGGCAGGACTGCTGCTGTTCA |
| | cytoplasmic tail_T2A | TCGGACTGGGCATCTTCTTTTGCGTGCGCTGTAGGCACCGGAGAAG |
| | | GCAGGGATCTGGAGAGGGAAGGGGAAGCCTGCTGACATGCGGCGA |
| | | CGTGGAGGAGAACCCAGGACCA |
| 10 | Cocal envelope | AATTTTCTGCTGCTGACCTTCATCGTGCTGCCTCTGTGCAGCCACGC |
| | nucleotide sequence | CAAGTTTTCCATCGTGTTCCCACAGTCCCAGAAGGGCAACTGGAAG |
| | | AATGTGCCCTCTAGCTACCACTATTGCCCTTCCTCTAGCGACCAGAA |
| | | CTGGCACAATGATCTGCTGGGCATCACAATGAAGGTGAAGATGCCC |
| | | AAGACCCACAAGGCCATCCAGGCAGATGGATGGATGTGCCACGCA |
| | | GCCAAGTGGATCACAACCTGTGACTTTCGGTGGTACGGCCCCAAGT |
| | | ATATCACACACTCCATCCACTCTATCCAGCCTACCTCCGAGCAGTGC |
| | | AAGGAGTCTATCAAGCAGACAAAGCAGGGCACCTGGATGAGCCCT |
| | | GGCTTCCCACCCCAGAACTGTGGCTACGCCACAGTGACCGACTCCG |
| | | TGGCAGTGGTGGTGCAGGCAACACCTCACCACGTGCTGGTGGATGA |
| | | GTATACCGGCGAGTGGATCGACAGCCAGTTTCCAAACGGCAAGTGC |
| | | GAGACAGAGGAGTGTGAGACCGTGCACAATTCTACAGTGTGGTAC |
| | | AGCGATTATAAGGTGACAGGCCTGTGCGACGCCACCCTGGTGGATA |
| | | CAGAGATCACCTTCTTTTCTGAGGACGGCAAGAAGGAGAGCATCGG |
| | | CAAGCCCAACACCGGCTACAGATCCAATTACTTCGCCTATGAGAAG |
| | | GGCGATAAGGTGTGCAAGATGAATTATTGTAAGCACGCCGGGGTGC |
| | | GGCTGCCTAGCGGCGTGTGGTTTGAGTTCGTGGACCAGGACGTGTA |
| | | CGCAGCAGCAAAGCTGCCTGAGTGCCCAGTGGGAGCAACCATCTCC |
| | | GCCCCAACACAGACCTCCGTGGACGTGTCTCTGATCCTGGATGTGG |
| | | AGCGCATCCTGGACTACAGCCTGTGCCAGGAGACCTGGAGCAAGAT |
| | | CCGGTCCAAGCAGCCCGTGTCCCCTGTGGACCTGTCTTACCTGGCA |
| | | CCAAAGAACCCAGGAACCGGACCAGCCTTTACAATCATCAATGGCA |
| | | CCCTGAAGTACTTCGAGACCCGCTATATCCGGATCGACATCGATAA |
| | | CCCTATCATCAGCAAGATGGTGGGCAAGATCTCTGGCAGCCAGACA |
| | | GAGAGAGAGCTGTGGACCGAGTGGTTCCCTTACGAGGGCGTGGAG |
| | | ATCGGCCCAAATGGCATCCTGAAGACACCAACCGGCTATAAGTTTC |
| | | CCCTGTTCATGATCGGCCACGGCATGCTGGACAGCGATCTGCACAA |
| | | GACCTCCCAGGCCGAGGTGTTTGAGCACCCACACCTGGCAGAGGCA |
| | | CCAAAGCAGCTGCCTGAGGAGGAGACACTGTTCTTTGGCGATACCG |
| | | GCATCTCTAAGAACCCCGTGGAGCTGATCGAGGGCTGGTTTTCCTC |
| | | TTGGAAGAGCACAGTGGTGACCTTCTTTTTCGCCATCGGCGTGTTCA |
| | | TCCTGCTGTACGTGGTGGCCAGAATCGTGATCGCCGTGAGATACAG |
| | | GTATCAGGGCTCCAACAATAAGAGGATCTATAATGACATCGAGATG |
| | | TCTCGCTTCCGGAAG |
| 11 | Gaussia luciferase | MGVKVLFALICIAVAEA |
| | SP | |
| 12 | Anti-CD3 scFv VL- | DIQMTQSPSSLSASVGDRVTITCSASSSVSYMNWYQQTPGKAPKRWIY |
| | G4S x 3 linker-VH | DTSKLASGVPSRFSGSGSGTDYTFTISSLQPEDIATYYCQQWSSNPFTFG |
| | | QGTKLQITRTSGGGGSGGGGSGGGGSQVQLVQSGGGVVQPGRSLRLS |
| | | CKASGYTFTRYTMHWVRQAPGKGLEWIGYINPSRGYTNYNQKVKDR |
| | | FTISRDNSKNTAFLQMDSLRPEDTGVYFCARYYDDHYCLDYWGQGTP |
| | | VTVSSAS |
| 13 | Short hinge-TM-CT | GVELIEGWFSSWKSTVVTFFFAIGVFILLYVVARIVIAVRYRYQGSNNK |
| | from Cocal Env | RIYNDIEMSRFRK |
| 14 | Gaussia luciferase | ATGGGCGTGAAAGTGCTGTTCGCCCTGATCTGCATCGCAGTTGCTG |
| | SP | AAGCC |
| 15 | Anti-CD3 scFv VL- | GACATCCAGATGACCCAGTCTCCTAGCAGCCTCAGCGCTAGCGTGG |
| | G4S x 3 linker-VH | GCGATAGAGTGACCATCACATGTAGCGCCAGCAGCAGCGTGTCCTA |
| | | CATGAACTGGTACCAGCAAACACCTGGAAAGGCCCCTAAAAGGTG |
| | | GATCTATGACACATCTAAGCTGGCTTCTGGAGTGCCATCTAGATTTT |
| | | CTGGCAGCGGCTCCGGCACTGATTATACATTCACCATCAGCAGCCT |
| | | GCAGCCCGAGGATATCGCCACCTACTACTGTCAGCAGTGGTCCTCT |
| | | AATCCCTTCACCTTCGGCCAGGGCACCAAGCTGCAGATCACCAGAA |
| | | CCAGCGGCGGGGGAGGAAGCGGCGGGGGAGGATCTGGCGGCGGCG |
| | | GCAGCCAGGTGCAGCTGGTGCAGAGCGGCGGCGGCGTGGTGCAAC |
| | | CTGGCAGAAGCCTGAGACTGAGCTGCAAGGCCTCTGGCTACACCTT |
| | | CACCCGGTACACCATGCATTGGGTGCGGCAGGCCCCTGGCAAGGGC |
| | | CTGGAATGGATTGGATACATCAACCCCAGCAGAGGCTACACCAACT |
| | | ACAACCAGAAGGTGAAGGACAGATTCACAATTTCTCGGGACAACA |
| | | GCAAGAATACCGCCTTCCTGCAAATGGACTCCCTGCGCCCAGAAGA |
| | | TACCGGCGTGTACTTCTGCGCTAGATATTACGACGACCACTACTGC |
| | | CTGGACTACTGGGGCCAGGGCACCCCTGTGACCGTGTCCAGCGCCT |
| | | CC |
| 16 | Short hinge-TM-CT | GGAGTGGAACTGATCGAGGGCTGGTTCAGCAGCTGGAAAAGCACC |
| | from Cocal Env | GTGGTTACATTCTTTTTCGCCATCGGCGTGTTCATCCTGCTGTACGT |
| | | GGTCGCCAGAATTGTGATCGCCGTGCGGTATAGATACCAGGGCAGC |
| | | AACAACAAGCGGATCTACAACGACATCGAGATGAGCAGATTCAGA |
| | | AAG |
| 17 | CD58 | MVAGSDAGRALGVLSVVCLLHCFGFISCFSQQIYGVVYGNVTFHVPSN |
| | | VPLKEVLWKKQKDKVAELENSEFRAFSSFKNRVYLDTVSGSLTIYNLT |
| | | SSDEDEYEMESPNITDTMKFFLYVLESLPSPTLTCALTNGSIEVQCMIPE |
| | | HYNSHRGLIMYSWDCPMEQCKRNSTSIYFKMENDLPQKIQCTLSNPLF |
| | | NTTSSIILTTCIPSSGHSRHRYALIPIPLAVITTCIVLYMNGILKCDRKPDR |
| | | TNSN |
| 18 | CD58 | atggttgctgggagcgacgcggggcgggccctgggggtcctcagcgtggtctgcctgctgcactgctttggttt |
| | | catcagctgtttttcccaacaaatatatggtgttgtgtatgggaatgtaactttccatgtaccaagcaatgtgc |
| | | ctttaaaagaggtcctatggaaaaaacaaaaggataaagttgcagaactggaaaattctgagttcagagcttt |
| | | ctcatcttttaaaaatagggtttatttagacactgtgtcaggtagcctcactatctacaacttaacatcatcag |
| | | atgaagatgagtatgaaatggaatcgccaaatattactgataccatgaagttctttctttatgtgcttgagtct |
| | | cttccatctcccacactaacttgtgcattgactaatggaagcattgaagtccaatgcatgataccagagcatta |
| | | caacagccatcgaggacttataatgtactcatgggattgtcctatggagcaatgtaaacgtaactcaaccagta |
| | | tatattttaagatggaaaatgatcttccacaaaaaatacagtgtactcttagcaatccattatttaatacaaca |
| | | tcatcaatcattttgacaacctgtatcccaagcagcggtcattcaagacacagatatgcacttatacccatacc |
| | | attagcagtaattacaacatgtattgtgctgtatatgaatggtattctgaaatgtgacagaaaaccagacagaa |
| | | ccaactccaat |
| 19 | Long hinge-TM-CT | SGFEHPHLAEAPKQLPEEETLFFGDTGISKNPVELIEGWFSSWKSTVVT |
| | from Cocal Env | FFFAIGVFILLYVVARIVIAVRYRYQGSNNKRIYNDIEMSRFRK |
| 20 | CD80 | MGHTRRQGTSPSKCPYLNFFQLLVLAGLSHFCSGVIHVTKEVKEVATL |
| | | SCGHNVSVEELAQTRIYWQKEKKMVLTMMSGDMNIWPEYKNRTIFDI |
| | | TNNLSIVILALRPSDEGTYECVVLKYEKDAFKREHLAEVTLSVKADFPT |
| | | PSISDFEIPTSNIRRIICSTSGGFPEPHLSWLENGEELNAINTTVSQDPETE |
| | | LYAVSSKLDFNMTTNHSFMCLIKYGHLRVNQTFNWNTTKQEHFPDNL |
| | | LPSWAITLISVNGIFVICCLTYCFAPRCRERRRNERLRRESVRPV |
| 21 | CD80 | ATGGGCCACACACGGAGGCAGGGAACATCACCATCCAAGTGTCCA |
| | | TACCTCAATTTCTTTCAGCTCTTGGTGCTGGCTGGTCTTTCTCACTTC |
| | | TGTTCAGGTGTTATCCACGTGACCAAGGAAGTGAAAGAAGTGGCAA |
| | | CGCTGTCCTGTGGTCACAATGTTTCTGTTGAAGAGCTGGCACAAAC |
| | | TCGCATCTACTGGCAAAAGGAGAAGAAAATGGTGCTGACTATGATG |
| | | TCTGGGGACATGAATATATGGCCCGAGTACAAGAACCGGACCATCT |
| | | TTGATATCACTAATAACCTCTCCATTGTGATCCTGGCTCTGCGCCCA |
| | | TCTGACGAGGGCACATACGAGTGTGTTGTTCTGAAGTATGAAAAAG |
| | | ACGCTTTCAAGCGGGAACACCTGGCTGAAGTGACGTTATCAGTCAA |
| | | AGCTGACTTCCCTACACCTAGTATATCTGACTTTGAAATTCCAACTT |
| | | CTAATATTAGAAGGATAATTTGCTCAACCTCTGGAGGTTTTCCAGA |
| | | GCCTCACCTCTCCTGGTTGGAAAATGGAGAAGAATTAAATGCCATC |
| | | AACACAACAGTTTCCCAAGATCCTGAAACTGAGCTCTATGCTGTTA |
| | | GCAGCAAACTGGATTTCAATATGACAACCAACCACAGCTTCATGTG |
| | | TCTCATCAAGTATGGACATTTAAGAGTGAATCAGACCTTCAACTGG |
| | | AATACAACCAAGCAAGAGCATTTTCCTGATAACCTGCTCCCATCCT |
| | | GGGCCATTACCTTAATCTCAGTAAATGGAATTTTTGTGATATGCTGC |
| | | CTGACCTACTGCTTTGCCCCAAGATGCAGAGAGAGAAGGAGGAAT |
| | | GAGAGATTGAGAAGGGAAAGTGTACGCCCTGTA |
| 22 | Long hinge-TM-CT | TCCGGATTCGAGCACCCCCACCTGGCCGAGGCCCCTAAGCAGCTGC |
| | from Cocal Env | CTGAAGAAGAGACACTGTTTTTCGGAGATACCGGCATCAGCAAAAA |
| | | CCCCGTGGAGCTGATCGAGGGCTGGTTCAGCTCTTGGAAGAGCACC |
| | | GTGGTCACATTCTTTTTCGCCATCGGCGTCTTTATCCTGCTGTACGT |
| | | GGTAGCCAGAATCGTGATCGCCGTGCGGTACAGATACCAGGGCAG |
| | | CAACAACAAGCGGATCTACAACGACATCGAGATGAGCCGGTTCAG |
| | | AAAG |
| 23 | CD86 | MDPQCTMGLSNILFVMAFLLSGAAPLKIQAYFNETADLPCQFANSQNQ |
| | | SLSELVVFWQDQENLVLNEVYLGKEKFDSVHSKYMGRTSFDSDSWTL |
| | | RLHNLQIKDKGLYQCIIHHKKPTGMIRIHQMNSELSVLANFSQPEIVPIS |
| | | NITENVYINLTCSSIHGYPEPKKMSVLLRTKNSTIEYDGVMQKSQDNVT |
| | | ELYDVSISLSVSFPDVTSNMTIFCILETDKTRLLSSPFSIELEDPQPPPDHI |
| | | PWITAVLPTVIICVMVFCLILWKWKKKKRPRNSYKCGTNTMEREESEQ |
| | | TKKREKIHIPERSDEAQRVFKSSKTSSCDKSDTCF |
| 24 | CD86 | ATGGATCCCCAGTGCACTATGGGACTGAGTAACATTCTCTTTGTGAT |
| | | GGCCTTCCTGCTCTCTGGTGCTGCTCCTCTGAAGATTCAAGCTTATT |
| | | TCAATGAGACTGCAGACCTGCCATGCCAATTTGCAAACTCTCAAAA |
| | | CCAAAGCCTGAGTGAGCTAGTAGTATTTTGGCAGGACCAGGAAAAC |
| | | TTGGTTCTGAATGAGGTATACTTAGGCAAAGAGAAATTTGACAGTG |
| | | TTCATTCCAAGTATATGGGCCGCACAAGTTTTGATTCGGACAGTTG |
| | | GACCCTGAGACTTCACAATCTTCAGATCAAGGACAAGGGCTTGTAT |
| | | CAATGTATCATCCATCACAAAAAGCCCACAGGAATGATTCGCATCC |
| | | ACCAGATGAACTCTGAACTGTCAGTGCTTGCTAACTTCAGTCAACC |
| | | TGAAATAGTACCAATTTCTAATATAACAGAAAATGTGTACATAAAT |
| | | TTGACCTGCTCATCTATACACGGTTACCCAGAACCTAAGAAGATGA |
| | | GTGTTTTGCTAAGAACCAAGAACTCAACTATCGAGTATGATGGTGT |
| | | TATGCAGAAATCTCAAGATAATGTCACAGAACTGTACGACGTTTCC |
| | | ATCAGCTTGTCTGTTTCATTCCCTGATGTTACGAGCAATATGACCAT |
| | | CTTCTGTATTCTGGAAACTGACAAGACGCGGCTTTTATCTTCACCTT |
| | | TCTCTATAGAGCTTGAGGACCCTCAGCCTCCCCCAGACCACATTCCT |
| | | TGGATTACAGCTGTACTTCCAACAGTTATTATATGTGTGATGGTTTT |
| | | CTGTCTAATTCTATGGAAATGGAAGAAGAAGAAGCGGCCTCGCAAC |
| | | TCTTATAAATGTGGAACCAACACAATGGAGAGGGAAGAGAGTGAA |
| | | CAGACCAAGAAAAGAGAAAAAATCCATATACCTGAAAGGTCTGAT |
| | | GAAGCCCAGCGTGTTTTTAAAAGTTCGAAGACATCTTCATGCGACA |
| | | AAAGTGATACATGTTTT |
| 25 | 218 linker_human | SGGSTSGSGKPGSGEGSTKGPEITLIIFGVMAGVIGTILLISYGIRRLALK |
| | Glycophorin A ecto- | YWWNLLQYWSQELKNSAVSLLNATAIAVAEGTDRVIEVVQGACRAIR |
| | TM_HIV Env CT | HIPRRIRQGLERILL |
| 26 | | |
| 27 | | |
| 28 | linker_human | TCCGGAGGATCTACAAGCGGCTCTGGCAAGCCTGGCAGCGGAGAA |
| | Glycophorin A ecto- | GGCAGCACCAAGGGCCCTGAGATCACACTGATCATCTTCGGCGTGA |
| | TM_HIV Env CT | TGGCCGGCGTCATCGGCACCATCCTGCTGATCAGCTACGGCATCAG |
| | | AAGACTGGCTCTGAAGTACTGGTGGAATCTGCTGCAATACTGGAGC |
| | | CAGGAGCTGAAAAACAGCGCCGTGTCCCTGCTCAACGCCACCGCCA |
| | | TCGCCGTGGCCGAGGGCACCGACAGAGTGATCGAGGTGGTGCAGG |
| | | GAGCCTGCAGAGCTATTCGGCACATCCCCAGACGGATCAGGCAGG |
| | | GCCTGGAAAGAATCCTGCTG |
| 29 | | |
| 30 | | |
| 31 | 218 linker_HIV Env- | SGGSTSGSGKPGSGEGSTKGNWLWYIRIFIIIVGSLIGLRIVFAVLSLVNR |
| | TM-CT | GWEALKYWWNLLQYWSQELKNSAVSLLNATAIAVAEGTDRVIEVVQ |
| | | GACRAIRHIPRRIRQGLERILL |
| 32 | | |
| 33 | | |
| 34 | 218 linker_HIV Env- | TCCGGAGGAAGCACCAGCGGCTCTGGCAAGCCTGGCAGCGGCGAG |
| | TM-CT | GGCTCTACCAAGGGCAATTGGCTGTGGTACATCAGAATCTTCATCA |
| | | TCATCGTGGGCAGCCTGATCGGCCTGAGAATCGTGTTCGCCGTGCT |
| | | GAGCCTGGTGAACCGGGGCTGGGAAGCTCTGAAGTACTGGTGGAA |
| | | CCTGCTGCAATACTGGTCCCAGGAGCTGAAAAACAGCGCTGTGTCC |
| | | CTGCTCAACGCCACCGCCATCGCCGTCGCCGAGGGAACAGACAGA |
| | | GTGATCGAGGTGGTGCAGGGAGCCTGCAGAGCCATTCGGCACATCC |
| | | CCAGACGCATCAGACAGGGCCTGGAAAGAATCCTGCTG |
| 35 | MND Promoter | GAACAGAGAAACAGGAGAATATGGGCCAAACAGGATATCTGTGGT |
| | | AAGCAGTTCCTGCCCCGGCTCAGGGCCAAGAACAGTTGGAACAGC |
| | | AGAATATGGGCCAAACAGGATATCTGTGGTAAGCAGTTCCTGCCCC |
| | | GGCTCAGGGCCAAGAACAGATGGTCCCCAGATGCGGTCCCGCCCTC |
| | | AGCAGTTTCTAGAGAACCATCAGATGTTTCCAGGGTGCCCCAAGGA |
| | | CCTGAAATGACCCTGTGCCTTATTTGAACTAACCAATCAGTTCGCTT |
| | | CTCGCTTCTGTTCGCGCGCTTCTGCTCCCCGAGCTCTATATAAGCAG |
| | | AGCTCGTTTAGTGAACCGTCAGATCGCTAGC |
| 36 | Anti-CD3 CDRH2 | YINPSRGYTNYNQKVKD |
| 37 | G4Sx3 linker_HIV | SGGGGGSGGGGSGGGGSYIRIFIIIVGSLIGLRIVFAVLSLVNRGWEALK |
| | Env-TM-CT | YWWNLLQYWSQELKNSAVSLLNATAIAVAEGTDRVIEVVQGACRAIR |
| | | HIPRRIRQGLERILL |
| 38 | Anti-CD3 CDRH3 | YYDDHYCLDY |
| 39 | Anti-CD19 CDRL1 | RASQDISKYLN |
| 40 | G4Sx3 linker_HIV | TCCGGAGGCGGTGGAGGCTCTGGTGGCGGAGGGAGCGGTGGCGGA |
| | Env-TM-CT | GGCAGCTACATCAGAATCTTCATCATCATCGTGGGCAGCCTGATCG |
| | | GCCTGAGAATCGTGTTCGCCGTTCTGAGCCTGGTGAACCGGGGCTG |
| | | GGAAGCCCTGAAGTACTGGTGGAATCTGCTCCAGTACTGGTCTCAG |
| | | GAGCTGAAGAACAGCGCCGTGTCCCTGCTGAACGCTACAGCTATCG |
| | | CCGTCGCCGAGGGCACCGACAGAGTGATCGAGGTGGTGCAGGGCG |
| | | CCTGCAGAGCCATCCGGCACATCCCTAGAAGGATTCGGCAAGGCCT |
| | | GGAAAGAATCCTGCTG |
| 41 | Anti-CD19 CDRL2 | HTSRLHS |
| 42 | Anti-CD19 CDRL3 | QQGNTLPYT |
| 43 | Short hinge-TM-CT | GVELIEGWFSSWKSTVVTFFFAIGVFILLYVVARIVIAVRYRYQGSNNK |
| | from Cocal Env_T2A | RIYNDIEMSRFRKGSGEGRGSLLTCGDVEENPGP |
| 44 | Anti-CD19 CDRH1 | DYGV |
| 45 | Anti-CD19 CDRH2 | VIWGSETTYYNSALKS |
| 46 | Anti-CD19 CDRH3 | HYYYGGSYAMDY |
| 47 | Short hinge-TM-CT | GGAGTGGAACTGATCGAGGGCTGGTTCAGCAGCTGGAAAAGCACC |
| | from Cocal Env_T2A | GTGGTTACATTCTTTTTCGCCATCGGCGTGTTCATCCTGCTGTACGT |
| | | GGTCGCCAGAATTGTGATCGCCGTGCGGTATAGATACCAGGGCAGC |
| | | AACAACAAGCGGATCTACAACGACATCGAGATGAGCAGATTCAGA |
| | | AAGGGATCTGGAGAGGGAAGGGGAAGCCTGCTGACATGCGGCGAC |
| | | GTGGAGGAGAACCCAGGACCA |
| 48 | Anti-CD3 CDRH1 | RYTMH |
| 49 | αCD19 | MLLLVTSLLLCELPHPAFLLIPDIQMTQTTSSLSASLGDRVTISCRASQDI |
| | CAR_FRB_RACR | SKYLNWYQQKPDGTVKLLIYHTSRLHSGVPSRFSGSGSGTDYSLTISNL |
| | lentiviral vector | EQEDIATYFCQQGNTLPYTFGGGTKLEITGSTSGSGKPGSGEGSTKGEV |
| | | KLQESGPGLVAPSQSLSVTCTVSGVSLPDYGVSWIRQPPRKGLEWLGV |
| | | IWGSETTYYNSALKSRLTIIKDNSKSQVFLKMNSLQTDDTAIYYCAKH |
| | | YYYGGSYAMDYWGQGTSVTVSSESKYGPPCPPCPMFWVLVVVGGVL |
| | | ACYSLLVTVAFIIFWVKRGRKKLLYIFKQPFMRPVQTTQEEDGCSCRFP |
| | | EEEEGGCELRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPRGSGATNFSLLKQAGDVEEN |
| | | PGPEMWHEGLEEASRLYFGERNVKGMFEVLEPLHAMMERGPQTLKET |
| | | SFNQAYGRDLMEAQEWCRKYMKSGNVKDLLQAWDLYYHVFRRISK |
| | | GSGATNFSLLKQAGDVEENPGPMPLGLLWLGLALLGALHAQAGVQV |
| | | ETISPGDGRTFPKRGQTCVVHYTGMLEDGKKFDSSRDRNKPFKFMLG |
| | | KQEVIRGWEEGVAQMSVGQRAKLTISPDYAYGATGHPGIIPPHATLVF |
| | | DVELLKLGEGSNTSKENPFLFALEAVVISVGSMGLIISLLCVYFWLERT |
| | | MPRIPTLKNLEDLVTEYHGNFSAWSGVSKGLAESLQPDYSERLCLVSEI |
| | | PPKGGALGEGPGASPCNQHSPYWAPPCYTLKPETGSGATNFSLLKQAG |
| | | DVEENPGPMALPVTALLLPLALLLHAARPILWHEMWHEGLEEASRLY |
| | | FGERNVKGMFEVLEPLHAMMERGPQTLKETSFNQAYGRDLMEAQEW |
| | | CRKYMKSGNVKDLLQAWDLYYHVFRRISKGKDTIPWLGHLLVGLSG |
| | | AFGFIILVYLLINCRNTGPWLKKVLKCNTPDPSKFFSQLSSEHGGDVQK |
| | | WLSSPFPSSSFSPGGLAPEISPLEVLERDKVTQLLLQQDKVPEPASLSSN |
| | | HSLTSCFTNQGYFFFHLPDALEIEACQVYFTYDPYSEEDPDEGVAGAPT |
| | | GSSPQPLQPLSGEDDAYCTFPSRDDLLLFSPSLLGGPSPPSTAPGGSGAG |
| | | EERMPPSLQERVPRDWDPQPLGPPTPGVPDLVDFQPPPELVLREAGEE |
| | | VPDAGPREGVSFPWSRPPGQGEFRALNARLPLNTDAYLSLQELQGQDP |
| | | THLV |
| 50 | Signal peptide | MLLLVTSLLLCELPHPAFLLIP |
| | (Human CSF2R) | |
| 51 | CD19 VL_linker_VH | DIQMTQTTSSLSASLGDRVTISCRASQDISKYLNWYQQKPDGTVKLLIY |
| | | HTSRLHSGVPSRFSGSGSGTDYSLTISNLEQEDIATYFCQQGNTLPYTFG |
| | | GGTKLEITGSTSGSGKPGSGEGSTKGEVKLQESGPGLVAPSQSLSVTCT |
| | | VSGVSLPDYGVSWIRQPPRKGLEWLGVIWGSETTYYNSALKSRLTIIKD |
| | | NSKSQVFLKMNSLQTDDTAIYYCAKHYYYGGSYAMDYWGQGTSVTV |
| | | SS |
| 52 | IgG4 linker-CD28 | ESKYGPPCPPCPMFWVLVVVGGVLACYSLLVTVAFIIFWV |
| | TM | |
| 53 | 4-1BB signal domain | KRGRKKLLYIFKQPFMRPVQTTQEEDGCSCRFPEEEEGGCEL |
| 54 | CD3zeta | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR |
| 55 | P2A self-cleaving | GSGATNFSLLKQAGDVEENPGP |
| | peptide | |
| 56 | Naked FRB | EMWHEGLEEASRLYFGERNVKGMFEVLEPLHAMMERGPQTLKETSFN |
| | | QAYGRDLMEAQEWCRKYMKSGNVKDLLQAWDLYYHVFRRISK |
| 57 | Signal peptide- | MPLGLLWLGLALLGALHAQAGVQVETISPGDGRTFPKRGQTCVVHYT |
| | FKBP12 | GMLEDGKKFDSSRDRNKPFKFMLGKQEVIRGWEEGVAQMSVGQRAK |
| | | LTISPDYAYGATGHPGIIPPHATLVFDVELLKL |
| 58 | −2R gamma TM- | GEGSNTSKENPFLFALEAVVISVGSMGLIISLLCVYFWLERTMPRIPTLK |
| | Cytoplasmic domain | NLEDLVTEYHGNFSAWSGVSKGLAESLQPDYSERLCLVSEIPPKGGAL |
| | | GEGPGASPCNQHSPYWAPPCYTLKPET |
| 59 | Signal peptide-FRB | MALPVTALLLPLALLLHAARPILWHEMWHEGLEEASRLYFGERNVKG |
| | | MFEVLEPLHAMMERGPQTLKETSFNQAYGRDLMEAQEWCRKYMKSG |
| | | NVKDLLQAWDLYYHVFRRISK |
| 60 | IL-2R beta TM- | GKDTIPWLGHLLVGLSGAFGFIILVYLLINCRNTGPWLKKVLKCNTPDP |
| | Cytoplasmic domain | SKFFSQLSSEHGGDVQKWLSSPFPSSSFSPGGLAPEISPLEVLERDKVTQ |
| | | LLLQQDKVPEPASLSSNHSLTSCFTNQGYFFFHLPDALEIEACQVYFTY |
| | | DPYSEEDPDEGVAGAPTGSSPQPLQPLSGEDDAYCTFPSRDDLLLFSPS |
| | | LLGGPSPPSTAPGGSGAGEERMPPSLQERVPRDWDPQPLGPPTPGVPDL |
| | | VDFQPPPELVLREAGEEVPDAGPREGVSFPWSRPPGQGEFRALNARLP |
| | | LNTDAYLSLQELQGQDPTHLV |
| 61 | αCD19 | ATGCTGCTGCTGGTGACCTCCCTGCTGCTGTGCGAGCTGCCTCACCC |
| | CAR_FRB_RACR | AGCCTTTCTGCTGATCCCCGACATCCAGATGACACAGACCACAAGC |
| | lentiviral vector | TCCCTGTCTGCCAGCCTGGGCGACAGAGTGACCATCTCCTGTAGGG |
| | | CCTCTCAGGATATCAGCAAGTACCTGAACTGGTATCAGCAGAAGCC |
| | | AGATGGCACAGTGAAGCTGCTGATCTACCACACCTCCAGGCTGCAC |
| | | TCTGGAGTGCCAAGCCGGTTCTCCGGATCTGGAAGCGGCACCGACT |
| | | ATTCCCTGACAATCTCTAACCTGGAGCAGGAGGATATCGCCACATA |
| | | CTTTTGCCAGCAGGGCAATACCCTGCCATATACATTCGGCGGAGGA |
| | | ACCAAGCTGGAGATCACCGGATCCACATCTGGAAGCGGCAAGCCA |
| | | GGAAGCGGAGAGGGATCCACAAAGGGAGAGGTGAAGCTGCAGGA |
| | | GAGCGGACCAGGACTGGTGGCACCATCCCAGTCTCTGAGCGTGACC |
| | | TGTACAGTGTCCGGCGTGTCTCTGCCTGACTACGGCGTGTCCTGGAT |
| | | CAGGCAGCCACCTAGGAAGGGACTGGAGTGGCTGGGCGTGATCTG |
| | | GGGCTCTGAGACCACATACTATAATTCTGCCCTGAAGAGCCGCCTG |
| | | ACCATCATCAAGGACAACTCCAAGTCTCAGGTGTTTCTGAAGATGA |
| | | ATAGCCTGCAGACCGACGATACAGCCATCTACTATTGCGCCAAGCA |
| | | CTACTATTACGGCGGCTCCTACGCCATGGATTATTGGGGCCAGGGC |
| | | ACCTCCGTGACAGTGTCTAGCGAGTCTAAGTATGGCCCACCCTGCC |
| | | CTCCATGTCCAATGTTCTGGGTGCTGGTGGTGGTGGGAGGCGTGCT |
| | | GGCCTGTTACTCCCTGCTGGTGACCGTGGCCTTTATCATCTTCTGGG |
| | | TGAAGAGAGGCAGGAAGAAGCTGCTGTATATCTTTAAGCAGCCCTT |
| | | CATGCGCCCTGTGCAGACCACACAGGAGGAGGACGGCTGCAGCTG |
| | | TCGGTTTCCAGAGGAGGAGGAGGGAGGATGCGAGCTGCGCGTGAA |
| | | GTTCAGCCGGTCCGCCGATGCCCCTGCCTACCAGCAGGGCCAGAAC |
| | | CAGCTGTATAACGAGCTGAATCTGGGCCGGAGAGAGGAGTACGAC |
| | | GTGCTGGATAAGAGGAGGGGAAGGGACCCAGAGATGGGAGGCAA |
| | | GCCTCGGAGAAAGAACCCACAGGAGGGCCTGTACAATGAGCTGCA |
| | | GAAGGACAAGATGGCCGAGGCCTATTCTGAGATCGGCATGAAGGG |
| | | AGAGAGGCGCCGGGGCAAGGGACACGATGGCCTGTACCAGGGCCT |
| | | GAGCACCGCCACAAAGGACACATATGATGCCCTGCACATGCAGGC |
| | | CCTGCCACCTAGGGGATCTGGAGCCACCAACTTTAGCCTGCTGAAG |
| | | CAGGCAGGCGATGTGGAGGAGAATCCAGGACCTGAGATGTGGCAC |
| | | GAGGGACTGGAGGAGGCAAGCAGGCTGTACTTTGGCGAGCGGAAT |
| | | GTGAAGGGCATGTTCGAGGTGCTGGAGCCACTGCACGCAATGATGG |
| | | AGAGGGGACCACAGACCCTGAAGGAGACATCCTTCAACCAGGCAT |
| | | ACGGAAGGGACCTGATGGAGGCACAGGAGTGGTGCCGGAAGTATA |
| | | TGAAGTCTGGCAATGTGAAGGACCTGCTGCAGGCCTGGGATCTGTA |
| | | TTACCACGTGTTTAGAAGGATCAGCAAGGGCTCCGGCGCCACCAAC |
| | | TTCTCCCTGCTGAAGCAGGCCGGCGATGTGGAAGAAAATCCAGGAC |
| | | CAATGCCTCTGGGACTGCTGTGGCTGGGACTGGCCCTGCTGGGCGC |
| | | CCTGCACGCCCAGGCCGGCGTGCAGGTGGAGACAATCAGCCCTGGC |
| | | GACGGCAGAACCTTTCCAAAGAGGGGCCAGACATGCGTGGTGCAC |
| | | TACACCGGCATGCTGGAGGATGGCAAGAAGTTCGACTCCTCTCGCG |
| | | ATCGGAACAAGCCCTTTAAGTTCATGCTGGGCAAGCAGGAAGTGAT |
| | | CAGAGGCTGGGAGGAGGGCGTGGCCCAGATGTCTGTGGGCCAGAG |
| | | GGCCAAGCTGACAATCAGCCCAGACTATGCATACGGAGCAACCGG |
| | | ACACCCTGGAATCATCCCACCACACGCCACACTGGTGTTCGATGTG |
| | | GAGCTGCTGAAGCTGGGCGAGGGCTCTAACACCAGCAAGGAGAAT |
| | | CCATTTCTGTTCGCACTGGAGGCAGTGGTCATCTCCGTGGGCTCTAT |
| | | GGGCCTGATCATCTCCCTGCTGTGCGTGTACTTTTGGCTGGAGAGA |
| | | ACAATGCCAAGGATCCCCACCCTGAAGAACCTGGAGGACCTGGTG |
| | | ACCGAGTACCACGGCAATTTCAGCGCCTGGTCCGGCGTGTCTAAGG |
| | | GACTGGCAGAGTCCCTGCAGCCAGATTATTCTGAGCGGCTGTGCCT |
| | | GGTGAGCGAGATCCCTCCAAAGGGAGGCGCCCTGGGAGAGGGACC |
| | | AGGAGCCAGCCCCTGCAACCAGCACTCCCCTTACTGGGCCCCCCCT |
| | | TGTTATACCCTGAAGCCAGAGACAGGCTCTGGCGCCACCAACTTCA |
| | | GCCTGCTGAAGCAAGCCGGCGACGTGGAAGAAAACCCAGGACCAA |
| | | TGGCACTGCCAGTGACCGCCCTGCTGCTGCCTCTGGCCCTGCTGCTG |
| | | CACGCAGCCAGACCCATCCTGTGGCACGAAATGTGGCATGAAGGCC |
| | | TGGAGGAGGCAAGCAGACTGTACTTTGGCGAGAGAAATGTGAAAG |
| | | GAATGTTTGAGGTGCTGGAGCCTCTGCACGCCATGATGGAGAGGGG |
| | | CCCTCAGACCCTGAAGGAGACATCCTTTAACCAGGCCTACGGCAGA |
| | | GACCTGATGGAGGCCCAGGAGTGGTGCAGGAAGTATATGAAGAGC |
| | | GGAAATGTGAAAGACCTGCTGCAGGCCTGGGATCTGTACTACCACG |
| | | TGTTCCGCCGGATCTCTAAGGGCAAGGATACAATCCCTTGGCTGGG |
| | | ACACCTGCTGGTGGGACTGAGCGGAGCCTTTGGCTTCATCATCCTG |
| | | GTGTATCTGCTGATCAACTGCAGAAATACAGGCCCATGGCTGAAGA |
| | | AGGTGCTGAAGTGTAACACCCCTGACCCATCCAAGTTCTTTTCTCAG |
| | | CTGAGCTCCGAGCACGGCGGCGATGTGCAGAAGTGGCTGTCTAGCC |
| | | CCTTTCCTTCCTCTAGCTTCAGCCCTGGAGGACTGGCACCTGAGATC |
| | | TCCCCACTGGAGGTGCTGGAGAGGGACAAGGTGACCCAGCTGCTGC |
| | | TGCAGCAGGATAAGGTGCCAGAGCCCGCCTCCCTGTCCTCTAACCA |
| | | CAGCCTGACCTCCTGCTTTACAAATCAGGGCTACTTCTTTTTCCACC |
| | | TGCCAGACGCACTGGAGATCGAGGCATGTCAGGTGTATTTCACATA |
| | | CGATCCCTATAGCGAGGAGGACCCTGATGAGGGAGTGGCCGGCGC |
| | | CCCAACCGGAAGCTCCCCTCAGCCACTGCAGCCACTGAGCGGAGAG |
| | | GACGATGCATATTGTACATTTCCTTCCCGCGACGATCTGCTGCTGTT |
| | | CTCTCCAAGCCTGCTGGGAGGACCATCTCCACCCAGCACCGCACCT |
| | | GGAGGATCCGGGGCAGGGGAGGAGCGGATGCCTCCATCTCTGCAG |
| | | GAGAGAGTGCCAAGGGACTGGGATCCACAGCCTCTGGGACCACCT |
| | | ACCCCTGGAGTGCCAGACCTGGTGGATTTCCAGCCACCCCCTGAGC |
| | | TGGTGCTGCGGGAGGCAGGAGAGGAGGTGCCAGACGCAGGACCTA |
| | | GAGAGGGCGTGAGCTTTCCCTGGTCCAGGCCACCAGGACAGGGAG |
| | | AGTTCCGCGCCCTGAACGCCCGGCTGCCCCTGAATACAGACGCCTA |
| | | CCTGTCTCTGCAGGAGCTGCAGGGCCAGGATCCTACCCACCTGGTG |
| | | TGA |
| 62 | Signal peptide | ATGCTGCTGCTGGTGACCTCCCTGCTGCTGTGCGAGCTGCCTCACCC |
| | (Human CSF2R) | AGCCTTTCTGCTGATCCCC |
| 63 | αCD19 scFv | GACATCCAGATGACACAGACCACAAGCTCCCTGTCTGCCAGCCTGG |
| | (VL_linker_VH) | GCGACAGAGTGACCATCTCCTGTAGGGCCTCTCAGGATATCAGCAA |
| | | GTACCTGAACTGGTATCAGCAGAAGCCAGATGGCACAGTGAAGCT |
| | | GCTGATCTACCACACCTCCAGGCTGCACTCTGGAGTGCCAAGCCGG |
| | | TTCTCCGGATCTGGAAGCGGCACCGACTATTCCCTGACAATCTCTA |
| | | ACCTGGAGCAGGAGGATATCGCCACATACTTTTGCCAGCAGGGCAA |
| | | TACCCTGCCATATACATTCGGCGGAGGAACCAAGCTGGAGATCACC |
| | | GGATCCACATCTGGAAGCGGCAAGCCAGGAAGCGGAGAGGGATCC |
| | | ACAAAGGGAGAGGTGAAGCTGCAGGAGAGCGGACCAGGACTGGTG |
| | | GCACCATCCCAGTCTCTGAGCGTGACCTGTACAGTGTCCGGCGTGT |
| | | CTCTGCCTGACTACGGCGTGTCCTGGATCAGGCAGCCACCTAGGAA |
| | | GGGACTGGAGTGGCTGGGCGTGATCTGGGGCTCTGAGACCACATAC |
| | | TATAATTCTGCCCTGAAGAGCCGCCTGACCATCATCAAGGACAACT |
| | | CCAAGTCTCAGGTGTTTCTGAAGATGAATAGCCTGCAGACCGACGA |
| | | TACAGCCATCTACTATTGCGCCAAGCACTACTATTACGGCGGCTCCT |
| | | ACGCCATGGATTATTGGGGCCAGGGCACCTCCGTGACAGTGTCTAG |
| | | C |
| 64 | IgG4 linker-CD28 | GAGTCTAAGTATGGCCCACCCTGCCCTCCATGTCCAATGTTCTGGGT |
| | TM | GCTGGTGGTGGTGGGAGGCGTGCTGGCCTGTTACTCCCTGCTGGTG |
| | | ACCGTGGCCTTTATCATCTTCTGGGTG |
| 65 | 4-1BB signal domain | AAGAGAGGCAGGAAGAAGCTGCTGTATATCTTTAAGCAGCCCTTCA |
| | | TGCGCCCTGTGCAGACCACACAGGAGGAGGACGGCTGCAGCTGTC |
| | | GGTTTCCAGAGGAGGAGGAGGGAGGATGCGAGCTG |
| 66 | CD3zeta | CGCGTGAAGTTCAGCCGGTCCGCCGATGCCCCTGCCTACCAGCAGG |
| | | GCCAGAACCAGCTGTATAACGAGCTGAATCTGGGCCGGAGAGAGG |
| | | AGTACGACGTGCTGGATAAGAGGAGGGGAAGGGACCCAGAGATGG |
| | | GAGGCAAGCCTCGGAGAAAGAACCCACAGGAGGGCCTGTACAATG |
| | | AGCTGCAGAAGGACAAGATGGCCGAGGCCTATTCTGAGATCGGCA |
| | | TGAAGGGAGAGAGGCGCCGGGGCAAGGGACACGATGGCCTGTACC |
| | | AGGGCCTGAGCACCGCCACAAAGGACACATATGATGCCCTGCACAT |
| | | GCAGGCCCTGCCACCTAGG |
| 67 | P2A self-cleaving | GGATCTGGAGCCACCAACTTTAGCCTGCTGAAGCAGGCAGGCGATG |
| | peptide | TGGAGGAGAATCCAGGACCT |
| 68 | Naked FRB | GAGATGTGGCACGAGGGACTGGAGGAGGCAAGCAGGCTGTACTTT |
| | | GGCGAGCGGAATGTGAAGGGCATGTTCGAGGTGCTGGAGCCACTG |
| | | CACGCAATGATGGAGAGGGGACCACAGACCCTGAAGGAGACATCC |
| | | TTCAACCAGGCATACGGAAGGGACCTGATGGAGGCACAGGAGTGG |
| | | TGCCGGAAGTATATGAAGTCTGGCAATGTGAAGGACCTGCTGCAGG |
| | | CCTGGGATCTGTATTACCACGTGTTTAGAAGGATCAGCAAG |
| 69 | P2A self-cleaving | GGCTCCGGCGCCACCAACTTCTCCCTGCTGAAGCAGGCCGGCGATG |
| | peptide | TGGAAGAAAATCCAGGACCA |
| 70 | Signal peptide- | ATGCCTCTGGGACTGCTGTGGCTGGGACTGGCCCTGCTGGGCGCCC |
| | FKBP12 | TGCACGCCCAGGCCGGCGTGCAGGTGGAGACAATCAGCCCTGGCG |
| | | ACGGCAGAACCTTTCCAAAGAGGGGCCAGACATGCGTGGTGCACT |
| | | ACACCGGCATGCTGGAGGATGGCAAGAAGTTCGACTCCTCTCGCGA |
| | | TCGGAACAAGCCCTTTAAGTTCATGCTGGGCAAGCAGGAAGTGATC |
| | | AGAGGCTGGGAGGAGGGCGTGGCCCAGATGTCTGTGGGCCAGAGG |
| | | GCCAAGCTGACAATCAGCCCAGACTATGCATACGGAGCAACCGGA |
| | | CACCCTGGAATCATCCCACCACACGCCACACTGGTGTTCGATGTGG |
| | | AGCTGCTGAAGCTG |
| 71 | IL-2R gamma TM- | GGCGAGGGCTCTAACACCAGCAAGGAGAATCCATTTCTGTTCGCAC |
| | Cytoplasmic domain | TGGAGGCAGTGGTCATCTCCGTGGGCTCTATGGGCCTGATCATCTC |
| | | CCTGCTGTGCGTGTACTTTTGGCTGGAGAGAACAATGCCAAGGATC |
| | | CCCACCCTGAAGAACCTGGAGGACCTGGTGACCGAGTACCACGGC |
| | | AATTTCAGCGCCTGGTCCGGCGTGTCTAAGGGACTGGCAGAGTCCC |
| | | TGCAGCCAGATTATTCTGAGCGGCTGTGCCTGGTGAGCGAGATCCC |
| | | TCCAAAGGGAGGCGCCCTGGGAGAGGGACCAGGAGCCAGCCCCTG |
| | | CAACCAGCACTCCCCTTACTGGGCCCCCCCTTGTTATACCCTGAAGC |
| | | CAGAGACA |
| 72 | P2A | GGCTCTGGCGCCACCAACTTCAGCCTGCTGAAGCAAGCCGGCGACG |
| | | TGGAAGAAAACCCAGGACCA |
| 73 | Signal peptide-FRB | ATGGCACTGCCAGTGACCGCCCTGCTGCTGCCTCTGGCCCTGCTGCT |
| | | GCACGCAGCCAGACCCATCCTGTGGCACGAAATGTGGCATGAAGG |
| | | CCTGGAGGAGGCAAGCAGACTGTACTTTGGCGAGAGAAATGTGAA |
| | | AGGAATGTTTGAGGTGCTGGAGCCTCTGCACGCCATGATGGAGAGG |
| | | GGCCCTCAGACCCTGAAGGAGACATCCTTTAACCAGGCCTACGGCA |
| | | GAGACCTGATGGAGGCCCAGGAGTGGTGCAGGAAGTATATGAAGA |
| | | GCGGAAATGTGAAAGACCTGCTGCAGGCCTGGGATCTGTACTACCA |
| | | CGTGTTCCGCCGGATCTCTAAG |
| 74 | IL-2R beta TM- | GGCAAGGATACAATCCCTTGGCTGGGACACCTGCTGGTGGGACTGA |
| | Cytoplasmic domain | GCGGAGCCTTTGGCTTCATCATCCTGGTGTATCTGCTGATCAACTGC |
| | | AGAAATACAGGCCCATGGCTGAAGAAGGTGCTGAAGTGTAACACC |
| | | CCTGACCCATCCAAGTTCTTTTCTCAGCTGAGCTCCGAGCACGGCG |
| | | GCGATGTGCAGAAGTGGCTGTCTAGCCCCTTTCCTTCCTCTAGCTTC |
| | | AGCCCTGGAGGACTGGCACCTGAGATCTCCCCACTGGAGGTGCTGG |
| | | AGAGGGACAAGGTGACCCAGCTGCTGCTGCAGCAGGATAAGGTGC |
| | | CAGAGCCCGCCTCCCTGTCCTCTAACCACAGCCTGACCTCCTGCTTT |
| | | ACAAATCAGGGCTACTTCTTTTTCCACCTGCCAGACGCACTGGAGA |
| | | TCGAGGCATGTCAGGTGTATTTCACATACGATCCCTATAGCGAGGA |
| | | GGACCCTGATGAGGGAGTGGCCGGCGCCCCAACCGGAAGCTCCCCT |
| | | CAGCCACTGCAGCCACTGAGCGGAGAGGACGATGCATATTGTACAT |
| | | TTCCTTCCCGCGACGATCTGCTGCTGTTCTCTCCAAGCCTGCTGGGA |
| | | GGACCATCTCCACCCAGCACCGCACCTGGAGGATCCGGGGCAGGG |
| | | GAGGAGCGGATGCCTCCATCTCTGCAGGAGAGAGTGCCAAGGGAC |
| | | TGGGATCCACAGCCTCTGGGACCACCTACCCCTGGAGTGCCAGACC |
| | | TGGTGGATTTCCAGCCACCCCCTGAGCTGGTGCTGCGGGAGGCAGG |
| | | AGAGGAGGTGCCAGACGCAGGACCTAGAGAGGGCGTGAGCTTTCC |
| | | CTGGTCCAGGCCACCAGGACAGGGAGAGTTCCGCGCCCTGAACGCC |
| | | CGGCTGCCCCTGAATACAGACGCCTACCTGTCTCTGCAGGAGCTGC |
| | | AGGGCCAGGATCCTACCCACCTGGTG |
| 75 | FRB_RACR_αCD19 | MEMWHEGLEEASRLYFGERNVKGMFEVLEPLHAMMERGPQTLKETS |
| | CAR lentiviral vector | FNQAYGRDLMEAQEWCRKYMKSGNVKDLLQAWDLYYHVFRRISKG |
| | | SGATNFSLLKQAGDVEENPGPMPLGLLWLGLALLGALHAQAGVQVET |
| | | ISPGDGRTFPKRGQTCVVHYTGMLEDGKKFDSSRDRNKPFKFMLGKQ |
| | | EVIRGWEEGVAQMSVGQRAKLTISPDYAYGATGHPGIIPPHATLVFDV |
| | | ELLKLGEGSNTSKENPFLFALEAVVISVGSMGLIISLLCVYFWLERTMP |
| | | RIPTLKNLEDLVTEYHGNFSAWSGVSKGLAESLQPDYSERLCLVSEIPP |
| | | KGGALGEGPGASPCNQHSPYWAPPCYTLKPETGSGATNFSLLKQAGD |
| | | VEENPGPMALPVTALLLPLALLLHAARPILWHEMWHEGLEEASRLYF |
| | | GERNVKGMFEVLEPLHAMMERGPQTLKETSFNQAYGRDLMEAQEWC |
| | | RKYMKSGNVKDLLQAWDLYYHVFRRISKGKDTIPWLGHLLVGLSGAF |
| | | GFIILVYLLINCRNTGPWLKKVLKCNTPDPSKFFSQLSSEHGGDVQKWL |
| | | SSPFPSSSFSPGGLAPEISPLEVLERDKVTQLLLQQDKVPEPASLSSNHSL |
| | | TSCFTNQGYFFFHLPDALEIEACQVYFTYDPYSEEDPDEGVAGAPTGSS |
| | | PQPLQPLSGEDDAYCTFPSRDDLLLFSPSLLGGPSPPSTAPGGSGAGEER |
| | | MPPSLQERVPRDWDPQPLGPPTPGVPDLVDFQPPPELVLREAGEEVPD |
| | | AGPREGVSFPWSRPPGQGEFRALNARLPLNTDAYLSLQELQGQDPTHL |
| | | VGSGATNFSLLKQAGDVEENPGPMLLLVTSLLLCELPHPAFLLIPDIQM |
| | | TQTTSSLSASLGDRVTISCRASQDISKYLNWYQQKPDGTVKLLIYHTSR |
| | | LHSGVPSRFSGSGSGTDYSLTISNLEQEDIATYFCQQGNTLPYTFGGGT |
| | | KLEITGSTSGSGKPGSGEGSTKGEVKLQESGPGLVAPSQSLSVTCTVSG |
| | | VSLPDYGVSWIRQPPRKGLEWLGVIWGSETTYYNSALKSRLTIIKDNSK |
| | | SQVFLKMNSLQTDDTAIYYCAKHYYYGGSYAMDYWGQGTSVTVSSE |
| | | SKYGPPCPPCPMFWVLVVVGGVLACYSLLVTVAFIIFWVKRGRKKLL |
| | | YIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 76 | Naked FRB_P2A | MEMWHEGLEEASRLYFGERNVKGMFEVLEPLHAMMERGPQTLKETS |
| | | FNQAYGRDLMEAQEWCRKYMKSGNVKDLLQAWDLYYHVFRRISKG |
| | | SGATNFSLLKQAGDVEENPGP |
| 77 | RACRg | MPLGLLWLGLALLGALHAQAGVQVETISPGDGRTFPKRGQTCVVHYT |
| | (FKBP12_IL-2R | GMLEDGKKFDSSRDRNKPFKFMLGKQEVIRGWEEGVAQMSVGQRAK |
| | gamma)_P2A | LTISPDYAYGATGHPGIIPPHATLVFDVELLKLGEGSNTSKENPFLFALE |
| | | AVVISVGSMGLIISLLCVYFWLERTMPRIPTLKNLEDLVTEYHGNFSAW |
| | | SGVSKGLAESLQPDYSERLCLVSEIPPKGGALGEGPGASPCNQHSPYW |
| | | APPCYTLKPETGSGATNFSLLKQAGDVEENPGP |
| 78 | RACRb (FRB_IL-2R | MALPVTALLLPLALLLHAARPILWHEMWHEGLEEASRLYFGERNVKG |
| | beta)_P2A | MFEVLEPLHAMMERGPQTLKETSFNQAYGRDLMEAQEWCRKYMKSG |
| | | NVKDLLQAWDLYYHVFRRISKGKDTIPWLGHLLVGLSGAFGFIILVYL |
| | | LINCRNTGPWLKKVLKCNTPDPSKFFSQLSSEHGGDVQKWLSSPFPSSS |
| | | FSPGGLAPEISPLEVLERDKVTQLLLQQDKVPEPASLSSNHSLTSCFTNQ |
| | | GYFFFHLPDALEIEACQVYFTYDPYSEEDPDEGVAGAPTGSSPQPLQPL |
| | | SGEDDAYCTFPSRDDLLLFSPSLLGGPSPPSTAPGGSGAGEERMPPSLQ |
| | | ERVPRDWDPQPLGPPTPGVPDLVDFQPPPELVLREAGEEVPDAGPREG |
| | | VSFPWSRPPGQGEFRALNARLPLNTDAYLSLQELQGQDPTHLVGSGAT |
| | | NFSLLKQAGDVEENPGP |
| 79 | αCD19 scFv | MLLLVTSLLLCELPHPAFLLIPDIQMTQTTSSLSASLGDRVTISCRASQDI |
| | | SKYLNWYQQKPDGTVKLLIYHTSRLHSGVPSRFSGSGSGTDYSLTISNL |
| | | EQEDIATYFCQQGNTLPYTFGGGTKLEITGSTSGSGKPGSGEGSTKGEV |
| | | KLQESGPGLVAPSQSLSVTCTVSGVSLPDYGVSWIRQPPRKGLEWLGV |
| | | IWGSETTYYNSALKSRLTIIKDNSKSQVFLKMNSLQTDDTAIYYCAKH |
| | | YYYGGSYAMDYWGQGTSVTVSS |
| 80 | IgG4 linker-CD28 | ESKYGPPCPPCPMFWVLVVVGGVLACYSLLVTVAFIIFWVKRGRKKLL |
| | TM_4-1BB-CD3zeta | YIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 81 | FRB_RACR_αCD19 | ATGGAGATGTGGCACGAGGGACTGGAGGAGGCAAGCAGACTGTAC |
| | CAR lentiviral | TTTGGCGAGAGGAACGTGAAGGGCATGTTCGAGGTGCTGGAGCCA |
| | vector | CTGCACGCAATGATGGAGAGGGGACCACAGACCCTGAAGGAGACA |
| | | TCTTTCAACCAGGCATACGGAAGGGACCTGATGGAGGCACAGGAG |
| | | TGGTGCCGGAAGTATATGAAGAGCGGCAATGTGAAGGACCTGCTG |
| | | CAGGCCTGGGATCTGTACTATCACGTGTTTCGGAGAATCTCCAAGG |
| | | GCTCTGGCGCCACCAACTTCTCCCTGCTGAAGCAGGCCGGCGATGT |
| | | GGAGGAGAATCCTGGACCAATGCCACTGGGACTGCTGTGGCTGGG |
| | | ACTGGCCCTGCTGGGCGCCCTGCACGCCCAGGCCGGCGTGCAGGTG |
| | | GAGACAATCAGCCCTGGCGACGGACGCACCTTTCCAAAGAGGGGA |
| | | CAGACATGCGTGGTGCACTACACCGGCATGCTGGAGGATGGCAAG |
| | | AAGTTCGACAGCTCCAGAGATAGGAATAAGCCCTTTAAGTTCATGC |
| | | TGGGCAAGCAGGAAGTGATCAGGGGATGGGAGGAGGGAGTGGCAC |
| | | AGATGTCTGTGGGACAGCGGGCCAAGCTGACAATCAGCCCAGACT |
| | | ATGCATACGGAGCAACCGGACACCCTGGAATCATCCCACCTCACGC |
| | | CACACTGGTGTTTGATGTGGAGCTGCTGAAGCTGGGCGAGGGCAGC |
| | | AACACCTCCAAGGAGAATCCATTTCTGTTCGCCCTGGAGGCCGTGG |
| | | TCATCTCTGTGGGCAGCATGGGCCTGATCATCTCCCTGCTGTGCGTG |
| | | TACTTTTGGCTGGAGCGCACAATGCCACGGATCCCCACCCTGAAGA |
| | | ACCTGGAGGACCTGGTGACCGAGTACCACGGCAATTTCTCCGCCTG |
| | | GTCTGGCGTGAGCAAGGGACTGGCAGAGTCTCTGCAGCCAGATTAT |
| | | AGCGAGCGGCTGTGCCTGGTGAGCGAGATCCCACCCAAGGGAGGC |
| | | GCCCTGGGAGAGGGACCAGGAGCCTCCCCTTGCAACCAGCACTCTC |
| | | CTTACTGGGCCCCTCCATGTTATACCCTGAAGCCAGAGACAGGCAG |
| | | CGGAGCTACTAACTTCTCCCTGCTGAAGCAAGCAGGCGACGTGGAA |
| | | GAAAATCCTGGACCAATGGCACTGCCAGTGACCGCCCTGCTGCTGC |
| | | CTCTGGCCCTGCTGCTGCACGCAGCCAGACCCATCCTGTGGCACGA |
| | | AATGTGGCATGAAGGCCTGGAGGAGGCAAGCAGGCTGTACTTTGG |
| | | CGAGCGGAATGTGAAAGGAATGTTTGAAGTGCTGGAGCCTCTGCAC |
| | | GCCATGATGGAGAGGGGCCCTCAGACCCTGAAGGAGACATCCTTTA |
| | | ACCAGGCCTACGGCAGAGACCTGATGGAGGCCCAGGAGTGGTGCA |
| | | GGAAGTATATGAAGTCTGGAAATGTGAAAGACCTGCTGCAGGCCTG |
| | | GGATCTGTATTATCACGTGTTCAGGCGCATCTCTAAGGGCAAGGAT |
| | | ACAATCCCTTGGCTGGGACACCTGCTGGTGGGACTGAGCGGAGCCT |
| | | TTGGCTTCATCATCCTGGTGTATCTGCTGATCAACTGCCGCAATACA |
| | | GGCCCATGGCTGAAGAAGGTGCTGAAGTGTAACACCCCCGACCCTT |
| | | CCAAGTTCTTTTCTCAGCTGTCTAGCGAGCACGGCGGCGATGTGCA |
| | | GAAGTGGCTGTCCTCTCCATTTCCCAGCTCCTCTTTCAGCCCAGGAG |
| | | GACTGGCACCAGAGATCTCCCCACTGGAGGTGCTGGAGAGGGACA |
| | | AGGTGACCCAGCTGCTGCTGCAGCAGGATAAGGTGCCTGAGCCAGC |
| | | CTCCCTGAGCTCCAACCACTCCCTGACCTCTTGCTTTACAAATCAGG |
| | | GCTACTTCTTTTTCCACCTGCCAGACGCACTGGAGATCGAGGCATGT |
| | | CAGGTGTATTTCACATACGATCCCTATAGCGAGGAGGACCCTGATG |
| | | AGGGAGTGGCCGGCGCCCCAACCGGATCTAGCCCACAGCCTCTGCA |
| | | GCCACTGAGCGGAGAGGACGATGCATATTGTACATTTCCTTCCCGC |
| | | GACGATCTGCTGCTGTTCTCTCCAAGCCTGCTGGGAGGACCAAGCC |
| | | CACCTTCCACCGCACCAGGCGGCTCCGGGGCAGGGGAGGAGCGGA |
| | | TGCCACCCTCTCTGCAGGAGAGAGTGCCAAGGGACTGGGATCCACA |
| | | GCCACTGGGACCTCCAACCCCTGGAGTGCCAGACCTGGTGGATTTC |
| | | CAGCCCCCTCCAGAGCTGGTGCTGAGAGAGGCAGGAGAGGAGGTG |
| | | CCTGACGCAGGACCAAGAGAGGGCGTGAGCTTTCCTTGGTCCAGGC |
| | | CACCTGGACAGGGAGAGTTCAGAGCCCTGAACGCCAGGCTGCCCCT |
| | | GAATACAGACGCCTACCTGTCTCTGCAGGAGCTGCAGGGCCAGGAT |
| | | CCTACACACCTGGTCGGATCTGGCGCCACCAACTTTAGCCTGCTGA |
| | | AGCAGGCAGGCGACGTGGAAGAGAACCCTGGACCAATGCTGCTGC |
| | | TGGTGACCAGCCTGCTGCTGTGCGAGCTGCCACACCCTGCCTTCCTG |
| | | CTGATCCCAGATATCCAGATGACACAGACCACATCCTCTCTGTCCG |
| | | CCTCTCTGGGCGACAGAGTGACCATCTCTTGTAGGGCCAGCCAGGA |
| | | TATCTCCAAGTACCTGAACTGGTATCAGCAGAAGCCTGACGGCACA |
| | | GTGAAGCTGCTGATCTACCACACCTCTAGGCTGCACAGCGGAGTGC |
| | | CATCCCGGTTCAGCGGATCCGGATCTGGAACAGACTATTCTCTGAC |
| | | CATCAGCAACCTGGAGCAGGAGGATATCGCCACATACTTTTGCCAG |
| | | CAGGGCAATACCCTGCCATATACATTCGGCGGAGGAACCAAGCTGG |
| | | AGATCACCGGAAGCACATCCGGATCTGGCAAGCCAGGATCCGGAG |
| | | AGGGATCTACAAAGGGAGAGGTGAAGCTGCAGGAGAGCGGACCAG |
| | | GACTGGTGGCACCCAGCCAGTCCCTGTCTGTGACCTGTACAGTGTC |
| | | TGGCGTGAGCCTGCCCGATTACGGCGTGTCCTGGATCAGACAGCCA |
| | | CCAAGGAAGGGACTGGAGTGGCTGGGCGTGATCTGGGGCTCTGAG |
| | | ACCACATACTATAATAGCGCCCTGAAGTCCCGGCTGACCATCATCA |
| | | AGGACAACAGCAAGTCCCAGGTGTTTCTGAAGATGAATAGCCTGCA |
| | | GACCGACGATACAGCCATCTACTATTGCGCCAAGCACTACTATTAC |
| | | GGCGGCTCCTACGCCATGGATTATTGGGGCCAGGGCACCTCCGTGA |
| | | CAGTGAGCTCCGAGTCTAAGTATGGCCCTCCATGCCCCCCTTGTCCT |
| | | ATGTTCTGGGTGCTGGTGGTGGTGGGAGGCGTGCTGGCCTGTTACT |
| | | CCCTGCTGGTGACCGTGGCCTTTATCATCTTCTGGGTGAAGCGCGGC |
| | | CGGAAGAAGCTGCTGTATATCTTTAAGCAGCCCTTCATGAGACCTG |
| | | TGCAGACCACACAGGAGGAGGACGGCTGCAGCTGTAGGTTTCCAG |
| | | AGGAGGAGGAGGGAGGATGCGAGCTGCGCGTGAAGTTCTCTCGGA |
| | | GCGCCGATGCCCCTGCCTACCAGCAGGGACAGAACCAGCTGTATAA |
| | | CGAGCTGAATCTGGGCCGGAGAGAGGAGTACGACGTGCTGGATAA |
| | | GAGGAGGGGAAGAGACCCAGAGATGGGAGGCAAGCCTCGGAGAA |
| | | AGAACCCACAGGAGGGCCTGTACAATGAGCTGCAGAAGGACAAGA |
| | | TGGCCGAGGCCTATTCCGAGATCGGCATGAAGGGAGAGAGGCGCC |
| | | GGGGCAAGGGACACGATGGCCTGTACCAGGGCCTGAGCACCGCCA |
| | | CAAAGGACACCTATGATGCCCTGCACATGCAGGCCCTGCCACCCAG |
| | | GTGA |
| 82 | Naked FRB_P2A | ATGGAGATGTGGCACGAGGGACTGGAGGAGGCAAGCAGACTGTAC |
| | | TTTGGCGAGAGGAACGTGAAGGGCATGTTCGAGGTGCTGGAGCCA |
| | | CTGCACGCAATGATGGAGAGGGGACCACAGACCCTGAAGGAGACA |
| | | TCTTTCAACCAGGCATACGGAAGGGACCTGATGGAGGCACAGGAG |
| | | TGGTGCCGGAAGTATATGAAGAGCGGCAATGTGAAGGACCTGCTG |
| | | CAGGCCTGGGATCTGTACTATCACGTGTTTCGGAGAATCTCCAAGG |
| | | GCTCTGGCGCCACCAACTTCTCCCTGCTGAAGCAGGCCGGCGATGT |
| | | GGAGGAGAATCCTGGACCA |
| 83 | RACRg | ATGCCACTGGGACTGCTGTGGCTGGGACTGGCCCTGCTGGGCGCCC |
| | (FKBP12_IL-2R | TGCACGCCCAGGCCGGCGTGCAGGTGGAGACAATCAGCCCTGGCG |
| | gamma)_P2A | ACGGACGCACCTTTCCAAAGAGGGGACAGACATGCGTGGTGCACT |
| | | ACACCGGCATGCTGGAGGATGGCAAGAAGTTCGACAGCTCCAGAG |
| | | ATAGGAATAAGCCCTTTAAGTTCATGCTGGGCAAGCAGGAAGTGAT |
| | | CAGGGGATGGGAGGAGGGAGTGGCACAGATGTCTGTGGGACAGCG |
| | | GGCCAAGCTGACAATCAGCCCAGACTATGCATACGGAGCAACCGG |
| | | ACACCCTGGAATCATCCCACCTCACGCCACACTGGTGTTTGATGTG |
| | | GAGCTGCTGAAGCTGGGCGAGGGCAGCAACACCTCCAAGGAGAAT |
| | | CCATTTCTGTTCGCCCTGGAGGCCGTGGTCATCTCTGTGGGCAGCAT |
| | | GGGCCTGATCATCTCCCTGCTGTGCGTGTACTTTTGGCTGGAGCGCA |
| | | CAATGCCACGGATCCCCACCCTGAAGAACCTGGAGGACCTGGTGAC |
| | | CGAGTACCACGGCAATTTCTCCGCCTGGTCTGGCGTGAGCAAGGGA |
| | | CTGGCAGAGTCTCTGCAGCCAGATTATAGCGAGCGGCTGTGCCTGG |
| | | TGAGCGAGATCCCACCCAAGGGAGGCGCCCTGGGAGAGGGACCAG |
| | | GAGCCTCCCCTTGCAACCAGCACTCTCCTTACTGGGCCCCTCCATGT |
| | | TATACCCTGAAGCCAGAGACAGGCAGCGGAGCTACTAACTTCTCCC |
| | | TGCTGAAGCAAGCAGGCGACGTGGAAGAAAATCCTGGACCA |
| 84 | RACRb (FRB_IL-2R | ATGGCACTGCCAGTGACCGCCCTGCTGCTGCCTCTGGCCCTGCTGCT |
| | beta)_P2A | GCACGCAGCCAGACCCATCCTGTGGCACGAAATGTGGCATGAAGG |
| | | CCTGGAGGAGGCAAGCAGGCTGTACTTTGGCGAGCGGAATGTGAA |
| | | AGGAATGTTTGAAGTGCTGGAGCCTCTGCACGCCATGATGGAGAGG |
| | | GGCCCTCAGACCCTGAAGGAGACATCCTTTAACCAGGCCTACGGCA |
| | | GAGACCTGATGGAGGCCCAGGAGTGGTGCAGGAAGTATATGAAGT |
| | | CTGGAAATGTGAAAGACCTGCTGCAGGCCTGGGATCTGTATTATCA |
| | | CGTGTTCAGGCGCATCTCTAAGGGCAAGGATACAATCCCTTGGCTG |
| | | GGACACCTGCTGGTGGGACTGAGCGGAGCCTTTGGCTTCATCATCC |
| | | TGGTGTATCTGCTGATCAACTGCCGCAATACAGGCCCATGGCTGAA |
| | | GAAGGTGCTGAAGTGTAACACCCCCGACCCTTCCAAGTTCTTTTCTC |
| | | AGCTGTCTAGCGAGCACGGCGGCGATGTGCAGAAGTGGCTGTCCTC |
| | | TCCATTTCCCAGCTCCTCTTTCAGCCCAGGAGGACTGGCACCAGAG |
| | | ATCTCCCCACTGGAGGTGCTGGAGAGGGACAAGGTGACCCAGCTGC |
| | | TGCTGCAGCAGGATAAGGTGCCTGAGCCAGCCTCCCTGAGCTCCAA |
| | | CCACTCCCTGACCTCTTGCTTTACAAATCAGGGCTACTTCTTTTTCC |
| | | ACCTGCCAGACGCACTGGAGATCGAGGCATGTCAGGTGTATTTCAC |
| | | ATACGATCCCTATAGCGAGGAGGACCCTGATGAGGGAGTGGCCGG |
| | | CGCCCCAACCGGATCTAGCCCACAGCCTCTGCAGCCACTGAGCGGA |
| | | GAGGACGATGCATATTGTACATTTCCTTCCCGCGACGATCTGCTGCT |
| | | GTTCTCTCCAAGCCTGCTGGGAGGACCAAGCCCACCTTCCACCGCA |
| | | CCAGGCGGCTCCGGGGCAGGGGAGGAGCGGATGCCACCCTCTCTG |
| | | CAGGAGAGAGTGCCAAGGGACTGGGATCCACAGCCACTGGGACCT |
| | | CCAACCCCTGGAGTGCCAGACCTGGTGGATTTCCAGCCCCCTCCAG |
| | | AGCTGGTGCTGAGAGAGGCAGGAGAGGAGGTGCCTGACGCAGGAC |
| | | CAAGAGAGGGCGTGAGCTTTCCTTGGTCCAGGCCACCTGGACAGGG |
| | | AGAGTTCAGAGCCCTGAACGCCAGGCTGCCCCTGAATACAGACGCC |
| | | TACCTGTCTCTGCAGGAGCTGCAGGGCCAGGATCCTACACACCTGG |
| | | TCGGATCTGGCGCCACCAACTTTAGCCTGCTGAAGCAGGCAGGCGA |
| | | CGTGGAAGAGAACCCTGGACCA |
| 85 | αCD19 scFv | ATGCTGCTGCTGGTGACCAGCCTGCTGCTGTGCGAGCTGCCACACC |
| | | CTGCCTTCCTGCTGATCCCAGATATCCAGATGACACAGACCACATC |
| | | CTCTCTGTCCGCCTCTCTGGGCGACAGAGTGACCATCTCTTGTAGGG |
| | | CCAGCCAGGATATCTCCAAGTACCTGAACTGGTATCAGCAGAAGCC |
| | | TGACGGCACAGTGAAGCTGCTGATCTACCACACCTCTAGGCTGCAC |
| | | AGCGGAGTGCCATCCCGGTTCAGCGGATCCGGATCTGGAACAGACT |
| | | ATTCTCTGACCATCAGCAACCTGGAGCAGGAGGATATCGCCACATA |
| | | CTTTTGCCAGCAGGGCAATACCCTGCCATATACATTCGGCGGAGGA |
| | | ACCAAGCTGGAGATCACCGGAAGCACATCCGGATCTGGCAAGCCA |
| | | GGATCCGGAGAGGGATCTACAAAGGGAGAGGTGAAGCTGCAGGAG |
| | | AGCGGACCAGGACTGGTGGCACCCAGCCAGTCCCTGTCTGTGACCT |
| | | GTACAGTGTCTGGCGTGAGCCTGCCCGATTACGGCGTGTCCTGGAT |
| | | CAGACAGCCACCAAGGAAGGGACTGGAGTGGCTGGGCGTGATCTG |
| | | GGGCTCTGAGACCACATACTATAATAGCGCCCTGAAGTCCCGGCTG |
| | | ACCATCATCAAGGACAACAGCAAGTCCCAGGTGTTTCTGAAGATGA |
| | | ATAGCCTGCAGACCGACGATACAGCCATCTACTATTGCGCCAAGCA |
| | | CTACTATTACGGCGGCTCCTACGCCATGGATTATTGGGGCCAGGGC |
| | | ACCTCCGTGACAGTGAGCTCC |
| 86 | IgG4 linker-CD28 | GAGTCTAAGTATGGCCCTCCATGCCCCCCTTGTCCTATGTTCTGGGT |
| | TM_4-1BB-CD3zeta | GCTGGTGGTGGTGGGAGGCGTGCTGGCCTGTTACTCCCTGCTGGTG |
| | | ACCGTGGCCTTTATCATCTTCTGGGTGAAGCGCGGCCGGAAGAAGC |
| | | TGCTGTATATCTTTAAGCAGCCCTTCATGAGACCTGTGCAGACCAC |
| | | ACAGGAGGAGGACGGCTGCAGCTGTAGGTTTCCAGAGGAGGAGGA |
| | | GGGAGGATGCGAGCTGCGCGTGAAGTTCTCTCGGAGCGCCGATGCC |
| | | CCTGCCTACCAGCAGGGACAGAACCAGCTGTATAACGAGCTGAATC |
| | | TGGGCCGGAGAGAGGAGTACGACGTGCTGGATAAGAGGAGGGGAA |
| | | GAGACCCAGAGATGGGAGGCAAGCCTCGGAGAAAGAACCCACAGG |
| | | AGGGCCTGTACAATGAGCTGCAGAAGGACAAGATGGCCGAGGCCT |
| | | ATTCCGAGATCGGCATGAAGGGAGAGAGGCGCCGGGGCAAGGGAC |
| | | ACGATGGCCTGTACCAGGGCCTGAGCACCGCCACAAAGGACACCT |
| | | ATGATGCCCTGCACATGCAGGCCCTGCCACCCAGG |
| 87 | FRB_αCD19 | MEMWHEGLEEASRLYFGERNVKGMFEVLEPLHAMMERGPQTLKETS |
| | CAR_TGFbDN | FNQAYGRDLMEAQEWCRKYMKSGNVKDLLQAWDLYYHVFRRISKG |
| | lentiviral vector | SGATNFSLLKQAGDVEENPGPMLLLVTSLLLCELPHPAFLLIPDIQMTQ |
| | | TTSSLSASLGDRVTISCRASQDISKYLNWYQQKPDGTVKLLIYHTSRLH |
| | | SGVPSRFSGSGSGTDYSLTISNLEQEDIATYFCQQGNTLPYTFGGGTKL |
| | | EITGSTSGSGKPGSGEGSTKGEVKLQESGPGLVAPSQSLSVTCTVSGVS |
| | | LPDYGVSWIRQPPRKGLEWLGVIWGSETTYYNSALKSRLTIIKDNSKSQ |
| | | VFLKMNSLQTDDTAIYYCAKHYYYGGSYAMDYWGQGTSVTVSSESK |
| | | YGPPCPPCPMFWVLVVVGGVLACYSLLVTVAFIIFWVKRGRKKLLYIF |
| | | KQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQ |
| | | KDKMAEAYSEIGMKGERRRGKGHDGLYQGLSTATKDTYDALHMQAL |
| | | PPRGSGATNFSLLKQAGDVEENPGPMGRGLLRGLWPLHIVLWTRIAST |
| | | IPPHVQKSVNNDMIVTDNNGAVKFPQLCKFCDVRFSTCDNQKSCMSN |
| | | CSITSICEKPQEVCVAVWRKNDENITLETVCHDPKLPYHDFILEDAASP |
| | | KCIMKEKKKPGETFFMCSCSSDECNDNIIFSEEYNTSNPDLLLVIFQVTG |
| | | ISLLPPLGVAISVIIIFYCYRVNRQQKRRR |
| 88 | Naked FRB_P2A | MEMWHEGLEEASRLYFGERNVKGMFEVLEPLHAMMERGPQTLKETS |
| | | FNQAYGRDLMEAQEWCRKYMKSGNVKDLLQAWDLYYHVFRRISKG |
| | | SGATNFSLLKQAGDVEENPGP |
| 89 | αCD19 scFv | MLLLVTSLLLCELPHPAFLLIPDIQMTQTTSSLSASLGDRVTISCRASQDI |
| | | SKYLNWYQQKPDGTVKLLIYHTSRLHSGVPSRFSGSGSGTDYSLTISNL |
| | | EQEDIATYFCQQGNTLPYTFGGGTKLEITGSTSGSGKPGSGEGSTKGEV |
| | | KLQESGPGLVAPSQSLSVTCTVSGVSLPDYGVSWIRQPPRKGLEWLGV |
| | | IWGSETTYYNSALKSRLTIIKDNSKSQVFLKMNSLQTDDTAIYYCAKH |
| | | YYYGGSYAMDYWGQGTSVTVSS |
| 90 | IgG4 linker-CD28 | ESKYGPPCPPCPMFWVLVVVGGVLACYSLLVTVAFIIFWVKRGRKKLL |
| | TM_4-1BB- | YIFKQPFMRPVQTTQEEDGCSCRFPEEEEGGCELRVKFSRSADAPAYQ |
| | CD3zeta_P2A | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPRGSGATNFSLLKQAGDVEENPGP |
| 91 | TGF beta DN | MGRGLLRGLWPLHIVLWTRIASTIPPHVQKSVNNDMIVTDNNGAVKFP |
| | | QLCKFCDVRFSTCDNQKSCMSNCSITSICEKPQEVCVAVWRKNDENIT |
| | | LETVCHDPKLPYHDFILEDAASPKCIMKEKKKPGETFFMCSCSSDECND |
| | | NIIFSEEYNTSNPDLLLVIFQVTGISLLPPLGVAISVIIIFYCYRVNRQQKR |
| | | RR |
| 92 | FRB_αCD19 | ATGGAGATGTGGCACGAGGGACTGGAGGAGGCATCCAGACTGTAC |
| | CAR_TGFbDN | TTCGGCGAGAGGAACGTGAAGGGCATGTTTGAGGTGCTGGAGCCA |
| | lentiviral vector | CTGCACGCCATGATGGAGAGAGGCCCCCAGACCCTGAAGGAGACA |
| | | TCTTTCAACCAGGCCTATGGAAGGGACCTGATGGAGGCACAGGAGT |
| | | GGTGCCGGAAGTACATGAAGAGCGGCAATGTGAAGGACCTGCTGC |
| | | AGGCCTGGGATCTGTACTATCACGTGTTCCGGAGAATCAGCAAGGG |
| | | CTCCGGCGCCACCAACTTTAGCCTGCTGAAGCAGGCAGGCGACGTG |
| | | GAGGAGAATCCAGGACCTATGCTGCTGCTGGTGACATCCCTGCTGC |
| | | TGTGCGAGCTGCCACACCCAGCCTTCCTGCTGATCCCCGATATCCA |
| | | GATGACCCAGACCACAAGCTCCCTGAGCGCCTCCCTGGGCGACAGG |
| | | GTGACAATCTCTTGTCGGGCCAGCCAGGATATCTCCAAGTATCTGA |
| | | ATTGGTACCAGCAGAAGCCCGACGGCACCGTGAAGCTGCTGATCTA |
| | | TCACACATCTAGACTGCACAGCGGCGTGCCTTCCAGGTTTTCTGGC |
| | | AGCGGCTCCGGCACCGACTACTCTCTGACAATCAGCAACCTGGAGC |
| | | AGGAGGATATCGCCACCTATTTCTGCCAGCAGGGCAATACCCTGCC |
| | | TTACACATTTGGCGGCGGCACAAAGCTGGAGATCACCGGCTCTACA |
| | | AGCGGATCCGGCAAGCCAGGATCCGGAGAGGGATCTACCAAGGGA |
| | | GAGGTGAAGCTGCAGGAGAGCGGACCTGGACTGGTGGCACCATCT |
| | | CAGAGCCTGTCCGTGACCTGTACAGTGTCTGGCGTGAGCCTGCCAG |
| | | ATTATGGCGTGAGCTGGATCAGGCAGCCACCTAGGAAGGGACTGG |
| | | AGTGGCTGGGCGTGATCTGGGGCTCCGAGACCACATACTATAACAG |
| | | CGCCCTGAAGTCCCGCCTGACCATCATCAAGGACAACTCTAAGAGC |
| | | CAGGTGTTCCTGAAGATGAATTCCCTGCAGACCGACGATACAGCCA |
| | | TCTACTATTGCGCCAAGCACTACTATTACGGCGGCTCTTATGCCATG |
| | | GATTACTGGGGCCAGGGCACCAGCGTGACAGTGTCTAGCGAGTCCA |
| | | AGTACGGCCCACCCTGCCCTCCATGTCCCATGTTTTGGGTGCTGGTG |
| | | GTGGTGGGAGGCGTGCTGGCCTGTTATTCCCTGCTGGTGACCGTGG |
| | | CCTTCATCATCTTTTGGGTGAAGCGCGGCCGGAAGAAGCTGCTGTA |
| | | CATCTTCAAGCAGCCCTTCATGAGACCCGTGCAGACCACACAGGAG |
| | | GAGGACGGCTGCAGCTGTAGGTTCCCAGAGGAGGAGGAGGGAGGA |
| | | TGCGAGCTGAGGGTGAAGTTTTCCCGGTCTGCCGATGCCCCTGCCT |
| | | ATCAGCAGGGCCAGAATCAGCTGTACAACGAGCTGAATCTGGGCA |
| | | GGCGCGAGGAGTACGACGTGCTGGATAAGAGGAGAGGAAGGGACC |
| | | CTGAGATGGGAGGCAAGCCAAGGCGCAAGAACCCTCAGGAGGGCC |
| | | TGTATAATGAGCTGCAGAAGGACAAGATGGCCGAGGCCTACTCCG |
| | | AGATCGGCATGAAGGGAGAGCGGAGAAGGGGCAAGGGACACGAT |
| | | GGCCTGTATCAGGGCCTGAGCACCGCCACAAAGGACACCTACGATG |
| | | CACTGCACATGCAGGCCCTGCCACCTAGAGGATCTGGAGCCACAAA |
| | | CTTCAGCCTGCTGAAGCAGGCCGGCGATGTGGAGGAGAATCCTGGA |
| | | CCAATGGGAAGAGGACTGCTGAGGGGACTGTGGCCACTGCACATC |
| | | GTGCTGTGGACCAGGATCGCCTCTACAATCCCACCCCACGTGCAGA |
| | | AGAGCGTGAACAATGACATGATCGTGACCGATAACAATGGCGCCG |
| | | TGAAGTTTCCCCAGCTGTGCAAGTTCTGTGACGTGCGCTTTTCCACC |
| | | TGTGATAACCAGAAGTCCTGCATGTCTAATTGTAGCATCACATCCA |
| | | TCTGCGAGAAGCCTCAGGAGGTGTGCGTGGCCGTGTGGCGGAAGA |
| | | ACGACGAGAATATCACCCTGGAGACAGTGTGCCACGATCCCAAGCT |
| | | GCCTTATCACGACTTCATCCTGGAGGATGCCGCCTCTCCTAAGTGTA |
| | | TCATGAAGGAGAAGAAGAAGCCAGGCGAGACCTTCTTTATGTGCA |
| | | GCTGTTCCTCTGACGAGTGCAACGATAATATCATCTTCTCCGAGGA |
| | | GTACAACACCTCTAATCCTGACCTGCTGCTGGTCATCTTTCAGGTGA |
| | | CAGGCATCTCCCTGCTGCCTCCACTGGGCGTGGCCATCTCTGTGATC |
| | | ATCATCTTTTATTGTTACAGAGTGAACAGGCAGCAGAAGCGCCGGC |
| | | GCTAG |
| 93 | Naked FRB_P2A | ATGGAGATGTGGCACGAGGGACTGGAGGAGGCATCCAGACTGTAC |
| | | TTCGGCGAGAGGAACGTGAAGGGCATGTTTGAGGTGCTGGAGCCA |
| | | CTGCACGCCATGATGGAGAGAGGCCCCCAGACCCTGAAGGAGACA |
| | | TCTTTCAACCAGGCCTATGGAAGGGACCTGATGGAGGCACAGGAGT |
| | | GGTGCCGGAAGTACATGAAGAGCGGCAATGTGAAGGACCTGCTGC |
| | | AGGCCTGGGATCTGTACTATCACGTGTTCCGGAGAATCAGCAAGGG |
| | | CTCCGGCGCCACCAACTTTAGCCTGCTGAAGCAGGCAGGCGACGTG |
| | | GAGGAGAATCCAGGACCT |
| 94 | αCD19 scFv | ATGCTGCTGCTGGTGACATCCCTGCTGCTGTGCGAGCTGCCACACC |
| | | CAGCCTTCCTGCTGATCCCCGATATCCAGATGACCCAGACCACAAG |
| | | CTCCCTGAGCGCCTCCCTGGGCGACAGGGTGACAATCTCTTGTCGG |
| | | GCCAGCCAGGATATCTCCAAGTATCTGAATTGGTACCAGCAGAAGC |
| | | CCGACGGCACCGTGAAGCTGCTGATCTATCACACATCTAGACTGCA |
| | | CAGCGGCGTGCCTTCCAGGTTTTCTGGCAGCGGCTCCGGCACCGAC |
| | | TACTCTCTGACAATCAGCAACCTGGAGCAGGAGGATATCGCCACCT |
| | | ATTTCTGCCAGCAGGGCAATACCCTGCCTTACACATTTGGCGGCGG |
| | | CACAAAGCTGGAGATCACCGGCTCTACAAGCGGATCCGGCAAGCC |
| | | AGGATCCGGAGAGGGATCTACCAAGGGAGAGGTGAAGCTGCAGGA |
| | | GAGCGGACCTGGACTGGTGGCACCATCTCAGAGCCTGTCCGTGACC |
| | | TGTACAGTGTCTGGCGTGAGCCTGCCAGATTATGGCGTGAGCTGGA |
| | | TCAGGCAGCCACCTAGGAAGGGACTGGAGTGGCTGGGCGTGATCT |
| | | GGGGCTCCGAGACCACATACTATAACAGCGCCCTGAAGTCCCGCCT |
| | | GACCATCATCAAGGACAACTCTAAGAGCCAGGTGTTCCTGAAGATG |
| | | AATTCCCTGCAGACCGACGATACAGCCATCTACTATTGCGCCAAGC |
| | | ACTACTATTACGGCGGCTCTTATGCCATGGATTACTGGGGCCAGGG |
| | | CACCAGCGTGACAGTGTCTAGC |
| 95 | IgG4 linker-CD28 | GAGTCCAAGTACGGCCCACCCTGCCCTCCATGTCCCATGTTTTGGGT |
| | TM_4-1BB- | GCTGGTGGTGGTGGGAGGCGTGCTGGCCTGTTATTCCCTGCTGGTG |
| | CD3zeta_P2A | ACCGTGGCCTTCATCATCTTTTGGGTGAAGCGCGGCCGGAAGAAGC |
| | | TGCTGTACATCTTCAAGCAGCCCTTCATGAGACCCGTGCAGACCAC |
| | | ACAGGAGGAGGACGGCTGCAGCTGTAGGTTCCCAGAGGAGGAGGA |
| | | GGGAGGATGCGAGCTGAGGGTGAAGTTTTCCCGGTCTGCCGATGCC |
| | | CCTGCCTATCAGCAGGGCCAGAATCAGCTGTACAACGAGCTGAATC |
| | | TGGGCAGGCGCGAGGAGTACGACGTGCTGGATAAGAGGAGAGGAA |
| | | GGGACCCTGAGATGGGAGGCAAGCCAAGGCGCAAGAACCCTCAGG |
| | | AGGGCCTGTATAATGAGCTGCAGAAGGACAAGATGGCCGAGGCCT |
| | | ACTCCGAGATCGGCATGAAGGGAGAGCGGAGAAGGGGCAAGGGAC |
| | | ACGATGGCCTGTATCAGGGCCTGAGCACCGCCACAAAGGACACCTA |
| | | CGATGCACTGCACATGCAGGCCCTGCCACCTAGAGGATCTGGAGCC |
| | | ACAAACTTCAGCCTGCTGAAGCAGGCCGGCGATGTGGAGGAGAAT |
| | | CCTGGACCA |
| 96 | TGF beta DN | ATGGGAAGAGGACTGCTGAGGGGACTGTGGCCACTGCACATCGTG |
| | | CTGTGGACCAGGATCGCCTCTACAATCCCACCCCACGTGCAGAAGA |
| | | GCGTGAACAATGACATGATCGTGACCGATAACAATGGCGCCGTGA |
| | | AGTTTCCCCAGCTGTGCAAGTTCTGTGACGTGCGCTTTTCCACCTGT |
| | | GATAACCAGAAGTCCTGCATGTCTAATTGTAGCATCACATCCATCT |
| | | GCGAGAAGCCTCAGGAGGTGTGCGTGGCCGTGTGGCGGAAGAACG |
| | | ACGAGAATATCACCCTGGAGACAGTGTGCCACGATCCCAAGCTGCC |
| | | TTATCACGACTTCATCCTGGAGGATGCCGCCTCTCCTAAGTGTATCA |
| | | TGAAGGAGAAGAAGAAGCCAGGCGAGACCTTCTTTATGTGCAGCT |
| | | GTTCCTCTGACGAGTGCAACGATAATATCATCTTCTCCGAGGAGTA |
| | | CAACACCTCTAATCCTGACCTGCTGCTGGTCATCTTTCAGGTGACAG |
| | | GCATCTCCCTGCTGCCTCCACTGGGCGTGGCCATCTCTGTGATCATC |
| | | ATCTTTTATTGTTACAGAGTGAACAGGCAGCAGAAGCGCCGGCGCT |
| | | AG |
| 97 | human Glycophorin | SGHFSEPEITLIIFGVMAGVIGTILLISYGIRRLIKKSPSDVKPLPSPDTDV |
| | A TM_ CT | PLSSVEIENPETSDQ |
| | | undcrlincd scqucncc dcnctcs thc transmcmbranc dcmain fragmcnt |
| 98 | linker_human | TCCGGACACTTCAGCGAGCCTGAGATCACCCTGATCATCTTCGGCG |
| | Glycophorin A ecto- | TGATGGCCGGAGTGATCGGCACAATCCTGCTGATCAGCTACGGCAT |
| | TM_HIV Env CT | CAGAAGACTGATTAAGAAATCCCCATCTGATGTGAAGCCTCTGCCT |
| | | TCTCCTGACACCGACGTCCCCCTGAGCAGCGTGGAAATCGAGAACC |
| | | CCGAAACCAGCGACCAG |
| 99 | Gag | MGARASVLSGGELDRWEKIRLRPGGKKKYKLKHIVWASRELERFAVN |
| | | PGLLETSEGCRQILGQLQPSLQTGSEELRSLYNTVATLYCVHQRIEIKDT |
| | | KEALDKIEEEQNKSKKKAQQAAADTGHSNQVSQNYPIVQNIQGQMVH |
| | | QAISPRTLNAWVKVVEEKAFSPEVIPMFSALSEGATPQDLNTMLNTVG |
| | | GHQAAMQMLKETINEEAAEWDRVHPVHAGPIAPGQMREPRGSDIAGT |
| | | TSTLQEQIGWMTHNPPIPVGEIYKRWIILGLNKIVRMYSPTSILDIRQGP |
| | | KEPFRDYVDRFYKTLRAEQASQEVKNWMTETLLVQNANPDCKTILKA |
| | | LGPGATLEEMMTACQGVGGPGHKARVLAEAMSQVTNPATIMIQKGN |
| | | FRNQRKTVKCFNCGKEGHIAKNCRAPRKKGCWKCGKEGHQMKDCTE |
| | | RQANFLGKIWPSHKGRPGNFLQSRPEPTAPPEESFRFGEETTTPSQKQEP |
| | | IDKELYPLASLRSLFGSDPSSQ |
| 100 | Pol | FFREDLAFPQGKAREFSSEQTRANSPTRRELQVWGRDNNSLSEAGADR |
| | | QGTVSFSFPQITLWQRPLVTIKIGGQLKEALLDTGADDTVLEEMNLPGR |
| | | WKPKMIGGIGGFIKVRQYDQILIEICGHKAIGTVLVGPTPVNIIGRNLLT |
| | | QIGCTLNFPISPIETVPVKLKPGMDGPKVKQWPLTEEKIKALVEICTEME |
| | | KEGKISKIGPENPYNTPVFAIKKKDSTKWRKLVDFRELNKRTQDFWEV |
| | | QLGIPHPAGLKQKKSVTVLDVGDAYFSVPLDKDFRKYTAFTIPSINNET |
| | | PGIRYQYNVLPQGWKGSPAIFQCSMTKILEPFRKQNPDIVIYQYMDDL |
| | | YVGSDLEIGQHRTKIEELRQHLLRWGFTTPDKKHQKEPPFLWMGYEL |
| | | HPDKWTVQPIVLPEKDSWTVNDIQKLVGKLNWASQIYAGIKVRQLCK |
| | | LLRGTKALTEVVPLTEEAELELAENREILKEPVHGVYYDPSKDLIAEIQ |
| | | KQGQGQWTYQIYQEPFKNLKTGKYARMKGAHTNDVKQLTEAVQKIA |
| | | TESIVIWGKTPKFKLPIQKETWEAWWTEYWQATWIPEWEFVNTPPLV |
| | | KLWYQLEKEPIIGAETFYVDGAANRETKLGKAGYVTDRGRQKVVPLT |
| | | DTTNQKTELQAIHLALQDSGLEVNIVTDSQYALGIIQAQPDKSESELVS |
| | | QIIEQLIKKEKVYLAWVPAHKGIGGNEQVDKLVSAGIRKVLFLDGIDK |
| | | AQEEHEKYHSNWRAMASDFNLPPVVAKEIVASCDKCQLKGEAMHGQ |
| | | VDCSPGIWQLDCTHLEGKVILVAVHVASGYIEAEVIPAETGQETAYFLL |
| | | KLAGRWPVKTVHTDNGSNFTSTTVKAACWWAGIKQEFGIPYNPQSQG |
| | | VIESMNKELKKIIGQVRDQAEHLKTAVQMAVFIHNFKRKGGIGGYSAG |
| | | ERIVDIIATDIQTKELQKQITKIQNFRVYYRDSRDPVWKGPAKLLWKGE |
| | | GAVVIQDNSDIKVVPRRKAKIIRDYGKQMAGDDCVASRQDED |
| 101 | Gag-pol | atgggcgcccgcgccagcgtgctttctggcggcgagctggacaggtgggagaagattcgcctgcggcctgga |
| | | ggaaagaaaaagtacaagctgaagcacatcgtgtgggcttctcgggaactggaaagattcgccgtgaaccctg |
| | | gactgctagagacctccgaaggctgcagacagatcctgggacagctgcaacctagcctgcagaccggcagcg |
| | | aggagctgagaagcctgtacaacaccgtcgccaccctgtattgtgtgcaccaaagaatcgagatcaaggacac |
| | | caaggaggctctggataagatcgaggaagagcagaacaagagcaagaaaaaagcccagcaggccgccgct |
| | | gataccggccattctaatcaggtgtcccagaactaccccattgtgcaaaatatccagggccagatggtccacca |
| | | ggccatcagccctagaaccctgaatgcctgggtgaaggtggtggaagagaaggccttttctccagaggtgatcc |
| | | ctatgttcagcgccctgagcgagggcgctacccctcaggacctgaacacaatgctgaacaccgtgggcggcca |
| | | ccaggccgccatgcagatgctgaaagaaaccatcaacgaggaggccgccgaatgggaccgggtgcacccc |
| | | gttcacgccggcccaatcgcccctggccagatgcgggaacctagaggcagcgacatcgccggcacaaccag |
| | | cacactgcaagagcagatcggatggatgacacacaacccccccatccccgtgggcgaaatctacaagcggtg |
| | | gatcattctgggactgaacaaaatcgttagaatgtacagccctaccagcatcctggatatcagacagggcccaa |
| | | aggagcctttccgggactacgtggacagattttacaagaccctgagagccgaacaggcctcccaagaggtgaag |
| | | aactggatgacagagaccctgctggtgcagaatgccaaccctgattgtaagacaatcctgaaggccctcggac |
| | | ctggcgctacactggaagaaatgatgaccgcctgccagggcgtgggcggccccggccacaaggccagagtg |
| | | ctggccgaggctatgagccaggtgacaaaccccgccacaatcatgatccagaagggcaacttcagaaaccag |
| | | cggaagaccgtgaaatgcttcaactgcggcaaggaaggccacatcgcaaagaactgcagagcccctaggaa |
| | | gaaaggctgttggaagtgcggaaaggaaggacaccaaatgaaagattgtactgagagacaggctaattttttag |
| | | ggaagatctggccttcccacaagggaaggccagggaattttcttcagagcagaccagagccaacagccccac |
| | | cagaagagagcttcaggtttggggaagagacaacaactccctctcagaagcaggagccgatagacaaggaac |
| | | tgtatcctttagcttccctcagatcactctttggcagcgacccctcgtcacaataaagatcggcggacagctga |
| | | aagaggccttgctggacaccggagccgatgacaccgtgctggaagaaatgaacctgcctggaagatggaaacct |
| | | aagatgatcggtggcatcggcggatttatcaaagtgcgacagtatgaccagatcctgatcgagatttgcggcca |
| | | caaagctatcggaacagtgctggtcggcccgacccccgtgaacatcattggccgcaacctgctgacacagatcg |
| | | gttgtacactgaactttcctatcagccctatcgaaaccgtgccggtcaagctgaagcccggcatggatggccct |
| | | aaggtgaagcagtggcccctgacagaggaaaagatcaaggcactggtggaaatctgcacagaaatggaaaaa |
| | | gagggcaagatttctaaaatcggcccagagaacccctacaacacccctgttttcgccatcaagaagaaagattc |
| | | caccaagtggaggaagctggtggactttcgggaactgaacaagcggacccaggatttctgggaggtgcagct |
| | | gggcatcccccaccctgccggcctgaaacaaaaaaaaagcgtgaccgtgctggacgtgggcgacgcctatttc |
| | | agcgtgcctctggataaggacttccggaaatacaccgcctttaccatccctagcatcaacaacgagacccctgg |
| | | catccggtaccagtacaacgtgctcccacagggctggaagggctcacccgccatcttccagtgcagcatgacc |
| | | aagatcctggagcctttcagaaagcagaatcctgacatcgtgatctaccagtacatggacgacctgtacgtggg |
| | | ctctgatctggagatcggacagcacagaacaaagatcgaagagctgagacagcatctgctgagatggggtttca |
| | | ccacccccgacaagaagcaccagaaggaacctccttttctgtggatgggctacgagctgcaccccgataagtg |
| | | gacagtgcagcccatcgtgctgcccgagaaggactcctggaccgtgaacgacattcagaagctggtcggaaa |
| | | gctgaattgggcttcccaaatctacgccggcatcaaggtgcggcagctgtgcaagcttctgcgcggcacaaag |
| | | gccctgacggaagtcgtgccactgaccgaggaagccgaattagagctggccgaaaacagagaaattctgaaa |
| | | gaacctgtgcacggcgtttactacgacccttctaaggacctgatcgccgaaatccagaaacaaggccagggcc |
| | | agtggacttaccaaatctaccaggagcctttcaaaaacctcaagaccggcaagtacgccagaatgaagggagc |
| | | ccatacaaacgacgtgaagcagctgacagaggctgttcagaagatcgccacagaaagcatcgtgatctgggg |
| | | caagaccccaaagttcaagctgcctatccaaaaggaaacctgggaggcctggtggaccgagtactggcaggc |
| | | cacctggattcctgaatgggagttcgtgaacacaccacctcttgtgaagctgtggtaccagctggaaaaggagc |
| | | caatcatcggcgccgagacattctacgtggacggcgccgccaaccgggagaccaaactgggaaaggccgga |
| | | tatgtgaccgacagaggcagacaaaaggtggtgcctctgaccgatacaactaaccagaaaacagagctgcag |
| | | gccattcacctggccctgcaagacagcggcctggaagtgaatatcgtgacagacagtcagtacgccctgggca |
| | | tcatccaggctcagcctgacaagagcgagagcgagctggtgtcccagatcatcgagcagctgatcaaaaagg |
| | | agaaggtttatctggcctgggtgcccgcccacaagggcatcggaggcaacgagcaggtggacaagctggtca |
| | | gcgccggcatccggaaggtgctgttcctggacggcatcgacaaggcccaggaggaacacgagaagtaccac |
| | | agcaactggcgggccatggccagcgacttcaacctgccacctgtggttgctaaggagatcgtcgcctcttgtga |
| | | taagtgccagctgaagggcgaggccatgcacggccaagtggattgcagccctgggatctggcagctagactgt |
| | | acccacctggagggcaaggtgatcctggtggcagtgcacgtggccagcggctacatcgaggctgaggtgatc |
| | | cccgccgaaacgggccaggagaccgcctactttctgctgaagctagccggccggtggcctgtgaagaccgtg |
| | | cacaccgataacggcagcaatttcaccagcacaaccgtgaaggctgcctgctggtgggctggaatcaagcag |
| | | gagttcggcatcccatacaatcctcagtctcagggcgtgatcgagagcatgaacaaggaactgaagaagatcat |
| | | tggtcaggtcagagatcaggccgagcacctgaaaaccgccgttcaaatggctgtgttcatccataacttcaaaa |
| | | gaaaaggcggcatcggcggctacagcgccggcgaaagaatcgtggatatcatcgcgaccgacatccaaacaa |
| | | aagagctgcaaaagcaaatcaccaagatccagaacttcagagtgtactacagagatagcagagatcctgtgtgg |
| | | aagggacctgccaagctgctgtggaagggcgagggcgccgtggtgatccaggacaatagcgacatcaaggt |
| | | cgtgcccagaagaaaggctaaaatcattagagactacggcaaacagatggccggagatgattgcgtggcttcta |
| | | gacaggacgaggactga |
| 102 | Rev | MAGRSGDSDEDLLKAVRLIKFLYQSNPPPNPEGTRQARRNRRRRWRE |
| | | RQRQIHSISERILSTYLGRSAEPVPLQLPPLERLTLDCNEDCGTSGTQGV |
| | | GSPQILVESPTILESGAKE* |
| 103 | Rev | ATGGCCGGCAGAAGCGGCGACAGCGACGAGGATCTGCTGAAAGCC |
| | | GTGCGGCTGATCAAGTTCCTGTACCAGAGCAACCCTCCTCCTAACC |
| | | CCGAGGGCACCAGACAGGCTAGACGGAACCGCAGAAGAAGGTGGC |
| | | GGGAACGGCAAAGACAGATCCACTCTATCAGCGAGAGAATCCTGA |
| | | GCACCTACCTGGGAAGATCCGCCGAGCCTGTCCCCCTGCAGCTGCC |
| | | TCCACTGGAAAGACTGACCCTGGATTGTAATGAGGACTGCGGCACA |
| | | AGCGGAACCCAGGGCGTGGGCAGCCCCCAGATTCTGGTGGAATCCC |
| | | CTACAATCCTCGAGTCTGGCGCCAAGGAATGA |
| 104 | Cocal envelope | ATGAACTTTCTGCTGCTGACCTTCATCGTGCTGCCTCTGTGCAGCCA |
| | nucleotide sequence | CGCCAAGTTTTCCATCGTGTTCCCACAGTCCCAGAAGGGCAACTGG |
| | | AAGAATGTGCCCAGCTCCTACCACTATTGTCCTTCTAGCTCCGACCA |
| | | GAACTGGCACAATGATCTGCTGGGCATCACCATGAAGGTGAAGATG |
| | | CCTAAGACACACAAGGCCATCCAGGCAGATGGATGGATGTGCCAC |
| | | GCAGCCAAGTGGATCACCACATGTGACTTTCGGTGGTACGGCCCCA |
| | | AGTATATCACCCACAGCATCCACTCCATCCAGCCTACAAGCGAGCA |
| | | GTGCAAGGAGTCCATCAAGCAGACCAAGCAGGGCACATGGATGTC |
| | | TCCCGGCTTCCCCCCTCAGAACTGTGGCTACGCCACCGTGACAGAT |
| | | AGCGTGGCAGTGGTGGTGCAGGCAACCCCACACCACGTGCTGGTGG |
| | | ATGAGTATACAGGCGAGTGGATCGACAGCCAGTTTCCCAACGGCAA |
| | | GTGCGAGACCGAGGAGTGTGAGACAGTGCACAATTCTACCGTGTGG |
| | | TACAGCGATTATAAGGTGACCGGCCTGTGCGACGCCACACTGGTGG |
| | | ATACCGAGATCACATTCTTTTCCGAGGACGGCAAGAAGGAGTCTAT |
| | | CGGCAAGCCCAACACCGGCTACAGGTCTAATTACTTCGCCTATGAG |
| | | AAGGGCGATAAGGTGTGCAAGATGAATTATTGTAAGCACGCCGGG |
| | | GTGCGGCTGCCAAGCGGCGTGTGGTTTGAGTTCGTGGACCAGGACG |
| | | TGTACGCAGCAGCAAAGCTGCCAGAGTGCCCAGTGGGAGCAACCA |
| | | TCAGCGCCCCCACCCAGACATCTGTGGACGTGAGCCTGATCCTGGA |
| | | TGTGGAGAGAATCCTGGACTACTCCCTGTGCCAGGAGACATGGTCC |
| | | AAGATCCGCTCTAAGCAGCCCGTGAGCCCAGTGGACCTGTCTTACC |
| | | TGGCACCAAAGAACCCTGGAACAGGACCTGCCTTTACCATCATCAA |
| | | TGGCACACTGAAGTACTTCGAGACCCGGTATATCAGAATCGACATC |
| | | GATAACCCAATCATCTCCAAGATGGTGGGCAAGATCTCCGGCTCTC |
| | | AGACCGAGAGAGAGCTGTGGACAGAGTGGTTCCCATACGAGGGCG |
| | | TGGAGATCGGCCCCAATGGCATCCTGAAGACCCCTACAGGCTATAA |
| | | GTTTCCACTGTTCATGATCGGCCACGGCATGCTGGACTCTGATCTGC |
| | | ACAAGACCAGCCAGGCCGAGGTGTTTGAGCACCCACACCTGGCAG |
| | | AGGCACCAAAGCAGCTGCCCGAGGAGGAGACCCTGTTCTTTGGCGA |
| | | TACAGGCATCTCCAAGAACCCTGTGGAGCTGATCGAGGGCTGGTTT |
| | | TCTAGCTGGAAGTCTACCGTGGTGACATTCTTTTTCGCCATCGGCGT |
| | | GTTCATCCTGCTGTACGTGGTGGCAAGGATCGTGATCGCCGTGCGG |
| | | TACAGATATCAGGGCAGCAACAATAAGAGAATCTATAATGACATC |
| | | GAGATGTCCAGGTTCCGCAAGTGA |
| 105 | Glycophorin A TM | HFSEPEITLIIFGVMAGVIGTILLISYGIRRLIKKSPSDVKPLPSPDTDVPLS |
| | amino acid sequence | SVEIENPETSDQ |
| 106 | human Glycophorin | CACTTCAGCGAGCCTGAGATCACCCTGATCATCTTCGGCGTGATGG |
| | A hinge-TM-CT | CCGGAGTGATCGGCACAATCCTGCTGATCAGCTACGGCATCAGAAG |
| | | ACTGATTAAGAAATCCCCATCTGATGTGAAGCCTCTGCCTTCTCCTG |
| | | ACACCGACGTCCCCCTGAGCAGCGTGGAAATCGAGAACCCCGAAA |
| | | CCAGCGACCAG |
| 107 | αCD3scFv_short | MGVKVLFALICIAVAEADIQMTQSPSSLSASVGDRVTITCSASSSVSYM |
| | hinge-TM-CT | NWYQQTPGKAPKRWIYDTSKLASGVPSRFSGSGSGTDYTFTISSLQPED |
| | | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASGVELIEGWFSSWKSTVVTFFFAIGVF |
| | | ILLYVVARIVIAVRYRYQGSNNKRIYNDIEMSRFRK |
| 108 | αCD3scFv_short | ATGGGCGTGAAAGTGCTGTTCGCCCTGATCTGCATCGCAGTTGCTG |
| | hinge-TM-CT | AAGCCGACATCCAGATGACCCAGTCTCCTAGCAGCCTCAGCGCTAG |
| | | CGTGGGCGATAGAGTGACCATCACATGTAGCGCCAGCAGCAGCGT |
| | | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGAGTGGAACTGATCGAGGGCTGGTTCAGCAGCTGGAA |
| | | AAGCACCGTGGTTACATTCTTTTTCGCCATCGGCGTGTTCATCCTGC |
| | | TGTACGTGGTCGCCAGAATTGTGATCGCCGTGCGGTATAGATACCA |
| | | GGGCAGCAACAACAAGCGGATCTACAACGACATCGAGATGAGCAG |
| | | ATTCAGAAAG |
| 109 | αCD3scFv_long | MGVKVLFALICIAVAEADIQMTQSPSSLSASVGDRVTITCSASSSVSYM |
| | hinge_TM_CT | NWYQQTPGKAPKRWIYDTSKLASGVPSRFSGSGSGTDYTFTISSLQPED |
| | | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASSGFEHPHLAEAPKQLPEEETLFFGDT |
| | | GISKNPVELIEGWFSSWKSTVVTFFFAIGVFILLYVVARIVIAVRYRYQG |
| | | SNNKRIYNDIEMSRFRK |
| 110 | αCD3scFv_long | ATGGGCGTGAAAGTGCTGTTCGCCCTGATCTGCATCGCAGTTGCTG |
| | hinge_TM_CT | AAGCCGACATCCAGATGACCCAGTCTCCTAGCAGCCTCAGCGCTAG |
| | | CGTGGGCGATAGAGTGACCATCACATGTAGCGCCAGCAGCAGCGT |
| | | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGATTCGAGCACCCCCACCTGGCCGAGGCCCCTAAGCA |
| | | GCTGCCTGAAGAAGAGACACTGTTTTTCGGAGATACCGGCATCAGC |
| | | AAAAACCCCGTGGAGCTGATCGAGGGCTGGTTCAGCTCTTGGAAGA |
| | | GCACCGTGGTCACATTCTTTTTCGCCATCGGCGTCTTTATCCTGCTG |
| | | TACGTGGTAGCCAGAATCGTGATCGCCGTGCGGTACAGATACCAGG |
| | | GCAGCAACAACAAGCGGATCTACAACGACATCGAGATGAGCCGGT |
| | | TCAGAAAG |
| 111 | αCD3scFv | MGVKVLFALICIAVAEADIQMTQSPSSLSASVGDRVTITCSASSSVSYM |
| | hGlycophorinA_TM_ | NWYQQTPGKAPKRWIYDTSKLASGVPSRFSGSGSGTDYTFTISSLQPED |
| | HIV Env CT | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASGGSTSGSGKPGSGEGSTKGPEITLIIF |
| | | GVMAGVIGTILLISYGIRRLALKYWWNLLQYWSQELKNSAVSLLNAT |
| | | AIAVAEGTDRVIEVVQGACRAIRHIPRRIRQGLERILL |
| 112 | αCD3scFv | ATGGGCGTGAAAGTGCTGTTCGCCCTGATCTGCATCGCAGTTGCTG |
| | hGlycophorinA_TM_ | AAGCCGACATCCAGATGACCCAGTCTCCTAGCAGCCTCAGCGCTAG |
| | HIV Env CT | CGTGGGCGATAGAGTGACCATCACATGTAGCGCCAGCAGCAGCGT |
| | | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGAGGATCTACAAGCGGCTCTGGCAAGCCTGGCAGCGG |
| | | AGAAGGCAGCACCAAGGGCCCTGAGATCACACTGATCATCTTCGGC |
| | | GTGATGGCCGGCGTCATCGGCACCATCCTGCTGATCAGCTACGGCA |
| | | TCAGAAGACTGGCTCTGAAGTACTGGTGGAATCTGCTGCAATACTG |
| | | GAGCCAGGAGCTGAAAAACAGCGCCGTGTCCCTGCTCAACGCCACC |
| | | GCCATCGCCGTGGCCGAGGGCACCGACAGAGTGATCGAGGTGGTG |
| | | CAGGGAGCCTGCAGAGCTATTCGGCACATCCCCAGACGGATCAGGC |
| | | AGGGCCTGGAAAGAATCCTGCTG |
| 113 | αCD3scFv_218 | MGVKVLFALICIAVAEADIQMTQSPSSLSASVGDRVTITCSASSSVSYM |
| | linker_HIV Env | NWYQQTPGKAPKRWIYDTSKLASGVPSRFSGSGSGTDYTFTISSLQPED |
| | ecto-TM-CT | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASGGSTSGSGKPGSGEGSTKGNWLWYI |
| | | RIFIIIVGSLIGLRIVFAVLSLVNRGWEALKYWWNLLQYWSQELKNSAV |
| | | SLLNATAIAVAEGTDRVIEVVQGACRAIRHIPRRIRQGLERILL |
| 114 | αCD3scFv_218 | ATGGGCGTGAAAGTGCTGTTCGCCCTGATCTGCATCGCAGTTGCTG |
| | linker_HIV Env | AAGCCGACATCCAGATGACCCAGTCTCCTAGCAGCCTCAGCGCTAG |
| | ecto-TM-CT | CGTGGGCGATAGAGTGACCATCACATGTAGCGCCAGCAGCAGCGT |
| | | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGAGGAAGCACCAGCGGCTCTGGCAAGCCTGGCAGCG |
| | | GCGAGGGCTCTACCAAGGGCAATTGGCTGTGGTACATCAGAATCTT |
| | | CATCATCATCGTGGGCAGCCTGATCGGCCTGAGAATCGTGTTCGCC |
| | | GTGCTGAGCCTGGTGAACCGGGGCTGGGAAGCTCTGAAGTACTGGT |
| | | GGAACCTGCTGCAATACTGGTCCCAGGAGCTGAAAAACAGCGCTGT |
| | | GTCCCTGCTCAACGCCACCGCCATCGCCGTCGCCGAGGGAACAGAC |
| | | AGAGTGATCGAGGTGGTGCAGGGAGCCTGCAGAGCCATTCGGCAC |
| | | ATCCCCAGACGCATCAGACAGGGCCTGGAAAGAATCCTGCTG |
| 115 | αCD3scFv_G4S | MGVKVLFALICIAVAEADIQMTQSPSSLSASVGDRVTITCSASSSVSYM |
| | linker_HIV Env | NWYQQTPGKAPKRWIYDTSKLASGVPSRFSGSGSGTDYTFTISSLQPED |
| | ecto-TM-CT | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASGGGGGSGGGGSGGGGSYIRIFIIIVGS |
| | | LIGLRIVFAVLSLVNRGWEALKYWWNLLQYWSQELKNSAVSLLNATA |
| | | IAVAEGTDRVIEVVQGACRAIRHIPRRIRQGLERILL |
| 116 | αCD3scFv_G4S | ATGGGCGTGAAAGTGCTGTTCGCCCTGATCTGCATCGCAGTTGCTG |
| | linker_HIV Env | AAGCCGACATCCAGATGACCCAGTCTCCTAGCAGCCTCAGCGCTAG |
| | ecto-TM-CT | CGTGGGCGATAGAGTGACCATCACATGTAGCGCCAGCAGCAGCGT |
| | | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGAGGCGGTGGAGGCTCTGGTGGCGGAGGGAGCGGTG |
| | | GCGGAGGCAGCTACATCAGAATCTTCATCATCATCGTGGGCAGCCT |
| | | GATCGGCCTGAGAATCGTGTTCGCCGTTCTGAGCCTGGTGAACCGG |
| | | GGCTGGGAAGCCCTGAAGTACTGGTGGAATCTGCTCCAGTACTGGT |
| | | CTCAGGAGCTGAAGAACAGCGCCGTGTCCCTGCTGAACGCTACAGC |
| | | TATCGCCGTCGCCGAGGGCACCGACAGAGTGATCGAGGTGGTGCA |
| | | GGGCGCCTGCAGAGCCATCCGGCACATCCCTAGAAGGATTCGGCAA |
| | | GGCCTGGAAAGAATCCTGCTG |
| 117 | anti-CD3scFv_short | MGVKVLFALICIAVAEADIQMTQSPSSLSASVGDRVTITCSASSSVSYM |
| | hinge_TM_CT_ | NWYQQTPGKAPKRWIYDTSKLASGVPSRFSGSGSGTDYTFTISSLQPED |
| | T2A_Cocal | IATYYCQQWSSNPFTFGQGTKLQITRTSGGGGSGGGGSGGGGSQVQLV |
| | envelope | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASGVELIEGWFSSWKSTVVTFFFAIGVF |
| | | ILLYVVARIVIAVRYRYQGSNNKRIYNDIEMSRFRKGSGEGRGSLLTCG |
| | | DVEENPGPNFLLLTFIVLPLCSHAKFSIVFPQSQKGNWKNVPSSYHYCP |
| | | SSSDQNWHNDLLGITMKVKMPKTHKAIQADGWMCHAAKWITTCDFR |
| | | WYGPKYITHSIHSIQPTSEQCKESIKQTKQGTWMSPGFPPQNCGYATVT |
| | | DSVAVVVQATPHHVLVDEYTGEWIDSQFPNGKCETEECETVHNSTVW |
| | | YSDYKVTGLCDATLVDTEITFFSEDGKKESIGKPNTGYRSNYFAYEKG |
| | | DKVCKMNYCKHAGVRLPSGVWFEFVDQDVYAAAKLPECPVGATISA |
| | | PTQTSVDVSLILDVERILDYSLCQETWSKIRSKQPVSPVDLSYLAPKNP |
| | | GTGPAFTIINGTLKYFETRYIRIDIDNPIISKMVGKISGSQTERELWTEWF |
| | | PYEGVEIGPNGILKTPTGYKFPLFMIGHGMLDSDLHKTSQAEVFEHPHL |
| | | AEAPKQLPEEETLFFGDTGISKNPVELIEGWFSSWKSTVVTFFFAIGVFI |
| | | LLYVVARIVIAVRYRYQGSNNKRIYNDIEMSRFRK |
| 118 | anti-CD3scFv_short | ATGGGCGTGAAAGTGCTGTTCGCCCTGATCTGCATCGCAGTTGCTG |
| | hinge_TM_CT_ | AAGCCGACATCCAGATGACCCAGTCTCCTAGCAGCCTCAGCGCTAG |
| | T2A_Cocal | CGTGGGCGATAGAGTGACCATCACATGTAGCGCCAGCAGCAGCGT |
| | envelope | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCGGAGTGGAACTGATCGAGGGCTGGTTCAGCAGCTGGAA |
| | | AAGCACCGTGGTTACATTCTTTTTCGCCATCGGCGTGTTCATCCTGC |
| | | TGTACGTGGTCGCCAGAATTGTGATCGCCGTGCGGTATAGATACCA |
| | | GGGCAGCAACAACAAGCGGATCTACAACGACATCGAGATGAGCAG |
| | | ATTCAGAAAGGGATCTGGAGAGGGAAGGGGAAGCCTGCTGACATG |
| | | CGGCGACGTGGAGGAGAACCCAGGACCAAATTTTCTGCTGCTGACC |
| | | TTCATCGTGCTGCCTCTGTGCAGCCACGCCAAGTTTTCCATCGTGTT |
| | | CCCACAGTCCCAGAAGGGCAACTGGAAGAATGTGCCCTCTAGCTAC |
| | | CACTATTGCCCTTCCTCTAGCGACCAGAACTGGCACAATGATCTGCT |
| | | GGGCATCACAATGAAGGTGAAGATGCCCAAGACCCACAAGGCCAT |
| | | CCAGGCAGATGGATGGATGTGCCACGCAGCCAAGTGGATCACAAC |
| | | CTGTGACTTTCGGTGGTACGGCCCCAAGTATATCACACACTCCATCC |
| | | ACTCTATCCAGCCTACCTCCGAGCAGTGCAAGGAGTCTATCAAGCA |
| | | GACAAAGCAGGGCACCTGGATGAGCCCTGGCTTCCCACCCCAGAAC |
| | | TGTGGCTACGCCACAGTGACCGACTCCGTGGCAGTGGTGGTGCAGG |
| | | CAACACCTCACCACGTGCTGGTGGATGAGTATACCGGCGAGTGGAT |
| | | CGACAGCCAGTTTCCAAACGGCAAGTGCGAGACAGAGGAGTGTGA |
| | | GACCGTGCACAATTCTACAGTGTGGTACAGCGATTATAAGGTGACA |
| | | GGCCTGTGCGACGCCACCCTGGTGGATACAGAGATCACCTTCTTTT |
| | | CTGAGGACGGCAAGAAGGAGAGCATCGGCAAGCCCAACACCGGCT |
| | | ACAGATCCAATTACTTCGCCTATGAGAAGGGCGATAAGGTGTGCAA |
| | | GATGAATTATTGTAAGCACGCCGGGGTGCGGCTGCCTAGCGGCGTG |
| | | TGGTTTGAGTTCGTGGACCAGGACGTGTACGCAGCAGCAAAGCTGC |
| | | CTGAGTGCCCAGTGGGAGCAACCATCTCCGCCCCAACACAGACCTC |
| | | CGTGGACGTGTCTCTGATCCTGGATGTGGAGCGCATCCTGGACTAC |
| | | AGCCTGTGCCAGGAGACCTGGAGCAAGATCCGGTCCAAGCAGCCC |
| | | GTGTCCCCTGTGGACCTGTCTTACCTGGCACCAAAGAACCCAGGAA |
| | | CCGGACCAGCCTTTACAATCATCAATGGCACCCTGAAGTACTTCGA |
| | | GACCCGCTATATCCGGATCGACATCGATAACCCTATCATCAGCAAG |
| | | ATGGTGGGCAAGATCTCTGGCAGCCAGACAGAGAGAGAGCTGTGG |
| | | ACCGAGTGGTTCCCTTACGAGGGCGTGGAGATCGGCCCAAATGGCA |
| | | TCCTGAAGACACCAACCGGCTATAAGTTTCCCCTGTTCATGATCGG |
| | | CCACGGCATGCTGGACAGCGATCTGCACAAGACCTCCCAGGCCGAG |
| | | GTGTTTGAGCACCCACACCTGGCAGAGGCACCAAAGCAGCTGCCTG |
| | | AGGAGGAGACACTGTTCTTTGGCGATACCGGCATCTCTAAGAACCC |
| | | CGTGGAGCTGATCGAGGGCTGGTTTTCCTCTTGGAAGAGCACAGTG |
| | | GTGACCTTCTTTTTCGCCATCGGCGTGTTCATCCTGCTGTACGTGGT |
| | | GGCCAGAATCGTGATCGCCGTGAGATACAGGTATCAGGGCTCCAAC |
| | | AATAAGAGGATCTATAATGACATCGAGATGTCTCGCTTCCGGAAG |
| 119 | αCD3scFv_human | MGVKVLFALICIAVAEADIQMTQSPSSLSASVGDRVTITCSASSSVSYM |
| | Glycophorin A | NWYQQTPGKAPKRWIYDTSKLASGVPSRFSGSGSGTDYTFTISSLQPED |
| | hinge-TM-CT | IATYYCQQWSSNPFTFGQGTKLQITRTSGGGGSGGGGSGGGGSQVQLV |
| | | QSGGGVVQPGRSLRLSCKASGYTFTRYTMHWVRQAPGKGLEWIGYIN |
| | | PSRGYTNYNQKVKDRFTISRDNSKNTAFLQMDSLRPEDTGVYFCARY |
| | | YDDHYCLDYWGQGTPVTVSSASHFSEPEITLIIFGVMAGVIGTILLISYG |
| | | IRRLIKKSPSDVKPLPSPDTDVPLSSVEIENPETSDQ |
| 120 | αCD3scFv_human | ATGGGCGTGAAAGTGCTGTTCGCCCTGATCTGCATCGCAGTTGCTG |
| | Glycophorin A | AAGCCGACATCCAGATGACCCAGTCTCCTAGCAGCCTCAGCGCTAG |
| | hinge-TM-CT | CGTGGGCGATAGAGTGACCATCACATGTAGCGCCAGCAGCAGCGT |
| | CAR comprising | GTCCTACATGAACTGGTACCAGCAAACACCTGGAAAGGCCCCTAAA |
| | | AGGTGGATCTATGACACATCTAAGCTGGCTTCTGGAGTGCCATCTA |
| | | GATTTTCTGGCAGCGGCTCCGGCACTGATTATACATTCACCATCAGC |
| | | AGCCTGCAGCCCGAGGATATCGCCACCTACTACTGTCAGCAGTGGT |
| | | CCTCTAATCCCTTCACCTTCGGCCAGGGCACCAAGCTGCAGATCAC |
| | | CAGAACCAGCGGCGGGGGAGGAAGCGGCGGGGGAGGATCTGGCG |
| | | GCGGCGGCAGCCAGGTGCAGCTGGTGCAGAGCGGCGGCGGCGTGG |
| | | TGCAACCTGGCAGAAGCCTGAGACTGAGCTGCAAGGCCTCTGGCTA |
| | | CACCTTCACCCGGTACACCATGCATTGGGTGCGGCAGGCCCCTGGC |
| | | AAGGGCCTGGAATGGATTGGATACATCAACCCCAGCAGAGGCTAC |
| | | ACCAACTACAACCAGAAGGTGAAGGACAGATTCACAATTTCTCGGG |
| | | ACAACAGCAAGAATACCGCCTTCCTGCAAATGGACTCCCTGCGCCC |
| | | AGAAGATACCGGCGTGTACTTCTGCGCTAGATATTACGACGACCAC |
| | | TACTGCCTGGACTACTGGGGCCAGGGCACCCCTGTGACCGTGTCCA |
| | | GCGCCTCCCACTTCAGCGAGCCTGAGATCACCCTGATCATCTTCGG |
| | | CGTGATGGCCGGAGTGATCGGCACAATCCTGCTGATCAGCTACGGC |
| | | ATCAGAAGACTGATTAAGAAATCCCCATCTGATGTGAAGCCTCTGC |
| | | CTTCTCCTGACACCGACGTCCCCCTGAGCAGCGTGGAAATCGAGAA |
| | | CCCCGAAACCAGCGACCAG |
| 121 | CAR comprising | tgtagtcttatgcaatactcttgtagtcttgcaacatggtaacgatgagttagcaacatgccttacaaggagag |
| | anti-CD19, | aaaaagcaccgtgcatgccgattggtggaagtaaggtggtacgatcgtgccttattaggaaggcaacagacggg |
| | cytosolic FRB, | tctgacatggattggacgaaccactgaattgccgcattgcagagatattgtatttaagtgcctagctcgataca |
| | RACR | taaacgggtctctctggttagaccagatctgagcctgggagctctctggctaactagggaacccactgcttaag |
| | | cctcaataaagcttgccttgagtgcttcaagtagtgtgtgcccgtctgttgtgtgactctggtaactagagatc |
| | | cctcagacccttttagtcagtgtggaaaatctctagcagtggcgcccgaacagggacttgaaagcgaaagggaa |
| | | accagaggagctctctcgacgcaggactcggcttgctgaagcgcgcacggcaagaggcgaggggcggcgactgg |
| | | tgagtacgccaaaaattttgactagcggaggctagaaggagagagatgggtgcgagagcgtcagtattaagcgg |
| | | gggagaattagatcgcgatgggaaaaaattcggttaaggccagggggaaagaaaaaatataaattaaaacatat |
| | | agtatgggcaagcagggagctagaacgattcgcagttaatcctggcctgttagaaacatcagaaggctgtagac |
| | | aaatactgggacagctacaaccatcccttcagacaggatcagaagaacttagatcattatataatacagtagca |
| | | accctctattgtgtgcatcaaaggatagagataaaagacaccaaggaagctttagacaagatagaggaagagca |
| | | aaacaaaagtaagaccaccgcacagcaagcggccgctgatcttcagacctggaggaggagatatgagggacaat |
| | | tggagaagtgaattatataaatataaagtagtaaaaattgaaccattaggagtagcacccaccaaggcaaagag |
| | | aagagtggtgcagagagaaaaaagagcagtgggaataggagctttgttccttgggttcttgggagcagcaggaa |
| | | gcactatgggcgcagcgtcaatgacgctgacggtacaggccagacaattattgtctggtatagtgcagcagcag |
| | | aacaatttgctgagggctattgaggcgcaacagcatctgttgcaactcacagtctggggcatcaagcagctcca |
| | | ggcaagaatcctggctgtggaaagatacctaaaggatcaacagctcctggggatttggggttgctctggaaaac |
| | | tcatttgcaccactgctgtgccttggaatgctagttggagtaataaatctctggaacagatttggaatcacacg |
| | | acctggatggagtgggacagagaaattaacaattacacaagcttaatacactccttaattgaagaatcgcaaaa |
| | | ccagcaagaaaagaatgaacaagaattattggaattagataaatgggcaagtttgtggaattggtttaacataa |
| | | caaattggctgtggtatataaaattattcataatgatagtaggaggcttggtaggtttaagaatagtttttgct |
| | | gtactttctatagtgaatagagttaggcagggatattcaccattatcgtttcagacccacctcccaaccccgag |
| | | gggacccgacaggcccgaaggaatagaagaagaaggtggagagagagacagagacagatccattcgattagtga |
| | | acggatctcgacggtatcggttaacttttaaaagaaaaggggggattggggggtacagtgcaggggaaagaata |
| | | gtagacataatagcaacagacatacaaactaaagaattacaaaaacaaattacaaaaattcaaaattttatcgg |
| | | ccgcggggtacctaggaacagagaaacaggagaatatgggccaaacaggatatctgtggtaagcagttcctgcc |
| | | ccggctcagggccaagaacagttggaacagcagaatatgggccaaacaggatatctgtggtaagcagttcctgc |
| | | cccggctcagggccaagaacagatggtccccagatgcggtcccgccctcagcagtttctagagaaccatcagat |
| | | gtttccagggtgccccaaggacctgaaatgaccctgtgccttatttgaactaaccaatcagttcgcttctcgct |
| | | tctgttcgcgcgcttctgctccccgagctctatataagcagagctcgtttagtgaaccgtcagatcgcctggag |
| | | acgccatccacgctgttttgacttccatagaagcctgcagggccgccaccatgctgctgctggtgacctccctg |
| | | ctgctgtgcgagctgcctcacccagcctttctgctgatccccgacatccagatgacacagaccacaagctccct |
| | | gtctgccagcctgggcgacagagtgaccatctcctgtagggcctctcaggatatcagcaagtacctgaactggt |
| | | atcagcagaagccagatggcacagtgaagctgctgatctaccacacctccaggctgcactctggagtgccaagc |
| | | cggttctccggatctggaagcggcaccgactattccctgacaatctctaacctggagcaggaggatatcgccac |
| | | atacttttgccagcagggcaataccctgccatatacattcggcggaggaaccaagctggagatcaccggatcca |
| | | catctggaagcggcaagccaggaagcggagagggatccacaaagggagaggtgaagctgcaggagagcggacca |
| | | ggactggtggcaccatcccagtctctgagcgtgacctgtacagtgtccggcgtgtctctgcctgactacggcgt |
| | | gtcctggatcaggcagccacctaggaagggactggagtggctgggcgtgatctggggctctgagaccacatact |
| | | ataattctgccctgaagagccgcctgaccatcatcaaggacaactccaagtctcaggtgtttctgaagatgaat |
| | | agcctgcagaccgacgatacagccatctactattgcgccaagcactactattacggcggctcctacgccatgga |
| | | ttattggggccagggcacctccgtgacagtgtctagcgagtctaagtatggcccaccctgccctccatgtccaa |
| | | tgttctgggtgctggtggtggtgggaggcgtgctggcctgttactccctgctggtgaccgtggcctttatcatc |
| | | ttctgggtgaagagaggcaggaagaagctgctgtatatctttaagcagcccttcatgcgccctgtgcagaccac |
| | | acaggaggaggacggctgcagctgtcggtttccagaggaggaggagggaggatgcgagctgcgcgtgaagttca |
| | | gccggtccgccgatgcccctgcctaccagcagggccagaaccagctgtataacgagctgaatctgggccggaga |
| | | gaggagtacgacgtgctggataagaggaggggaagggacccagagatgggaggcaagcctcggagaaagaaccc |
| | | acaggagggcctgtacaatgagctgcagaaggacaagatggccgaggcctattctgagatcggcatgaagggag |
| | | agaggcgccggggcaagggacacgatggcctgtaccagggcctgagcaccgccacaaaggacacatatgatgcc |
| | | ctgcacatgcaggccctgccacctaggggatctggagccaccaactttagcctgctgaagcaggcaggcgatgt |
| | | ggaggagaatccaggacctgagatgtggcacgagggactggaggaggcaagcaggctgtactttggcgagcg |
| | | gaatgtgaagggcatgttcgaggtgctggagccactgcacgcaatgatggagaggggaccacagaccctgaa |
| | | ggagacatccttcaaccaggcatacggaagggacctgatggaggcacaggagtggtgccggaagtatatgaa |
| | | gtctggcaatgtgaaggacctgctgcaggcctgggatctgtattaccacgtgtttagaaggatcagcaagggct |
| | | ccggcgccaccaacttctccctgctgaagcaggccggcgatgtggaagaaaatccaggaccaatgcctctggg |
| | | actgctgtggctgggactggccctgctgggcgccctgcacgcccaggccggcgtgcaggtggagacaatcag |
| | | ccctggcgacggcagaacctttccaaagaggggccagacatgcgtggtgcactacaccggcatgctggagga |
| | | tggcaagaagttcgactcctctcgcgatcggaacaagccctttaagttcatgctgggcaagcaggaagtgatca |
| | | gaggctgggaggagggcgtggcccagatgtctgtgggccagagggccaagctgacaatcagcccagactat |
| | | gcatacggagcaaccggacaccctggaatcatcccaccacacgccacactggtgttcgatgtggagctgctga |
| | | agctgggcgagggctctaacaccagcaaggagaatccatttctgttcgcactggaggcagtggtcatctccgtg |
| | | ggctctatgggcctgatcatctccctgctgtgcgtgtacttttggctggagagaacaatgccaaggatccccac |
| | | cctgaagaacctggaggacctggtgaccgagtaccacggcaatttcagcgcctggtccggcgtgtctaagggac |
| | | tggcagagtccctgcagccagattattctgagcggctgtgcctggtgagcgagatccctccaaagggaggcgcc |
| | | ctgggagagggaccaggagccagcccctgcaaccagcactccccttactgggcccccccttgttataccctga |
| | | agccagagacaggctctggcgccaccaacttcagcctgctgaagcaagccggcgacgtggaagaaaaccca |
| | | ggaccaatggcactgccagtgaccgccctgctgctgcctctggccctgctgctgcacgcagccagacccatcc |
| | | tgtggcacgaaatgtggcatgaaggcctggaggaggcaagcagactgtactttggcgagagaaatgtgaaag |
| | | gaatgtttgaggtgctggagcctctgcacgccatgatggagaggggccctcagaccctgaaggagacatccttt |
| | | aaccaggcctacggcagagacctgatggaggcccaggagtggtgcaggaagtatatgaagagcggaaatgt |
| | | gaaagacctgctgcaggcctgggatctgtactaccacgtgttccgccggatctctaagggcaaggatacaatcc |
| | | cttggctgggacacctgctggtgggactgagcggagcctttggcttcatcatcctggtgtatctgctgatcaac |
| | | tgcagaaatacaggcccatggctgaagaaggtgctgaagtgtaacacccctgacccatccaagttcttttctca |
| | | gctgagctccgagcacggcggcgatgtgcagaagtggctgtctagcccctttccttcctctagcttcagccctg |
| | | gaggactggcacctgagatctccccactggaggtgctggagagggacaaggtgacccagctgctgctgcagcag |
| | | gataaggtgccagagcccgcctccctgtcctctaaccacagcctgacctcctgctttacaaatcagggctactt |
| | | ctttttccacctgccagacgcactggagatcgaggcatgtcaggtgtatttcacatacgatccctatagcgagg |
| | | aggaccctgatgagggagtggccggcgccccaaccggaagctcccctcagccactgcagccactgagcggagag |
| | | gacgatgcatattgtacatttccttcccgcgacgatctgctgctgttctctccaagcctgctgggaggaccatc |
| | | tccacccagcaccgcacctggaggatccggggcaggggaggagcggatgcctccatctctgcaggagagagtgc |
| | | caagggactgggatccacagcctctgggaccacctacccctggagtgccagacctggtggatttccagccaccc |
| | | cctgagctggtgctgcgggaggcaggagaggaggtgccagacgcaggacctagagagggcgtgagctttcc |
| | | ctggtccaggccaccaggacagggagagttccgcgccctgaacgcccggctgcccctgaatacagacgccta |
| | | cctgtctctgcaggagctgcagggccaggatcctacccacctggtgtgacgccggcgctagtgtcgacaatca |
| | | acctctggattacaaaatttgtgaaagattgactggtattcttaactatgttgctccttttacgctatgtggat |
| | | acgctgctttaatgcctttgtatcatgctattgcttcccgtatggctttcattttctcctccttgtataaatcc |
| | | tggttgctgtctctttatgaggagttgtggcccgttgtcaggcaacgtggcgtggtgtgcactgtgtttgctga |
| | | cgcaacccccactggttggggcattgccaccacctgtcagctcctttccgggactttcgctttccccctcccta |
| | | ttgccacggcggaactcatcgccgcctgccttgcccgctgctggacaggggctcggctgttgggcactgacaat |
| | | tccgtggtgttgtcggggaagctgacgtcctttccatggctgctcgcctgtgttgccacctggattctgcgcgg |
| | | gacgtccttctgctacgtcccttcggccctcaatccagcggaccttccttcccgcggcctgctgccggctctgc |
| | | ggcctcttccgcgtcttcgccttcgccctcagacgagtcggatctccctttgggccgcctccccgcctgtttaa |
| | | gaccaatgacttacaaggcagctgtagatcttagccactttttaaaagaaaaggggggactggaagggctaatt |
| | | cactcccaacgaagacaagatctgctttttgcttgtactgggtctctctggttagaccagatctgagcctggga |
| | | gctctctggctaactagggaacccactgcttaagcctcaataaagcttgccttgagtgcttcaagtagtgtgtg |
| | | cccgtctgttgtgtgactctggtaactagagatccctcagacccttttagtcagtgtggaaaatctctagca |
| 122 | anti-CD19 CAR, | gacattgattattgactagttattaatagtaatcaattacggggtcattagttcatagcccatatatggagttc |
| | cytosolic FRB, | cgcgttacataacttacggtaaatggcccgcctggctgaccgcccaacgacccccgcccattgacgtcaataat |
| | and RACR | gacgtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatggccc |
| | | gcctggcattatgcccagtacatgaccttatgggactttcctacttggcagtacatctacgtattagtcatcgc |
| | | tattaccatggtgatgcggttttggcagtacatcaatgggcgtggatagcggtttgactcacggggatttccaa |
| | | gtctccaccccattgacgtcaatgggagtttgttttggcaccaaaatcaacgggactttccaaaatgtcgtaac |
| | | aactccgccccattgacgcaaatgggcggtaggcgtgtacggtgggaggtctatataagcagagctcgtttagt |
| | | gaaccgggtctctctggttagaccagatctgagcctgggagctctctggctaactagggaacccactgcttaag |
| | | cctcaataaagcttgccttgagtgcttcaagtagtgtgtgcccgtctgttgtgtgactctggtaactagagatc |
| | | cctcagacccttttagtcagtgtggaaaatctctagcagtggcgcccgaacagggacttgaaagcgaaagggaa |
| | | accagaggagctctctcgacgcaggactcggcttgctgaagcgcgcacggcaagaggcgaggggcggcgactgg |
| | | tgagtacgccaaaaattttgactagcggaggctagaaggagagagatgggtgcgagagcgtcagtattaagcgg |
| | | gggagaattagatcgcgatgggaaaaaattcggttaaggccagggggaaagaaaaaatataaattaaaacatat |
| | | agtatgggcaagcagggagctagaacgattcgcagttaatcctggcctgttagaaacatcagaaggctgtagac |
| | | aaatactgggacagctacaaccatcccttcagacaggatcagaagaacttagatcattatataatacagtagca |
| | | accctctattgtgtgcatcaaaggatagagataaaagacaccaaggaagctttagacaagatagaggaagagca |
| | | aaacaaaagtaagaccaccgcacagcaagcggccgctgatcttcagacctggaggaggagatatgagggacaat |
| | | tggagaagtgaattatataaatataaagtagtaaaaattgaaccattaggagtagcacccaccaaggcaaagag |
| | | aagagtggtgcagagagaaaaaagagcagtgggaataggagctttgttccttgggttcttgggagcagcaggaa |
| | | gcactatgggcgcagcgtcaatgacgctgacggtacaggccagacaattattgtctggtatagtgcagcagcag |
| | | aacaatttgctgagggctattgaggcgcaacagcatctgttgcaactcacagtctggggcatcaagcagctcca |
| | | ggcaagaatcctggctgtggaaagatacctaaaggatcaacagctcctggggatttggggttgctctggaaaac |
| | | tcatttgcaccactgctgtgccttggaatgctagttggagtaataaatctctggaacagatttggaatcacacg |
| | | acctggatggagtgggacagagaaattaacaattacacaagcttaatacactccttaattgaagaatcgcaaaa |
| | | ccagcaagaaaagaatgaacaagaattattggaattagataaatgggcaagtttgtggaattggtttaacataa |
| | | caaattggctgtggtatataaaattattcataatgatagtaggaggcttggtaggtttaagaatagtttttgct |
| | | gtactttctatagtgaatagagttaggcagggatattcaccattatcgtttcagacccacctcccaaccccgag |
| | | gggacccgacaggcccgaaggaatagaagaagaaggtggagagagagacagagacagatccattcgattagtga |
| | | acggatctcgacggtatcggttaacttttaaaagaaaaggggggattggggggtacagtgcaggggaaagaata |
| | | gtagacataatagcaacagacatacaaactaaagaattacaaaaacaaattacaaaaattcaaaattttatcga |
| | | ttgcctgacgcgtaatgaaagaccccacctgtaggtttggcaagctaggatcaaggtcaggaacagagagacag |
| | | cagaatatgggccaaacaggatatctgtggtaagcagttcctgccccggctcagggccaagaacagttggaaca |
| | | gcagaatatgggccaaacaggatatctgtggtaagcagttcctgccccggctcagggccaagaacagatggtcc |
| | | ccagatgcggtcccgccctcagcagtttctagagaaccatcagatgtttccagggtgccccaaggacctgaaat |
| | | gaccctgtgccttatttgaactaaccaatcagttcgcttctcgcttctgttcgcgcgcttctgctccccgagct |
| | | ctatataagagcccacaacccctcactcggcgcgtgaacacaattctgcagtcgaaggcgtaccgtcacttacg |
| | | agtcggtagcctgcagggccgccaccatgctgctgctggtgacctccctgctgctgtgcgagctgcctcaccca |
| | | gcctttctgctgatccccgacatccagatgacacagaccacaagctccctgtctgccagcctgggcgacagagt |
| | | gaccatctcctgtagggcctctcaggatatcagcaagtacctgaactggtatcagcagaagccagatggcacag |
| | | tgaagctgctgatctaccacacctccaggctgcactctggagtgccaagccggttctccggatctggaagcggc |
| | | accgactattccctgacaatctctaacctggagcaggaggatatcgccacatacttttgccagcagggcaatac |
| | | cctgccatatacattcggcggaggaaccaagctggagatcaccggatccacatctggaagcggcaagccaggaa |
| | | gcggagagggatccacaaagggagaggtgaagctgcaggagagcggaccaggactggtggcaccatcccagtct |
| | | ctgagcgtgacctgtacagtgtccggcgtgtctctgcctgactacggcgtgtcctggatcaggcagccacctag |
| | | gaagggactggagtggctgggcgtgatctggggctctgagaccacatactataattctgccctgaagagccgcc |
| | | tgaccatcatcaaggacaactccaagtctcaggtgtttctgaagatgaatagcctgcagaccgacgatacagcc |
| | | atctactattgcgccaagcactactattacggcggctcctacgccatggattattggggccagggcacctccgt |
| | | gacagtgtctagcgagtctaagtatggcccaccctgccctccatgtccaatgttctgggtgctggtggtggtgg |
| | | gaggcgtgctggcctgttactccctgctggtgaccgtggcctttatcatcttctgggtgaagagaggcaggaag |
| | | aagctgctgtatatctttaagcagcccttcatgcgccctgtgcagaccacacaggaggaggacggctgcagctg |
| | | tcggtttccagaggaggaggagggaggatgcgagctgcgcgtgaagttcagccggtccgccgatgcccctgcct |
| | | accagcagggccagaaccagctgtataacgagctgaatctgggccggagagaggagtacgacgtgctggataag |
| | | aggaggggaagggacccagagatgggaggcaagcctcggagaaagaacccacaggagggcctgtacaatgagct |
| | | gcagaaggacaagatggccgaggcctattctgagatcggcatgaagggagagaggcgccggggcaagggacacg |
| | | atggcctgtaccagggcctgagcaccgccacaaaggacacatatgatgccctgcacatgcaggccctgccacct |
| | | aggggatctggagccaccaactttagcctgctgaagcaggcaggcgatgtggaggagaatccaggacctgagat |
| | | gtggcacgagggactggaggaggcaagcaggctgtactttggcgagcggaatgtgaagggcatgttcgaggtgc |
| | | tggagccactgcacgcaatgatggagaggggaccacagaccctgaaggagacatccttcaaccaggcatacgga |
| | | agggacctgatggaggcacaggagtggtgccggaagtatatgaagtctggcaatgtgaaggacctgctgcaggc |
| | | ctgggatctgtattaccacgtgtttagaaggatcagcaagggctccggcgccaccaacttctccctgctgaagc |
| | | aggccggcgatgtggaagaaaatccaggaccaatgcctctgggactgctgtggctgggactggccctgctgggc |
| | | gccctgcacgcccaggccggcgtgcaggtggagacaatcagccctggcgacggcagaacctttccaaagagggg |
| | | ccagacatgcgtggtgcactacaccggcatgctggaggatggcaagaagttcgactcctctcgcgatcggaaca |
| | | agccctttaagttcatgctgggcaagcaggaagtgatcagaggctgggaggagggcgtggcccagatgtctgtg |
| | | ggccagagggccaagctgacaatcagcccagactatgcatacggagcaaccggacaccctggaatcatcccacc |
| | | acacgccacactggtgttcgatgtggagctgctgaagctgggcgagggctctaacaccagcaaggagaatccat |
| | | ttctgttcgcactggaggcagtggtcatctccgtgggctctatgggcctgatcatctccctgctgtgcgtgtac |
| | | ttttggctggagagaacaatgccaaggatccccaccctgaagaacctggaggacctggtgaccgagtaccacgg |
| | | caatttcagcgcctggtccggcgtgtctaagggactggcagagtccctgcagccagattattctgagcggctgt |
| | | gcctggtgagcgagatccctccaaagggaggcgccctgggagagggaccaggagccagcccctgcaaccagc |
| | | actccccttactgggcccccccttgttataccctgaagccagagacaggctctggcgccaccaacttcagcctg |
| | | ctgaagcaagccggcgacgtggaagaaaacccaggaccaatggcactgccagtgaccgccctgctgctgcctc |
| | | tggccctgctgctgcacgcagccagacccatcctgtggcacgaaatgtggcatgaaggcctggaggaggcaa |
| | | gcagactgtactttggcgagagaaatgtgaaaggaatgtttgaggtgctggagcctctgcacgccatgatggag |
| | | aggggccctcagaccctgaaggagacatcctttaaccaggcctacggcagagacctgatggaggcccagga |
| | | gtggtgcaggaagtatatgaagagcggaaatgtgaaagacctgctgcaggcctgggatctgtactaccacgtgt |
| | | tccgccggatctctaagggcaaggatacaatcccttggctgggacacctgctggtgggactgagcggagccttt |
| | | ggcttcatcatcctggtgtatctgctgatcaactgcagaaatacaggcccatggctgaagaaggtgctgaagtg |
| | | taacacccctgacccatccaagttcttttctcagctgagctccgagcacggcggcgatgtgcagaagtggctgt |
| | | ctagcccctttccttcctctagcttcagccctggaggactggcacctgagatctccccactggaggtgctggag |
| | | agggacaaggtgacccagctgctgctgcagcaggataaggtgccagagcccgcctccctgtcctctaaccacag |
| | | cctgacctcctgctttacaaatcagggctacttctttttccacctgccagacgcactggagatcgaggcatgtc |
| | | aggtgtatttcacatacgatccctatagcgaggaggaccctgatgagggagtggccggcgccccaaccggaagc |
| | | tcccctcagccactgcagccactgagcggagaggacgatgcatattgtacatttccttcccgcgacgatctgct |
| | | gctgttctctccaagcctgctgggaggaccatctccacccagcaccgcacctggaggatccggggcaggggagg |
| | | agcggatgcctccatctctgcaggagagagtgccaagggactgggatccacagcctctgggaccacctacccct |
| | | ggagtgccagacctggtggatttccagccaccccctgagctggtgctgcgggaggcaggagaggaggtgccaga |
| | | cgcaggacctagagagggcgtgagctttccctggtccaggccaccaggacagggagagttccgcgccctgaa |
| | | cgcccggctgcccctgaatacagacgcctacctgtctctgcaggagctgcagggccaggatcctacccacctg |
| | | gtgtgacgccggcgctagtgtcgacgtagtggtacctttaagaccaatgacttacaaggcagctgtagatctta |
| | | gccactttttaaaagaaaaggggggactggaagggctaattcactcccaacgaagacaagatctgctttttgct |
| | | tgtactgggtctctctggttagaccagatctgagcctgggagctctctggctaactagggaacccactgcttaa |
| | | gcctcaataaagcttgccttgagtgcttcaatgtgtgtgttggttttttgtgtgt |
| 123 | Cocal envelope | ctagttattaatagtaatcaattacggggtcattagttcatagcccatatatggagttccgcgttacataactt |
| | plasmid | acggtaaatggcccgcctggctgaccgcccaacgacccccgcccattgacgtcaataatgacgtatgttcccat |
| | | agtaacgccaatagggactttccattgacgtcaatgggtggagtatttacggtaaactgcccacttggcagtac |
| | | atcaagtgtatcatatgccaagtacgccccctattgacgtcaatgacggtaaatggcccgcctggcattatgcc |
| | | cagtacatgaccttatgggactttcctacttggcagtacatctacgtattagtcatcgctattaccatggtgat |
| | | gcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattg |
| | | acgcaaatgggcggtaggcgtgtacggtgggaggtctatataagcagagctcgtttagtgaaccgtcagatcgc |
| | | ctggagacgccatccacgctgttttgacctccatagaagacaccgggcgagctcggatcctgagaacttcaggg |
| | | tgagtctatgggacccttgatgttttctttccccttcttttctatggttaagttcatgtcataggaaggggaga |
| | | agtaacagggtacacatattgaccaaatcagggtaattttgcatttgtaattttaaaaaatgctttcttctttt |
| | | aatatacttttttgtttatcttatttctaatactttccctaatctctttctttcagggcaataatgatacaatg |
| | | tatcatgcctctttgcaccattctaaagaataacagtgataatttctgggttaaggcaatagcaatatttctgc |
| | | atataaatatttctgcatataaattgtaactgatgtaagaggtttcatattgctaatagcagctacaatccagc |
| | | taccattctgcttttattttatggttgggataaggctggattattctgagtccaagctaggcccttttgctaat |
| | | catgttcatacctcttatcttcctcccacagctcctgggcaacgtgctggtctgtgtgctggcccatcactttg |
| | | gcaaagcacgtgagatctgtctgacatcctgcagggccgccaccatgaactttctgctgctgaccttcatcgtg |
| | | ctgcctctgtgcagccacgccaagttttccatcgtgttcccacagtcccagaagggcaactggaagaatgtgcc |
| | | cagctcctaccactattgtccttctagctccgaccagaactggcacaatgatctgctgggcatcaccatgaagg |
| | | tgaagatgcctaagacacacaaggccatccaggcagatggatggatgtgccacgcagccaagtggatcaccaca |
| | | tgtgactttcggtggtacggccccaagtatatcacccacagcatccactccatccagcctacaagcgagcagtg |
| | | caaggagtccatcaagcagaccaagcagggcacatggatgtctcccggcttcccccctcagaactgtggctacg |
| | | ccaccgtgacagatagcgtggcagtggtggtgcaggcaaccccacaccacgtgctggtggatgagtatacaggc |
| | | gagtggatcgacagccagtttcccaacggcaagtgcgagaccgaggagtgtgagacagtgcacaattctaccgt |
| | | gtggtacagcgattataaggtgaccggcctgtgcgacgccacactggtggataccgagatcacattcttttccg |
| | | aggacggcaagaaggagtctatcggcaagcccaacaccggctacaggtctaattacttcgcctatgagaagggc |
| | | gataaggtgtgcaagatgaattattgtaagcacgccggggtgcggctgccaagcggcgtgtggtttgagttcgt |
| | | aggaccaggacgtgtacgcagcagcaaagctgccagagtgcccagtgggagcaacctcagcgcccccacccaga |
| | | catctgtggacgtgagcctgatcctggatgtggagagaatcctggactactccctgtgccaggagacatggtcc |
| | | aagatccgctctaagcagcccgtgagcccagtggacctgtcttacctggcaccaaagaaccctggaacaggacc |
| | | tgcctttaccatcatcaatggcacactgaagtacttcgagacccggtatatcagaatcgacatcgataacccaa |
| | | tcatctccaagatggtgggcaagatctccggctctcagaccgagagagagctgtggacagagtggttcccatac |
| | | gagggcgtggagatcggccccaatggcatcctgaagacccctacaggctataagtttccactgttcatgatcgg |
| | | ccacggcatgctggactctgatctgcacaagaccagccaggccgaggtgtttgagcacccacacctggcagagg |
| | | caccaaagcagctgcccgaggaggagaccctgttctttggcgatacaggcatctccaagaaccctgtggagctg |
| | | atcgagggctggttttctagctggaagtctaccgtggtgacattctttttcgccatcggcgtgttcatcctgct |
| | | gtacgtggtggcaaggatcgtgatcgccgtgcggtacagatatcagggcagcaacaataagagaatctataatg |
| | | acatcgagatgtccaggttccgcaagtgacgccggcgtcgacgctcaacagcctcgactgtgccttctagttgc |
| | | cagccatctgttgtttgcccctcccccgtgccttccttgaccctggaaggtgccactcccactgtcctttccta |
| | | ataaaatgaggaaattgcatcgcattgtctgagtaggtgtcattctattctggggggtggggtggggcaggaca |
| | | gcaagggggaggattgggaagacaatagcaggcatgctggggatgcggtgggctctatgg |
| 124 | helper plasmid- | gacattgattattgactagttattaatagtaatcaattacggggtcattagttcatagcccatatatggagttc |
| | gag/pol | cgcgttacataacttacggtaaatggcccgcctggctgaccgcccaacgacccccgcccattgacgtcaataat |
| | | gacgtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatggccc |
| | | gcctggcattatgcccagtacatgaccttatgggactttcctacttggcagtacatctacgtattagtcatcgc |
| | | tattaccatggtgatgcggttttggcagtacatcaatgggcgtggatagcggtttgactcacggggatttccaa |
| | | gtctccaccccattgacgtcaatgggagtttgttttggcaccaaaatcaacgggactttccaaaatgtcgtaac |
| | | aactccgccccattgacgcaaatgggggtaggcgtgtacggtgggaggtctatataagcagagctcgtttagtg |
| | | aaccgtcagatcactagaagcttagctgcgaagttggtcgtgaggcactgggcaggtaagtatcaaggttacaa |
| | | gacaggtttaaggagaccaatagaaactgggcttgtcgagacagagaagactcttgcgtttctgataggcacct |
| | | attggtcttactgacatccactttgcctttctctccacaggtgtccactcccagttcaattacagctcttaagg |
| | | ctaggatccgccgccaccatgggcgcccgcgccagcgtgctttctggcggcgagctggacaggtgggagaagat |
| | | tcgcctgcggcctggaggaaagaaaaagtacaagctgaagcacatcgtgtgggcttctcgggaactggaaagat |
| | | tcgccgtgaaccctggactgctagagacctccgaaggctgcagacagatcctgggacagctgcaacctagcctg |
| | | cagaccggcagcgaggagctgagaagcctgtacaacaccgtcgccaccctgtattgtgtgcaccaaagaatcga |
| | | gatcaaggacaccaaggaggctctggataagatcgaggaagagcagaacaagagcaagaaaaaagcccagcagg |
| | | ccgccgctgataccggccattctaatcaggtgtcccagaactaccccattgtgcaaaatatccagggccagatg |
| | | gtccaccaggccatcagccctagaaccctgaatgcctgggtgaaggtggtggaagagaaggccttttctccaga |
| | | ggtgatccctatgttcagcgccctgagcgagggcgctacccctcaggacctgaacacaatgctgaacaccgtgg |
| | | gcggccaccaggccgccatgcagatgctgaaagaaaccatcaacgaggaggccgccgaatgggaccgggtgcac |
| | | cccgttcacgccggcccaatcgcccctggccagatgcgggaacctagaggcagcgacatcgccggcacaaccag |
| | | cacactgcaagagcagatcggatggatgacacacaacccccccatccccgtgggcgaaatctacaagcggtgga |
| | | tcattctgggactgaacaaaatcgttagaatgtacagccctaccagcatcctggatatcagacagggcccaaag |
| | | gagcctttccgggactacgtggacagattttacaagaccctgagagccgaacaggcctcccaagaggtgaagaa |
| | | ctggatgacagagaccctgctggtgcagaatgccaaccctgattgtaagacaatcctgaaggccctcggacctg |
| | | gcgctacactggaagaaatgatgaccgcctgccagggcgtgggcggccccggccacaaggccagagtgctg |
| | | gccgaggctatgagccaggtgacaaaccccgccacaatcatgatccagaagggcaacttcagaaaccagcgg |
| | | aagaccgtgaaatgcttcaactgcggcaaggaaggccacatcgcaaagaactgcagagcccctaggaagaaa |
| | | ggctgttggaagtgcggaaaggaaggacaccaaatgaaagattgtactgagagacaggctaattttttagggaa |
| | | gatctggccttcccacaagggaaggccagggaattttcttcagagcagaccagagccaacagccccaccaga |
| | | agagagcttcaggtttggggaagagacaacaactccctctcagaagcaggagccgatagacaaggaactgtat |
| | | cctttagcttccctcagatcactctttggcagcgacccctcgtcacaataaagatcggcggacagctgaaagag |
| | | gccttgctggacaccggagccgatgacaccgtgctggaagaaatgaacctgcctggaagatggaaacctaagat |
| | | gatcggtggcatcggcggatttatcaaagtgcgacagtatgaccagatcctgatcgagatttgcggccacaaag |
| | | ctatcggaacagtgctggtcggcccgacccccgtgaacatcattggccgcaacctgctgacacagatcggttgt |
| | | acactgaactttcctatcagccctatcgaaaccgtgccggtcaagctgaagcccggcatggatggccctaaggt |
| | | gaagcagtggcccctgacagaggaaaagatcaaggcactggtggaaatctgcacagaaatggaaaaagagg |
| | | gcaagatttctaaaatcggcccagagaacccctacaacacccctgttttcgccatcaagaagaaagattccacc |
| | | aagtggaggaagctggtggactttcgggaactgaacaagcggacccaggatttctgggaggtgcagctgggcat |
| | | cccccaccctgccggcctgaaacaaaaaaaaagcgtgaccgtgctggacgtgggcgacgcctatttcagcgtg |
| | | cctctggataaggacttccggaaatacaccgcctttaccatccctagcatcaacaacgagacccctggcatccg |
| | | gtaccagtacaacgtgctcccacagggctggaagggctcacccgccatcttccagtgcagcatgaccaagatc |
| | | ctggagcctttcagaaagcagaatcctgacatcgtgatctaccagtacatggacgacctgtacgtgggctctga |
| | | tctggagatcggacagcacagaacaaagatcgaagagctgagacagcatctgctgagatggggtttcaccacc |
| | | cccgacaagaagcaccagaaggaacctccttttctgtggatgggctacgagctgcaccccgataagtggacag |
| | | tgcagcccatcgtgctgcccgagaaggactcctggaccgtgaacgacattcagaagctggtcggaaagctgaa |
| | | ttgggcttcccaaatctacgccggcatcaaggtgcggcagctgtgcaagcttctgcgcggcacaaaggccctg |
| | | acggaagtcgtgccactgaccgaggaagccgaattagagctggccgaaaacagagaaattctgaaagaacct |
| | | gtgcacggcgtttactacgacccttctaaggacctgatcgccgaaatccagaaacaaggccagggccagtgga |
| | | cttaccaaatctaccaggagcctttcaaaaacctcaagaccggcaagtacgccagaatgaagggagcccatac |
| | | aaacgacgtgaagcagctgacagaggctgttcagaagatcgccacagaaagcatcgtgatctggggcaagac |
| | | cccaaagttcaagctgcctatccaaaaggaaacctgggaggcctggtggaccgagtactggcaggccacctg |
| | | gattcctgaatgggagttcgtgaacacaccacctcttgtgaagctgtggtaccagctggaaaaggagccaatca |
| | | tcggcgccgagacattctacgtggacggcgccgccaaccgggagaccaaactgggaaaggccggatatgtga |
| | | ccgacagaggcagacaaaaggtggtgcctctgaccgatacaactaaccagaaaacagagctgcaggccattc |
| | | acctggccctgcaagacagcggcctggaagtgaatatcgtgacagacagtcagtacgccctgggcatcatcca |
| | | ggctcagcctgacaagagcgagagcgagctggtgtcccagatcatcgagcagctgatcaaaaaggagaaggt |
| | | ttatctggcctgggtgcccgcccacaagggcatcggaggcaacgagcaggtggacaagctggtcagcgccg |
| | | gcatccggaaggtgctgttcctggacggcatcgacaaggcccaggaggaacacgagaagtaccacagcaact |
| | | ggcgggccatggccagcgacttcaacctgccacctgtggttgctaaggagatcgtcgcctcttgtgataagtgc |
| | | cagctgaagggcgaggccatgcacggccaagtggattgcagccctgggatctggcagctagactgtacccac |
| | | ctggagggcaaggtgatcctggtggcagtgcacgtggccagcggctacatcgaggctgaggtgatccccgcc |
| | | gaaacgggccaggagaccgcctactttctgctgaagctagccggccggtggcctgtgaagaccgtgcacacc |
| | | gataacggcagcaatttcaccagcacaaccgtgaaggctgcctgctggtgggctggaatcaagcaggagttcg |
| | | gcatcccatacaatcctcagtctcagggcgtgatcgagagcatgaacaaggaactgaagaagatcattggtcag |
| | | gtcagagatcaggccgagcacctgaaaaccgccgttcaaatggctgtgttcatccataacttcaaaagaaaagg |
| | | cggcatcggcggctacagcgccggcgaaagaatcgtggatatcatcgcgaccgacatccaaacaaaagagct |
| | | gcaaaagcaaatcaccaagatccagaacttcagagtgtactacagagatagcagagatcctgtgtggaaggga |
| | | cctgccaagctgctgtggaagggcgagggcgccgtggtgatccaggacaatagcgacatcaaggtcgtgccc |
| | | agaagaaaggctaaaatcattagagactacggcaaacagatggccggagatgattgcgtggcttctagacagg |
| | | acgaggactgataagaattccatgtcgacgctcaacagcctcgactgtgccttctagttgccagccatctgttg |
| | | tttgcccctcccccgtgccttccttgaccctggaaggtgccactcccactgtcctttcctaataaaatgaggaa |
| | | attgcatcgcattgtctgagtaggtgtcattctattctggggggtggggggggcaggacagcaagggggaggat |
| | | tgggaagacaatagcaggcatgctggggatgcggtgggctctatgg |
| 125 | helper plasmid- | gacattgattattgactagttattaatagtaatcaattacggggtcattagttcatagcccatatatggagttc |
| | Rev | cgcgttacataacttacggtaaatggcccgcctggctgaccgcccaacgacccccgcccattgacgtcaataat |
| | | gacgtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatggccc |
| | | gcctggcattatgcccagtacatgaccttatgggactttcctacttggcagtacatctacgtattagtcatcgc |
| | | tattaccatggtgatgcggttttggcagtacatcaatgggcgtggatagcggtttgactcacggggatttccaa |
| | | gtctccaccccattgacgtcaatgggagtttgttttggcaccaaaatcaacgggactttccaaaatgtcgtaac |
| | | aactccgccccattgacgcaaatgggcggtaggcgtgtacggtgggaggtctatataagcagagctcgtttagt |
| | | gaaccgtcagatcgcctggagacgccatccacgctgttttgacctccatagaagacaccgggccaccatggccg |
| | | gcagaagcggcgacagcgacgaggatctgctgaaagccgtgcggctgatcaagttcctgtaccagagcaaccct |
| | | cctcctaaccccgagggcaccagacaggctagacggaaccgcagaagaaggtgggggaacggcaaagacagatc |
| | | cactctatcagcgagagaatcctgagcacctacctgggaagatccgccgagcctgtccccctgcagctgcctcc |
| | | actggaaagactgaccctggattgtaatgaggactgcggcacaagcggaacccagggcgtgggcagcccccaga |
| | | ttctggtggaatcccctacaatcctcgagtctggcgccaaggaatgataactagcacctgctttatttgtgaaa |
| | | tttgtgatgctattgctttatttgtaaccattataagctgcaataaacaagttaacaacaacaattgcattcat |
| | | tttatgtttcaggttcagggggaggtgtgggaggttttttaa |
| 126 | anti-CD3 plasmid | ctagttattaatagtaatcaattacggggtcattagttcatagcccatatatggagttccgcgttacataactt |
| | | acggtaaatggcccgcctggctgaccgcccaacgacccccgcccattgacgtcaataatgacgtatgttcccat |
| | | agtaacgccaatagggactttccattgacgtcaatgggtggagtatttacggtaaactgcccacttggcagtac |
| | | atcaagtgtatcatatgccaagtacgccccctattgacgtcaatgacggtaaatggcccgcctggcattatgcc |
| | | cagtacatgaccttatgggactttcctacttggcagtacatctacgtattagtcatcgctattaccatggtgat |
| | | gcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattg |
| | | acgcaaatgggcggtaggcgtgtacggtgggaggtctatataagcagagctcgtttagtgaaccgtcagatcgc |
| | | ctggagacgccatccacgctgttttgacctccatagaagacaccgggcgagctcggatcctgagaacttcaggg |
| | | tgagtctatgggacccttgatgttttctttccccttcttttctatggttaagttcatgtcataggaaggggaga |
| | | agtaacagggtacacatattgaccaaatcagggtaattttgcatttgtaattttaaaaaatgctttcttctttt |
| | | aatatacttttttgtttatcttatttctaatactttccctaatctctttctttcagggcaataatgatacaatg |
| | | tatcatgcctctttgcaccattctaaagaataacagtgataatttctgggttaaggcaatagcaatatttctgc |
| | | atataaatatttctgcatataaattgtaactgatgtaagaggtttcatattgctaatagcagctacaatccag |
| | | ctaccattctgcttttattttatggttgggataaggctggattattctgagtccaagctaggcccttttgctaa |
| | | tcatgttcatacctcttatcttcctcccacagctcctgggcaacgtgctggtctgtgtgctggcccatcacttt |
| | | ggcaaagcacgtgagatctgccaccatgggcgtgaaagtgctgttcgccctgatctgcatcgcagttgctgaag |
| | | ccgacatccagatgacccagtctcctagcagcctcagcgctagcgtgggcgatagagtgaccatcacatgtagc |
| | | gccagcagcagcgtgtcctacatgaactggtaccagcaaacacctggaaaggcccctaaaaggtggatctatga |
| | | cacatctaagctggcttctggagtgccatctagattttctggcagcggctccggcactgattatacattcacca |
| | | tcagcagcctgcagcccgaggatatcgccacctactactgtcagcagtggtcctctaatcccttcaccttcggc |
| | | cagggcaccaagctgcagatcaccagaaccagcggcgggggaggaagcggcgggggaggatctggcggcggcgg |
| | | cagccaggtgcagctggtgcagagcggcggcggcgtggtgcaacctggcagaagcctgagactgagctgcaagg |
| | | cctctggctacaccttcacccggtacaccatgcattgggtgcggcaggcccctggcaagggcctggaatggatt |
| | | ggatacatcaaccccagcagaggctacaccaactacaaccagaaggtgaaggacagattcacaatttctcggga |
| | | caacagcaagaataccgccttcctgcaaatggactccctgcgcccagaagataccggcgtgtacttctgcgcta |
| | | gatattacgacgaccactactgcctggactactggggccagggcacccctgtgaccgtgtccagcgcctccgga |
| | | gtggaactgatcgagggctggttcagcagctggaaaagcaccgtggttacattctttttcgccatcggcgtgtt |
| | | catcctgctgtacgtggtcgccagaattgtgatcgccgtgcggtatagataccagggcagcaacaacaagcgga |
| | | tctacaacgacatcgagatgagcagattcagaaagtaatgactcgaggtgaattcgcgccggcgtcgacgctca |
| | | acagcctcgactgtgccttctagttgccagccatctgttgtttgcccctcccccgtgccttccttgaccctgga |
| | | aggtgccactcccactgtcctttcctaataaaatgaggaaattgcatcgcattgtctgagtaggtgtcattcta |
| | | ttctggggggtggggggggcaggacagcaagggggaggattgggaagacaatagcaggcatgctggggatgcgg |
| | | tgggctctatgg |
| 127 | anti-CD3 plasmid | ctagttattaatagtaatcaattacggggtcattagttcatagcccatatatggagttccgcgttacataactt |
| | | acggtaaatggcccgcctggctgaccgcccaacgacccccgcccattgacgtcaataatgacgtatgttcccat |
| | | agtaacgccaatagggactttccattgacgtcaatgggtggagtatttacggtaaactgcccacttggcagtac |
| | | atcaagtgtatcatatgccaagtacgccccctattgacgtcaatgacggtaaatggcccgcctggcattatgcc |
| | | cagtacatgaccttatgggactttcctacttggcagtacatctacgtattagtcatcgctattaccatggtgat |
| | | gcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattg |
| | | acgcaaatgggcggtaggcgtgtacggtgggaggtctatataagcagagctcgtttagtgaaccgtcagatcgc |
| | | ctggagacgccatccacgctgttttgacctccatagaagacaccgggcgagctcggatcctgagaacttcaggg |
| | | tgagtctatgggacccttgatgttttctttccccttcttttctatggttaagttcatgtcataggaaggggaga |
| | | agtaacagggtacacatattgaccaaatcagggtaattttgcatttgtaattttaaaaaatgctttcttctttt |
| | | aatatacttttttgtttatcttatttctaatactttccctaatctctttctttcagggcaataatgatacaatg |
| | | tatcatgcctctttgcaccattctaaagaataacagtgataatttctgggttaaggcaatagcaatatttctgc |
| | | atataaatatttctgcatataaattgtaactgatgtaagaggtttcatattgctaatagcagctacaatccagc |
| | | taccattctgcttttattttatggttgggataaggctggattattctgagtccaagctaggcccttttgctaat |
| | | catgttcatacctcttatcttcctcccacagctcctgggcaacgtgctggtctgtgtgctggcccatcactttg |
| | | gcaaagcacgtgagatctgccaccatgggcgtgaaagtgctgttcgccctgatctgcatcgcagttgctgaagc |
| | | cgacatccagatgacccagtctcctagcagcctcagcgctagcgtgggcgatagagtgaccatcacatgtagcg |
| | | ccagcagcagcgtgtcctacatgaactggtaccagcaaacacctggaaaggcccctaaaaggtggatctatgac |
| | | acatctaagctggcttctggagtgccatctagattttctggcagcggctccggcactgattatacattcaccat |
| | | cagcagcctgcagcccgaggatatcgccacctactactgtcagcagtggtcctctaatcccttcaccttcggcc |
| | | agggcaccaagctgcagatcaccagaaccagcggcgggggaggaagcggcgggggaggatctggcggcggcggc |
| | | agccaggtgcagctggtgcagagcggcggcggcgtggtgcaacctggcagaagcctgagactgagctgcaaggc |
| | | ctctggctacaccttcacccggtacaccatgcattgggtgcggcaggcccctggcaagggcctggaatggattg |
| | | gatacatcaaccccagcagaggctacaccaactacaaccagaaggtgaaggacagattcacaatttctcgggac |
| | | aacagcaagaataccgccttcctgcaaatggactccctgcgcccagaagataccggcgtgtacttctgcgctag |
| | | atattacgacgaccactactgcctggactactggggccagggcacccctgtgaccgtgtccagcgcctccggac |
| | | acttcagcgagcctgagatcaccctgatcatcttcggcgtgatggccggagtgatcggcacaatcctgctgatc |
| | | agctacggcatcagaagactgattaagaaatccccatctgatgtgaagcctctgccttctcctgacaccgacgt |
| | | ccccctgagcagcgtggaaatcgagaaccccgaaaccagcgaccagtgataagaattctagtcgacgctcaaca |
| | | gcctcgactgtgccttctagttgccagccatctgttgtttgcccctcccccgtgccttccttgaccctggaagg |
| | | tgccactcccactgtcctttcctaataaaatgaggaaattgcatcgcattgtctgagtaggtgtcattctattc |
| | | tggggggtggggggggcaggacagcaagggggaggattgggaagacaatagcaggcatgctggggatgcggtgg |
| | | gctctatgg |
| 128 | KanR_p57M-MND- | gaacagagaaacaggagaatatgggccaaacaggatatctgtggtaagcagttcctgccccggctcagggcc |
| | (anti)medCD3-Cocal- | aagaacagttggaacagcagaatatgggccaaacaggatatctgtggtaagcagttcctgccccggctcaggg |
| | wPREw-BGHpA-001 | ccaagaacagatggtccccagatgcggtcccgccctcagcagtttctagagaaccatcagatgtttccagggtg |
| | | ccccaaggacctgaaatgaccctgtgccttatttgaactaaccaatcagttcgcttctcgcttctgttcgcgcg |
| | | cttctgctccccgagctctatataagcagagctcgtttagtgaaccgtcagatcgcctggagacgccatccacg |
| | | ctgttttgacttccatagaagcctgcagggccgccaccatggcactgcctgtgacagccctgctgctgccactg |
| | | gccctgctgctgcacgcagcacgcccagatatccagatgacccagtccccaagctccctgagcgcctccgtggg |
| | | cgaccgggtgacaatcacctgcagcgcctctagctccgtgtcctacatgaactggtatcagcagacacctggca |
| | | aggccccaaagagatggatctacgataccagcaagctggcctccggcgtgccttctaggttttctggcagcggc |
| | | tccggcacagattatacattcaccatctctagcctgcagccagaggacatcgccacctactattgccagcagtg |
| | | gtcctctaatccctttacattcggccagggcaccaagctgcagatcacaagaacctctggaggaggaggaagcg |
| | | gaggaggaggatccggcggcggcggctctcaggtgcagctggtgcagagcggaggaggagtggtgcagccaggc |
| | | agaagcctgaggctgtcctgtaaggcctctggctacacattcaccagatatacaatgcactgggtgaggcaggc |
| | | accaggcaagggactggagtggatcggctacatcaacccctccaggggctacaccaactataatcagaaggtg |
| | | aaggatcggttcaccatcagcagggacaactccaagaataccgccttcctgcagatggacagcctgaggccag |
| | | aggataccggcgtgtacttttgcgcccggtactatgacgatcactactgtctggattattggggccagggaaca |
| | | ccagtgaccgtgagctccgccgcagcaaagcctaccacaacccctgccccaaggccacctacacccgccccta |
| | | ccatcgcctctcagccactgagcctgaggccagaggcatccaggcctgccgcagggggggccgtgcacacc |
| | | cggggcctggactttgcctctgatatggcactgatcgtgctgggaggagtggcaggactgctgctgttcatcgg |
| | | actgggcatcttcttttgcgtgcgctgtaggcaccggagaaggcagggatctggagagggaaggggaagcctg |
| | | ctgacatgcggcgacgtggaggagaacccaggaccaaattttctgctgctgaccttcatcgtgctgcctctgtg |
| | | cagccacgccaagttttccatcgtgttcccacagtcccagaagggcaactggaagaatgtgccctctagctacc |
| | | actattgcccttcctctagcgaccagaactggcacaatgatctgctgggcatcacaatgaaggtgaagatgccc |
| | | aagacccacaaggccatccaggcagatggatggatgtgccacgcagccaagtggatcacaacctgtgactttcg |
| | | gtggtacggccccaagtatatcacacactccatccactctatccagcctacctccgagcagtgcaaggagtcta |
| | | tcaagcagacaaagcagggcacctggatgagccctggcttcccaccccagaactgtggctacgccacagtgacc |
| | | gactccgtggcagtggtggtgcaggcaacacctcaccacgtgctggtggatgagtataccggcgagtggatcga |
| | | cagccagtttccaaacggcaagtgcgagacagaggagtgtgagaccgtgcacaattctacagtgtggtacagc |
| | | gattataaggtgacaggcctgtgcgacgccaccctggtggatacagagatcaccttcttttctgaggacggcaa |
| | | gaaggagagcatcggcaagcccaacaccggctacagatccaattacttcgcctatgagaagggcgataaggtgt |
| | | gcaagatgaattattgtaagcacgccggggtgcggctgcctagcggcgtgtggtttgagttcgtggaccaggac |
| | | gtgtacgcagcagcaaagctgcctgagtgcccagtgggagcaaccatctccgccccaacacagacctccgtg |
| | | gacgtgtctctgatcctggatgtggagcgcatcctggactacagcctgtgccaggagacctggagcaagatccg |
| | | gtccaagcagcccgtgtcccctgtggacctgtcttacctggcaccaaagaacccaggaaccggaccagccttta |
| | | caatcatcaatggcaccctgaagtacttcgagacccgctatatccggatcgacatcgataaccctatcatcagc |
| | | aagatggtgggcaagatctctggcagccagacagagagagagctgtggaccgagtggttcccttacgagggcg |
| | | tggagatcggcccaaatggcatcctgaagacaccaaccggctataagtttcccctgttcatgatcggccacggc |
| | | atgctggacagcgatctgcacaagacctcccaggccgaggtgtttgagcacccacacctggcagaggcacca |
| | | aagcagctgcctgaggaggagacactgttctttggcgataccggcatctctaagaaccccgtggagctgatcga |
| | | gggctggttttcctcttggaagagcacagtggtgaccttctttttcgccatcggcgtgttcatcctgctgtacg |
| | | tggtggccagaatcgtgatcgccgtgagatacaggtatcagggctccaacaataagaggatctataatgacatc |
| | | gagatgtctcgcttccggaagtgacgccggcgtcgacaatcaacctctggattacaaaatttgtgaaagattga |
| | | ctggtattcttaactatgttgctccttttacgctatgtggatacgctgctttaatgcctttgtatcatgctatt |
| | | gcttcccgtatggctttcattttctcctccttgtataaatcctggttgctgtctctttatgaggagttgtggcc |
| | | cgttgtcaggcaacgtggcgtggtgtgcactgtgtttgctgacgcaacccccactggttggggcattgccacca |
| | | cctgtcagctcctttccgggactttcgctttccccctccctattgccacggcggaactcatcgccgcctgcctt |
| | | gcccgctgctggacaggggctcggctgttgggcactgacaattccgtggtgttgtcggggaagctgacgtcctt |
| | | tccatggctgctcgcctgtgttgccacctggattctgcgcgggacgtccttctgctacgtcccttcggccctca |
| | | atccagcggaccttccttcccgcggcctgctgccggctctgcggcctcttccgcgtcttcgccttcgccctcag |
| | | acgagtcggatctccctttgggccgcctccccgcctgtgtgccttctagttgccagccatctgttgtttgcccc |
| | | tcccccgtgccttccttgaccctggaaggtgccactcccactgtcctttcctaataaaatgaggaaattgcatc |
| | | gcattgtctgagtaggtgtcattctattctggggggtggggtggggcaggacagcaagggggaggattgggaag |
| | | acaatagcaggcatgctggggatgcggtgggctctatgg |
| 129 | p57M-CMV-BGi- | gacattgattattgactagttattaatagtaatcaattacggggtcattagttcatagcccatatatggagttc |
| | (anti)-med-hCD3- | cgcgttacataacttacggtaaatggcccgcctggctgaccgcccaacgacccccgcccattgacgtcaataat |
| | Cocal-BG-polyA | gacgtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatggccc |
| | | tgcctggcattagcccagtacatgaccttatgggactttcctacttggcagtacatctacgtattagtcatcgc |
| | | tattaccatggtgatgcggttttggcagtacatcaatgggcgtggatagcggtttgactcacggggatttccaa |
| | | gtctccaccccattgacgtcaatgggagtttgttttggcaccaaaatcaacgggactttccaaaatgtcgtaac |
| | | aactccgccccattgacgcaaatgggcggtaggcgtgtacggtgggaggtctatataagcagagctcgtttagt |
| | | gaaccgtcagatcgcctggagacgccatccacgctgttttgacctccatagaagacaccgggaccgatccagcc |
| | | tcccctcgaagcttacatgtggtaccgagctcggatcctgagaacttcagggtgagtctatgggacccttgatg |
| | | ttttctttccccttcttttctatggttaagttcatgtcataggaaggggagaagtaacagggtacacatattga |
| | | ccaaatcagggtaattttgcatttgtaattttaaaaaatgctttcttcttttaatatacttttttgtttatctt |
| | | atttctaatactttccctaatctctttctttcagggcaataatgatacaatgtatcatgcctctttgcaccatt |
| | | ctaaagaataacagtgataatttctgggttaaggcaatagcaatatttctgcatataaatatttctgcatataa |
| | | attgtaactgatgtaagaggtttcatattgctaatagcagctacaatccagctaccattctgcttttattttat |
| | | ggttgggataaggctggattattctgagtccaagctaggcccttttgctaatcatgttcatacctcttatcttc |
| | | ctcccacagctcctgggcaacgtgctggtctgtgtgctggcccatcactttggcaaagcacgtgagatctgaat |
| | | tctgacactcctgcagggccgccaccatggcactgcctgtgacagccctgctgctgccactggccctgctgctg |
| | | cacgcagcacgcccagatatccagatgacccagtccccaagctccctgagcgcctccgtgggcgaccgggtgac |
| | | aatcacctgcagcgcctctagctccgtgtcctacatgaactggtatcagcagacacctggcaa |
| | | ggccccaaagagatggatctacgataccagcaagctggcctccggcgtgccttctaggttttctggcagcggct |
| | | ccggcacagattatacattcaccatctctagcctgcagccagaggacatcgccacctactattgccagcagtgg |
| | | tcctctaatccctttacattcggccagggcaccaagctgcagatcacaagaacctctggaggaggaggaagcgg |
| | | aggaggaggatccggcggcggcggctctcaggtgcagctggtgcagagcggaggaggagtggtgcagcca |
| | | ggcagaagcctgaggctgtcctgtaaggcctctggctacacattcaccagatatacaatgcactgggtgaggca |
| | | ggcaccaggcaagggactggagtggatcggctacatcaacccctccaggggctacaccaactataatcagaa |
| | | ggtgaaggatcggttcaccatcagcagggacaactccaagaataccgccttcctgcagatggacagcctgagg |
| | | ccagaggataccggcgtgtacttttgcgcccggtactatgacgatcactactgtctggattattggggccaggg |
| | | aacaccagtgaccgtgagctccgccgcagcaaagcctaccacaacccctgccccaaggccacctacacccgcc |
| | | cctaccatcgcctctcagccactgagcctgaggccagaggcatccaggcctgccgcagggggggccgtgca |
| | | cacccggggcctggactttgcctctgatatggcactgatcgtgctgggaggagtggcaggactgctgctgttca |
| | | tcggactgggcatcttcttttgcgtgcgctgtaggcaccggagaaggcagggatctggagagggaaggggaag |
| | | cctgctgacatgcggcgacgtggaggagaacccaggaccaaattttctgctgctgaccttcatcgtgctgcctc |
| | | tgtgcagccacgccaagttttccatcgtgttcccacagtcccagaagggcaactggaagaatgtgccctctagc |
| | | taccactattgcccttcctctagcgaccagaactggcacaatgatctgctgggcatcacaatgaaggtgaagat |
| | | gcccaagacccacaaggccatccaggcagatggatggatgtgccacgcagccaagtggatcacaacctgtgact |
| | | ttcggtggtacggccccaagtatatcacacactccatccactctatccagcctacctccgagcagtgcaaggag |
| | | tctatcaagcagacaaagcagggcacctggatgagccctggcttcccaccccagaactgtggctacgccacagt |
| | | gaccgactccgtggcagtggtggtgcaggcaacacctcaccacgtgctggtggatgagtataccggcgagtgga |
| | | tcgacagccagtttccaaacggcaagtgcgagacagaggagtgtgagaccgtgcacaattctacagtgtggta |
| | | cagcgattataaggtgacaggcctgtgcgacgccaccctggtggatacagagatcaccttcttttctgaggacg |
| | | gcaagaaggagagcatcggcaagcccaacaccggctacagatccaattacttcgcctatgagaagggcgataa |
| | | ggtgtgcaagatgaattattgtaagcacgccggggtgcggctgcctagcggcgtgtggtttgagttcgtggac |
| | | caggacgtgtacgcagcagcaaagctgcctgagtgcccagtgggagcaaccatctccgccccaacacagacct |
| | | ccgtggacgtgtctctgatcctggatgtggagcgcatcctggactacagcctgtgccaggagacctggagcaa |
| | | gatccggtccaagcagcccgtgtcccctgtggacctgtcttacctggcaccaaagaacccaggaaccggacca |
| | | gcctttacaatcatcaatggcaccctgaagtacttcgagacccgctatatccggatcgacatcgataaccctat |
| | | catcagcaagatggtgggcaagatctctggcagccagacagagagagagctgtggaccgagtggttcccttacg |
| | | agggcgtggagatcggcccaaatggcatcctgaagacaccaaccggctataagtttcccctgttcatgatcggc |
| | | cacggcatgctggacagcgatctgcacaagacctcccaggccgaggtgtttgagcacccacacctggcagagg |
| | | caccaaagcagctgcctgaggaggagacactgttctttggcgataccggcatctctaagaaccccgtggagctg |
| | | atcgagggctggttttcctcttggaagagcacagtggtgaccttctttttcgccatcggcgtgttcatcctgct |
| | | gtacgtggtggccagaatcgtgatcgccgtgagatacaggtatcagggctccaacaataagaggatctataatg |
| | | acatcgagatgtctcgcttccggaagtgacgccggcgctcaaatcctgcacaacagattcttcatgtttggacc |
| | | aaatcaacttgtgataccatgctcaaagaggcctcaattatatttgagtttttaatttttatgaaaaaaaaaaa |
| | | aaaaaacggaattcaccccaccagtgcaggctgcctatcagaaagtggtggctggtgtggctaatgccctggcc |
| | | cacaagtatcactaagctcgctttcttgctgtccaatttctattaaaggttcctttgttccctaagtccaacta |
| | | ctaaactgggggatattatgaagggccttgagcatctggattctgcctaataaaaaacatttattttcattgca |
| | | atgatgtatttaaattatttctgaatattttactaaaaagggaatgtgggaggtcagtgcatttaaaacataaa |
| | | gaaatgaagagctagttcaaaccttgggaaaatacactatatcttaaactccatgaaagaaggtgaggctgcaa |
| | | acagctaatgcacattggcaacagcccctgatgcctatgccttattcatccctcagaaaaggattcaagtagag |
| | | gcttgatttggaggttaaagttttgctaatgctgtatttta |
| 130 | Cocal envelope | gaacagagaaacaggagaatatgggccaaacaggatatctgtggtaagcagttcctgccccggctcagggcc |
| | plasmid | aagaacagttggaacagcagaatatgggccaaacaggatatctgtggtaagcagttcctgccccggctcaggg |
| | | ccaagaacagatggtccccagatgcggtcccgccctcagcagtttctagagaaccatcagatgtttccagggtg |
| | | ccccaaggacctgaaatgaccctgtgccttatttgaactaaccaatcagttcgcttctcgcttctgttcgcgcg |
| | | cttctgctccccgagctctatataagcagagctcgtttagtgaaccgtcagatcgcctggagacgccatccacg |
| | | ctgttttgacttccatagaagcctgcagggccgccaccatgaactttctgctgctgaccttcatcgtgctgcct |
| | | ctgtgcagccacgccaagttttccatcgtgttcccacagtcccagaagggcaactggaagaatgtgcccagctc |
| | | ctaccactattgtccttctagctccgaccagaactggcacaatgatctgctgggcatcaccatgaaggtgaaga |
| | | tgcctaagacacacaaggccatccaggcagatggatggatgtgccacgcagccaagtggatcaccacatgtgac |
| | | tttcggtggtacggccccaagtatatcacccacagcatccactccatccagcctacaagcgagcagtgcaagga |
| | | gtccatcaagcagaccaagcagggcacatggatgtctcccggcttcccccctcagaactgtggctacgccaccg |
| | | tgacagatagcgtggcagtggtggtgcaggcaaccccacaccacgtgctggtggatgagtatacaggcgagtgg |
| | | atcgacagccagtttcccaacggcaagtgcgagaccgaggagtgtgagacagtgcacaattctaccgtgtggta |
| | | cagcgattataaggtgaccggcctgtgcgacgccacactggtggataccgagatcacattcttttccgaggacg |
| | | gcaagaaggagtctatcggcaagcccaacaccggctacaggtctaattacttcgcctatgagaagggcgataag |
| | | gtgtgcaagatgaattattgtaagcacgccggggtgcggctgccaagcggcgtgtggtttgagttcgtggacca |
| | | ggacgtgtacgcagcagcaaagctgccagagtgcccagtgggagcaaccatcagcgcccccacccagacatctg |
| | | tggacgtgagcctgatcctggatgtggagagaatcctggactactccctgtgccaggagacatggtccaagatc |
| | | cgctctaagcagcccgtgagcccagtggacctgtcttacctggcaccaaagaaccctggaacaggacctgcctt |
| | | taccatcatcaatggcacactgaagtacttcgagacccggtatatcagaatcgacatcgataacccaatcatct |
| | | ccaagatggtgggcaagatctccggctctcagaccgagagagagctgtggacagagtggttcccatacgagggc |
| | | gtggagatcggccccaatggcatcctgaagacccctacaggctataagtttccactgttcatgatcggccacgg |
| | | catgctggactctgatctgcacaagaccagccaggccgaggtgtttgagcacccacacctggcagaggcaccaa |
| | | agcagctgcccgaggaggagaccctgttctttggcgatacaggcatctccaagaaccctgtggagctgatcgag |
| | | ggctggttttctagctggaagtctaccgtggtgacattctttttcgccatcggcgtgttcatcctgctgtacgt |
| | | ggtggcaaggatcgtgatcgccgtgcggtacagatatcagggcagcaacaataagagaatctataatgacatcg |
| | | agatgtccaggttccgcaagtgacgccggcgtcgacaatcaacctctggattacaaaatttgtgaaagattgac |
| | | tggtattcttaactatgttgctccttttacgctatgtggatacgctgctttaatgcctttgtatcatgctattg |
| | | cttcccgtatggctttcattttctcctccttgtataaatcctggttgctgtctctttatgaggagttgtggccc |
| | | gttgtcaggcaacgtggcgtggtgtgcactgtgtttgctgacgcaacccccactggttggggcattgccaccac |
| | | ctgtcagctcctttccgggactttcgctttccccctccctattgccacggcggaactcatcgccgcctgccttg |
| | | cccgctgctggacaggggctcggctgttgggcactgacaattccgtggtgttgtcggggaagctgacgtccttt |
| | | ccatggctgctcgcctgtgttgccacctggattctgcgcgggacgtccttctgctacgtcccttcggccctcaa |
| | | tccagcggaccttccttcccgcggcctgctgccggctctgcggcctcttccgcgtcttcgccttcgccctcaga |
| | | cgagtcggatctccctttgggccgcctccccgcctgtgtgccttctagttgccagccatctgttgtttgcccct |
| | | cccccgtgccttccttgaccctggaaggtgccactcccactgtcctttcctaataaaatgaggaaattgcatcg |
| | | cattgtctgagtaggtgtcattctattctggggggtggggtggggcaggacagcaagggggaggattgggaaga |
| | | caatagcaggcatgctggggatgcggtgggctctatgg |
| 131 | helper plasmid | ttgacattgattattgactagttattaatagtaatcaattacggggtcattagttcatagcccatatatggagt |
| | gag/pol | tccgcgttacataacttacggtaaatggcccgcctggctgaccgcccaacgacccccgcccattgacgtcaata |
| | | atgacgtatgttcccatagtaacgccaatagggactttccattgacgtcaatgggtggagtatttacggtaaac |
| | | tgcccacttggcagtacatcaagtgtatcatatgccaagtacgccccctattgacgtcaatgacggtaaatggc |
| | | ccgcctggcattatgcccagtacatgaccttatgggactttcctacttggcagtacatctacgtattagtcatc |
| | | gctattaccatggtgatgcggttttggcagtacatcaatgggcgtggatagcggtttgactcacggggatttcc |
| | | aagtctccaccccattgacgtcaatgggagtttgttttggcaccaaaatcaacgggactttccaaaatgtcgta |
| | | acaactccgccccattgacgcaaatgggcggtaggcgtgtacggtgggaggtctatataagcagagctcgttta |
| | | gtgaaccgtcagatcgcctggagacgccatccacgctgttttgacctccatagaagacaccgggaccgatccag |
| | | cctcccctcgaagcttacatgtggtaccgagctcggatcctgagaacttcagggtgagtctatgggacccttga |
| | | tgttttctttccccttcttttctatggttaagttcatgtcataggaaggggagaagtaacagggtacacatatt |
| | | gaccaaatcagggtaattttgcatttgtaattttaaaaaatgctttcttcttttaatatacttttttgtttatc |
| | | ttatttctaatactttccctaatctctttctttcagggcaataatgatacaatgtatcatgcctctttgcacca |
| | | ttctaaagaataacagtgataatttctgggttaaggcaatagcaatatttctgcatataaatatttctgcatat |
| | | aaattgtaactgatgtaagaggtttcatattgctaatagcagctacaatccagctaccattctgcttttatttt |
| | | atggttgggataaggctggattattctgagtccaagctaggcccttttgctaatcatgttcatacctcttatct |
| | | tcctcccacagctcctgggcaacgtgctggtctgtgtgctggcccatcactttggcaaagcacgtgagatctga |
| | | attcgagatctgccgccgccatgggtgcgagagcgtcagtattaagcgggggagaattagatcgatgggaaaaa |
| | | attcggttaaggccagggggaaagaaaaaatataaattaaaacatatagtatgggcaagcagggagctagaacg |
| | | attcgcagttaatcctggcctgttagaaacatcagaaggctgtagacaaatactgggacagctacaaccatccc |
| | | ttcagacaggatcagaagaacttagatcattatataatacagtagcaaccctctattgtgtgcatcaaaggata |
| | | gagataaaagacaccaaggaagctttagacaagatagaggaagagcaaaacaaaagtaagaaaaaagcacagca |
| | | agcagcagctgacacaggacacagcaatcaggtcagccaaaattaccctatagtgcagaacatccaggggcaaa |
| | | tggtacatcaggccatatcacctagaactttaaatgcatgggtaaaagtagtagaagagaaggctttcagccca |
| | | gaagtgatacccatgttttcagcattatcagaaggagccaccccacaagatttaaacaccatgctaaacacagt |
| | | ggggggacatcaagcagccatgcaaatgttaaaagagaccatcaatgaggaagctgcagaatgggatagagtgc |
| | | atccagtgcatgcagggcctattgcaccaggccagatgagagaaccaaggggaagtgacatagcaggaactact |
| | | agtacccttcaggaacaaataggatggatgacacataatccacctatcccagtaggagaaatctataaaagatg |
| | | gataatcctgggattaaataaaatagtaagaatgtatagccctaccagcattctggacataagacaaggaccaa |
| | | aggaaccctttagagactatgtagaccgattctataaaactctaagagccgagcaagcttcacaagaggtaaaa |
| | | aattggatgacagaaaccttgttggtccaaaatgcgaacccagattgtaagactattttaaaagcattgggacc |
| | | aggagcgacactagaagaaatgatgacagcatgtcagggagtggggggacccggccataaagcaagagttttgg |
| | | ctgaagcaatgagccaagtaacaaatccagctaccataatgatacagaaaggcaattttaggaaccaaagaaag |
| | | actgttaagtgtttcaattgtggcaaagaagggcacatagccaaaaattgcagggcccctaggaaaaagggctg |
| | | ttggaaatgtggaaaggaaggacaccaaatgaaagattgtactgagagacaggctaattttttagggaagatct |
| | | ggccttcccacaagggaaggccagggaattttcttcagagcagaccagagccaacagccccaccagaagagagc |
| | | ttcaggtttggggaagagacaacaactccctctcagaagcaggagccgatagacaaggaactgtatcctttagc |
| | | ttccctcagatcactctttggcagcgacccctcgtcacaataaagataggggggcaattaaaggaagctctatt |
| | | agatacaggagcagatgatacagtattagaagaaatgaatttgccaggaagatggaaaccaaaaat |
| | | gatagggggaattggaggttttatcaaagtaagacagtatgatcagatactcatagaaatctgcggacataaa |
| | | gctataggtacagtattagtaggacctacacctgtcaacataattggaagaaatctgttgactcagattggct |
| | | gcactttaaattttcccattagtcctattgagactgtaccagtaaaattaaagccaggaatggatggcccaaaa |
| | | gttaaacaatggccattgacagaagaaaaaataaaagcattagtagaaatttgtacagaaatggaaaaggaagg |
| | | aaaaatttcaaaaattgggcctgaaaatccatacaatactccagtatttgccataaagaaaaaagacagtacta |
| | | aatggagaaaattagtagatttcagagaacttaataagagaactcaagatttctgggaagttcaattaggaata |
| | | ccacatcctgcagggttaaaacagaaaaaatcagtaacagtactggatgtgggcgatgcatatttttcagttcc |
| | | cttagataaagacttcaggaagtatactgcatttaccatacctagtataaacaatgagacaccagggattagat |
| | | atcagtacaatgtgcttccacagggatggaaaggatcaccagcaatattccagtgtagcatgacaaaaatctta |
| | | gagccttttagaaaaaaaatccagacatagtcatctatcaatacatggatgatttgtatgtaggatctgactt |
| | | agaaatagggcagcatagaacaaaaatagaggaactgagacaacatctgttgaggtggggatttaccacacca |
| | | gacaaaaaacatcagaaagaacctccattcctttggatgggttatgaactccatcctgataaatggacagtaca |
| | | gcctatagtgctgccagaaaaggacagctggactgtcaatgacatacagaaattagtgggaaaattgaattggg |
| | | caagtcagatttatgcagggattaaagtaaggcaattatgtaaacttcttaggggaaccaaagcactaacagaa |
| | | gtagtaccactaacagaagaagcagagctagaactggcagaaaacagggagattctaaaagaaccggtacatgg |
| | | agtgtattatgacccatcaaaagacttaatagcagaaatacagaagcaggggcaaggccaatggacatatcaaa |
| | | tttatcaagagccatttaaaaatctgaaaacaggaaagtatgcaagaatgaagggtgcccacactaatgatgtg |
| | | aaacaattaacagaggcagtacaaaaaatagccacagaaagcatagtaatatggggaaagactcctaaatttaa |
| | | attacccatacaaaaggaaacatgggaagcatggtggacagagtattggcaagccacctggattcctgagtggg |
| | | agtttgtcaatacccctcccttagtgaagttatggtaccagttagagaaagaacccataataggagcagaaact |
| | | ttctatgtagatggggcagccaatagggaaactaaattaggaaaagcaggatatgtaactgacagaggaagaca |
| | | aaaagttgtccccctaacggacacaacaaatcagaagactgagttacaagcaattcatctagctttgcaggatt |
| | | cgggattagaagtaaacatagtgacagactcacaatatgcattgggaatcattcaagcacaaccagataagagt |
| | | gaatcagagttagtcagtcaaataatagagcagttaataaaaaaggaaaaagtctacctggcatgggtaccagc |
| | | acacaaaggaattggaggaaatgaacaagtagataaattggtcagtgctggaatcaggaaagtactatttttag |
| | | atggaatagataaggcccaagaagaacatgagaaatatcacagtaattggagagcaatggctagtgattttaac |
| | | ctaccacctgtagtagcaaaagaaatagtagccagctgtgataaatgtcagctaaaaggggaagccatgcatgg |
| | | acaagtagactgtagcccaggaatatggcagctagattgtacacatttagaaggaaaagttatcttggtagcag |
| | | ttcatgtagccagtggatatatagaagcagaagtaattccagcagagacagggcaagaaacagcatacttcctc |
| | | ttaaaattagcaggaagatggccagtaaaaacagtacatacagacaatggcagcaatttcaccagtactacagt |
| | | taaggccgcctgttggtgggcggggatcaagcaggaatttggcattccctacaatccccaaagtcaaggagtaa |
| | | tagaatctatgaataaagaattaaagaaaattataggacaggtaagagatcaggctgaacatcttaagacagca |
| | | gtacaaatggcagtattcatccacaattttaaaagaaaaggggggattggggggtacagtgcaggggaaagaat |
| | | agtagacataatagcaacagacatacaaactaaagaattacaaaaacaaattacaaaaattcaaaattttcggg |
| | | tttattacagggacagcagagatccagtttggaaaggaccagcaaagctcctctggaaaggtgaaggggcagta |
| | | gtaatacaagataatagtgacataaaagtagtgccaagaagaaaagcaaagatcatcagggattatggaaaaca |
| | | gatggcaggtgatgattgtgtggcaagtagacaggatgaggattaacacatggaattccggagcggccgcagga |
| | | gctttgttccttgggttcttgggagcagcaggaagcactatgggcgcagcgtcaatgacgctgacggtacaggc |
| | | cagacaattattgtctggtatagtgcagcagcagaacaatttgctgagggctattgaggcgcaacagcatctgt |
| | | tgcaactcacagtctggggcatcaagcagctccaggcaagaatcctggctgtggaaagatacctaaaggatcaa |
| | | cagctcctggggatttggggttgctctggaaaactcatttgcaccactgctgtgccttggaatgctagttggag |
| | | taataaatctctggaacagatttggaatcacacgacctggatggagtgggacagagaaattaacaattacacaa |
| | | gcttccgcggaattcaccccaccagtgcaggctgcctatcagaaagtggtggctggtgtggctaatgccctggc |
| | | ccacaagtatcactaagctcgctttcttgctgtccaatttctattaaaggttcctttgttccctaagtccaact |
| | | actaaactgggggatattatgaagggccttgagcatctggattctgcctaataaaaaacatttattttcattgc |
| | | aatgatgtatttaaattatttctgaatattttactaaaaagggaatgtgggaggtcagtgcatttaaaacataa |
| | | agaaatgaagagctagttcaaaccttgggaaaatacactatatcttaaactccatgaaagaaggtgaggctgca |
| | | aacagctaatgcacattggcaacagcccctgatgcctatgccttattcatccctcagaaaaggattcaagtaga |
| | | ggcttgatttggaggttaaagttttgctatgctgtatttta |
| 132 | helper plasmid-Rev | aatgtagtcttatgcaatactcttgtagtcttgcaacatggtaacgatgagttagcaacatgccttacaaggag |
| | | agaaaaagcaccgtgcatgccgattggtggaagtaaggtggtacgatcgtgccttattaggaaggcaacagacg |
| | | ggtctgacatggattggacgaaccactgaattccgcattgcagagatattgtatttaagtgcctagctcgatac |
| | | aataaacgccatttgaccattcaccacattggtgtgcacctccaagctcgagctcgtttagtgaaccgtcagat |
| | | cgcctggagacgccatccacgctgttttgacctccatagaagacaccgggaccgatccagcctcccctcgaagc |
| | | tagtcgattaggcatctcctatggcaggaagaagcggagacagcgacgaagacctcctcaaggcagtcagactc |
| | | atcaagtttctctatcaaagcaacccacctcccaatcccgaggggacccgacaggcccgaaggaatagaagaag |
| | | aaggtggagagagagacagagacagatccattcgattagtgaacggatccttagcacttatctgggacgatctg |
| | | cggagcctgtgcctcttcagctaccaccgcttgagagacttactcttgattgtaacgaggattgtggaacttct |
| | | gggacgcaggggggggaagccctcaaatattggtggaatctcctacaatattggagtcaggagctaaagaatag |
| | | tgctgttagcttgctcaatgccacagctatagcagtagctgaggggacagatagggttatagaagtagtacaag |
| | | aagcttggcactggccgtcgttttacaacgtcgtgatctgagcctgggagatctctggctaactagggaaccca |
| | | ctgcttaagcctcaataaagcttgccttgagtgcttcaagtagtgtgtgcccgtctgttgtgtgactctggta |
| | | actagagatc |
| 133 | Anti-CD3 CDRL1 | SASSSVSYMN |
| 134 | Anti-CD3 CDRL2 | DTSKLASG |
| 135 | Anti-CD3 CDRL3 | QQWSSNPFT |
| 136 | OKT3 CDRH1 | GYTFTRY |
| 137 | OKT3 CDRH2 | NPSRGY |
| 138 | OKT3 CDRH3 | YYDDHYCLDY |
| 139 | OKT3 CDRL1 | SASSSVSYMN |
| 140 | OKT3 CDRL2 | DTSKLAS |
| 141 | OKT3 CDRL3 | QQWSSNPFT |
| 142 | 15E8 CDRH1 | GFSLTSY |
| 143 | 15E8 CDRH2 | WAGGS |
| 144 | 15E8 CDRH3 | DKRAPGKLYYGYPDY |
| 145 | 15E8 CDRL1 | RASESVEYYVTSLMQ |
| 146 | 15E8 CDRL2 | AASNYES |
| 147 | 15E8 CDRL3 | QQTRKVPST |
| 148 | TGN1412 CDRH1 | GYTFSY |
| 149 | TGN1412 CDRH2 | YPGNVN |
| 150 | TGN1412 CDRH3 | SHYGLDWNFDV |
| 151 | TGN1412 CDRL1 | HASQNIYVLN |
| 152 | TGN1412 CDRL2 | KASNLHT |
| 153 | TGN1412 CDRL3 | QQGQTYPYT |
| 154 | T2A | GSGEGRGSLLTCGDVEENPGP |
| 155 | E2 scFv nucleotide | TCCGTGCTGACCCAGCCTAGCTCCGTGTCTGCCGCACCAGGACAGA |
| | | AGGTGACAATCAGCTGTTCCGGCTCTACCAGCAACATCGGCAACAA |
| | | TTACGTGAGCTGGTACCAGCAGCACCCTGGCAAGGCCCCAAAGCTG |
| | | ATGATCTACGACGTGTCCAAGAGGCCATCTGGAGTGCCTGATCGGT |
| | | TCTCCGGCTCTAAGAGCGGCAATTCCGCCTCTCTGGACATCAGCGG |
| | | ACTGCAGTCCGAGGACGAGGCAGATTACTATTGCGCCGCCTGGGAC |
| | | GATAGCCTGTCCGAGTTTCTGTTCGGCACCGGCACAAAGCTGACCG |
| | | TGCTGGGCTCTACAAGCGGATCCGGCAAGCCAGGATCTGGAGAGG |
| | | GCAGCACAAAGGGACAGGTGCAGCTGGTGGAGAGCGGAGGAAACC |
| | | TGGTGCAGCCAGGAGGCTCCCTGCGCCTGTCTTGTGCCGCCAGCGG |
| | | CTTTACCTTCGGCTCTTTTAGCATGTCCTGGGTGCGCCAGGCACCTG |
| | | GAGGAGGACTGGAGTGGGTGGCCGGCCTGAGCGCCCGGTCTAGCC |
| | | TGACACACTATGCCGACTCCGTGAAGGGCCGCTTCACCATCTCCCG |
| | | GGATAACGCCAAGAATAGCGTGTACCTGCAGATGAATAGCCTGCG |
| | | GGTGGAGGACACAGCCGTGTACTATTGCGCCAGGCGCTCCTATGAT |
| | | TCCTCTGGCTACTGGGGCCACTTTTACTCTTATATGGACGTGTGGGG |
| | | ACAGGGCACCCTGGTGACAGTGAGCTCC |
| 156 | E2 scFv polypeptide | SVLTQPSSVSAAPGQKVTISCSGSTSNIGNNYVSWYQQHPGKAPKLMI |
| | | YDVSKRPSGVPDRFSGSKSGNSASLDISGLQSEDEADYYCAAWDDSLS |
| | | EFLFGTGTKLTVLGSTSGSGKPGSGEGSTKGQVQLVESGGNLVQPGGS |
| | | LRLSCAASGFTFGSFSMSWVRQAPGGGLEWVAGLSARSSLTHYADSV |
| | | KGRFTISRDNAKNSVYLQMNSLRVEDTAVYYCARRSYDSSGYWGHFY |
| | | SYMDVWGQGTLVTVSS |
| 157 | E2 scFv VL | TCCGTGCTGACCCAGCCTAGCTCCGTGTCTGCCGCACCAGGACAGA |
| | nucleotide | AGGTGACAATCAGCTGTTCCGGCTCTACCAGCAACATCGGCAACAA |
| | | TTACGTGAGCTGGTACCAGCAGCACCCTGGCAAGGCCCCAAAGCTG |
| | | ATGATCTACGACGTGTCCAAGAGGCCATCTGGAGTGCCTGATCGGT |
| | | TCTCCGGCTCTAAGAGCGGCAATTCCGCCTCTCTGGACATCAGCGG |
| | | ACTGCAGTCCGAGGACGAGGCAGATTACTATTGCGCCGCCTGGGAC |
| | | GATAGCCTGTCCGAGTTTCTGTTCGGCACCGGCACAAAGCTGACCG |
| | | TGCTG |
| 158 | E2 scFv VL | SVLTQPSSVSAAPGQKVTISCSGSTSNIGNNYVSWYQQHPGKAPKLMI |
| | polypeptide | YDVSKRPSGVPDRFSGSKSGNSASLDISGLQSEDEADYYCAAWDDSLS |
| | | EFLFGTGTKLTVL |
| 159 | E2 scFv linker | GGCTCTACAAGCGGATCCGGCAAGCCAGGATCTGGAGAGGGCAGC |
| | nucleotide | ACAAAGGGA |
| 160 | E2 scFv linker | GSTSGSGKPGSGEGSTKG |
| | polypeptide | |
| 161 | E2 scFv VH | CAGGTGCAGCTGGTGGAGAGCGGAGGAAACCTGGTGCAGCCAGGA |
| | nucleotide | GGCTCCCTGCGCCTGTCTTGTGCCGCCAGCGGCTTTACCTTCGGCTC |
| | | TTTTAGCATGTCCTGGGTGCGCCAGGCACCTGGAGGAGGACTGGAG |
| | | TGGGTGGCCGGCCTGAGCGCCCGGTCTAGCCTGACACACTATGCCG |
| | | ACTCCGTGAAGGGCCGCTTCACCATCTCCCGGGATAACGCCAAGAA |
| | | TAGCGTGTACCTGCAGATGAATAGCCTGCGGGTGGAGGACACAGCC |
| | | GTGTACTATTGCGCCAGGCGCTCCTATGATTCCTCTGGCTACTGGGG |
| | | CCACTTTTACTCTTATATGGACGTGTGGGGACAGGGCACCCTGGTG |
| | | ACAGTGAGCTCC |
| 162 | E2 scFv VH | QVQLVESGGNLVQPGGSLRLSCAASGFTFGSFSMSWVRQAPGGGLEW |
| | polypeptide | VAGLSARSSLTHYADSVKGRFTISRDNAKNSVYLQMNSLRVEDTAVY |
| | | YCARRSYDSSGYWGHFYSYMDVWGQGTLVTVSS |
| 163 | E2 scFv nucleotide | TCCGTGCTGACCCAGCCTAGCTCCGTGTCTGCCGCACCAGGACAGA |
| | | AGGTGACAATCAGCTGTTCCGGCTCTACCAGCAACATCGGCAACAA |
| | | TTACGTGAGCTGGTACCAGCAGCACCCTGGCAAGGCCCCAAAGCTG |
| | | ATGATCTACGACGTGTCCAAGAGGCCATCTGGAGTGCCTGATCGGT |
| | | TCTCCGGCTCTAAGAGCGGCAATTCCGCCTCTCTGGACATCAGCGG |
| | | ACTGCAGTCCGAGGACGAGGCAGATTACTATTGCGCCGCCTGGGAC |
| | | GATAGCCTGTCCGAGTTTCTGTTCGGCACCGGCACAAAGCTGACCG |
| | | TGCTGGGCTCTACAAGCGGATCCGGCAAGCCAGGATCTGGAGAGG |
| | | GCAGCACAAAGGGACAGGTGCAGCTGGTGGAGAGCGGAGGAAACC |
| | | TGGTGCAGCCAGGAGGCTCCCTGCGCCTGTCTTGTGCCGCCAGCGG |
| | | CTTTACCTTCGGCTCTTTTAGCATGTCCTGGGTGCGCCAGGCACCTG |
| | | GAGGAGGACTGGAGTGGGTGGCCGGCCTGAGCGCCCGGTCTAGCC |
| | | TGACACACTATGCCGACTCCGTGAAGGGCCGCTTCACCATCTCCCG |
| | | GGATAACGCCAAGAATAGCGTGTACCTGCAGATGAATAGCCTGCG |
| | | GGTGGAGGACACAGCCGTGTACTATTGCGCCAGGCGCTCCTATGAT |
| | | TCCTCTGGCTACTGGGGCCACTTTTACTCTTATATGGACGTGTGGGG |
| | | ACAGGGCACCCTGGTGACAGTGAGCTCC |
| 164 | E2 scFv VL | TCCGTGCTGACCCAGCCTAGCTCCGTGTCTGCCGCACCAGGACAGA |
| | nucleotide | AGGTGACAATCAGCTGTTCCGGCTCTACCAGCAACATCGGCAACAA |
| | | TTACGTGAGCTGGTACCAGCAGCACCCTGGCAAGGCCCCAAAGCTG |
| | | ATGATCTACGACGTGTCCAAGAGGCCATCTGGAGTGCCTGATCGGT |
| | | TCTCCGGCTCTAAGAGCGGCAATTCCGCCTCTCTGGACATCAGCGG |
| | | ACTGCAGTCCGAGGACGAGGCAGATTACTATTGCGCCGCCTGGGAC |
| | | GATAGCCTGTCCGAGTTTCTGTTCGGCACCGGCACAAAGCTGACCG |
| | | TGCTG |
| 165 | E2 scFv linker | GGCTCTACAAGCGGATCCGGCAAGCCAGGATCTGGAGAGGGCAGC |
| | nucleotide | ACAAAGGGA |
| 166 | E2 scFv VH | CAGGTGCAGCTGGTGGAGAGCGGAGGAAACCTGGTGCAGCCAGGA |
| | nucleotide | GGCTCCCTGCGCCTGTCTTGTGCCGCCAGCGGCTTTACCTTCGGCTC |
| | | TTTTAGCATGTCCTGGGTGCGCCAGGCACCTGGAGGAGGACTGGAG |
| | | TGGGTGGCCGGCCTGAGCGCCCGGTCTAGCCTGACACACTATGCCG |
| | | ACTCCGTGAAGGGCCGCTTCACCATCTCCCGGGATAACGCCAAGAA |
| | | TAGCGTGTACCTGCAGATGAATAGCCTGCGGGTGGAGGACACAGCC |
| | | GTGTACTATTGCGCCAGGCGCTCCTATGATTCCTCTGGCTACTGGGG |
| | | CCACTTTTACTCTTATATGGACGTGTGGGGACAGGGCACCCTGGTG |
| | | ACAGTGAGCTCC |
| 167 | E2 CDR-L1 | TSNIGNNYVS |
| 168 | E2 CDR-L2 | LMIYDVSKRPS |
| 169 | E2 CDR-L3 | AAWDDSLSEF |
| 170 | E2 CDR-H1 | FTFGSFSMS |
| 171 | E2 CDR-H2 | WVAGLSARSSLTHY |
| 172 | E2 CDR-H3 | RRSYDSSGYWGHFYSYMDV |

1. A viral particle, comprising a viral envelope comprising on the surface of the viral envelope at least one T-cell adhesion molecule, at least one co-stimulatory protein, or combination thereof, and an immune cell-activating protein.

2. The viral particle of claim 1, wherein the at least one T-cell adhesion molecule is selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof.

3. The viral particle of claim 1 or claim 2, wherein the at least one T cell-adhesion molecule is CD58.

4. The viral particle of any one of claims 1-3, wherein the at least one co-stimulatory molecule is selected from CD45, CD2, CD4, CD5, CD8, CD9, CD16, CD22, CD33, CD28, CD37, CD64, CD80, CD86, CD134, CD137, CD154, OX40, 4-1BB, CD40L, and any combination thereof.

5. The viral particle of any one of claims 1-4, wherein the at least one co-stimulatory molecule is CD80, CD86, or CD80 and CD86.

6. The viral particle of any one of claims 1-5, wherein the immune cell-activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, TCR α chain, TCR β chain, TCR ζ chain, TCR γ chain, TCR δ chain, CD3 ε TCR subunit, CD3 γ TCR subunit, CD3 δ TCR subunit, or NKp80.

7. The viral particle of any one of claims 1-6, wherein the immune cell-activating protein is a protein that specifically binds CD3.

8. The viral particle of any one of claims 1-7, wherein the immune cell-activating protein is an antibody or antigen binding fragment thereof that binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80.

9. The viral particle of any one of claims 1-8, wherein the immune cell-activating protein is an antibody or antigen binding fragment thereof that binds CD3.

10. The viral particle of claim 9, wherein the antibody or antigen binding fragment thereof that binds CD3 is an anti-CD3 scFv.

11. The viral particle of any one of claims 1-10, wherein the T-cell adhesion molecule is CD58 and the co-stimulatory molecule is CD80.

12. The viral particle of any one of claims 1-10, wherein the T-cell adhesion molecule is CD58 and the co-stimulatory molecule is CD86.

13. The viral particle of any one of claims 1-10, wherein the T-cell adhesion molecule is CD58, the immune cell-activating protein is an anti-CD3 antibody or antigen binding fragment thereof, and the co-stimulatory molecule is CD80.

14. The viral particle of any one of claims 1-10, wherein the T-cell adhesion molecule is CD58, the immune cell-activating protein is an anti-CD3 antibody or antigen binding fragment thereof, and the co-stimulatory molecule is CD86.

15. The viral particle of any one of claims 1-14, comprising a payload.

16. The viral particle of claim 15, wherein the payload is a nucleic acid.

17. The viral particle of claim 16, wherein the nucleic acid is a non-coding nucleic acid, optionally wherein the non-coding nucleic acid is an siRNA, an miRNA, or an shRNA.

18. The viral particle of claim 17, wherein the nucleic acid comprises a nucleotide sequence encoding a polypeptide of interest.

19. The viral particle of any one of claims 1-14, comprising a vector genome comprising at least one nucleotide sequence encoding a polypeptide of interest.

20. A viral particle comprising (i) a viral envelope comprising on the surface of the viral envelope (a) an immune cell-activating protein, wherein the immune cell-activating protein binds a T cell receptor, (b) a co-stimulatory molecule, and (c) a T cell adhesion molecule, and (ii) a vector genome comprising at least one nucleotide sequence encoding a polypeptide of interest.

21. The viral particle of claim 20, wherein (a) the immune cell-activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80, (b) the co-stimulatory molecule is selected from CD45, CD2, CD4, CD5, CD8, CD9, CD16, CD22, CD33, CD28, CD37, CD64, CD80, CD86, CD134, CD137, CD154, OX40, 4-1BB, CD40L, and any combination thereof, and (c) the T cell adhesion molecule is selected from CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, and any combination thereof.

22. The viral particle of claim 20 or 21, wherein (a) the immune cell-activating protein is an antibody that specifically binds CD3, or an antigen binding fragment thereof, (b) the co-stimulatory molecule is CD80 or CD86, and (c) the T cell adhesion molecule is CD58.

23. The viral particle of any one of claims 1-22, wherein the viral envelope comprises a membrane-bound cytokine.

24. The viral particle of claim 23, wherein the membrane-bound cytokine is selected from IL-2, IL-7, IL-12, IL-15, IL-18, or IL-21.

25. The viral particle of any one of claims 1-24, wherein the viral envelope comprises a viral envelope protein.

26. The viral particle of claim 25, wherein the viral envelope protein is a VSV-G envelope protein, a measles virus envelope protein, a nipha virus envelope protein, or a cocal virus G protein.

27. The viral particle of claim 26, wherein the viral envelope comprises a Cocal glycoprotein or functional variant thereof.

28. The viral particle of claim 27, wherein the Cocal glycoprotein comprises an R354Q mutation compared to SEQ ID NO: 5.

29. The viral particle of claim 27 or 28, wherein the Cocal glycoprotein comprises an amino acid sequence at least 80%, at least 85%, at least 90%, at least 95%, at least 96%, at least 97%, at least 98%, or at least 99% identical to any one of SEQ ID NOs: 5, 13, and 19.

30. The viral particle of claim 27 or 28, wherein the Cocal glycoprotein comprises an amino acid sequence selected from SEQ ID NOs: 5, 13, and 19.

31. The viral particle of any one of claims 9-19 and 22-30, wherein the antibody that binds anti-CD3 or antigen binding fragment thereof comprises an amino acid sequence at least 80%, at least 85%, at least 90%, at least 95%, at least 96%, at least 97%, at least 98%, or at least 99% identical to SEQ ID NO: 2 or 12.

32. The viral particle of any one of claims 9-19 and 22-30, wherein the antibody that binds anti-CD3 or antigen binding fragment thereof comprises SEQ ID NO: 2 or SEQ ID NO: 12.

33. The viral particle of any one of claims 19-32, wherein the at least one nucleotide sequence encodes a multipartite cell-surface receptor.

34. The viral particle of claim 33, wherein the multipartite cell-surface receptor comprises a FKBP-rapamycin complex binding domain (FRB domain) and a FK506 binding protein domain (FKBP).

35. The viral particle of claim 33, wherein the multipartite cell-surface receptor is a rapamycin-activated cell-surface receptor.

36. The viral particle of any one of claims 19-35, wherein the at least one nucleotide sequence encodes a chimeric antigen receptor (CAR).

37. The viral particle of any one of claims 19-32, comprising a nucleotide sequence encoding a rapamycin activated cell-surface receptor and a nucleotide sequence encoding a CAR.

38. The viral particle of claim 36 or 37, wherein the CAR comprises an antigen binding domain specific for a cancer-associated antigen.

39. The viral particle of claim 37, wherein the cancer associated antigen is CD19, BCMA, GPRC5D, ROR1, FcRL5, alpha-fetoprotein, or Her2.

40. The viral particle of claim 36 or 37, wherein the CAR is a universal CAR.

41. The viral particle of claim 36 or 37, wherein the CAR comprises a hapten binding domain.

42. The viral particle of any one of claims 19-32, wherein the vector genome comprises from 5′ to 3′: a nucleotide sequence encoding a CAR and a nucleotide sequence encoding a multipartite cell-surface receptor.

43. The viral particle of claim 42, wherein the nucleotide sequences are operably linked.

44. The viral particle of claim 42 or 43, wherein the CAR comprises an antigen binding domain specific for a cancer-associated antigen, and wherein the multipartite cell-surface receptor is a rapamycin-activated cell-surface receptor.

45. The viral particle of claim 44, wherein the cancer-associated antigen is CD19, BCMA, GPRC5D, ROR1, FcRL5, alpha-fetoprotein, or Her2.

46. A viral particle comprising (i) a viral envelope comprising on the surface of the viral envelope (a) an immune cell-activating protein that specifically binds CD3, (b) a co-stimulatory molecule, wherein the co-stimulatory molecule binds CD28, and (c) a T cell adhesion molecule, and (ii) a vector genome comprising (a) a nucleotide sequence encoding a rapamycin-activated cell-surface receptor, and (b) a nucleotide sequence encoding a CAR, wherein the CAR comprises an antigen binding domain specific for a cancer-associated antigen, optionally wherein the nucleotide sequences are operably linked.

47. The viral particle of any one of claims 2-19 and 21-46, wherein CD58 comprises an amino acid sequence at least 80%, at least 85%, at least 90%, at least 95%, at least 96%, at least 97%, at least 98%, or at least 99% identical to SEQ ID NO: 17.

48. The viral particle of any one of claims 2-19 and 21-46, wherein CD58 comprises the amino acid sequence of SEQ ID NO: 17.

49. The viral particle of any one of claims 4-11, 13, 15-19, 21-45, 47-48, wherein CD80 comprises an amino acid sequence at least 80%, at least 85%, at least 90%, at least 95%, at least 96%, at least 97%, at least 98%, or at least 99% identical to SEQ ID NO: 20.

50. The viral particle of any one of claims 4-11, 13, 15-19, 21-45, 47-48, wherein CD80 comprises the amino acid sequence of SEQ ID NO: 20.

51. The viral particle of any one of 4-10, 12, 14, 15-19, 21-45, 47-48, wherein CD86 comprises an amino acid sequence at least 80%, at least 85%, at least 90%, at least 95%, at least 96%, at least 97%, at least 98%, or at least 99% identical to SEQ ID NO: 23.

52. The viral particle of any one of claims 4-10, 12, 14, 15-19, 21-45, 47-48, wherein CD86 comprises the amino acid sequence of SEQ ID NO: 23.

53. The viral particle of any one of claims 33-45 and 47-52, wherein the multipartite cell-surface receptor comprises an amino acid sequence at least 80%, at least 85%, at least 90%, at least 95%, at least 96%, at least 97%, at least 98%, or at least 99% identical to SEQ ID NOs: 77, 78, or 77 and 78.

54. The viral particle of any one of claims 33-45 and 47-52, wherein the multipartite cell-surface receptor comprises the amino acid sequence of SEQ ID NOs: 77, 78, or 77 and 78.

55. The viral particle of any one of claims 33-45 and 47-54, wherein the multipartite cell-surface receptor is encoded by a nucleotide sequence at least 80%, at least 85%, at least 90%, at least 95%, at least 96%, at least 97%, at least 98%, or at least 99% identical to SEQ ID NOs: 83, 84, or 83 and 84.

56. The viral particle of any one of claims 33-45 and 47-54, wherein the multipartite cell-surface receptor is encoded by the nucleotide sequence of SEQ ID NOs: 83, 84, or 83 and 84.

57. The viral particle of any one of claims 19-56, wherein the vector genome comprises a promoter.

58. The viral particle of claim 57, wherein the promoter is an MND promoter, a CAG promoter, an SV40 promoter, an SV40/CD43 promoter, or an EF-1α promoter.

59. The viral particle of claim 57, wherein the promoter is an inducible promoter.

60. The viral particle of any one of claims 1-59, wherein the viral particle is a lentiviral particle.

61. The viral particle of any one of claims 1-60, wherein the viral particle transduces T cells in vivo.

62. The viral particle of any one of claims 1-61, wherein the viral particle activates a T cell population comprising at least a 50% CD25(+) cells, at least a 70% CD25(+) cells, or at least 90% CD25(+) cells.

63. A pharmaceutical composition comprising the viral particle of any one of claims 1-62, and a pharmaceutically acceptable carrier.

64. A method of transducing a population of T cells in vivo in a subject, comprising administering to the subject the viral particle of any one of claims 1-62 or the pharmaceutical composition of claim 63, wherein the viral particle comprises a nucleotide sequence encoding a polypeptide of interest, and wherein the polypeptide of interest is expressed in the population of T cells after administration.

65. The method of claim 64, wherein the population of T cells secretes (i) at least 2×10⁴ pg/ml of TNFα, (ii) at least 2×10⁴ pg/ml of IL-2, (iii) at least 2×10⁵ pg/ml of IFNγ, or (iv) any combination of (i)-(iii), at least three days after administration of the lentiviral particle.

66. A method of generating an immune cell expressing a chimeric antigen receptor in a subject in need thereof, comprising administering the viral particle of any one of claims 1-62 or the pharmaceutical composition of claim 63 to the subject, wherein the viral particle comprises a nucleotide sequence encoding the chimeric antigen receptor.

67. A method of treating a disease or disorder in a subject in need thereof, comprising administering the viral particle of any one of claims 1-62 or the pharmaceutical composition of claim 63 to the subject, wherein the viral particle comprises a nucleotide sequence encoding a therapeutic polypeptide.

68. The method of any one of claims 64-67, wherein the viral particle is administered by intraperitoneal, subcutaneous, or intranodal injection.

69. The method of claim 68, wherein the viral particle is administered by intra-nodal injection, via inguinal lymph node.

70. The method of any one of claims 64-69, where the subject in need thereof has a disease or disorder, wherein the disease or disorder comprises B-cell malignancy, relapsed/refractory CD19-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

71. A kit comprising a container comprising the viral particle of any one of claims 1-62, and optionally a pharmaceutically acceptable carrier, and instructions for transducing T cells in vivo in a subject, comprising administering the viral particle to the subject.

72. A kit comprising a container comprising the viral particle of any one of claims 1-62, and optionally a pharmaceutically acceptable carrier, and instructions for treating a subject in need thereof, comprising administering the viral particle to subject.

73. The kit of claim 71 or 72, wherein the subject has a disease or disorder.

74. The kit of any one of claims 71-73, wherein the instructions comprise administering the viral particle by intraperitoneal, subcutaneous, or intranodal injection.

75. The viral particle of any one of claims 1-62 for use in a method of transducing T cells in vivo in a subject, comprising administering the viral particle to the subject.

76. The viral particle of any one of claims 1-62 for use in a method of treating a subject with a disease or a disorder, comprising administering the viral particle to the subject.

77. Use of the viral particle of any one of claims 1-62 for the manufacture of a medicament for transducing T cells in vivo in a subject, comprising administering the viral particle to the subject.

78. Use of the viral particle of any one of claims 1-62 for the manufacture of a medicament for treating a subject with a disease or a disorder, comprising administering the viral particle to the subject.

79. A method of transducing a population of T cells ex vivo in a subject, comprising contacting a population of T cells with the viral particle of any one of claims 1-62 or the pharmaceutical composition of claim 63, wherein the viral particle comprises a nucleotide sequence encoding a polypeptide of interest, wherein the polypeptide of interest is expressed in the population of T cells after administration, and wherein the contacting is performed ex vivo.

80. The method of claim 79, wherein the contacting is performed during a closed-loop manufacturing process.

81. The method of claim 79 or 80, wherein the T cells have not been previously contacted with an exogenous activation agent during the manufacturing process.

82. The viral particle of any one of claims 27, 31-45 and 47-62, wherein the Cocal glycoprotein comprises a K47Q mutation compared to SEQ ID NO: 5.

83. A method of activating a population of cells in a subject in vivo, comprising administering to the subject the viral particle of any one of claims 1-62 or the pharmaceutical composition of claim 63, wherein the viral particle comprises a nucleotide sequence encoding a polypeptide of interest, wherein the polypeptide of interest is expressed in a first subset of cells in the population of cells after administration, wherein the polypeptide of interest is not expressed in a second subset of cells in the population of cells after administration, and wherein the first and second subsets of cells are each activated by the viral particle.


SEQUENCE LISTING (210 sequences)

SEQ ID NO: 1 - protein, 21 aa, synthetic construct
MALPVTALLLPLALLLHAARP

SEQ ID NO: 2 - protein, 248 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAAAKP

SEQ ID NO: 3 - protein, 45 aa, synthetic construct
TTTPAPRPPTPAPTIASQPLSLRPEASRPAAGGAVHTRGLDFASD

SEQ ID NO: 4 - protein, 53 aa, synthetic construct
MALIVLGGVAGLLLFIGLGIFFCVRCRHRRRQGSGEGRGSLLTCGDVEENPGP

SEQ ID NO: 5 - protein, 511 aa, synthetic construct
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 6 - DNA, 63 bp, synthetic construct
  source 1..63: mol_type = other DNA
atggcactgcctgtgacagccctgctgctgccactggccctgctgctgcacgcagcacgccca

SEQ ID NO: 7 - DNA, 744 bp, synthetic construct
  source 1..744: mol_type = other DNA
gatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcct

SEQ ID NO: 8 - DNA, 135 bp, synthetic construct
  source 1..135: mol_type = other DNA
accacaacccctgccccaaggccacctacacccgcccctaccatcgcctctcagccactgagcctgaggccagaggcatccaggcctgccgcagggggggccgtgcacacccggggcctggactttgcctctgat

SEQ ID NO: 9 - DNA, 159 bp, synthetic construct
  source 1..159: mol_type = other DNA
atggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggacca

SEQ ID NO: 10 - DNA, 1533 bp, synthetic construct
  source 1..1533: mol_type = other DNA
aattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaag

SEQ ID NO: 11 - protein, 17 aa, synthetic construct
MGVKVLFALICIAVAEA

SEQ ID NO: 12 - protein, 245 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAS

SEQ ID NO: 13 - protein, 62 aa, synthetic construct
GVELIEGWFSSWKSTVVTFFFAIGVFILLYVVARIVIAVRYRYQGSNNKRIYNDIEMSRFRK

SEQ ID NO: 14 - DNA, 51 bp, synthetic construct
  source 1..51: mol_type = other DNA
atgggcgtgaaagtgctgttcgccctgatctgcatcgcagttgctgaagcc

SEQ ID NO: 15 - DNA, 735 bp, synthetic construct
  source 1..735: mol_type = other DNA
gacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctcc

SEQ ID NO: 16 - DNA, 186 bp, synthetic construct
  source 1..186: mol_type = other DNA
ggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaag

SEQ ID NO: 17 - protein, 250 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 18 - DNA, 750 bp, synthetic construct
  source 1..750: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 19 - protein, 93 aa, synthetic construct
SGFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 20 - protein, 288 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 21 - DNA, 864 bp, synthetic construct
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 22 - DNA, 279 bp, synthetic construct
  source 1..279: mol_type = other DNA
tccggattcgagcacccccacctggccgaggcccctaagcagctgcctgaagaagagacactgtttttcggagataccggcatcagcaaaaaccccgtggagctgatcgagggctggttcagctcttggaagagcaccgtggtcacattctttttcgccatcggcgtctttatcctgctgtacgtggtagccagaatcgtgatcgccgtgcggtacagataccagggcagcaacaacaagcggatctacaacgacatcgagatgagccggttcagaaag

SEQ ID NO: 23 - protein, 329 aa, synthetic construct
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 24 - DNA, 987 bp, synthetic construct
  source 1..987: mol_type = other DNA
atggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaactctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaactcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaaggtctgatgaagcccagcgtgtttttaaaagttcgaagacatcttcatgcgacaaaagtgatacatgtttt

SEQ ID NO: 25 - protein, 113 aa, synthetic construct
SGGSTSGSGKPGSGEGSTKGPEITLIIFGVMAGVIGTILLISYGIRRLALKYWWNLLQYWSQELKNSAVSLLNATAIAVAEGTDRVIEVVQGACRAIRHIPRRIRQGLERILL

SEQ ID NO: 26-27 - skipped (2 entries)

SEQ ID NO: 28 - DNA, 339 bp, synthetic construct
  source 1..339: mol_type = other DNA
tccggaggatctacaagcggctctggcaagcctggcagcggagaaggcagcaccaagggccctgagatcacactgatcatcttcggcgtgatggccggcgtcatcggcaccatcctgctgatcagctacggcatcagaagactggctctgaagtactggtggaatctgctgcaatactggagccaggagctgaaaaacagcgccgtgtccctgctcaacgccaccgccatcgccgtggccgagggcaccgacagagtgatcgaggtggtgcagggagcctgcagagctattcggcacatccccagacggatcaggcagggcctggaaagaatcctgctg

SEQ ID NO: 29-30 - skipped (2 entries)

SEQ ID NO: 31 - protein, 119 aa, synthetic construct
SGGSTSGSGKPGSGEGSTKGNWLWYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 32-33 - skipped (2 entries)

SEQ ID NO: 34 - DNA, 357 bp, synthetic construct
  source 1..357: mol_type = other DNA
tccggaggaagcaccagcggctctggcaagcctggcagcggcgagggctctaccaagggcaattggctgtggtacatcagaatcttcatcatcatcgtgggcagcctgatcggcctgagaatcgtgttcgccgtgctgagcctggtgaaccggggctgggaagctctgaagtactggtggaacctgctgcaatactggtcccaggagctgaaaaacagcgctgtgtccctgctcaacgccaccgccatcgccgtcgccgagggaacagacagagtgatcgaggtggtgcagggagcctgcagagccattcggcacatccccagacgcatcagacagggcctggaaagaatcctgctg

SEQ ID NO: 35 - DNA, 353 bp, synthetic construct
  source 1..353: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagc

SEQ ID NO: 36 - protein, 17 aa, synthetic construct
YINPSRGYTNYNQKVKD

SEQ ID NO: 37 - protein, 112 aa, synthetic construct
SGGGGGSGGGGSGGGGSYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 38 - protein, 10 aa, synthetic construct
YYDDHYCLDY

SEQ ID NO: 39 - protein, 11 aa, synthetic construct
RASQDISKYLN

SEQ ID NO: 40 - DNA, 336 bp, synthetic construct
  source 1..336: mol_type = other DNA
tccggaggcggtggaggctctggtggcggagggagcggtggcggaggcagctacatcagaatcttcatcatcatcgtgggcagcctgatcggcctgagaatcgtgttcgccgttctgagcctggtgaaccggggctgggaagccctgaagtactggtggaatctgctccagtactggtctcaggagctgaagaacagcgccgtgtccctgctgaacgctacagctatcgccgtcgccgagggcaccgacagagtgatcgaggtggtgcagggcgcctgcagagccatccggcacatccctagaaggattcggcaaggcctggaaagaatcctgctg

SEQ ID NO: 41 - protein, 7 aa, synthetic construct
HTSRLHS

SEQ ID NO: 42 - protein, 9 aa, synthetic construct
QQGNTLPYT

SEQ ID NO: 43 - protein, 83 aa, synthetic construct
GVELIEGWFSSWKSTVVTFFFAIGVFILLYVVARIVIAVRYRYQGSNNKRIYNDIEMSRFRKGSGEGRGSLLTCGDVEENPGP

SEQ ID NO: 44 - protein, 4 aa, synthetic construct
DYGV

SEQ ID NO: 45 - protein, 16 aa, synthetic construct
VIWGSETTYYNSALKS

SEQ ID NO: 46 - protein, 12 aa, synthetic construct
HYYYGGSYAMDY

SEQ ID NO: 47 - DNA, 249 bp, synthetic construct
  source 1..249: mol_type = other DNA
ggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagggatctggagagggaaggggaagcctgctgacatgcggcgacgtggaggagaacccaggacca

SEQ ID NO: 48 - protein, 5 aa, synthetic construct
RYTMH

SEQ ID NO: 49 - protein, 1296 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 50 - protein, 22 aa, synthetic construct
MLLLVTSLLLCELPHPAFLLIP

SEQ ID NO: 51 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 52 - protein, 40 aa, synthetic construct
ESKYGPPCPPCPMFWVLVVVGGVLACYSLLVTVAFIIFWV

SEQ ID NO: 53 - protein, 42 aa, synthetic construct
KRGRKKLLYIFKQPFMRPVQTTQEEDGCSCRFPEEEEGGCEL

SEQ ID NO: 54 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 22 aa, synthetic construct
GSGATNFSLLKQAGDVEENPGP

SEQ ID NO: 56 - protein, 89 aa, synthetic construct
EMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 57 - protein, 126 aa, synthetic construct
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 58 - protein, 125 aa, synthetic construct
GEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 59 - protein, 114 aa, synthetic construct
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 60 - protein, 315 aa, synthetic construct
GKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 61 - DNA, 3891 bp, synthetic construct
  source 1..3891: mol_type = other DNA
atgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtga

SEQ ID NO: 62 - DNA, 66 bp, synthetic construct
  source 1..66: mol_type = other DNA
atgctgctgctggtgacctccctgctgctgtgcgagctgcctcacccagcctttctgctgatcccc

SEQ ID NO: 63 - DNA, 735 bp, synthetic construct
  source 1..735: mol_type = other DNA
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagc

SEQ ID NO: 64 - DNA, 120 bp, synthetic construct
  source 1..120: mol_type = other DNA
gagtctaagtatggcccaccctgccctccatgtccaatgttctgggtgctggtggtggtgggaggcgtgctggcctgttactccctgctggtgaccgtggcctttatcatcttctgggtg

SEQ ID NO: 65 - DNA, 126 bp, synthetic construct
  source 1..126: mol_type = other DNA
aagagaggcaggaagaagctgctgtatatctttaagcagcccttcatgcgccctgtgcagaccacacaggaggaggacggctgcagctgtcggtttccagaggaggaggagggaggatgcgagctg

SEQ ID NO: 66 - DNA, 336 bp, synthetic construct
  source 1..336: mol_type = other DNA
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 67 - DNA, 66 bp, synthetic construct
  source 1..66: mol_type = other DNA
ggatctggagccaccaactttagcctgctgaagcaggcaggcgatgtggaggagaatccaggacct

SEQ ID NO: 68 - DNA, 267 bp, synthetic construct
  source 1..267: mol_type = other DNA
gagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaag

SEQ ID NO: 69 - DNA, 66 bp, synthetic construct
  source 1..66: mol_type = other DNA
ggctccggcgccaccaacttctccctgctgaagcaggccggcgatgtggaagaaaatccaggacca

SEQ ID NO: 70 - DNA, 378 bp, synthetic construct
  source 1..378: mol_type = other DNA
atgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctg

SEQ ID NO: 71 - DNA, 375 bp, synthetic construct
  source 1..375: mol_type = other DNA
ggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagaca

SEQ ID NO: 72 - DNA, 66 bp, synthetic construct
  source 1..66: mol_type = other DNA
ggctctggcgccaccaacttcagcctgctgaagcaagccggcgacgtggaagaaaacccaggacca

SEQ ID NO: 73 - DNA, 342 bp, synthetic construct
  source 1..342: mol_type = other DNA
atggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaag

SEQ ID NO: 74 - DNA, 945 bp, synthetic construct
  source 1..945: mol_type = other DNA
ggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtg

SEQ ID NO: 75 - protein, 1297 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 112 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGP

SEQ ID NO: 77 - protein, 273 aa, synthetic construct
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGP

SEQ ID NO: 78 - protein, 451 aa, synthetic construct
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGP

SEQ ID NO: 79 - protein, 267 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 80 - protein, 194 aa, synthetic construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 3894 bp, synthetic construct
  source 1..3894: mol_type = other DNA
atggagatgtggcacgagggactggaggaggcaagcagactgtactttggcgagaggaacgtgaagggcatgttcgaggtgctggagccactgcacgcaatgatggagaggggaccacagaccctgaaggagacatctttcaaccaggcatacggaagggacctgatggaggcacaggagtggtgccggaagtatatgaagagcggcaatgtgaaggacctgctgcaggcctgggatctgtactatcacgtgtttcggagaatctccaagggctctggcgccaccaacttctccctgctgaagcaggccggcgatgtggaggagaatcctggaccaatgccactgggactgctgtggctgggactggccctgctgggcgccctgcacgcccaggccggcgtgcaggtggagacaatcagccctggcgacggacgcacctttccaaagaggggacagacatgcgtggtgcactacaccggcatgctggaggatggcaagaagttcgacagctccagagataggaataagccctttaagttcatgctgggcaagcaggaagtgatcaggggatgggaggagggagtggcacagatgtctgtgggacagcgggccaagctgacaatcagcccagactatgcatacggagcaaccggacaccctggaatcatcccacctcacgccacactggtgtttgatgtggagctgctgaagctgggcgagggcagcaacacctccaaggagaatccatttctgttcgccctggaggccgtggtcatctctgtgggcagcatgggcctgatcatctccctgctgtgcgtgtacttttggctggagcgcacaatgccacggatccccaccctgaagaacctggaggacctggtgaccgagtaccacggcaatttctccgcctggtctggcgtgagcaagggactggcagagtctctgcagccagattatagcgagcggctgtgcctggtgagcgagatcccacccaagggaggcgccctgggagagggaccaggagcctccccttgcaaccagcactctccttactgggcccctccatgttataccctgaagccagagacaggcagcggagctactaacttctccctgctgaagcaagcaggcgacgtggaagaaaatcctggaccaatggcactgccagtgaccgccctgctgctgcctctggccctgctgctgcacgcagccagacccatcctgtggcacgaaatgtggcatgaaggcctggaggaggcaagcaggctgtactttggcgagcggaatgtgaaaggaatgtttgaagtgctggagcctctgcacgccatgatggagaggggccctcagaccctgaaggagacatcctttaaccaggcctacggcagagacctgatggaggcccaggagtggtgcaggaagtatatgaagtctggaaatgtgaaagacctgctgcaggcctgggatctgtattatcacgtgttcaggcgcatctctaagggcaaggatacaatcccttggctgggacacctgctggtgggactgagcggagcctttggcttcatcatcctggtgtatctgctgatcaactgccgcaatacaggcccatggctgaagaaggtgctgaagtgtaacacccccgacccttccaagttcttttctcagctgtctagcgagcacggcggcgatgtgcagaagtggctgtcctctccatttcccagctcctctttcagcccaggaggactggcaccagagatctccccactggaggtgctggagagggacaaggtgacccagctgctgctgcagcaggataaggtgcctgagccagcctccctgagctccaaccactccctgacctcttgctttacaaatcagggctacttctttttccacctgccagacgcactggagatcgaggcatgtcaggtgtatttcacatacgatccctatagcgaggaggaccctgatgagggagtggccggcgccccaaccggatctagcccacagcctctgcagccactgagcggagaggacgatgcatattgtacatttccttcccgcgacgatctgctgctgttctctccaagcctgctgggaggaccaagcccaccttccaccgcaccaggcggctccggggcaggggaggagcggatgccaccctctctgcaggagagagtgccaagggactgggatccacagccactgggacctccaacccctggagtgccagacctggtggatttccagccccctccagagctggtgctgagagaggcaggagaggaggtgcctgacgcaggaccaagagagggcgtgagctttccttggtccaggccacctggacagggagagttcagagccctgaacgccaggctgcccctgaatacagacgcctacctgtctctgcaggagctgcagggccaggatcctacacacctggtcggatctggcgccaccaactttagcctgctgaagcaggcaggcgacgtggaagagaaccctggaccaatgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctccgagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccaggtga

SEQ ID NO: 82 - DNA, 336 bp, synthetic construct
  source 1..336: mol_type = other DNA
atggagatgtggcacgagggactggaggaggcaagcagactgtactttggcgagaggaacgtgaagggcatgttcgaggtgctggagccactgcacgcaatgatggagaggggaccacagaccctgaaggagacatctttcaaccaggcatacggaagggacctgatggaggcacaggagtggtgccggaagtatatgaagagcggcaatgtgaaggacctgctgcaggcctgggatctgtactatcacgtgtttcggagaatctccaagggctctggcgccaccaacttctccctgctgaagcaggccggcgatgtggaggagaatcctggacca

SEQ ID NO: 83 - DNA, 819 bp, synthetic construct
  source 1..819: mol_type = other DNA
atgccactgggactgctgtggctgggactggccctgctgggcgccctgcacgcccaggccggcgtgcaggtggagacaatcagccctggcgacggacgcacctttccaaagaggggacagacatgcgtggtgcactacaccggcatgctggaggatggcaagaagttcgacagctccagagataggaataagccctttaagttcatgctgggcaagcaggaagtgatcaggggatgggaggagggagtggcacagatgtctgtgggacagcgggccaagctgacaatcagcccagactatgcatacggagcaaccggacaccctggaatcatcccacctcacgccacactggtgtttgatgtggagctgctgaagctgggcgagggcagcaacacctccaaggagaatccatttctgttcgccctggaggccgtggtcatctctgtgggcagcatgggcctgatcatctccctgctgtgcgtgtacttttggctggagcgcacaatgccacggatccccaccctgaagaacctggaggacctggtgaccgagtaccacggcaatttctccgcctggtctggcgtgagcaagggactggcagagtctctgcagccagattatagcgagcggctgtgcctggtgagcgagatcccacccaagggaggcgccctgggagagggaccaggagcctccccttgcaaccagcactctccttactgggcccctccatgttataccctgaagccagagacaggcagcggagctactaacttctccctgctgaagcaagcaggcgacgtggaagaaaatcctggacca

SEQ ID NO: 84 - DNA, 1353 bp, synthetic construct
  source 1..1353: mol_type = other DNA
atggcactgccagtgaccgccctgctgctgcctctggccctgctgctgcacgcagccagacccatcctgtggcacgaaatgtggcatgaaggcctggaggaggcaagcaggctgtactttggcgagcggaatgtgaaaggaatgtttgaagtgctggagcctctgcacgccatgatggagaggggccctcagaccctgaaggagacatcctttaaccaggcctacggcagagacctgatggaggcccaggagtggtgcaggaagtatatgaagtctggaaatgtgaaagacctgctgcaggcctgggatctgtattatcacgtgttcaggcgcatctctaagggcaaggatacaatcccttggctgggacacctgctggtgggactgagcggagcctttggcttcatcatcctggtgtatctgctgatcaactgccgcaatacaggcccatggctgaagaaggtgctgaagtgtaacacccccgacccttccaagttcttttctcagctgtctagcgagcacggcggcgatgtgcagaagtggctgtcctctccatttcccagctcctctttcagcccaggaggactggcaccagagatctccccactggaggtgctggagagggacaaggtgacccagctgctgctgcagcaggataaggtgcctgagccagcctccctgagctccaaccactccctgacctcttgctttacaaatcagggctacttctttttccacctgccagacgcactggagatcgaggcatgtcaggtgtatttcacatacgatccctatagcgaggaggaccctgatgagggagtggccggcgccccaaccggatctagcccacagcctctgcagccactgagcggagaggacgatgcatattgtacatttccttcccgcgacgatctgctgctgttctctccaagcctgctgggaggaccaagcccaccttccaccgcaccaggcggctccggggcaggggaggagcggatgccaccctctctgcaggagagagtgccaagggactgggatccacagccactgggacctccaacccctggagtgccagacctggtggatttccagccccctccagagctggtgctgagagaggcaggagaggaggtgcctgacgcaggaccaagagagggcgtgagctttccttggtccaggccacctggacagggagagttcagagccctgaacgccaggctgcccctgaatacagacgcctacctgtctctgcaggagctgcagggccaggatcctacacacctggtcggatctggcgccaccaactttagcctgctgaagcaggcaggcgacgtggaagagaaccctggacca

SEQ ID NO: 85 - DNA, 801 bp, synthetic construct
  source 1..801: mol_type = other DNA
atgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctcc

SEQ ID NO: 86 - DNA, 582 bp, synthetic construct
  source 1..582: mol_type = other DNA
gagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccagg

SEQ ID NO: 87 - protein, 794 aa, synthetic construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKRRR

SEQ ID NO: 88 - protein, 112 aa, synthetic construct; identical to SEQ ID NO: 76

SEQ ID NO: 89 - protein, 267 aa, synthetic construct; identical to SEQ ID NO: 79

SEQ ID NO: 90 - protein, 216 aa, synthetic construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 91 - protein, 199 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKRRR

SEQ ID NO: 92 - DNA, 2385 bp, synthetic construct
  source 1..2385: mol_type = other DNA
atggagatgtggcacgagggactggaggaggcatccagactgtacttcggcgagaggaacgtgaagggcatgtttgaggtgctggagccactgcacgccatgatggagagaggcccccagaccctgaaggagacatctttcaaccaggcctatggaagggacctgatggaggcacaggagtggtgccggaagtacatgaagagcggcaatgtgaaggacctgctgcaggcctgggatctgtactatcacgtgttccggagaatcagcaagggctccggcgccaccaactttagcctgctgaagcaggcaggcgacgtggaggagaatccaggacctatgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagcgagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggaccaatgggaagaggactgctgaggggactgtggccactgcacatcgtgctgtggaccaggatcgcctctacaatcccaccccacgtgcagaagagcgtgaacaatgacatgatcgtgaccgataacaatggcgccgtgaagtttccccagctgtgcaagttctgtgacgtgcgcttttccacctgtgataaccagaagtcctgcatgtctaattgtagcatcacatccatctgcgagaagcctcaggaggtgtgcgtggccgtgtggcggaagaacgacgagaatatcaccctggagacagtgtgccacgatcccaagctgccttatcacgacttcatcctggaggatgccgcctctcctaagtgtatcatgaaggagaagaagaagccaggcgagaccttctttatgtgcagctgttcctctgacgagtgcaacgataatatcatcttctccgaggagtacaacacctctaatcctgacctgctgctggtcatctttcaggtgacaggcatctccctgctgcctccactgggcgtggccatctctgtgatcatcatcttttattgttacagagtgaacaggcagcagaagcgccggcgctag

SEQ ID NO: 93 - DNA, 336 bp, synthetic construct
  source 1..336: mol_type = other DNA
atggagatgtggcacgagggactggaggaggcatccagactgtacttcggcgagaggaacgtgaagggcatgtttgaggtgctggagccactgcacgccatgatggagagaggcccccagaccctgaaggagacatctttcaaccaggcctatggaagggacctgatggaggcacaggagtggtgccggaagtacatgaagagcggcaatgtgaaggacctgctgcaggcctgggatctgtactatcacgtgttccggagaatcagcaagggctccggcgccaccaactttagcctgctgaagcaggcaggcgacgtggaggagaatccaggacct

SEQ ID NO: 94 - DNA, 801 bp, synthetic construct
  source 1..801: mol_type = other DNA
atgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagc

SEQ ID NO: 95 - DNA, 648 bp, synthetic construct
  source 1..648: mol_type = other DNA
gagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggacca

SEQ ID NO: 96 - DNA, 600 bp, synthetic construct
  source 1..600: mol_type = other DNA
atgggaagaggactgctgaggggactgtggccactgcacatcgtgctgtggaccaggatcgcctctacaatcccaccccacgtgcagaagagcgtgaacaatgacatgatcgtgaccgataacaatggcgccgtgaagtttccccagctgtgcaagttctgtgacgtgcgcttttccacctgtgataaccagaagtcctgcatgtctaattgtagcatcacatccatctgcgagaagcctcaggaggtgtgcgtggccgtgtggcggaagaacgacgagaatatcaccctggagacagtgtgccacgatcccaagctgccttatcacgacttcatcctggaggatgccgcctctcctaagtgtatcatgaaggagaagaagaagccaggcgagaccttctttatgtgcagctgttcctctgacgagtgcaacgataatatcatcttctccgaggagtacaacacctctaatcctgacctgctgctggtcatctttcaggtgacaggcatctccctgctgcctccactgggcgtggccatctctgtgatcatcatcttttattgttacagagtgaacaggcagcagaagcgccggcgctag

SEQ ID NO: 97 - protein, 67 aa, synthetic construct
SGHFSEPEITLIIFGVMAGVIGTILLISYGIRRLIKKSPSDVKPLPSPDTDVPLSSVEIENPETSDQ

SEQ ID NO: 98 - DNA, 201 bp, synthetic construct
  source 1..201: mol_type = other DNA
tccggacacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 99 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 100 - protein, 1003 aa, synthetic construct
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 101 - DNA, 4307 bp, synthetic construct
  source 1..4307: mol_type = other DNA
atgggcgcccgcgccagcgtgctttctggcggcgagctggacaggtgggagaagattcgcctgcggcctggaggaaagaaaaagtacaagctgaagcacatcgtgtgggcttctcgggaactggaaagattcgccgtgaaccctggactgctagagacctccgaaggctgcagacagatcctgggacagctgcaacctagcctgcagaccggcagcgaggagctgagaagcctgtacaacaccgtcgccaccctgtattgtgtgcaccaaagaatcgagatcaaggacaccaaggaggctctggataagatcgaggaagagcagaacaagagcaagaaaaaagcccagcaggccgccgctgataccggccattctaatcaggtgtcccagaactaccccattgtgcaaaatatccagggccagatggtccaccaggccatcagccctagaaccctgaatgcctgggtgaaggtggtggaagagaaggccttttctccagaggtgatccctatgttcagcgccctgagcgagggcgctacccctcaggacctgaacacaatgctgaacaccgtgggcggccaccaggccgccatgcagatgctgaaagaaaccatcaacgaggaggccgccgaatgggaccgggtgcaccccgttcacgccggcccaatcgcccctggccagatgcgggaacctagaggcagcgacatcgccggcacaaccagcacactgcaagagcagatcggatggatgacacacaacccccccatccccgtgggcgaaatctacaagcggtggatcattctgggactgaacaaaatcgttagaatgtacagccctaccagcatcctggatatcagacagggcccaaaggagcctttccgggactacgtggacagattttacaagaccctgagagccgaacaggcctcccaagaggtgaagaactggatgacagagaccctgctggtgcagaatgccaaccctgattgtaagacaatcctgaaggccctcggacctggcgctacactggaagaaatgatgaccgcctgccagggcgtgggcggccccggccacaaggccagagtgctggccgaggctatgagccaggtgacaaaccccgccacaatcatgatccagaagggcaacttcagaaaccagcggaagaccgtgaaatgcttcaactgcggcaaggaaggccacatcgcaaagaactgcagagcccctaggaagaaaggctgttggaagtgcggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggcggacagctgaaagaggccttgctggacaccggagccgatgacaccgtgctggaagaaatgaacctgcctggaagatggaaacctaagatgatcggtggcatcggcggatttatcaaagtgcgacagtatgaccagatcctgatcgagatttgcggccacaaagctatcggaacagtgctggtcggcccgacccccgtgaacatcattggccgcaacctgctgacacagatcggttgtacactgaactttcctatcagccctatcgaaaccgtgccggtcaagctgaagcccggcatggatggccctaaggtgaagcagtggcccctgacagaggaaaagatcaaggcactggtggaaatctgcacagaaatggaaaaagagggcaagatttctaaaatcggcccagagaacccctacaacacccctgttttcgccatcaagaagaaagattccaccaagtggaggaagctggtggactttcgggaactgaacaagcggacccaggatttctgggaggtgcagctgggcatcccccaccctgccggcctgaaacaaaaaaaaagcgtgaccgtgctggacgtgggcgacgcctatttcagcgtgcctctggataaggacttccggaaatacaccgcctttaccatccctagcatcaacaacgagacccctggcatccggtaccagtacaacgtgctcccacagggctggaagggctcacccgccatcttccagtgcagcatgaccaagatcctggagcctttcagaaagcagaatcctgacatcgtgatctaccagtacatggacgacctgtacgtgggctctgatctggagatcggacagcacagaacaaagatcgaagagctgagacagcatctgctgagatggggtttcaccacccccgacaagaagcaccagaaggaacctccttttctgtggatgggctacgagctgcaccccgataagtggacagtgcagcccatcgtgctgcccgagaaggactcctggaccgtgaacgacattcagaagctggtcggaaagctgaattgggcttcccaaatctacgccggcatcaaggtgcggcagctgtgcaagcttctgcgcggcacaaaggccctgacggaagtcgtgccactgaccgaggaagccgaattagagctggccgaaaacagagaaattctgaaagaacctgtgcacggcgtttactacgacccttctaaggacctgatcgccgaaatccagaaacaaggccagggccagtggacttaccaaatctaccaggagcctttcaaaaacctcaagaccggcaagtacgccagaatgaagggagcccatacaaacgacgtgaagcagctgacagaggctgttcagaagatcgccacagaaagcatcgtgatctggggcaagaccccaaagttcaagctgcctatccaaaaggaaacctgggaggcctggtggaccgagtactggcaggccacctggattcctgaatgggagttcgtgaacacaccacctcttgtgaagctgtggtaccagctggaaaaggagccaatcatcggcgccgagacattctacgtggacggcgccgccaaccgggagaccaaactgggaaaggccggatatgtgaccgacagaggcagacaaaaggtggtgcctctgaccgatacaactaaccagaaaacagagctgcaggccattcacctggccctgcaagacagcggcctggaagtgaatatcgtgacagacagtcagtacgccctgggcatcatccaggctcagcctgacaagagcgagagcgagctggtgtcccagatcatcgagcagctgatcaaaaaggagaaggtttatctggcctgggtgcccgcccacaagggcatcggaggcaacgagcaggtggacaagctggtcagcgccggcatccggaaggtgctgttcctggacggcatcgacaaggcccaggaggaacacgagaagtaccacagcaactggcgggccatggccagcgacttcaacctgccacctgtggttgctaaggagatcgtcgcctcttgtgataagtgccagctgaagggcgaggccatgcacggccaagtggattgcagccctgggatctggcagctagactgtacccacctggagggcaaggtgatcctggtggcagtgcacgtggccagcggctacatcgaggctgaggtgatccccgccgaaacgggccaggagaccgcctactttctgctgaagctagccggccggtggcctgtgaagaccgtgcacaccgataacggcagcaatttcaccagcacaaccgtgaaggctgcctgctggtgggctggaatcaagcaggagttcggcatcccatacaatcctcagtctcagggcgtgatcgagagcatgaacaaggaactgaagaagatcattggtcaggtcagagatcaggccgagcacctgaaaaccgccgttcaaatggctgtgttcatccataacttcaaaagaaaaggcggcatcggcggctacagcgccggcgaaagaatcgtggatatcatcgcgaccgacatccaaacaaaagagctgcaaaagcaaatcaccaagatccagaacttcagagtgtactacagagatagcagagatcctgtgtggaagggacctgccaagctgctgtggaagggcgagggcgccgtggtgatccaggacaatagcgacatcaaggtcgtgcccagaagaaaggctaaaatcattagagactacggcaaacagatggccggagatgattgcgtggcttctagacaggacgaggactga

SEQ ID NO: 102 - protein, 116 aa, synthetic construct
MAGRSGDSDEDLLKAVRLIKFLYQSNPPPNPEGTRQARRNRRRRWRERQRQIHSISERILSTYLGRSAEPVPLQLPPLERLTLDCNEDCGTSGTQGVGSPQILVESPTILESGAKE

SEQ ID NO: 103 - DNA, 351 bp, synthetic construct
  source 1..351: mol_type = other DNA
atggccggcagaagcggcgacagcgacgaggatctgctgaaagccgtgcggctgatcaagttcctgtaccagagcaaccctcctcctaaccccgagggcaccagacaggctagacggaaccgcagaagaaggtggcgggaacggcaaagacagatccactctatcagcgagagaatcctgagcacctacctgggaagatccgccgagcctgtccccctgcagctgcctccactggaaagactgaccctggattgtaatgaggactgcggcacaagcggaacccagggcgtgggcagcccccagattctggtggaatcccctacaatcctcgagtctggcgccaaggaatga

SEQ ID NO: 104 - DNA, 1539 bp, synthetic construct
  source 1..1539: mol_type = other DNA
atgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtga

SEQ ID NO: 105 - protein, 65 aa, synthetic construct
HFSEPEITLIIFGVMAGVIGTILLISYGIRRLIKKSPSDVKPLPSPDTDVPLSSVEIENPETSDQ

SEQ ID NO: 106 - DNA, 195 bp, synthetic construct
  source 1..195: mol_type = other DNA
cacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 107 - protein, 324 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGVELIEGWFSSWKSTVVTFFFAIGVFILLYVVARIVIAVRYRYQGSNNKRIYNDIEMSRFRK

SEQ ID NO: 108 - DNA, 972 bp, synthetic construct
  source 1..972: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaag

SEQ ID NO: 109 - protein, 355 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASSGFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 110 - DNA, 1062 bp, synthetic construct
  source 1..1062: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggattcgagcacccccacctggccgaggcccctaagcagctgcctgaagaagagacactgtttttcggagataccggcatcagcaaaaaccccgtggagctgatcgagggctggttcagctcttggaagagcaccgtggtcacattctttttcgccatcggcgtctttatcctgctgtacgtggtagccagaatcgtgatcgccgtgcggtacagataccagggcagcaacaacaagcggatctacaacgacatcgagatgagccggttcagaaag

SEQ ID NO: 111 - protein, 374 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGSTSGSGKPGSGEGSTKGPEITLIIFGVMAGVIGTILLISYGIRRLALKYWWNLLQYWSQELKNSAVSLLNATAIAVAEGTDRVIEVVQGACRAIRHIPRRIRQGLERILL

SEQ ID NO: 112 - DNA, 1122 bp, synthetic construct
  source 1..1122: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggatctacaagcggctctggcaagcctggcagcggagaaggcagcaccaagggccctgagatcacactgatcatcttcggcgtgatggccggcgtcatcggcaccatcctgctgatcagctacggcatcagaagactggctctgaagtactggtggaatctgctgcaatactggagccaggagctgaaaaacagcgccgtgtccctgctcaacgccaccgccatcgccgtggccgagggcaccgacagagtgatcgaggtggtgcagggagcctgcagagctattcggcacatccccagacggatcaggcagggcctggaaagaatcctgctg

SEQ ID NO: 113 - protein, 380 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGSTSGSGKPGSGEGSTKGNWLWYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 114 - DNA, 1140 bp, synthetic construct
  source 1..1140: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggaagcaccagcggctctggcaagcctggcagcggcgagggctctaccaagggcaattggctgtggtacatcagaatcttcatcatcatcgtgggcagcctgatcggcctgagaatcgtgttcgccgtgctgagcctggtgaaccggggctgggaagctctgaagtactggtggaacctgctgcaatactggtcccaggagctgaaaaacagcgctgtgtccctgctcaacgccaccgccatcgccgtcgccgagggaacagacagagtgatcgaggtggtgcagggagcctgcagagccattcggcacatccccagacgcatcagacagggcctggaaagaatcctgctg

SEQ ID NO: 115 - protein, 373 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGGGGSGGGGSGGGGSYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 116 - DNA, 1119 bp, synthetic construct
  source 1..1119: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggcggtggaggctctggtggcggagggagcggtggcggaggcagctacatcagaatcttcatcatcatcgtgggcagcctgatcggcctgagaatcgtgttcgccgttctgagcctggtgaaccggggctgggaagccctgaagtactggtggaatctgctccagtactggtctcaggagctgaagaacagcgccgtgtccctgctgaacgctacagctatcgccgtcgccgagggcaccgacagagtgatcgaggtggtgcagggcgcctgcagagccatccggcacatccctagaaggattcggcaaggcctggaaagaatcctgctg

SEQ ID NO: 117 - protein, 856 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGVELIEGWFSSWKSTVVTFFFAIGVFILLYVVARIVIAVRYRYQGSNNKRIYNDIEMSRFRKGSGEGRGSLLTCGDVEENPGPNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 118 - DNA, 2568 bp, synthetic construct
  source 1..2568: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaag

SEQ ID NO: 119 - protein, 327 aa, synthetic construct
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASHFSEPEITLIIFGVMAGVIGTILLISYGIRRLIKKSPSDVKPLPSPDTDVPLSSVEIENPETSDQ

SEQ ID NO: 120 - DNA, 981 bp, synthetic construct
  source 1..981: mol_type = other DNA
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctcccacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 121 - DNA, 7145 bp, synthetic construct
  source 1..7145: mol_type = other DNA
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcggccgcggggtacctaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtgacgccggcgctagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 122 - DNA, 6926 bp, synthetic construct
  source 1..6926: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgattgcctgacgcgtaatgaaagaccccacctgtaggtttggcaagctaggatcaaggtcaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagagcccacaacccctcactcggcgcgtgaacacaattctgcagtcgaaggcgtaccgtcacttacgagtcggtagcctgcagggccgccaccatgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtgacgccggcgctagtgtcgacgtagtggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaatgtgtgtgttggttttttgtgtgt

SEQ ID NO: 123 - DNA, 3020 bp, synthetic construct
  source 1..3020: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgtctgacatcctgcagggccgccaccatgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtgacgccggcgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 124 - DNA, 5398 bp, synthetic construct
  source 1..5398: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcactagaagcttagctgcgaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctaggatccgccgccaccatgggcgcccgcgccagcgtgctttctggcggcgagctggacaggtgggagaagattcgcctgcggcctggaggaaagaaaaagtacaagctgaagcacatcgtgtgggcttctcgggaactggaaagattcgccgtgaaccctggactgctagagacctccgaaggctgcagacagatcctgggacagctgcaacctagcctgcagaccggcagcgaggagctgagaagcctgtacaacaccgtcgccaccctgtattgtgtgcaccaaagaatcgagatcaaggacaccaaggaggctctggataagatcgaggaagagcagaacaagagcaagaaaaaagcccagcaggccgccgctgataccggccattctaatcaggtgtcccagaactaccccattgtgcaaaatatccagggccagatggtccaccaggccatcagccctagaaccctgaatgcctgggtgaaggtggtggaagagaaggccttttctccagaggtgatccctatgttcagcgccctgagcgagggcgctacccctcaggacctgaacacaatgctgaacaccgtgggcggccaccaggccgccatgcagatgctgaaagaaaccatcaacgaggaggccgccgaatgggaccgggtgcaccccgttcacgccggcccaatcgcccctggccagatgcgggaacctagaggcagcgacatcgccggcacaaccagcacactgcaagagcagatcggatggatgacacacaacccccccatccccgtgggcgaaatctacaagcggtggatcattctgggactgaacaaaatcgttagaatgtacagccctaccagcatcctggatatcagacagggcccaaaggagcctttccgggactacgtggacagattttacaagaccctgagagccgaacaggcctcccaagaggtgaagaactggatgacagagaccctgctggtgcagaatgccaaccctgattgtaagacaatcctgaaggccctcggacctggcgctacactggaagaaatgatgaccgcctgccagggcgtgggcggccccggccacaaggccagagtgctggccgaggctatgagccaggtgacaaaccccgccacaatcatgatccagaagggcaacttcagaaaccagcggaagaccgtgaaatgcttcaactgcggcaaggaaggccacatcgcaaagaactgcagagcccctaggaagaaaggctgttggaagtgcggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggcggacagctgaaagaggccttgctggacaccggagccgatgacaccgtgctggaagaaatgaacctgcctggaagatggaaacctaagatgatcggtggcatcggcggatttatcaaagtgcgacagtatgaccagatcctgatcgagatttgcggccacaaagctatcggaacagtgctggtcggcccgacccccgtgaacatcattggccgcaacctgctgacacagatcggttgtacactgaactttcctatcagccctatcgaaaccgtgccggtcaagctgaagcccggcatggatggccctaaggtgaagcagtggcccctgacagaggaaaagatcaaggcactggtggaaatctgcacagaaatggaaaaagagggcaagatttctaaaatcggcccagagaacccctacaacacccctgttttcgccatcaagaagaaagattccaccaagtggaggaagctggtggactttcgggaactgaacaagcggacccaggatttctgggaggtgcagctgggcatcccccaccctgccggcctgaaacaaaaaaaaagcgtgaccgtgctggacgtgggcgacgcctatttcagcgtgcctctggataaggacttccggaaatacaccgcctttaccatccctagcatcaacaacgagacccctggcatccggtaccagtacaacgtgctcccacagggctggaagggctcacccgccatcttccagtgcagcatgaccaagatcctggagcctttcagaaagcagaatcctgacatcgtgatctaccagtacatggacgacctgtacgtgggctctgatctggagatcggacagcacagaacaaagatcgaagagctgagacagcatctgctgagatggggtttcaccacccccgacaagaagcaccagaaggaacctccttttctgtggatgggctacgagctgcaccccgataagtggacagtgcagcccatcgtgctgcccgagaaggactcctggaccgtgaacgacattcagaagctggtcggaaagctgaattgggcttcccaaatctacgccggcatcaaggtgcggcagctgtgcaagcttctgcgcggcacaaaggccctgacggaagtcgtgccactgaccgaggaagccgaattagagctggccgaaaacagagaaattctgaaagaacctgtgcacggcgtttactacgacccttctaaggacctgatcgccgaaatccagaaacaaggccagggccagtggacttaccaaatctaccaggagcctttcaaaaacctcaagaccggcaagtacgccagaatgaagggagcccatacaaacgacgtgaagcagctgacagaggctgttcagaagatcgccacagaaagcatcgtgatctggggcaagaccccaaagttcaagctgcctatccaaaaggaaacctgggaggcctggtggaccgagtactggcaggccacctggattcctgaatgggagttcgtgaacacaccacctcttgtgaagctgtggtaccagctggaaaaggagccaatcatcggcgccgagacattctacgtggacggcgccgccaaccgggagaccaaactgggaaaggccggatatgtgaccgacagaggcagacaaaaggtggtgcctctgaccgatacaactaaccagaaaacagagctgcaggccattcacctggccctgcaagacagcggcctggaagtgaatatcgtgacagacagtcagtacgccctgggcatcatccaggctcagcctgacaagagcgagagcgagctggtgtcccagatcatcgagcagctgatcaaaaaggagaaggtttatctggcctgggtgcccgcccacaagggcatcggaggcaacgagcaggtggacaagctggtcagcgccggcatccggaaggtgctgttcctggacggcatcgacaaggcccaggaggaacacgagaagtaccacagcaactggcgggccatggccagcgacttcaacctgccacctgtggttgctaaggagatcgtcgcctcttgtgataagtgccagctgaagggcgaggccatgcacggccaagtggattgcagccctgggatctggcagctagactgtacccacctggagggcaaggtgatcctggtggcagtgcacgtggccagcggctacatcgaggctgaggtgatccccgccgaaacgggccaggagaccgcctactttctgctgaagctagccggccggtggcctgtgaagaccgtgcacaccgataacggcagcaatttcaccagcacaaccgtgaaggctgcctgctggtgggctggaatcaagcaggagttcggcatcccatacaatcctcagtctcagggcgtgatcgagagcatgaacaaggaactgaagaagatcattggtcaggtcagagatcaggccgagcacctgaaaaccgccgttcaaatggctgtgttcatccataacttcaaaagaaaaggcggcatcggcggctacagcgccggcgaaagaatcgtggatatcatcgcgaccgacatccaaacaaaagagctgcaaaagcaaatcaccaagatccagaacttcagagtgtactacagagatagcagagatcctgtgtggaagggacctgccaagctgctgtggaagggcgagggcgccgtggtgatccaggacaatagcgacatcaaggtcgtgcccagaagaaaggctaaaatcattagagactacggcaaacagatggccggagatgattgcgtggcttctagacaggacgaggactgataagaattccatgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 125 - DNA, 1153 bp, synthetic construct
  source 1..1153: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggccaccatggccggcagaagcggcgacagcgacgaggatctgctgaaagccgtgcggctgatcaagttcctgtaccagagcaaccctcctcctaaccccgagggcaccagacaggctagacggaaccgcagaagaaggtggcgggaacggcaaagacagatccactctatcagcgagagaatcctgagcacctacctgggaagatccgccgagcctgtccccctgcagctgcctccactggaaagactgaccctggattgtaatgaggactgcggcacaagcggaacccagggcgtgggcagcccccagattctggtggaatcccctacaatcctcgagtctggcgccaaggaatgataactagcacctgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 126 - DNA, 2454 bp, synthetic construct
  source 1..2454: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgccaccatgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagtaatgactcgaggtgaattcgcgccggcgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 127 - DNA, 2452 bp, synthetic construct
  source 1..2452: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgccaccatgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggacacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccagtgataagaattctagtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 128 - DNA, 3871 bp, synthetic construct
  source 1..3871: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatggcactgcctgtgacagccctgctgctgccactggccctgctgctgcacgcagcacgcccagatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcctaccacaacccctgccccaaggccacctacacccgcccctaccatcgcctctcagccactgagcctgaggccagaggcatccaggcctgccgcagggggggccgtgcacacccggggcctggactttgcctctgatatggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaagtgacgccggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 129 - DNA, 4525 bp, synthetic construct
  source 1..4525: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactcctgcagggccgccaccatggcactgcctgtgacagccctgctgctgccactggccctgctgctgcacgcagcacgcccagatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcctaccacaacccctgccccaaggccacctacacccgcccctaccatcgcctctcagccactgagcctgaggccagaggcatccaggcctgccgcagggggggccgtgcacacccggggcctggactttgcctctgatatggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaagtgacgccggcgctcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctaatgctgtatttta

SEQ ID NO: 130 - DNA, 2773 bp, synthetic construct
  source 1..2773: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtgacgccggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 131 - DNA, 6468 bp, synthetic construct
  source 1..6468: mol_type = other DNA
ttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 132 - DNA, 972 bp, synthetic construct
  source 1..972: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatc

SEQ ID NO: 133 - protein, 10 aa, synthetic construct
SASSSVSYMN

SEQ ID NO: 134 - protein, 8 aa, synthetic construct
DTSKLASG

SEQ ID NO: 135 - protein, 9 aa, synthetic construct
QQWSSNPFT

SEQ ID NO: 136 - protein, 7 aa, synthetic construct
GYTFTRY

SEQ ID NO: 137 - protein, 6 aa, synthetic construct
NPSRGY

SEQ ID NO: 138 - protein, 10 aa, synthetic construct
YYDDHYCLDY

SEQ ID NO: 139 - protein, 10 aa, synthetic construct
SASSSVSYMN

SEQ ID NO: 140 - protein, 7 aa, synthetic construct
DTSKLAS

SEQ ID NO: 141 - protein, 9 aa, synthetic construct
QQWSSNPFT

SEQ ID NO: 142 - protein, 7 aa, synthetic construct
GFSLTSY

SEQ ID NO: 143 - protein, 5 aa, synthetic construct
WAGGS

SEQ ID NO: 144 - protein, 15 aa, synthetic construct
DKRAPGKLYYGYPDY

SEQ ID NO: 145 - protein, 15 aa, synthetic construct
RASESVEYYVTSLMQ

SEQ ID NO: 146 - protein, 7 aa, synthetic construct
AASNYES

SEQ ID NO: 147 - protein, 9 aa, synthetic construct
QQTRKVPST

SEQ ID NO: 148 - protein, 6 aa, synthetic construct
GYTFSY

SEQ ID NO: 149 - protein, 6 aa, synthetic construct
YPGNVN

SEQ ID NO: 150 - protein, 11 aa, synthetic construct
SHYGLDWNFDV

SEQ ID NO: 151 - protein, 10 aa, synthetic construct
HASQNIYVLN

SEQ ID NO: 152 - protein, 7 aa, synthetic construct
KASNLHT

SEQ ID NO: 153 - protein, 9 aa, synthetic construct
QQGQTYPYT

SEQ ID NO: 154 - protein, 21 aa, synthetic construct
GSGEGRGSLLTCGDVEENPGP

SEQ ID NO: 155 - DNA, 762 bp, synthetic construct
  source 1..762: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 156 - protein, 254 aa, synthetic construct
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 157 - DNA, 327 bp, synthetic construct
  source 1..327: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctg

SEQ ID NO: 158 - protein, 109 aa, synthetic construct
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 159 - DNA, 54 bp, synthetic construct
  source 1..54: mol_type = other DNA
ggctctacaagcggatccggcaagccaggatctggagagggcagcacaaaggga

SEQ ID NO: 160 - protein, 18 aa, synthetic construct
GSTSGSGKPGSGEGSTKG

SEQ ID NO: 161 - DNA, 381 bp, synthetic construct
  source 1..381: mol_type = other DNA
caggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 162 - protein, 127 aa, synthetic construct
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 163 - DNA, 762 bp, synthetic construct; identical to SEQ ID NO: 155
  source 1..762: mol_type = other DNA

SEQ ID NO: 164 - DNA, 327 bp, synthetic construct; identical to SEQ ID NO: 157
  source 1..327: mol_type = other DNA

SEQ ID NO: 165 - DNA, 54 bp, synthetic construct; identical to SEQ ID NO: 159
  source 1..54: mol_type = other DNA

SEQ ID NO: 166 - DNA, 381 bp, synthetic construct; identical to SEQ ID NO: 161
  source 1..381: mol_type = other DNA

SEQ ID NO: 167 - protein, 10 aa, synthetic construct
TSNIGNNYVS

SEQ ID NO: 168 - protein, 11 aa, synthetic construct
LMIYDVSKRPS

SEQ ID NO: 169 - protein, 10 aa, synthetic construct
AAWDDSLSEF

SEQ ID NO: 170 - protein, 9 aa, synthetic construct
FTFGSFSMS

SEQ ID NO: 171 - protein, 14 aa, synthetic construct
WVAGLSARSSLTHY

SEQ ID NO: 172 - protein, 19 aa, synthetic construct
RRSYDSSGYWGHFYSYMDV

SEQ ID NO: 173 - protein, 208 aa, synthetic construct [sequence omitted]

SEQ ID NO: 174 - protein, 224 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 175 - protein, 92 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 176 - protein, 323 aa, synthetic construct
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNK

SEQ ID NO: 177 - protein, 453 aa, synthetic construct
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 178 - protein, 133 aa, synthetic construct
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 179 - protein, 184 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 180 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 181 - protein, 323 aa, synthetic construct
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 182 - protein, 155 aa, synthetic construct
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 183 - protein, 161 aa, synthetic construct
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSS

SEQ ID NO: 184 - protein, 128 aa, synthetic construct
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 185 - protein, 172 aa, synthetic construct
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 186 - protein, 193 aa, synthetic construct
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLAR

SEQ ID NO: 187 - protein, 407 aa, synthetic construct
KDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTK

SEQ ID NO: 188 - protein, 204 aa, synthetic construct
KDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTG

SEQ ID NO: 189 - protein, 204 aa, synthetic construct
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTK

SEQ ID NO: 190 - protein, 283 aa, synthetic construct
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSH

SEQ ID NO: 191 - protein, 243 aa, synthetic construct
EPHSLRYNLMVLSQDGSVQSGFLAEGHLDGQPFLRYDRQKRRAKPQGQWAEDVLGAETWDTETEDLTENGQDLRRTLTHIKDQKGVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTD

SEQ ID NO: 192 - protein, 244 aa, synthetic construct
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 193 - protein, 246 aa, synthetic construct
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 194 - protein, 244 aa, synthetic construct
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 195 - protein, 195 aa, synthetic construct
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 196 - protein, 198 aa, synthetic construct
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 197 - protein, 246 aa, synthetic construct
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 198 - protein, 238 aa, synthetic construct [sequence omitted]

SEQ ID NO: 199 - protein, 238 aa, synthetic construct
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 200 - protein, 162 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 201 - protein, 220 aa, synthetic construct
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 202 - protein, 244 aa, synthetic construct
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASTTENLYFQG

SEQ ID NO: 203 - protein, 128 aa, synthetic construct
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 204 - protein, 133 aa, synthetic construct [sequence omitted]

SEQ ID NO: 205 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 206 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 207 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 208 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 209 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 210 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS